(19) United States
(12) Patent Application Publication
(10) Pub. No.: US 2024/0401085 A1
(43) Pub. Date: Dec. 5, 2024

(54) TRANSPOSON COMPOSITIONS AND METHODS OF USE THEREOF

(71) Applicant: Poseida Therapeutics, Inc., San Diego, CA (US)

(72) Inventors: Joseph S. LUCAS, San Diego, CA (US); Blair B. MADISON, San Diego, CA (US)

(21) Appl. No.: 18/698,514

(22) PCT Filed: Oct. 4, 2022
(86) PCT No.: PCT/US2022/077544

§ 371 Date: Apr. 4, 2024

Related U.S. Application Data
(60) Provisional application No. 63/252,030, filed on Oct. 4, 2021.

(51) Int. Cl. C12N 15/90; C12N 9/22

(52) U.S. Cl. CPC C12N 15/907; C12N 9/22; C12N 2800/90

(57) ABSTRACT

This disclosure provides compositions and methods of improved transposition for use in gene therapy. Disclosed are transposon compositions which comprise non-naturally occurring structural improvements of the inverted terminal repeat (ITR) regions, in order to improve the efficacy of transposition. Disclosed are polynucleotides encoding a transposon comprising a first inverted terminal repeat (ITR) and a second ITR, wherein the first ITR and/or the second ITR comprise at least one nucleic acid substitution which confers an increase in transposition activity. Also disclosed are methods of making and methods of using these transposon compositions and polynucleotides.

### CROSS-REFERENCE TO RELATED APPLICATIONS

This application claims priority to, and the benefit of, U.S. Provisional Application No. 63/252,030, filed Oct. 4, 2021. The contents of this application are incorporated herein by reference in their entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “POTH-071_001WO_SeqList_ST26.” The XML file is 779,850 bytes in size, created on Oct. 2, 2022, and is being submitted electronically via USPTO Patent Center.

### FIELD OF THE DISCLOSURE

The disclosure is directed to molecular biology, and more, specifically, to transposons, cell compositions comprising transposons, methods of making and methods of using the same.

### BACKGROUND OF THE INVENTION

There has been a long-felt but unmet need in the art for compositions and methods of improved transposition for use in gene therapy. The disclosure provides transposon compositions, methods of making and methods of using these compositions which comprise non-naturally occurring structural improvements to the inverted terminal repeat sequences (ITR) of transposons to improve the efficacy and frequency of transposition, particularly for use in human cells as a method of modifying cells for gene therapy.

### SUMMARY OF THE INVENTION

The present disclosure provides a polynucleotide encoding a transposon comprising: a) a first inverted terminal repeat (ITR) comprising a nucleic acid sequence having at least 95% identity to SEQ ID NO: 4; and b) a second ITR comprising a nucleic acid sequence having at least 95% identity to SEQ ID NO: 5; and wherein the first ITR comprises a nucleic acid substitution in at least one of nucleic acid position 31 and position 33 of SEQ ID NO: 4. In some aspects, the first ITR is a left end ITR (LE ITR) of a transposon. In some aspects, the second ITR is a right end ITR (RE ITR) of a transposon.

The present disclosure provides a polynucleotide encoding a transposon comprising: a) a first inverted terminal repeat (ITR) comprising a nucleic acid sequence having at least 99% identity to SEQ ID NO: 4; and b) a second ITR comprising a nucleic acid sequence having at least 99% identity to SEQ ID NO: 5; and wherein the first ITR comprises a nucleic acid substitution in at least one of nucleic acid position 31 and position 33 of SEQ ID NO: 4. In some aspects, the first ITR is a left end ITR (LE ITR) of a transposon. In some aspects, the second ITR is a right end ITR (RE ITR) of a transposon.

In some aspects, the first ITR comprises the nucleic acid substitution of 31G>T. In some aspects, the first ITR comprises the nucleic acid sequence of SEQ ID NO: 14. In some aspects, the first ITR comprises the nucleic acid substitution of 33A>C. In some aspects, the first ITR comprises the nucleic acid sequence of SEQ ID NO: 15. In some aspects, the first ITR comprises the nucleic acid substitution of 31G>T and 33A>C. In some aspects, the first ITR comprises the nucleic acid sequence of SEQ ID NO: 16.

In some aspects, the transposon is a piggyBac transposon or a piggyBac-like transposon. In some aspects, the piggyBac transposon comprises any of the nucleic acid sequences set forth in SEQ ID NOs: 144-156 and 158.

In some aspects, the transposon has at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% increase in transposition activity in comparison to a polynucleotide encoding a transposon having a first ITR of SEQ ID NO: 4 and a second ITR of SEQ ID NO: 5. In some aspects, the transposition activity is excision. In some aspects, the transposition activity is integration. In some aspects, the transposition activity is excision and integration.

In some aspects, the polynucleotide further comprises a gene encoding a selectable marker. In some aspects, the polynucleotide further comprises a promoter. In some aspects, the polynucleotide further comprises a sequence encoding an insulator.

In some aspects, the polynucleotide further comprises at least one exogenous nucleic acid sequence. In some aspects, the at least one exogenous nucleic acid sequence encodes a non-naturally occurring antigen receptor, a sequence encoding a therapeutic polypeptide, or a combination thereof. In some aspects, at least one exogenous nucleic acid sequence encodes a non-naturally occurring antigen receptor comprising a chimeric antigen receptor. In some aspects, the at least one exogenous nucleic acid sequence encodes a therapeutic polypeptide.

The present disclosure also provides a vector comprising any one of the polynucleotides encoding a transposon disclosed herein. The present disclosure also provides a composition comprising any one of the vectors disclosed herein. The present disclosure also provides a composition comprising any one of the polynucleotides encoding a transposon disclosed herein. The present disclosure also provides a cell comprising any one of the polynucleotides encoding a transposon or any one of the vectors disclosed herein. The present disclosure also provides a pharmaceutical composition comprising any one of the cells disclosed herein and a pharmaceutically acceptable carrier.

The present disclosure provides a method of producing a population of modified cells comprising delivering to a population of cells, a) any one of the polynucleotides encoding a transposon disclosed herein, and b) a nucleic acid or amino acid sequence comprising a sequence encoding a transposase enzyme, thereby inducing transposition activity and producing a population of modified cells, and wherein the transposition activity of the polynucleotide is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% in comparison to a polynucleotide comprising a first ITR comprising a nucleic acid sequence of SEQ ID NO: 4 and a second ITR comprising a nucleic acid sequence of SEQ ID NO: 5.

The present disclosure provides a method of producing a population of modified cells comprising delivering to a population of cells, a) any one of the polynucleotides encoding a transposon disclosed herein, and b) a nucleic acid or amino acid sequence comprising a sequence encoding a transposase enzyme, thereby inducing transposition activity and producing a population of modified cells, and wherein the transposition activity of the polynucleotide is increased by at least 1.0 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold in comparison to a polynucleotide comprising a first ITR comprising a nucleic acid sequence of SEQ ID NO: 4 and a second ITR comprising a nucleic acid sequence of SEQ ID NO: 5. In some aspects, the transposition activity of the polynucleotide is increased by at least 2.0 fold. In some aspects, the transposition activity of the polynucleotide is increased by at least 2.5 fold.

In some aspects, the transposase enzyme is a piggyBac transposase or a piggy-Bac like transposase. In some aspects, the transposase enzyme is a Super piggyBac™ (SPB) transposase enzyme. In some aspects, the transposase enzyme comprises an amino acid sequence comprising SEQ ID NO: 107 In some aspects, the transposase enzyme comprises an amino acid sequence comprising SEQ ID NO: 109.

The present disclosure also provides a composition comprising the population of modified cells produced according to any one of the methods of producing a population of modified cells described herein.

The present disclosure also provides method of treating a disease or disorder in a subject in need thereof comprising administering to the subject at least one therapeutically effective dose of a polynucleotide, a vector, a cell and/or a composition of any of the preceding claims.

In some aspects, the disease or disorder is cancer. In some aspects, the cancer is a BCMA-positive cancer, a MUC-1-C positive cancer or a PSMA-positive cancer. In some aspects, the cancer is a lung cancer, a brain cancer, a head and neck cancer, a breast cancer, a skin cancer, a liver cancer, a pancreatic cancer, a stomach cancer, a colon cancer, a rectal cancer, a uterine cancer, a cervical cancer, an ovarian cancer, a prostate cancer, a testicular cancer, a skin cancer, an esophageal cancer, a lymphoma, a leukemia, acute leukemia, acute lymphoblastic leukemia (ALL), acute lymphocytic leukemia, acute myeloid leukemia (AML), acute myelogenous leukemia, chronic myelocytic leukemia (CML), chronic lymphocytic leukemia (CLL), hairy cell leukemia, myelodyplastic syndrome (MDS), Hodgkin's disease, non-Hodgkin's lymphoma, or multiple myeloma.

In some aspects, the disease or disorder is a liver disease or disorder. In some aspects, the liver disease or disorder is a urea cycle disorder. In some aspects, the liver disease or disorder is a metabolic liver disorder. In some aspects, the metabolic liver disorder is MLD is N-Acetylglutamate Synthetase (NAGS) Deficiency, Carbamoylphosphate Synthetase I Deficiency (CPSI Deficiency), Ornithine Transcarbamylase (OTC) Deficiency, Argininosuccinate Synthetase Deficiency (ASSD) (Citrullinemia I), Citrin Deficiency (Citrullinemia II), Argininosuccinate Lyase Deficiency (Argininosuccinic Aciduria), Arginase Deficiency (Hyperargininemia), Ornithine Translocase Deficiency (HHH Syndrome), progressive familial intrahepatic cholestasis type 1 (PFIC1), progressive familial intrahepatic cholestasis type 1 (PFIC2), progressive familial intrahepatic cholestasis type 1 (PFIC3), methylmalonic acidemia (MMA) or any combination thereof.

In some aspects, the disease or disorder is a hemophilia disease. In some aspects, the hemophilia disease is hemophilia A, hemophilia B, hemophilia C or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram showing the screening strategy of transposon libraries containing a mutated LE ITR and/or a mutated RE ITR.

FIG. 2A is a schematic diagram showing the mutational strategy of a 13 bp window around the Dimerization and DNA Binding Domain (DDBD) binding site of the LE ITR.

FIG. 2B is a graph showing the enrichment score of each of the 13 base pairs around the DDBD binding site of the LE ITR. The wildtype LE ITR sequence is shown in the top row above the graph. The wildtype nucleotide is not always the most enriched.

FIG. 3 is a graph showing a mutational strategy for the 5′ left end repeated region, the 5′ internal repeated region and the 3′ right end repeated region of a transposon sequence. Substitutions, insertions and deletions were used to generate mutant libraries for each of these three regions.

FIG. 4 is a schematic depiction of the mutational library design for the LE ITR. Substitutions, deletions and insertions of 1 bp, 2 bp or 3 bp in size were generated at each position of the LE ITR. Substitution—1 bp (SEQ ID NOS: 561-583); Substitution—2 bp (SEQ ID NOS: 584-607); Substitution—3 bp (SEQ ID NOS: 454-477, 584 and 585); Deletion—1 bp (SEQ ID NOS: 610-632); Deletion—2 bp (SEQ ID NOS: 633-656); Deletion—3 bp (SEQ ID NOS: 657-681).

FIG. 5 is a series of graphs showing the enrichment scores for each deletion mutation library of the 5′ left end repeated region, the 5′ internal repeated region and the 3′ right end repeated region.

FIG. 6 is a series of graphs showing the enrichment scores for each insertion mutation library of the 5′ left end repeated region, the 5′ internal repeated region and the 3′ right end repeated region.

FIG. 7 is a series of graphs showing the enrichment scores for each substitution mutation library of the 5′ left end repeated region, the 5′ internal repeated region and the 3′ right end repeated region.

FIG. 8 is a schematic diagram of the mutational library design for a LE ITR and RE ITR of a transposon. Two libraries of individual trimer substitutions were generated. The LE ITR mutant library (Library 5) has 1888 mutants (Exemplary sequences SEQ ID NOs: 454-486 are shown). The RE ITR mutant library (Library 6) has 3232 mutants (Exemplary sequences SEQ ID NOs: 487-547 are shown). N represents any of the four nucleotides. NNN represents 64 different combinations of trimer substitutions.

FIG. 9A is a graph showing the stacked enrichment scores for transposition at each position of the LE ITR of a Piggybac transposon following mutational analysis. The transposon positions are overlayed with the predicted binding sites of the Dimerization and DNA Binding Domains (DDBD1 and DDBD2) and Cysteine Rich Domains (CRD1 and CRD2) of a PiggyBac transposase. Only certain regions of the LE ITR tolerate mutations and a subset of these mutations improve transposition.

FIG. 9B is a graph showing the stacked enrichment scores for transposition at each position of the RE ITR of a Piggybac transposon following mutational analysis. The transposon positions are overlayed with the predicted binding sites of the DDBD1, DDBD2, CRD1 and CRD2 of a PiggyBac transposase. Only certain regions of the RE ITR tolerate mutations and many of these mutations improve transposition. The enriched mutations of the RE ITR were tested in combination with enriched mutations of the LE ITR and did not show additional improvement in transposition.

FIG. 10 is a graph showing a subset of LE ITR transposon mutants that increased transposition relative to a transposon with a wildtype LE ITR. Each transposon with mutant LE ITRs were transfected individually into HEK 293T cells along with Super Piggybac transposase. The percent GFP positive cells was measured by flow cytometry 7 days later. The y-axis represents Day 7 integration normalized to transfection efficiency. Certain mutant LE ITR transposons increased the percent of transposed cells by at least 5%. Exemplary mutant LE ITR sequences of SEQ ID NO: 1, 4, 16, 160-163, 165-166, 169, 171, 174-175, 177, 178-181, 183-185, 187, 189, 191, 193, 196, 199-200, 203-204, 206, 208, 210, 212-215, 219-220, 224, 226-232, 236-237, 239, 241, 245-247, 249, 250-251, 253, 256, 260-261, 263, 265, and 517 are shown.

FIG. 11 is a schematic depiction of the dual reporter plasmid design used to confirm the rates of excision and integration using each mutant transposon. Using an H-2kk GFP transposon reporter (Reporter 1), an increase in H2kk expression is observed if there is an increase in excision of the transposon. Using Reporter 2, an increase in GFP expression is observed if there is an increase in the integration of the transposon. In an alternative design of Reporter 2, an increase in Firefly luciferase expression is observed if there is an increase in excision of the transposon and an increase in NanoLuc is observed if there is an increase in the integration of the transposon.

FIG. 12 is a schematic depiction of an H-2kk GFP transposon reporter (Reporter 1) (SEQ ID NO: 452). Structural features of the transposon are shown both in a circular map and a linear map. An increase in H2kk expression is observed if there is an increase in excision of the transposon and an increase in GFP is observed if there is an increase in integration of the transposon.

FIG. 13 is a schematic depiction of a Firefly luciferase Nano Luc transposon reporter (SEQ ID NO: 453). Structural features of the transposon are shown both in a circular map and a linear map. Firefly luciferase expression is observed if there is an increase in excision of the transposon and an increase in NanoLuc is observed if there is an increase in the integration of the transposon.

FIG. 14 is a series of graphs showing the excision and integration rate of each mutated transposon. While many mutants showed improvement over wildtype excision and integration, mutant transposons with a 31TCC mutation on the LE LTR (i.e. substitutions 31G>T and 33A>C) showed the highest increase of cells with of excision and/or integration. Exemplary mutant LE ITRs of SEQ ID NO: 166, 16, 548-550, 16, 551, 4 and 552 are shown from top to bottom. Exemplary mutant RE ITRs of SEQ ID NO: 5 and 553-556 are shown from top to bottom.

FIG. 15 is a series of graphs showing improved transposition using mutant transposons and Super Piggy bac transposase in K562 cells, HEK293T cells and primary T cells.

All documents cited herein, including any cross referenced or related patent or application is hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### DETAILED DESCRIPTION OF THE INVENTION

The disclosure provides transposons, compositions and cells comprising transposons, methods of making transposons and methods of using the transposons, compositions and cells described herein.

The transposons of the disclosure are designed to optimize the left end inverted terminal repeat (LE ITR) and the right end inverted repeat (RE ITR), thereby increasing transposition efficacy and efficiency. The transposons and compositions comprising transposons of the disclosure are effective in every cell type; however, they are particularly effective for use in human cells.

As described herein, transposons of the disclosure may be used to increase the frequency of transposition, thereby resulting in a higher percentage of genetically engineered cells with a desired gene transfer or a desired site specific mutation in a population of genetically edited cells. Transposons of the disclosure may also be used decrease the dose of donor plasmid that is required for gene editing, thereby increasing cell viability.

Without wishing to be bound by theory, a change in binding affinity between the ITR region and the corresponding transposase may alter the ability of the transposase to bring both ITR sequences together in comparison to a wildtype ITR, thereby resulting in increased excision of the intra-ITR sequence from the transposon and/or an increased integration of the intra-ITR sequence into a target site.

### Compositions of the Disclosure

The present disclosure provides a polynucleotide encoding a transposon comprising: a) a first inverted terminal repeat (ITR) and b) a second ITR, wherein the first ITR and/or the second ITR comprises at least one nucleic acid substitution relative to a wildtype ITR of a transposon. In some aspects, the transposon is a nanotransposon. In some aspects, the first ITR is the left end ITR. In some aspects, the at least one nucleic acid substitution relative to a wildtype ITR of the transposon provides an increase in transposition activity. In some aspects, the increase in transposition activity is an increase in excision of a transposon in a cell. In some aspects, the increase in transposition activity is an increase in integration of a transposon in a cell. In some aspects, the increase in transposition activity is an increase in excision and an increase in integration of a transposon in a cell.

In some aspects, a transposon of the present disclosure can comprise at least one inverted terminal repeat (ITR) sequence, or a reverse complement thereof. In some aspects, a circular single-stranded DNA polynucleotide can comprise two ITR sequences, or complements thereof.

In some aspects, an ITR sequence can comprise any ITR sequence known in the art. In some aspects, an ITR sequence can comprise, consist essentially of, or consist of any piggyBac transposon ITR sequence known in the art. In some aspects, an ITR sequence can comprise, consist essentially of, or consist of a piggyBac transposon ITR sequence, Sleeping Beauty transposon ITR, a Helraiser transposon ITR, a Tol2 transposon ITR, a TcBuster transposon ITR or any combination thereof.

The sequence encoding a first ITR or the sequence encoding a second ITR can comprise a TTAA, a TTAT, or a TTAX recognition sequence. The sequence encoding a first ITR or the sequence encoding a second ITR can comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 25, at least 30, at least 35, at least 40, at least 45, at least 50, at least 55, at least 60, at least 65, at least 70, at least 75, at least 80 number of nucleotides (or any number of nucleotides in between).

In some aspects, a piggyBac ITR sequence of the present disclosure can be flanked on either or both ends by at least one of the following sequences: 5′-CTAA-3′, 5′-TTAG-3′, 5′-ATAA-3′, 5′-TCAA-3′, 5′AGTT-3′, 5′-ATTA-3′, 5′-GTTA-3′, 5′-TTGA-3′, 5′-TTTA-3′, 5′-TTAC-3′, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3″, 5′-CCAG-3′, 5′-CCCA-3′, 5′-CGTA-3′, 5′-GTCC-3′, 5′-TAAG-3′, 5′-TCTA-3′, 5′-TGAG-3″, 5′-TGTT-3′, 5′-TTCA-3′5′-TTCT-3′ and 5′-TTTT-3′. In some aspects, a piggyBac ITR sequence can be flanked by 5′-TTAA-3′. Thus, any partially double-stranded DNA molecule of the present disclosure can further comprise any one of: 5′-CTAA-3′, 5′-TTAG-3′, 5′-ATAA-3″, 5′-TCAA-3′, 5′AGTT-3′, 5′-ATTA-3′, 5′-GTTA-3′, 5′-TTGA-3′, 5′-TTTA-3′, 5′-TTAC-3″, 5′-ACTA-3′, 5′-AGGG-3′, 5′-CTAG-3′, 5′-TGAA-3′, 5′-AGGT-3′, 5′-ATCA-3′, 5′-CTCC-3′, 5′-TAAA-3′, 5′-TCTC-3′, 5′TGAA-3′, 5′-AAAT-3′, 5′-AATC-3′, 5′-ACAA-3′, 5′-ACAT-3′, 5′-ACTC-3′, 5′-AGTG-3′, 5′-ATAG-3′, 5′-CAAA-3′, 5′-CACA-3′, 5′-CATA-3′, 5′-CCAG-3″, 5′-CCCA-3′, 5′-CGTA-3′, 5′-GTCC-3′, 5′-TAAG-3′, 5′-TCTA-3′, 5′-TGAG-3′, 5′-TGTT-3″, 5′-TTCA-3′5′-TTCT-3′ and 5′-TTTT-3′ flanking a piggyBac ITR sequence.

In some aspects, a piggyBac ITR sequence of the present disclosure can be flanked on either or both ends by at least one of the following sequences, or a reverse complement thereof: 5′-NTAA-3′, 5′-TNAA-3′, 5′-TTNA-3′ an 5′-TTAN-3′, wherein N is any one of A, T, C or G. Thus, any partially double-stranded DNA molecule of the present disclosure can further comprise any one of 5′-NTAA-3′, 5′-TNAA-3′, 5′-TTNA-3′ an 5′-TTAN-3′, wherein N is any one of A, T, C or G, or a reverse complement thereof.

In some aspects, a piggyBac ITR sequence can comprise any piggyBac ITR sequence known in the art. In some aspects, a piggyBac ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOS: 1-5, or a reverse complement thereof.

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 4.

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 11. The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 1. The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 2. The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 3.

Exemplary Mutant Left End (LE) ITR sequences of the disclosure and shown in Table 1. The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to any one of SEQ ID NOS: 14-16 or 160-163, 165-265.

TABLE 1
Mutant LE ITR Sequences

| | | Mutation | |
|---|---|---|---|
| | | Position | |
| SEQ ID | | relative to | |
| NO: | Mutant LE ITR Sequence | SEQ ID NO: 4 | Mutation |
| 4 | CCCTAGAAAGATAGTCTGCGTAAAATTGACGCATG | n/a | n/a |
| 14 | CCCTAGAAAGATAGTCTGCGTAAAATTGACTCATG | 31 | TCA |
| 15 | CCCTAGAAAGATAGTCTGCGTAAAATTGACGCCTG | 33 | CTG |
| 16 | CCCTAGAAAGATAGTCTGCGTAAAATTGACTCCTG | 31, 33 | TCC |
| 160 | CCCTAGAAAGATAGTCTGCGTAAAATTGACTTCTG | 31, 32, 33 | TTC |
| 161 | CCCTAGAAAAACAGTCTGCGTAAAATTGACGCATG | 10, 12 | AAC |
| 162 | CCCTAGAAAGATAGCATGCGTAAAATTGACGCATG | 15, 16 | GCA |
| 163 | CCCTAGTACGATAGTCTGCGTAAAATTGACGCATG | 7, 9 | TAC |
| 165 | CCCTAGAAAGATAGTCTGCGTAAAATTGACCTCTG | 31, 32, 33 | CTC |
| 166 | CCCTAGTCCGATAGTCTGCGTAAAATTGACGCATG | 7, 8, 9 | TCC |
| 167 | CCCTAGAAAGATAGTCTGCGTAAAATTGATCTATG | 30, 31, 32 | TCT |
| 168 | CCCTAGAAAGATAGTCTGCGTAAAATTGACTTATG | 30, 31, 32 | CTT |
| 169 | CCCTAGAAAGATAGTCTGCGTAAAATTGGCTCATG | 29, 31 | GCT |
| 170 | CCCTAGAAAGATAGTCTGCGTAAAATTGACGCATC | 35 | CGG |
| 171 | CCCTAGCATGATAGTCTGCGTAAAATTGACGCATG | 7, 9 | CAT |
| 172 | CCCTAGAAAGATAGTCTGCGTAAAATTGACGCGCC | 33, 34, 35 | GCC |
| 173 | CCCTAGAAAGACCATCTGCGTAAAATTGACGCATG | 12, 13, 14 | CCA |
| 174 | CCCTAGAAAGATCAACTGCGTAAAATTGACGCATG | 13, 14, 15 | CAA |
| 175 | CCCTAGAAAGATCTTCTGCGTAAAATTGACGCATG | 13, 14 | TCT |
| 176 | CCCTAGTCAGATAGTCTGCGTAAAATTGACGCATG | 7, 8 | GTC |
| 177 | CCCTAGAAAGATAGTCTGCGTAAAATTGGTGCATG | 29, 30 | GGT |
| 178 | CCCTAGAAAGATAGTCTGCGTAAAATTGTCACATG | 29, 31 | TCA |
| 179 | CCCTAGAAAGATAGTCTGCGTAAAATTGACGCCCT | 33, 34, 35 | CCT |
| 180 | CCCTAGAAAGGGGGTCTGCGTAAAATTGACGCATG | 11, 12, 13 | GGG |
| 181 | CCCTCAGAAGATAGTCTGCGTAAAATTGACGCATG | 5, 6, 7 | CAG |
| 182 | CCCTAGAAAGATTTACTGCGTAAAATTGACGCATG | 13, 14, 15 | TTA |
| 183 | CCCTAGAAAGATACAATGCGTAAAATTGACGCATG | 14, 15, 16 | CAA |
| 184 | CCCTAGAGCAATAGTCTGCGTAAAATTGACGCATG | 8, 9, 10 | GCA |
| 185 | CCCTAGAAAGATAGTCTGCGTAAAATTGTACCATG | 29, 30, 31 | TAC |
| 186 | CCCTAGAAAGATAGTCTGCGTAAAATTGACGCATC | 35 | CAG |
| 187 | CCCTAGAAAGATAGTATACGTAAAATTGACGCATG | 16 | ATA |
| 188 | CCCTAGAAAGATAGTGTACGTAAAATTGACGCATG | 16, 18 | GTA |
| 189 | CCCTAGAAACCCAGTCTGCGTAAAATTGACGCATG | 10, 11, 12 | CCC |
| 190 | CCCTAGAAATACAGTCTGCGTAAAATTGACGCATG | 10, 12 | TAC |
| 191 | CCCTAGAAAGATAGTCTGCGTAAAATTGACGTCGG | 32, 33, 34 | TCG |
| 192 | CCCTAGAAAGATAGTCTGCGTAAAATTGACGCACC | 34, 35 | ACC |
| 193 | CCCTAGAAAGATAGTCTGCGTAAATCTGACGCATG | 25, 26 | ATC |
| 194 | CCCTAGCTAGATAGTCTGCGTAAAATTGACGCATG | 7, 8 | GCT |
| 195 | CCCTAGAACGTTAGTCTGCGTAAAATTGACGCATG | 9, 11 | CGT |
| 196 | CCCTAGAAAGATAGTCTGCGTAATCTTGACGCATG | 24, 25 | ATC |
| 197 | CCCTAGAAAGATAGTCTGCGTAAAATTGACGGGCG | 32, 33, 34 | GGC |
| 198 | CCCTAGAAAGATAGTCTGCGTAAAATTGACGGGGG | 32, 33, 34 | GGG |
| 199 | CCCTTAAAAGATAGTCTGCGTAAAATTGACGCATG | 5, 6 | TTA |
| 200 | CCCTAGAAAGATACAGTGCGTAAAATTGACGCATG | 14, 15, 16 | CAG |
| 201 | CCCTCGCAAGATAGTCTGCGTAAAATTGACGCATG | 5, 7 | CGC |
| 202 | CCCTAGAAAGATAGTCTGCGTAAAATCTGCGCATG | 27, 28, 29 | CTG |
| 203 | CCCTAGAAAGATAGTCTGCGTAATTGTGACGCATG | 24, 25, 26 | TTG |
| 204 | CCCTAGAAAGAAGATCTGCGTAAAATTGACGCATG | 12, 13, 14 | AGA |
| 206 | CCCTAGAAAGATAGTCTGCGTAAAGCGGACGCATG | 25, 26, 27 | GCG |
| 207 | CCCTAGAAAGATAGTCTGCGTAAAATTGACGCAGA | 34, 35 | GAG |
| 208 | CCCTAGAAAGTAGGTCTGCGTAAAATTGACGCATG | 11, 12, 13 | TAG |
| 209 | CCCCTGAAAGATAGTCTGCGTAAAATTGACGCATG | 4, 5 | CCT |
| 210 | CCCTAGAGGTATAGTCTGCGTAAAATTGACGCATG | 8, 9, 10 | GGT |
| 211 | CCCTAGAAAGATAGTCTGCGTAAAACTCACGCATG | 26, 28 | CTC |
| 212 | CCCTAAGCAGATAGTCTGCGTAAAATTGACGCATG | 6, 7, 8 | AGC |
| 213 | CCCTCCAAAGATAGTCTGCGTAAAATTGACGCATG | 5, 6 | TCC |
| 214 | CCCTAGAAAGATAGTCTGCGTAAATTCGACGCATG | 25, 27 | TTC |
| 215 | CCCCAGAAAGATAGTCTGCGTAAAATTGACGCATG | 4 | CCC |
| 216 | CCCTAGAAAGATAGTCTGCGTAAAAGCCACGCATG | 26, 27, 28 | GCC |
| 217 | CCCTGCGAAGATAGTCTGCGTAAAATTGACGCATG | 5, 6, 7 | GCG |
| 218 | CCCTAGAAAGATAGTCTGCGTAAAATTGACGCATT | 35 | TGG |
| 219 | CCCTAGATCCATAGTCTGCGTAAAATTGACGCATG | 8, 9, 10 | TCC |
| 220 | CCCTAGAAAGATAGTCTGCGTAACGCTGACGCATG | 24, 25, 26 | CGC |
| 221 | CCCTAGAAAGATAGTCTGCGTAAAATGGGCGCATG | 27, 29 | GGG |
| 222 | CCCTAGAAAGATAGTCTGCGTAGAATTGACGCATG | 21, 22 | TAG |
| 223 | CCCTAGAAAGTCGGTCTGCGTAAAATTGACGCATG | 11, 12, 13 | TCG |
| 224 | CCCCTCAAAGATAGTCTGCGTAAAATTGACGCATG | 4, 5, 6 | CTC |
| 225 | CCCTAGAACTCTAGTCTGCGTAAAATTGACGCATG | 9, 10, 11 | CTC |
| 226 | CCCAAGAAAGATAGTCTGCGTAAAATTGACGCATG | 4 | CCA |
| 227 | CCCTAGAAAGATAGTACGCGTAAAATTGACGCATG | 15, 16 | TAC |
| 228 | CCCTAGAAAGATAGTCTGCGTGAAATTGACGCATG | 21 | GTG |
| 229 | CCCTAGAAAGATAGTCTGCGTACGGTTGACGCATG | 23, 24, 25 | CGG |
| 230 | CCCTAGAAAGATAGTCTGCGTATCCTTGACGCATG | 23, 24, 25 | TCC |
| 231 | CCCTAGAAAGATGGACTGCGTAAAATTGACGCATG | 13, 15 | GGA |
| 232 | CCCTAGAAAGATAGTCTGCGTCAGATTGACGCATG | 22, 24 | CAG |
| 233 | CCCTAGAAAGATAGTCTGCGTAAAATTGACGCAGT | 34, 35 | GTA |
| 234 | CCCTAGAAAGATAGTCTGCGTCTAATTGACGCATG | 21, 22, 23 | TCT |
| 235 | CCCTAGAAAGATAGTCTGCGTAAAATCTCCGCATG | 27, 28, 29 | CTC |
| 236 | CCCAGGAAAGATAGTCTGCGTAAAATTGACGCATG | 4, 5 | CAG |
| 237 | CCCTAGAAAGATAGTCTGCGTAAAATATACGCATG | 27, 28 | TAT |
| 238 | CCCTAGAAAGATAGTCTGCGTCGTATTGACGCATG | 22, 23, 24 | CGT |
| 239 | CCCTAGAAAGATAGTCTGCGTAAAATTGATTAATG | 30, 31, 32 | TTA |
| 240 | CCCTAGAAAGATAGTCTGCGTAAACTGGACGCATG | 25, 27 | CTG |
| 241 | CCCTAGAAAGATAGTCTGCGCTAAATTGACGCATG | 20, 21 | GCT |
| 242 | CCCGAGAAAGATAGTCTGCGTAAAATTGACGCATG | 4 | CCG |
| 243 | CCCTAGAAGGTTAGTCTGCGTAAAATTGACGCATG | 9, 11 | GGT |
| 244 | CCCTAGAAAGATAGTCTGCGTAAAATTGACGCAGA | 34, 35 | GAT |
| 245 | CCCTAGAAAGATAGTCTGCGGAAAATTGACGCATG | 21 | CGG |
| 246 | CCTTAGAAAGATAGTCTGCGTAAAATTGACGCATG | 3 | CCT |
| 247 | CCGTGGAAAGATAGTCTGCGTAAAATTGACGCATG | 3, 5 | GTG |
| 248 | CCCTAGAAAGATAGTCTGCGTAAAATTCTAGCATG | 28, 29, 30 | CTA |
| 249 | CCCTAGAAAGATAGTCCACGTAAAATTGACGCATG | 17, 18 | CCA |
| 250 | CCCTAGAAAGATAGTCTGCGCCAAATTGACGCATG | 20, 21 | GCC |
| 251 | CCCTAGAAAGATAGTCCGCGTAAAATTGACGCATG | 15 | TCC |
| 252 | CGGTAGAAAGATAGTCTGCGTAAAATTGACGCATG | 2, 3 | CGG |
| 253 | CCCTAGAAAGATAGACCGCGTAAAATTGACGCATG | 17 | ACC |
| 254 | CCCTAGAAAGATAGTCACAGTAAAATTGACGCATG | 17, 18, 19 | ACA |
| 255 | CTGTAGAAAGATAGTCTGCGTAAAATTGACGCATG | 2, 3 | CTG |
| 256 | CCCTAGAAAGATAGTCTGCGTCCGATTGACGCATG | 22, 23, 24 | CCG |
| 257 | CCCTAGAAAGATAGTCTGCGTAAAATTTTAGCATG | 28, 29, 30 | TTA |
| 258 | CCCTAGAAAGATAGTCCGTGTAAAATTGACGCATG | 17, 19 | CGT |
| 259 | CCCTAGAAAGATAGTCTGCGGTGAATTGACGCATG | 21, 22, 23 | GTG |
| 260 | CCCTAGAAAGATAGTCAGTGTAAAATTGACGCATG | 17, 19 | AGT |
| 261 | CCCTAGAAAGATAGTCTGGGCAAAATTGACGCATG | 19, 21 | GGC |
| 262 | CCCTAGAAAGATAGTCTGGAGAAAATTGACGCATG | 19, 20, 21 | GAG |
| 263 | CCCTAGAAAGATAGTCTAGTTAAAATTGACGCATG | 18, 19, 20 | AGT |
| 264 | CCCTAGAAAGATAGTCTTGATAAAATTGACGCATG | 18, 19, 20 | TGA |
| 265 | CCCTAGAAAGATAGTCTAGATAAAATTGACGCATG | 18, 19, 20 | AGA |

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 14.

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 15.

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 16.

In some aspects, the sequence encoding the first ITR comprises a nucleic acid substitution in at least one of nucleic acid position 31 and position 33 of SEQ ID NO: 4. In some aspects, the first ITR comprises the nucleic acid substitution of 31 G>T. In some aspects, the first ITR comprises the nucleic acid substitution of 33A>C. In some aspects, the first ITR comprises the nucleic acid substitution of 31G>T and 33A>C. In some aspects, the first ITR comprises the nucleic acid sequence of SEQ ID NO: 14. In some aspects, the first ITR comprises the nucleic acid sequence of SEQ ID NO: 15. In some aspects, the first ITR comprises the nucleic acid sequence of SEQ ID NO: 16. In some aspects the “31TCC” mutant LE ITR comprises the nucleic acid sequence of SEQ ID NO: 16.

In some aspects, the first ITR comprises the nucleic acid sequence of SEQ ID NO: 160. In some aspects, the first ITR comprises the nucleic acid sequence of SEQ ID NO: 161. In some aspects, the first ITR comprises the nucleic acid sequence of SEQ ID NO: 162. In some aspects, the first ITR comprises the nucleic acid sequence of SEQ ID NO: 163. In some aspects, the first ITR comprises the nucleic acid sequence of SEQ ID NO: 165. In some aspects, the first ITR comprises the nucleic acid sequence of SEQ ID NO: 166. In some aspects, the first ITR comprises the nucleic acid sequence of SEQ ID NO: 167. In some aspects, the first ITR comprises the nucleic acid sequence of SEQ ID NO: 168. In some aspects, the first ITR comprises the nucleic acid sequence of SEQ ID NO: 169. In some aspects, the first ITR comprises the nucleic acid sequence of SEQ ID NO: 170. In some aspects, the first ITR comprises the nucleic acid sequence of SEQ ID NO: 171. In some aspects, the first ITR comprises the nucleic acid sequence of SEQ ID NO: 172. In some aspects, the first ITR comprises the nucleic acid sequence of SEQ ID NO: 173. In some aspects, the first ITR comprises the nucleic acid sequence of SEQ ID NO: 174. In some aspects, the first ITR comprises the nucleic acid sequence of SEQ ID NO: 175. In some aspects, the first ITR comprises the nucleic acid sequence of SEQ ID NO: 176. In some aspects, the first ITR comprises the nucleic acid sequence of SEQ ID NO: 177. In some aspects, the first ITR comprises the nucleic acid sequence of SEQ ID NO: 178. In some aspects, the first ITR comprises the nucleic acid sequence of SEQ ID NO: 179. In some aspects, the first ITR comprises the nucleic acid sequence of SEQ ID NO: 180. In some aspects, the first ITR comprises the nucleic acid sequence of SEQ ID NO: 181. In some aspects, the first ITR comprises the nucleic acid sequence of SEQ ID NO: 182. In some aspects, the first ITR comprises the nucleic acid sequence of SEQ ID NO: 183. In some aspects, the first ITR comprises the nucleic acid sequence of SEQ ID NO: 184. In some aspects, the first ITR comprises the nucleic acid sequence of SEQ ID NO: 185. In some aspects, the first ITR comprises the nucleic acid sequence of SEQ ID NO: 186. In some aspects, the first ITR comprises the nucleic acid sequence of SEQ ID NO: 187. In some aspects, the first ITR comprises the nucleic acid sequence of SEQ ID NO: 188. In some aspects, the first ITR comprises the nucleic acid sequence of SEQ ID NO: 189. In some aspects, the first ITR comprises the nucleic acid sequence of SEQ ID NO: 190. In some aspects, the first ITR comprises the nucleic acid sequence of SEQ ID NO: 191. In some aspects, the first ITR comprises the nucleic acid sequence of SEQ ID NO: 192. In some aspects, the first ITR comprises the nucleic acid sequence of SEQ ID NO: 193. In some aspects, the first ITR comprises the nucleic acid sequence of SEQ ID NO: 194. In some aspects, the first ITR comprises the nucleic acid sequence of SEQ ID NO: 195. In some aspects, the first ITR comprises the nucleic acid sequence of SEQ ID NO: 196. In some aspects, the first ITR comprises the nucleic acid sequence of SEQ ID NO: 197. In some aspects, the first ITR comprises the nucleic acid sequence of SEQ ID NO: 198. In some aspects, the first ITR comprises the nucleic acid sequence of SEQ ID NO: 199. In some aspects, the first ITR comprises the nucleic acid sequence of SEQ ID NO: 200. In some aspects, the first ITR comprises the nucleic acid sequence of SEQ ID NO: 201. In some aspects, the first ITR comprises the nucleic acid sequence of SEQ ID NO: 202. In some aspects, the first ITR comprises the nucleic acid sequence of SEQ ID NO: 203. In some aspects, the first ITR comprises the nucleic acid sequence of SEQ ID NO: 204. In some aspects, the first ITR comprises the nucleic acid sequence of SEQ ID NO: 205. In some aspects, the first ITR comprises the nucleic acid sequence of SEQ ID NO: 206. In some aspects, the first ITR comprises the nucleic acid sequence of SEQ ID NO: 207. In some aspects, the first ITR comprises the nucleic acid sequence of SEQ ID NO: 208. In some aspects, the first ITR comprises the nucleic acid sequence of SEQ ID NO: 209. In some aspects, the first ITR comprises the nucleic acid sequence of SEQ ID NO: 210. In some aspects, the first ITR comprises the nucleic acid sequence of SEQ ID NO: 211. In some aspects, the first ITR comprises the nucleic acid sequence of SEQ ID NO: 212. In some aspects, the first ITR comprises the nucleic acid sequence of SEQ ID NO: 213. In some aspects, the first ITR comprises the nucleic acid sequence of SEQ ID NO: 214. In some aspects, the first ITR comprises the nucleic acid sequence of SEQ ID NO: 215. In some aspects, the first ITR comprises the nucleic acid sequence of SEQ ID NO: 216. In some aspects, the first ITR comprises the nucleic acid sequence of SEQ ID NO: 217. In some aspects, the first ITR comprises the nucleic acid sequence of SEQ ID NO: 218. In some aspects, the first ITR comprises the nucleic acid sequence of SEQ ID NO: 219. In some aspects, the first ITR comprises the nucleic acid sequence of SEQ ID NO: 220. In some aspects, the first ITR comprises the nucleic acid sequence of SEQ ID NO: 221. In some aspects, the first ITR comprises the nucleic acid sequence of SEQ ID NO: 222. In some aspects, the first ITR comprises the nucleic acid sequence of SEQ ID NO: 223. In some aspects, the first ITR comprises the nucleic acid sequence of SEQ ID NO: 224. In some aspects, the first ITR comprises the nucleic acid sequence of SEQ ID NO: 225. In some aspects, the first ITR comprises the nucleic acid sequence of SEQ ID NO: 226. In some aspects, the first ITR comprises the nucleic acid sequence of SEQ ID NO: 227. In some aspects, the first ITR comprises the nucleic acid sequence of SEQ ID NO: 228. In some aspects, the first ITR comprises the nucleic acid sequence of SEQ ID NO: 229. In some aspects, the first ITR comprises the nucleic acid sequence of SEQ ID NO: 230. In some aspects, the first ITR comprises the nucleic acid sequence of SEQ ID NO: 231. In some aspects, the first ITR comprises the nucleic acid sequence of SEQ ID NO: 232. In some aspects, the first ITR comprises the nucleic acid sequence of SEQ ID NO: 233. In some aspects, the first ITR comprises the nucleic acid sequence of SEQ ID NO: 234. In some aspects, the first ITR comprises the nucleic acid sequence of SEQ ID NO: 235. In some aspects, the first ITR comprises the nucleic acid sequence of SEQ ID NO: 236. In some aspects, the first ITR comprises the nucleic acid sequence of SEQ ID NO: 237. In some aspects, the first ITR comprises the nucleic acid sequence of SEQ ID NO: 238. In some aspects, the first ITR comprises the nucleic acid sequence of SEQ ID NO: 239. In some aspects, the first ITR comprises the nucleic acid sequence of SEQ ID NO: 240. In some aspects, the first ITR comprises the nucleic acid sequence of SEQ ID NO: 241. In some aspects, the first ITR comprises the nucleic acid sequence of SEQ ID NO: 242. In some aspects, the first ITR comprises the nucleic acid sequence of SEQ ID NO: 243. In some aspects, the first ITR comprises the nucleic acid sequence of SEQ ID NO: 244. In some aspects, the first ITR comprises the nucleic acid sequence of SEQ ID NO: 245. In some aspects, the first ITR comprises the nucleic acid sequence of SEQ ID NO: 246. In some aspects, the first ITR comprises the nucleic acid sequence of SEQ ID NO: 247. In some aspects, the first ITR comprises the nucleic acid sequence of SEQ ID NO: 248. In some aspects, the first ITR comprises the nucleic acid sequence of SEQ ID NO: 249. In some aspects, the first ITR comprises the nucleic acid sequence of SEQ ID NO: 250. In some aspects, the first ITR comprises the nucleic acid sequence of SEQ ID NO: 251. In some aspects, the first ITR comprises the nucleic acid sequence of SEQ ID NO: 252. In some aspects, the first ITR comprises the nucleic acid sequence of SEQ ID NO: 253. In some aspects, the first ITR comprises the nucleic acid sequence of SEQ ID NO: 254. In some aspects, the first ITR comprises the nucleic acid sequence of SEQ ID NO: 255. In some aspects, the first ITR comprises the nucleic acid sequence of SEQ ID NO: 256. In some aspects, the first ITR comprises the nucleic acid sequence of SEQ ID NO: 257. In some aspects, the first ITR comprises the nucleic acid sequence of SEQ ID NO: 258. In some aspects, the first ITR comprises the nucleic acid sequence of SEQ ID NO: 259. In some aspects, the first ITR comprises the nucleic acid sequence of SEQ ID NO: 260. In some aspects, the first ITR comprises the nucleic acid sequence of SEQ ID NO: 261. In some aspects, the first ITR comprises the nucleic acid sequence of SEQ ID NO: 262. In some aspects, the first ITR comprises the nucleic acid sequence of SEQ ID NO: 263. In some aspects, the first ITR comprises the nucleic acid sequence of SEQ ID NO: 264. In some aspects, the first ITR comprises the nucleic acid sequence of SEQ ID NO: 265.

The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to SEQ ID NO: 5.

Exemplary Mutant Right End (RE) ITR sequences of the disclosure and shown in Table 2. The sequence encoding a first ITR or the sequence encoding a second ITR can comprise, can consist essentially of, or can consist of a nucleic acid sequence at least 70%, 75%, 80%, 85%, 90%, 95%, 96%, 97%, 98%, 99% or 100% (or any percentage in between) identical to any one of SEQ ID NOS: 267-277, 279-329 or 331-451.

TABLE 2
Mutant RE ITR Sequences

| | | Mutation | |
|---|---|---|---|
| | | Position | |
| | | relative to | |
| SEQ ID | | SEQ ID NO: | |
| NO: | Mutant RE ITR Sequence | 5 | Mutation |
| 5 | CCCTAGAAAGATAATCATATTGTGACGTACGTTAAAGATA | n/a | n/a |
| | ATCATGCGTAAAATTGACGCATG | | |
| 267 | CCCTAGAAAGATAATCATATTGTGACGTATGCTAAAGATA | 30, 32 | TGC |
| | ATCATGCGTAAAATTGACGCATG | | |
| 268 | CCCTAGAAAGATAATCATATTGTGACGTACGTTAAAGATA | 50, 51, 52 | TTT |
| | ATCATGCGTTTTATTGACGCATG | | |
| 269 | CCCTAGAAAGATAATCATATTGTGACGTACGTTAAAGATA | 52, 53 | ATT |
| | ATCATGCGTAATTTTGACGCATG | | |
| 270 | CCCTAGAAAGATAATCATATTGTGACGTACGTTAAAGATA | 52, 53, 54 | GCC |
| | ATCATGCGTAAGCCTGACGCATG | | |
| 271 | CCCTAGAAAGATAATCATATTGTGACGTACGTTAAAGATA | 59, 60, 61 | TGG |
| | ATCATGCGTAAAATTGACTGGTG | | |
| 272 | CCCTAGAAAGATAATCATATTGTGACGTACGTTAAAGATA | 55, 56 | TAT |
| | ATCATGCGTAAAATATACGCATG | | |
| 273 | CCCTAGAAAGATAATCATATTGTGACGTACGTTAAAGATA | 49, 50, 51 | CCG |
| | ATCATGCGCCGAATTGACGCATG | | |
| 274 | CCCTAGAAAGATAATCATATTGTGACGTACGTTAAAGATA | 57, 58, 59 | CTA |
| | ATCATGCGTAAAATTGCTACATG | | |
| 275 | CCCTAGAAAGATAATCATATTGTGACGTACGTTAAAGATA | 55, 56 | TGA |
| | ATCATGCGTAAAATGAACGCATG | | |
| 276 | CCCTAGAAAGATAATCATATTGTGACGTACGTTAAAGATA | 57, 58, 59 | GTC |
| | ATCATGCGTAAAATTGGTCCATG | | |
| 277 | CCCTAGAAAGATAATCATATTGTGATCGACGTTAAAGATA | 26, 27, 28 | TCG |
| | ATCATGCGTAAAATTGACGCATG | | |
| 279 | CCCTAGAAAGATAATCATATTGTGACGTACGTTAAAGATA | 42, 44 | ACT |
| | AACTTGCGTAAAATTGACGCATG | | |
| 280 | CCCTAGAAAGATAATCATATTGTGACGTACGTTAAAGATA | 45, 46, 47 | GTA |
| | ATCAGTAGTAAAATTGACGCATG | | |
| 281 | CCCTAGAAAGATAATCATATTGTGACGTACGTTAAAGATA | 52, 53, 54 | TGA |
| | ATCATGCGTAATGATGACGCATG | | |
| 282 | CCCTAGAAAGATAATCATATTGTGACGTACGTTAAAGATA | 59, 60, 61 | TAC |
| | ATCATGCGTAAAATTGACTACTG | | |
| 283 | CCCTAGAAAGATAATCATATTGTGACGTACGTTAAAGATA | 53, 54, 55 | GGC |
| | ATCATGCGTAAAGGCGACGCATG | | |
| 284 | CCCTAGAAAGATAATCATATTGTGTGCTACGTTAAAGATA | 25, 26, 27 | TGC |
| | ATCATGCGTAAAATTGACGCATG | | |
| 285 | CCCTAGAAAGATAATCATATTGTGACGTACGTTAAAGATA | 49, 50, 51 | AGT |
| | ATCATGCGAGTAATTGACGCATG | | |
| 286 | CCCTCCAAAGATAATCATATTGTGACGTACGTTAAAGATA | 5, 6 | TCC |
| | ATCATGCGTAAAATTGACGCATG | | |
| 287 | CCCTAGAAAGATAATCATATTGTGACGTACGTTAAAGATA | 45, 46 | AGT |
| | ATCAGTCGTAAAATTGACGCATG | | |
| 288 | CCCTAGAAAGATAATCATATTGTGACGTACGTTAAAGATA | 61, 62, 63 | GGT |
| | ATCATGCGTAAAATTGACGCGGT | | |
| 289 | CCCTAGAAAGATAATCATATTGTGACGTACGCAGAAGATA | 32, 33, 34 | CAG |
| | ATCATGCGTAAAATTGACGCATG | | |
| 290 | CCCTAGAAAGATAATCATATTGTGGGCTACGTTAAAGATA | 25, 26, 27 | GGC |
| | ATCATGCGTAAAATTGACGCATG | | |
| 291 | CCCTAGAAAGATAATCATATTTGCACGTACGTTAAAGATA | 22, 23, 24 | TGC |
| | ATCATGCGTAAAATTGACGCATG | | |
| 292 | CCCTAGAAAGATAATCATATTGTGACGTACGTTAAAGATA | 48, 49, 50 | CCT |
| | ATCATGCCCTAAATTGACGCATG | | |
| 293 | CCCTAGAAAGATAATCATATTGTGACGTACGCTCAAGATA | 32, 34 | CTC |
| | ATCATGCGTAAAATTGACGCATG | | |
| 294 | CCCTAGAAAGATAATCATATTGTGACGTACGTTAAAGATA | 41, 42 | AGC |
| | GCCATGCGTAAAATTGACGCATG | | |
| 295 | CCCTAGAAAGATAATCATATTGTGACGTACGTTAAAGATA | 55, 56, 57 | CTC |
| | ATCATGCGTAAAATCTCCGCATG | | |
| 296 | CCCTAGAAAGATAATCATATTGTGACGTACGTTAAAGCTG | 38, 40 | CTG |
| | ATCATGCGTAAAATTGACGCATG | | |
| 297 | CCCTAGAAAGATAATCATATTGTGACGTACGTTAAAGATA | 41, 42, 43 | CCG |
| | CCGATGCGTAAAATTGACGCATG | | |
| 298 | CCCTAGAAAGATAATCATATTGTGACGTACGTTAAAGATA | 57, 58 | GGT |
| | ATCATGCGTAAAATTGGTGCATG | | |
| 299 | CCCTAGAAAGATAATCATATGGCGACGTACGTTAAAGATA | 21, 23 | GGC |
| | ATCATGCGTAAAATTGACGCATG | | |
| 300 | CCCTAGAAAGATAATCATATTGTGACGTACGTTAAAGATG | 40, 41, 42 | GGA |
| | GACATGCGTAAAATTGACGCATG | | |
| 301 | CCCTAGAAAGATAATCATATTGTGACGCTTGTTAAAGATA | 28, 29, 30 | CTT |
| | ATCATGCGTAAAATTGACGCATG | | |
| 302 | CCCTAGAAAGATAATCATATTGTGACGTACGTTGCCGATA | 34, 35, 36 | GCC |
| | ATCATGCGTAAAATTGACGCATG | | |
| 303 | CCCTAGAAAGATAATCATATTGTGACGTACGTTAAAGATA | 50, 51, 52 | GCT |
| | ATCATGCGTGCTATTGACGCATG | | |
| 304 | CCCTAGAAAGATAATCATATTGTGACGTACGTTAAAGCCA | 38, 39 | GCC |
| | ATCATGCGTAAAATTGACGCATG | | |
| 305 | CCCTAGAAAGATAATCATATTGTGACGTTAATTAAAGATA | 29, 30, 31 | TAA |
| | ATCATGCGTAAAATTGACGCATG | | |
| 306 | CCCTAGAAAGATAATCATATTGTGACGTACGTTAAAGATA | 48, 49, 50 | CCC |
| | ATCATGCCCCAAATTGACGCATG | | |
| 307 | CCCTAGAAAGATAATCATATTGTGACGTACGTTAAAGATA | 44, 45, 46 | TAA |
| | ATCTAACGTAAAATTGACGCATG | | |
| 308 | CCCTAGAAAGATAATCATATTTTAACGTACGTTAAAGATA | 22, 24 | TTA |
| | ATCATGCGTAAAATTGACGCATG | | |
| 309 | CCCTAGAAAGATAATCATATTGTGACGTACGTTAAAGATA | 46, 47, 48 | AGC |
| | ATCATAGCTAAAATTGACGCATG | | |
| 310 | CCCTAGAAAGATAATCATATTGTGACGTACGTTAAAGATA | 57, 58 | GCG |
| | ATCATGCGTAAAATTGCGGCATG | | |
| 311 | CCCTAGAAAGATAATCATATTGTGACGTACGTTAAAGGTT | 38, 40 | GTT |
| | ATCATGCGTAAAATTGACGCATG | | |
| 312 | CCCTAGAAAGATAATCATATTGTGACCACCGTTAAAGATA | 27, 28, 29 | CAC |
| | ATCATGCGTAAAATTGACGCATG | | |
| 313 | CCCTAGAAAGATAATCATATTGTGACGTACGTTAAAGATA | 55, 56, 57 | GCC |
| | ATCATGCGTAAAATGCCCGCATG | | |
| 314 | CCCTAGAAAGATAATCATATTGTGACGTACGTTAATCATA | 36, 37 | ATC |
| | ATCATGCGTAAAATTGACGCATG | | |
| 315 | CCCTAGAAAGATAATCATATTGTGACGTACGTTAAAGATA | 47, 49 | AGG |
| | ATCATGAGGAAAATTGACGCATG | | |
| 316 | CCCTAGAAAGATAATCATATTGTGACGTACGTTAAAGATA | 51, 52, 53 | TTG |
| | ATCATGCGTATTGTTGACGCATG | | |
| 317 | CCCTAGAAAGATAATCATATTGTGACGTACGTGTGAGATA | 33, 34, 35 | GTG |
| | ATCATGCGTAAAATTGACGCATG | | |
| 318 | CCCTAGAAAGATAATCATATTGTGACGTACGTTAAAGATA | 61, 62 | CCG |
| | ATCATGCGTAAAATTGACGCCGG | | |
| 319 | CCCTAGAAAGATAATCATATTGTGAGGCACGTTAAAGATA | 26, 28 | GGC |
| | ATCATGCGTAAAATTGACGCATG | | |
| 320 | CCCTAGAAAGATAATCATATTGTGACGTACGTTAAAGATA | 60, 61, 62 | AGC |
| | ATCATGCGTAAAATTGACGAGCG | | |
| 321 | CCCTAGAAAGATAATCATATTGTGAAGCACGTTAAAGATA | 26, 28 | AGC |
| | ATCATGCGTAAAATTGACGCATG | | |
| 322 | CCCTAGAAAGATAATCATATTGTGACGTACGTTAAAGATA | 52, 53, 54 | TTG |
| | ATCATGCGTAATTGTGACGCATG | | |
| 323 | CCCTAGAAAGATAATCATATTGTGACGTCCTTTAAAGATA | 29, 31 | CCT |
| | ATCATGCGTAAAATTGACGCATG | | |
| 324 | CCCTAGAAAGATAATCATATTGTGACGTACGTTAGCCATA | 35, 36, 37 | GCC |
| | ATCATGCGTAAAATTGACGCATG | | |
| 325 | CCCTAGAAAGATAATCATATTGTGACGTACTCCAAAGATA | 31, 32, 33 | TCC |
| | ATCATGCGTAAAATTGACGCATG | | |
| 326 | CCCTAGAAAGATAATCATATTGTGACAACCGTTAAAGATA | 27, 28, 29 | AAC |
| | ATCATGCGTAAAATTGACGCATG | | |
| 327 | CCCTAGAAAGATAATCATATTGTGACGTACGTTGGAGATA | 34, 35 | TGG |
| | ATCATGCGTAAAATTGACGCATG | | |
| 328 | CCCTAGAAAGATAATCATACTTTGACGTACGTTAAAGATA | 20, 22 | CTT |
| | ATCATGCGTAAAATTGACGCATG | | |
| 329 | CCCTAGAAAGATAATCATATTGTGACGTACGTTAAAGATA | 57, 58, 59 | CTT |
| | ATCATGCGTAAAATTGCTTCATG | | |
| 331 | CCCTAGAAAGATAATCATATTGTGACGTACGTTAAAGATA | 58, 59, 60 | TCA |
| | ATCATGCGTAAAATTGATCAATG | | |
| 332 | CCCCGGAAAGATAATCATATTGTGACGTACGTTAAAGATA | 4, 5 | CCG |
| | ATCATGCGTAAAATTGACGCATG | | |
| 333 | CCCTAGAAAGATAATCATATTGTGACGTACGTTAAAGTGA | 38, 39 | GTG |
| | ATCATGCGTAAAATTGACGCATG | | |
| 334 | CCCTAGAAAGATAATCATATTGGTCCGTACGTTAAAGATA | 23, 24, 25 | GTC |
| | ATCATGCGTAAAATTGACGCATG | | |
| 335 | CCCTAGAAAGATAATCATATTGTGCATTACGTTAAAGATA | 25, 26, 27 | CAT |
| | ATCATGCGTAAAATTGACGCATG | | |
| 336 | CCCTAGAAAGATAATCATATTGTGACGTACGTTAAAGATA | 55, 57 | GGG |
| | ATCATGCGTAAAATGGGCGCATG | | |
| 337 | CCCTAGAAAGATAATCATATTGTGACGTAAACTAAAGATA | 30, 31, 32 | AAC |
| | ATCATGCGTAAAATTGACGCATG | | |
| 338 | CCCTAGAAAGATAATCATATTGTGACGTACGTTAAAGATA | 50, 51 | TCC |
| | ATCATGCGTCCAATTGACGCATG | | |
| 339 | CCCTAGAAAGATAATCATATTGTGACGTACGTTAAAGATA | 56, 58 | AAT |
| | ATCATGCGTAAAATTAATGCATG | | |
| 340 | CCCTAGAAAGATAATCATATTGTGACGTACGTTAAAGATA | 43, 45 | TAC |
| | ATTACGCGTAAAATTGACGCATG | | |
| 341 | CCCTAGAAAGATAATCATATTGTGACGTACGTTTGCGATA | 34, 35, 36 | TGC |
| | ATCATGCGTAAAATTGACGCATG | | |
| 342 | CCCTAGAAAGATAATCATATTGTGACGTACATCAAAGATA | 31, 33 | ATC |
| | ATCATGCGTAAAATTGACGCATG | | |
| 343 | CCCTAGAAAGATAATCATATTGTGACGTACGTTAAAGATA | 45, 46, 47 | GAG |
| | ATCAGAGGTAAAATTGACGCATG | | |
| 344 | CCCTAGAAAGATAATCATATTGTGACGTACGTTAAAGATA | 58, 59, 60 | GAA |
| | ATCATGCGTAAAATTGAGAAATG | | |
| 345 | CCCTCAGAAGATAATCATATTGTGACGTACGTTAAAGATA | 5, 6, 7 | CAG |
| | ATCATGCGTAAAATTGACGCATG | | |
| 346 | CCCTAGAAAGATAATCATATTGTGACGTACGTATGAGATA | 33, 34, 35 | ATG |
| | ATCATGCGTAAAATTGACGCATG | | |
| 347 | CCCTAGAAAGATAATCATATTGTGACGCGTGTTAAAGATA | 28, 29, 30 | CGT |
| | ATCATGCGTAAAATTGACGCATG | | |
| 348 | CCCTAGAAAGATAATCATATTGTGGTGTACGTTAAAGATA | 25, 26 | GGT |
| | ATCATGCGTAAAATTGACGCATG | | |
| 349 | CCCTAGAAAGATAATCATAGCCTGACGTACGTTAAAGATA | 20, 21, 22 | GCC |
| | ATCATGCGTAAAATTGACGCATG | | |
| 350 | CCCTAGAAAGATAATCATATTGTGACGTACGTTAAAGATA | 42, 43, 44 | CTC |
| | ACTCTGCGTAAAATTGACGCATG | | |
| 351 | CCCTAGAAAGATAATCATATTGTGACGTACGTTAAAGATA | 51, 52, 53 | GCC |
| | ATCATGCGTAGCCTTGACGCATG | | |
| 352 | CCCTAGAAAGATAATCATATTGTGACGTACGTTAAAGATA | 54, 55, 56 | ACC |
| | ATCATGCGTAAAAACCACGCATG | | |
| 353 | CCCTAGAAAGATAATCATATTGTGACGTACGTTAAAGATA | 45, 46, 47 | ACA |
| | ATCAACAGTAAAATTGACGCATG | | |
| 354 | CCCTAGAAAGATAATCATATTGTGACGTACGTTAAAGATA | 44, 45 | CTC |
| | ATCTCGCGTAAAATTGACGCATG | | |
| 355 | CCCTAGAAAGATAATCATATTGTGACGTACGTTAAAGATA | 42, 44 | CCC |
| | ACCCTGCGTAAAATTGACGCATG | | |
| 356 | CCCTAGAAAGATAATCATATTGTGACGTACGTTAAAGATA | 59, 60, 61 | AGC |
| | ATCATGCGTAAAATTGACAGCTG | | |
| 357 | CCCTAGAAAGATAATCATATTGTGACGCTCGTTAAAGATA | 28, 29 | GCT |
| | ATCATGCGTAAAATTGACGCATG | | |
| 358 | CCCTAGAAAGATAATCATATTGTGACGTACGTTTTCGATA | 34, 35, 36 | TTC |
| | ATCATGCGTAAAATTGACGCATG | | |
| 359 | CCCTAGAAAGATAATCATATTGTGACGTACGTTAAAGAGA | 38, 40 | GAT |
| | TTCATGCGTAAAATTGACGCATG | | |
| 360 | CCCTAGAAAGATAATCATATTGTGACGTACGTTAAAGATA | 50, 52 | GAC |
| | ATCATGCGTGACATTGACGCATG | | |
| 361 | CCCTAGAAAGATAATCATATTGTGACGTACGTTAAAGATA | 61, 62, 63 | CCA |
| | ATCATGCGTAAAATTGACGCCCA | | |
| 362 | CCCTAGAAAGATAATCATATTGTGACGTACGTTAAGCGTA | 36, 37, 38 | GCG |
| | ATCATGCGTAAAATTGACGCATG | | |
| 363 | CCCTAGAAAGATAATCATATTGTACCGTACGTTAAAGATA | 24, 25 | TAC |
| | ATCATGCGTAAAATTGACGCATG | | |
| 364 | CCCTAGAAAGATAATCATATTGTGACGTACGTTAAAGATA | 46, 47, 48 | TGC |
| | ATCATTGCTAAAATTGACGCATG | | |
| 365 | CCCTAGAAAGATAATCATATTGTGACGTACGTTAAAGATA | 61, 62 | CCC |
| | ATCATGCGTAAAATTGACGCCCG | | |
| 366 | CCCTAGAAAGATAATCATATTGTGACGTACGTTAAAGATA | 48, 50 | TTG |
| | ATCATGCTTGAAATTGACGCATG | | |
| 367 | CCCTAGAAAGATAATCATATTCACACGTACGTTAAAGATA | 22, 23, 24 | CAC |
| | ATCATGCGTAAAATTGACGCATG | | |
| 368 | CCCTAGAAAGATAATCATATTGTGACGTAGGATAAAGATA | 30, 32 | GGA |
| | ATCATGCGTAAAATTGACGCATG | | |
| 369 | CCCTAGAAAGATAATCTCATTGTGACGTACGTTAAAGATA | 17, 18 | CTC |
| | ATCATGCGTAAAATTGACGCATG | | |
| 370 | CCCTGGAAAGATAATCATATTGTGACGTACGTTAAAGATA | 5 | CTG |
| | ATCATGCGTAAAATTGACGCATG | | |
| 371 | CCCTAGAAAGATAATCATATTGTGACGTACGTTAAAGAGG | 39, 40, 41 | GGG |
| | GTCATGCGTAAAATTGACGCATG | | |
| 372 | CCCTAGAAAGATAATCATATTGTGACGTACGTTAAACGGA | 37, 38, 39 | CGG |
| | ATCATGCGTAAAATTGACGCATG | | |
| 373 | CCCTTACAAGATAATCATATTGTGACGTACGTTAAAGATA | 5, 6, 7 | TAC |
| | ATCATGCGTAAAATTGACGCATG | | |
| 374 | CCCTAGAAAGATAATCATATTGTGACGTACTAGAAAGATA | 31, 32, 33 | TAG |
| | ATCATGCGTAAAATTGACGCATG | | |
| 375 | CCCTAGAAAGATAATCTAGTTGTGACGTACGTTAAAGATA | 17, 18, 19 | TAG |
| | ATCATGCGTAAAATTGACGCATG | | |
| 376 | CCCTAGAAAGATAATCATATACGGACGTACGTTAAAGATA | 21, 22, 23 | ACG |
| | ATCATGCGTAAAATTGACGCATG | | |
| 377 | CCCTAGAAAGATAATCATATTGTGACGTACGTTAAAGATA | 53, 54, 55 | TAC |
| | ATCATGCGTAAATACGACGCATG | | |
| 378 | CCCTAGAAAGATAATCATATTGTGACGTACGTTAAAGATA | 44, 45 | CTG |
| | ATCTGGCGTAAAATTGACGCATG | | |
| 379 | CCCTGCAAAGATAATCATATTGTGACGTACGTTAAAGATA | 5, 6 | TGC |
| | ATCATGCGTAAAATTGACGCATG | | |
| 380 | CCCTAGAAAGATAATCATATTGTGACGTACGTTAAAGATA | 58, 60 | AGA |
| | ATCATGCGTAAAATTGAAGAATG | | |
| 381 | CCCTAGAAAGATAATCATATTGTGACGTACGTTAAAGATG | 40, 41 | TGC |
| | CTCATGCGTAAAATTGACGCATG | | |
| 382 | CCCTAGAAAGATAATCATATTGTGACGTACGTTAAAGATA | 47, 48 | GAT |
| | ATCATGATTAAAATTGACGCATG | | |
| 383 | CCCTAGAAAGATAATCATATTGTGACGTACGTTAAAGGGG | 38, 39, 40 | GGG |
| | ATCATGCGTAAAATTGACGCATG | | |
| 384 | CCCTAGAAAGATAATCATATTGTGACGTACGTTAAAGATA | 47, 48, 49 | GCA |
| | ATCATGGCAAAAATTGACGCATG | | |
| 385 | CCCTAGAAAGATAATCATATTGCCTCGTACGTTAAAGATA | 23, 24, 25 | CCT |
| | ATCATGCGTAAAATTGACGCATG | | |
| 386 | CCCCAGAAAGATAATCATATTGTGACGTACGTTAAAGATA | 4 | CCC |
| | ATCATGCGTAAAATTGACGCATG | | |
| 387 | CCCTAGAAAGATAATCATATTGTGACGTACGTTAAAGATA | 61, 63 | GTC |
| | ATCATGCGTAAAATTGACGCGTC | | |
| 388 | CCCTAGAAAGATAATCTATTTGTGACGTACGTTAAAGATA | 17, 18, 19 | TAT |
| | ATCATGCGTAAAATTGACGCATG | | |
| 389 | CCCTAGAAAGATAATCATATTGTGACGTACGCATAAGATA | 32, 33, 34 | CAT |
| | ATCATGCGTAAAATTGACGCATG | | |
| 390 | CCCTCGAAAGATAATCATATTGTGACGTACGTTAAAGATA | 5 | CTC |
| | ATCATGCGTAAAATTGACGCATG | | |
| 391 | CCCTAGTGAGATAATCATATTGTGACGTACGTTAAAGATA | 7, 8 | GTG |
| | ATCATGCGTAAAATTGACGCATG | | |
| 392 | CCCTAGAAAGATAATCATATTGTGACGTACGTTAAAGATA | 44, 46 | CTT |
| | ATCCTTCGTAAAATTGACGCATG | | |
| 393 | CCCTAGAAAGATAATCATAGTTTGACGTACGTTAAAGATA | 20, 22 | GTT |
| | ATCATGCGTAAAATTGACGCATG | | |
| 394 | CCCTAGAAAGATAATCCTATTGTGACGTACGTTAAAGATA | 17 | TCC |
| | ATCATGCGTAAAATTGACGCATG | | |
| 395 | CCCTAGAAAGATAATCATATTGTGACGTACGTTAAAGATA | 47, 48, 49 | ATG |
| | ATCATGATGAAAATTGACGCATG | | |
| 396 | CCCTAGAAAGATAATCATATTGTGACGCAGGTTAAAGATA | 28, 30 | CAG |
| | ATCATGCGTAAAATTGACGCATG | | |
| 397 | CCCTAGAAAGATAATCTTATTGTGACGTACGTTAAAGATA | 17 | TCT |
| | ATCATGCGTAAAATTGACGCATG | | |
| 398 | CCCTAGAAAGATAATCTTTTTGTGACGTACGTTAAAGATA | 17, 19 | TTT |
| | ATCATGCGTAAAATTGACGCATG | | |
| 399 | CCCTAGAAAGATAATCATATTGTGACGTTTCTTAAAGATA | 29, 30, 31 | TTC |
| | ATCATGCGTAAAATTGACGCATG | | |
| 400 | CCCTAGAAAGATAATCATATTGTCATGTACGTTAAAGATA | 24, 26 | CAT |
| | ATCATGCGTAAAATTGACGCATG | | |
| 401 | CCCAAGAAAGATAATCATATTGTGACGTACGTTAAAGATA | 4 | CCA |
| | ATCATGCGTAAAATTGACGCATG | | |
| 402 | CCCTAGAAAGATAATCATATTGTGACGTACGTTAAAGATA | 41, 43 | CTT |
| | CTTATGCGTAAAATTGACGCATG | | |
| 403 | CCCTAGAAAGATAATCATTAGGTGACGTACGTTAAAGATA | 19, 20, 21 | TAG |
| | ATCATGCGTAAAATTGACGCATG | | |
| 404 | CCCTAGAAAGATAATCAATGTGTGACGTACGTTAAAGATA | 18, 19, 20 | ATG |
| | ATCATGCGTAAAATTGACGCATG | | |
| 405 | CCCTAGAAAGATAATCATATTGTGACGTACGTTAAAGATA | 53, 55 | TTA |
| | ATCATGCGTAAATTAGACGCATG | | |
| 406 | CCGTAGAAAGATAATCATATTGTGACGTACGTTAAAGATA | 3 | CCG |
| | ATCATGCGTAAAATTGACGCATG | | |
| 407 | CCCTTTAAAGATAATCATATTGTGACGTACGTTAAAGATA | 5, 6 | TTT |
| | ATCATGCGTAAAATTGACGCATG | | |
| 408 | CCCTAGATGGATAATCATATTGTGACGTACGTTAAAGATA | 8, 9 | ATG |
| | ATCATGCGTAAAATTGACGCATG | | |
| 409 | CCCTAGAAAGATAATCACTCTGTGACGTACGTTAAAGATA | 18, 19, 20 | CTC |
| | ATCATGCGTAAAATTGACGCATG | | |
| 410 | CCCTAGAAAGATAATCATATTGTGACGTACGTTAAAGATA | 41, 42, 43 | TAG |
| | TAGATGCGTAAAATTGACGCATG | | |
| 411 | CCCTAGAAAGATAATCATATTGTGACGTACGTTAAGTGTA | 36, 37, 38 | GTG |
| | ATCATGCGTAAAATTGACGCATG | | |
| 412 | CCCTAGAAAGATAATCATGCTGTGACGTACGTTAAAGATA | 19, 20 | TGC |
| | ATCATGCGTAAAATTGACGCATG | | |
| 413 | CCCTAGAAAGATAATCATATTGTTGAGTACGTTAAAGATA | 24, 25, 26 | TGA |
| | ATCATGCGTAAAATTGACGCATG | | |
| 414 | CCCTACCAAGATAATCATATTGTGACGTACGTTAAAGATA | 6, 7 | ACC |
| | ATCATGCGTAAAATTGACGCATG | | |
| 415 | CCCTAGAAAGATAATCATAGGGTGACGTACGTTAAAGATA | 20, 21 | AGG |
| | ATCATGCGTAAAATTGACGCATG | | |
| 416 | CCCTAGAAAGATAATCATCAGGTGACGTACGTTAAAGATA | 19, 20, 21 | CAG |
| | ATCATGCGTAAAATTGACGCATG | | |
| 417 | CCCTAGTCGGATAATCATATTGTGACGTACGTTAAAGATA | 7, 8, 9 | TCG |
| | ATCATGCGTAAAATTGACGCATG | | |
| 418 | CCTTAGAAAGATAATCATATTGTGACGTACGTTAAAGATA | 3 | CCT |
| | ATCATGCGTAAAATTGACGCATG | | |
| 419 | CCCTACATAGATAATCATATTGTGACGTACGTTAAAGATA | 6, 8 | CAT |
| | ATCATGCGTAAAATTGACGCATG | | |
| 420 | CCCTAGAAAGATAAACATATTGTGACGTACGTTAAAGATA | 15 | AAA |
| | ATCATGCGTAAAATTGACGCATG | | |
| 421 | CCCTAGAAAGATAATCATATGTAGACGTACGTTAAAGATA | 21, 22, 23 | GTA |
| | ATCATGCGTAAAATTGACGCATG | | |
| 422 | CCCTAGAAAGATAATCTAATTGTGACGTACGTTAAAGATA | 17, 18 | CTA |
| | ATCATGCGTAAAATTGACGCATG | | |
| 423 | CCCTAGCTAGATAATCATATTGTGACGTACGTTAAAGATA | 7, 8 | GCT |
| | ATCATGCGTAAAATTGACGCATG | | |
| 424 | CCCTAGAAAGATTATCATATTGTGACGTACGTTAAAGATA | 13 | ATT |
| | ATCATGCGTAAAATTGACGCATG | | |
| 425 | CCCTAGAAAGATAATTATATTGTGACGTACGTTAAAGATA | 16 | ATT |
| | ATCATGCGTAAAATTGACGCATG | | |
| 426 | CCCTAGAAAGTTAATCATATTGTGACGTACGTTAAAGATA | 11 | AGT |
| | ATCATGCGTAAAATTGACGCATG | | |
| 427 | CCCTAGCGGGATAATCATATTGTGACGTACGTTAAAGATA | 7, 8, 9 | CGG |
| | ATCATGCGTAAAATTGACGCATG | | |
| 428 | CCCTAGAAAGGTAATCATATTGTGACGTACGTTAAAGATA | 11 | AGG |
| | ATCATGCGTAAAATTGACGCATG | | |
| 429 | CACCAGAAAGATAATCATATTGTGACGTACGTTAAAGATA | 2, 4 | ACC |
| | ATCATGCGTAAAATTGACGCATG | | |
| 430 | CCCTAGAAAGATATTCATATTGTGACGTACGTTAAAGATA | 14 | TAT |
| | ATCATGCGTAAAATTGACGCATG | | |
| 431 | CCCTAGAAAGATAATGCTATTGTGACGTACGTTAAAGATA | 16, 17 | TGC |
| | ATCATGCGTAAAATTGACGCATG | | |
| 432 | CGCTAGAAAGATAATCATATTGTGACGTACGTTAAAGATA | 2 | CGC |
| | ATCATGCGTAAAATTGACGCATG | | |
| 433 | CCCTAGAAAGATAATGATATTGTGACGTACGTTAAAGATA | 16 | ATG |
| | ATCATGCGTAAAATTGACGCATG | | |
| 434 | CCCTAGAAAGCTAATCATATTGTGACGTACGTTAAAGATA | 11 | AGC |
| | ATCATGCGTAAAATTGACGCATG | | |
| 435 | CCCTAGACTCATAATCATATTGTGACGTACGTTAAAGATA | 8, 9, 10 | CTC |
| | ATCATGCGTAAAATTGACGCATG | | |
| 436 | CCCTAGAAAGATAATCATATTGTGACGTACGTTAACCTTA | 36, 37, 38 | CCT |
| | ATCATGCGTAAAATTGACGCATG | | |
| 437 | CCCTAGAAAGATAATCATATTGTGACGTACGTTACCTATA | 35, 36, 37 | CCT |
| | ATCATGCGTAAAATTGACGCATG | | |
| 438 | CCCTAGAAAGATAATCCAATTGTGACGTACGTTAAAGATA | 17, 18 | CCA |
| | ATCATGCGTAAAATTGACGCATG | | |
| 439 | CCCTAGAAAGAGAATCATATTGTGACGTACGTTAAAGATA | 12 | GAG |
| | ATCATGCGTAAAATTGACGCATG | | |
| 440 | CCCTAGAAAGATAATCATATTGTGACGTACGTTAAAGATC | 40, 41, 42 | CGA |
| | GACATGCGTAAAATTGACGCATG | | |
| 441 | CCCTAGAAAGATGATCATATTGTGACGTACGTTAAAGATA | 13 | ATG |
| | ATCATGCGTAAAATTGACGCATG | | |
| 442 | CCCTAGAAAGGGAATCATATTGTGACGTACGTTAAAGATA | 11, 12 | GGG |
| | ATCATGCGTAAAATTGACGCATG | | |
| 443 | CCCTAGAAAGTGAATCATATTGTGACGTACGTTAAAGATA | 11, 12 | GTG |
| | ATCATGCGTAAAATTGACGCATG | | |
| 444 | CCCTAGAGATATAATCATATTGTGACGTACGTTAAAGATA | 8, 10 | GAT |
| | ATCATGCGTAAAATTGACGCATG | | |
| 445 | CCCTAGAAGTATAATCATATTGTGACGTACGTTAAAGATA | 9, 10 | AGT |
| | ATCATGCGTAAAATTGACGCATG | | |
| 446 | CCCTAGAAAGATAAATATATTGTGACGTACGTTAAAGATA | 15, 16 | AAT |
| | ATCATGCGTAAAATTGACGCATG | | |
| 447 | CCCTAGAAAGAGTATCATATTGTGACGTACGTTAAAGATA | 12, 13 | AGT |
| | ATCATGCGTAAAATTGACGCATG | | |
| 448 | CCCTAGAAAGATAGTCATATTGTGACGTACGTTAAAGATA | 14 | TAG |
| | ATCATGCGTAAAATTGACGCATG | | |
| 449 | CCCTAGAAAGATGAACATATTGTGACGTACGTTAAAGATA | 13, 15 | GAA |
| | ATCATGCGTAAAATTGACGCATG | | |
| 450 | CCCTAGAAAGAGATTCATATTGTGACGTACGTTAAAGATA | 12, 14 | GAT |
| | ATCATGCGTAAAATTGACGCATG | | |
| 451 | CCCTAGAAAGATAAGCATATTGTGACGTACGTTAAAGATA | 15 | AAG |
| | ATCATGCGTAAAATTGACGCATG | | |

In some aspects, the second ITR comprises the nucleic acid sequence of SEQ ID NO: 267. In some aspects, the second ITR comprises the nucleic acid sequence of SEQ ID NO: 268. In some aspects, the second ITR comprises the nucleic acid sequence of SEQ ID NO: 269. In some aspects, the second ITR comprises the nucleic acid sequence of SEQ ID NO: 270. In some aspects, the second ITR comprises the nucleic acid sequence of SEQ ID NO: 271. In some aspects, the second ITR comprises the nucleic acid sequence of SEQ ID NO: 272. In some aspects, the second ITR comprises the nucleic acid sequence of SEQ ID NO: 273. In some aspects, the second ITR comprises the nucleic acid sequence of SEQ ID NO: 274. In some aspects, the second ITR comprises the nucleic acid sequence of SEQ ID NO: 275. In some aspects, the second ITR comprises the nucleic acid sequence of SEQ ID NO: 276. In some aspects, the second ITR comprises the nucleic acid sequence of SEQ ID NO: 277. In some aspects, the second ITR comprises the nucleic acid sequence of SEQ ID NO: 279. In some aspects, the second ITR comprises the nucleic acid sequence of SEQ ID NO: 280. In some aspects, the second ITR comprises the nucleic acid sequence of SEQ ID NO: 281. In some aspects, the second ITR comprises the nucleic acid sequence of SEQ ID NO: 282. In some aspects, the second ITR comprises the nucleic acid sequence of SEQ ID NO: 283. In some aspects, the second ITR comprises the nucleic acid sequence of SEQ ID NO: 284. In some aspects, the second ITR comprises the nucleic acid sequence of SEQ ID NO: 285. In some aspects, the second ITR comprises the nucleic acid sequence of SEQ ID NO: 286. In some aspects, the second ITR comprises the nucleic acid sequence of SEQ ID NO: 287. In some aspects, the second ITR comprises the nucleic acid sequence of SEQ ID NO: 288. In some aspects, the second ITR comprises the nucleic acid sequence of SEQ ID NO: 289. In some aspects, the second ITR comprises the nucleic acid sequence of SEQ ID NO: 290. In some aspects, the second ITR comprises the nucleic acid sequence of SEQ ID NO: 291. In some aspects, the second ITR comprises the nucleic acid sequence of SEQ ID NO: 292. In some aspects, the second ITR comprises the nucleic acid sequence of SEQ ID NO: 293. In some aspects, the second ITR comprises the nucleic acid sequence of SEQ ID NO: 294. In some aspects, the second ITR comprises the nucleic acid sequence of SEQ ID NO: 295. In some aspects, the second ITR comprises the nucleic acid sequence of SEQ ID NO: 296. In some aspects, the second ITR comprises the nucleic acid sequence of SEQ ID NO: 297. In some aspects, the second ITR comprises the nucleic acid sequence of SEQ ID NO: 298. In some aspects, the second ITR comprises the nucleic acid sequence of SEQ ID NO: 299. In some aspects, the second ITR comprises the nucleic acid sequence of SEQ ID NO: 300. In some aspects, the second ITR comprises the nucleic acid sequence of SEQ ID NO: 301. In some aspects, the second ITR comprises the nucleic acid sequence of SEQ ID NO: 302. In some aspects, the second ITR comprises the nucleic acid sequence of SEQ ID NO: 303. In some aspects, the second ITR comprises the nucleic acid sequence of SEQ ID NO: 304. In some aspects, the second ITR comprises the nucleic acid sequence of SEQ ID NO: 305. In some aspects, the second ITR comprises the nucleic acid sequence of SEQ ID NO: 306. In some aspects, the second ITR comprises the nucleic acid sequence of SEQ ID NO: 307. In some aspects, the second ITR comprises the nucleic acid sequence of SEQ ID NO: 308. In some aspects, the second ITR comprises the nucleic acid sequence of SEQ ID NO: 309. In some aspects, the second ITR comprises the nucleic acid sequence of SEQ ID NO: 310. In some aspects, the second ITR comprises the nucleic acid sequence of SEQ ID NO: 311. In some aspects, the second ITR comprises the nucleic acid sequence of SEQ ID NO: 312. In some aspects, the second ITR comprises the nucleic acid sequence of SEQ ID NO: 313. In some aspects, the second ITR comprises the nucleic acid sequence of SEQ ID NO: 314. In some aspects, the second ITR comprises the nucleic acid sequence of SEQ ID NO: 315. In some aspects, the second ITR comprises the nucleic acid sequence of SEQ ID NO: 316. In some aspects, the second ITR comprises the nucleic acid sequence of SEQ ID NO: 317. In some aspects, the second ITR comprises the nucleic acid sequence of SEQ ID NO: 318. In some aspects, the second ITR comprises the nucleic acid sequence of SEQ ID NO: 319. In some aspects, the second ITR comprises the nucleic acid sequence of SEQ ID NO: 320. In some aspects, the second ITR comprises the nucleic acid sequence of SEQ ID NO: 321. In some aspects, the second ITR comprises the nucleic acid sequence of SEQ ID NO: 322. In some aspects, the second ITR comprises the nucleic acid sequence of SEQ ID NO: 323. In some aspects, the second ITR comprises the nucleic acid sequence of SEQ ID NO: 324. In some aspects, the second ITR comprises the nucleic acid sequence of SEQ ID NO: 325. In some aspects, the second ITR comprises the nucleic acid sequence of SEQ ID NO: 326. In some aspects, the second ITR comprises the nucleic acid sequence of SEQ ID NO: 327. In some aspects, the second ITR comprises the nucleic acid sequence of SEQ ID NO: 328. In some aspects, the second ITR comprises the nucleic acid sequence of SEQ ID NO: 329. In some aspects, the second ITR comprises the nucleic acid sequence of SEQ ID NO: 330. In some aspects, the second ITR comprises the nucleic acid sequence of SEQ ID NO: 331. In some aspects, the second ITR comprises the nucleic acid sequence of SEQ ID NO: 332. In some aspects, the second ITR comprises the nucleic acid sequence of SEQ ID NO: 333. In some aspects, the second ITR comprises the nucleic acid sequence of SEQ ID NO: 334. In some aspects, the second ITR comprises the nucleic acid sequence of SEQ ID NO: 335. In some aspects, the second ITR comprises the nucleic acid sequence of SEQ ID NO: 336. In some aspects, the second ITR comprises the nucleic acid sequence of SEQ ID NO: 337. In some aspects, the second ITR comprises the nucleic acid sequence of SEQ ID NO: 338. In some aspects, the second ITR comprises the nucleic acid sequence of SEQ ID NO: 339. In some aspects, the second ITR comprises the nucleic acid sequence of SEQ ID NO: 340. In some aspects, the second ITR comprises the nucleic acid sequence of SEQ ID NO: 341. In some aspects, the second ITR comprises the nucleic acid sequence of SEQ ID NO: 342. In some aspects, the second ITR comprises the nucleic acid sequence of SEQ ID NO: 343. In some aspects, the second ITR comprises the nucleic acid sequence of SEQ ID NO: 344. In some aspects, the second ITR comprises the nucleic acid sequence of SEQ ID NO: 345. In some aspects, the second ITR comprises the nucleic acid sequence of SEQ ID NO: 346. In some aspects, the second ITR comprises the nucleic acid sequence of SEQ ID NO: 347. In some aspects, the second ITR comprises the nucleic acid sequence of SEQ ID NO: 348. In some aspects, the second ITR comprises the nucleic acid sequence of SEQ ID NO: 349. In some aspects, the second ITR comprises the nucleic acid sequence of SEQ ID NO: 350. In some aspects, the second ITR comprises the nucleic acid sequence of SEQ ID NO: 351. In some aspects, the second ITR comprises the nucleic acid sequence of SEQ ID NO: 352. In some aspects, the second ITR comprises the nucleic acid sequence of SEQ ID NO: 353. In some aspects, the second ITR comprises the nucleic acid sequence of SEQ ID NO: 354. In some aspects, the second ITR comprises the nucleic acid sequence of SEQ ID NO: 355. In some aspects, the second ITR comprises the nucleic acid sequence of SEQ ID NO: 356. In some aspects, the second ITR comprises the nucleic acid sequence of SEQ ID NO: 357. In some aspects, the second ITR comprises the nucleic acid sequence of SEQ ID NO: 358. In some aspects, the second ITR comprises the nucleic acid sequence of SEQ ID NO: 359. In some aspects, the second ITR comprises the nucleic acid sequence of SEQ ID NO: 360. In some aspects, the second ITR comprises the nucleic acid sequence of SEQ ID NO: 361. In some aspects, the second ITR comprises the nucleic acid sequence of SEQ ID NO: 362. In some aspects, the second ITR comprises the nucleic acid sequence of SEQ ID NO: 363. In some aspects, the second ITR comprises the nucleic acid sequence of SEQ ID NO: 364. In some aspects, the second ITR comprises the nucleic acid sequence of SEQ ID NO: 365. In some aspects, the second ITR comprises the nucleic acid sequence of SEQ ID NO: 366. In some aspects, the second ITR comprises the nucleic acid sequence of SEQ ID NO: 367. In some aspects, the second ITR comprises the nucleic acid sequence of SEQ ID NO: 368. In some aspects, the second ITR comprises the nucleic acid sequence of SEQ ID NO: 369. In some aspects, the second ITR comprises the nucleic acid sequence of SEQ ID NO: 370. In some aspects, the second ITR comprises the nucleic acid sequence of SEQ ID NO: 371. In some aspects, the second ITR comprises the nucleic acid sequence of SEQ ID NO: 372. In some aspects, the second ITR comprises the nucleic acid sequence of SEQ ID NO: 373. In some aspects, the second ITR comprises the nucleic acid sequence of SEQ ID NO: 374. In some aspects, the second ITR comprises the nucleic acid sequence of SEQ ID NO: 375. In some aspects, the second ITR comprises the nucleic acid sequence of SEQ ID NO: 376. In some aspects, the second ITR comprises the nucleic acid sequence of SEQ ID NO: 377. In some aspects, the second ITR comprises the nucleic acid sequence of SEQ ID NO: 378. In some aspects, the second ITR comprises the nucleic acid sequence of SEQ ID NO: 379. In some aspects, the second ITR comprises the nucleic acid sequence of SEQ ID NO: 380. In some aspects, the second ITR comprises the nucleic acid sequence of SEQ ID NO: 381. In some aspects, the second ITR comprises the nucleic acid sequence of SEQ ID NO: 382. In some aspects, the second ITR comprises the nucleic acid sequence of SEQ ID NO: 383. In some aspects, the second ITR comprises the nucleic acid sequence of SEQ ID NO: 384. In some aspects, the second ITR comprises the nucleic acid sequence of SEQ ID NO: 385. In some aspects, the second ITR comprises the nucleic acid sequence of SEQ ID NO: 386. In some aspects, the second ITR comprises the nucleic acid sequence of SEQ ID NO: 387. In some aspects, the second ITR comprises the nucleic acid sequence of SEQ ID NO: 388. In some aspects, the second ITR comprises the nucleic acid sequence of SEQ ID NO: 389. In some aspects, the second ITR comprises the nucleic acid sequence of SEQ ID NO: 390. In some aspects, the second ITR comprises the nucleic acid sequence of SEQ ID NO: 391. In some aspects, the second ITR comprises the nucleic acid sequence of SEQ ID NO: 392. In some aspects, the second ITR comprises the nucleic acid sequence of SEQ ID NO: 393. In some aspects, the second ITR comprises the nucleic acid sequence of SEQ ID NO: 394. In some aspects, the second ITR comprises the nucleic acid sequence of SEQ ID NO: 395. In some aspects, the second ITR comprises the nucleic acid sequence of SEQ ID NO: 396. In some aspects, the second ITR comprises the nucleic acid sequence of SEQ ID NO: 397. In some aspects, the second ITR comprises the nucleic acid sequence of SEQ ID NO: 398. In some aspects, the second ITR comprises the nucleic acid sequence of SEQ ID NO: 399. In some aspects, the second ITR comprises the nucleic acid sequence of SEQ ID NO: 400. In some aspects, the second ITR comprises the nucleic acid sequence of SEQ ID NO: 401. In some aspects, the second ITR comprises the nucleic acid sequence of SEQ ID NO: 402. In some aspects, the second ITR comprises the nucleic acid sequence of SEQ ID NO: 403. In some aspects, the second ITR comprises the nucleic acid sequence of SEQ ID NO: 404. In some aspects, the second ITR comprises the nucleic acid sequence of SEQ ID NO: 405. In some aspects, the second ITR comprises the nucleic acid sequence of SEQ ID NO: 406. In some aspects, the second ITR comprises the nucleic acid sequence of SEQ ID NO: 407. In some aspects, the second ITR comprises the nucleic acid sequence of SEQ ID NO: 408. In some aspects, the second ITR comprises the nucleic acid sequence of SEQ ID NO: 409. In some aspects, the second ITR comprises the nucleic acid sequence of SEQ ID NO: 410. In some aspects, the second ITR comprises the nucleic acid sequence of SEQ ID NO: 411. In some aspects, the second ITR comprises the nucleic acid sequence of SEQ ID NO: 412. In some aspects, the second ITR comprises the nucleic acid sequence of SEQ ID NO: 413. In some aspects, the second ITR comprises the nucleic acid sequence of SEQ ID NO: 414. In some aspects, the second ITR comprises the nucleic acid sequence of SEQ ID NO: 415. In some aspects, the second ITR comprises the nucleic acid sequence of SEQ ID NO: 416. In some aspects, the second ITR comprises the nucleic acid sequence of SEQ ID NO: 417. In some aspects, the second ITR comprises the nucleic acid sequence of SEQ ID NO: 418. In some aspects, the second ITR comprises the nucleic acid sequence of SEQ ID NO: 419. In some aspects, the second ITR comprises the nucleic acid sequence of SEQ ID NO: 420. In some aspects, the second ITR comprises the nucleic acid sequence of SEQ ID NO: 421. In some aspects, the second ITR comprises the nucleic acid sequence of SEQ ID NO: 422. In some aspects, the second ITR comprises the nucleic acid sequence of SEQ ID NO: 423. In some aspects, the second ITR comprises the nucleic acid sequence of SEQ ID NO: 424. In some aspects, the second ITR comprises the nucleic acid sequence of SEQ ID NO: 425. In some aspects, the second ITR comprises the nucleic acid sequence of SEQ ID NO: 426. In some aspects, the second ITR comprises the nucleic acid sequence of SEQ ID NO: 427. In some aspects, the second ITR comprises the nucleic acid sequence of SEQ ID NO: 428. In some aspects, the second ITR comprises the nucleic acid sequence of SEQ ID NO: 429. In some aspects, the second ITR comprises the nucleic acid sequence of SEQ ID NO: 430. In some aspects, the second ITR comprises the nucleic acid sequence of SEQ ID NO: 431. In some aspects, the second ITR comprises the nucleic acid sequence of SEQ ID NO: 432. In some aspects, the second ITR comprises the nucleic acid sequence of SEQ ID NO: 433. In some aspects, the second ITR comprises the nucleic acid sequence of SEQ ID NO: 434. In some aspects, the second ITR comprises the nucleic acid sequence of SEQ ID NO: 435. In some aspects, the second ITR comprises the nucleic acid sequence of SEQ ID NO: 436. In some aspects, the second ITR comprises the nucleic acid sequence of SEQ ID NO: 437. In some aspects, the second ITR comprises the nucleic acid sequence of SEQ ID NO: 438. In some aspects, the second ITR comprises the nucleic acid sequence of SEQ ID NO: 439. In some aspects, the second ITR comprises the nucleic acid sequence of SEQ ID NO: 440. In some aspects, the second ITR comprises the nucleic acid sequence of SEQ ID NO: 441. In some aspects, the second ITR comprises the nucleic acid sequence of SEQ ID NO: 442. In some aspects, the second ITR comprises the nucleic acid sequence of SEQ ID NO: 443. In some aspects, the second ITR comprises the nucleic acid sequence of SEQ ID NO: 444. In some aspects, the second ITR comprises the nucleic acid sequence of SEQ ID NO: 445. In some aspects, the second ITR comprises the nucleic acid sequence of SEQ ID NO: 446. In some aspects, the second ITR comprises the nucleic acid sequence of SEQ ID NO: 447. In some aspects, the second ITR comprises the nucleic acid sequence of SEQ ID NO: 448. In some aspects, the second ITR comprises the nucleic acid sequence of SEQ ID NO: 449. In some aspects, the second ITR comprises the nucleic acid sequence of SEQ ID NO: 450. In some aspects, the second ITR comprises the nucleic acid sequence of SEQ ID NO: 451. In an aspect, the first sequence encoding a first ITR comprises the nucleic acid sequence of SEQ ID NOS: 4, 14-16, 160-163 or 165-265 and the second sequence encoding a second ITR comprises the nucleic acid sequence of SEQ ID NOS: 5, 11, 267-277, 279-329 or 331-451.

In an aspect, the first sequence encoding a first ITR comprises the nucleic acid sequence of SEQ ID NO: 4 and the second sequence encoding a second ITR comprises the nucleic acid sequence of SEQ ID NO: 5. In an aspect, the first sequence encoding a first ITR comprises the nucleic acid sequence of SEQ ID NO: 14 and the second sequence encoding a second ITR comprises the nucleic acid sequence of SEQ ID NO: 5. In an aspect, the first sequence encoding a first ITR comprises the nucleic acid sequence of SEQ ID NO: 15 and the second sequence encoding a second ITR comprises the nucleic acid sequence of SEQ ID NO: 5. In an aspect, the first sequence encoding a first ITR comprises the nucleic acid sequence of SEQ ID NO: 16 and the second sequence encoding a second ITR comprises the nucleic acid sequence of SEQ ID NO: 5. In an aspect, the first sequence encoding a first ITR comprises the nucleic acid sequence of SEQ ID NO: 160 and the second sequence encoding a second ITR comprises the nucleic acid sequence of SEQ ID NO: 5. In an aspect, the first sequence encoding a first ITR comprises the nucleic acid sequence of SEQ ID NO: 161 and the second sequence encoding a second ITR comprises the nucleic acid sequence of SEQ ID NO: 5. In an aspect, the first sequence encoding a first ITR comprises the nucleic acid sequence of SEQ ID NO: 162 and the second sequence encoding a second ITR comprises the nucleic acid sequence of SEQ ID NO: 5. In an aspect, the first sequence encoding a first ITR comprises the nucleic acid sequence of SEQ ID NO: 163 and the second sequence encoding a second ITR comprises the nucleic acid sequence of SEQ ID NO: 5. In an aspect, the first sequence encoding a first ITR comprises the nucleic acid sequence of SEQ ID NO: 165 and the second sequence encoding a second ITR comprises the nucleic acid sequence of SEQ ID NO: 5. In an aspect, the first sequence encoding a first ITR comprises the nucleic acid sequence of SEQ ID NO: 166 and the second sequence encoding a second ITR comprises the nucleic acid sequence of SEQ ID NO: 5. In an aspect, the first sequence encoding a first ITR comprises the nucleic acid sequence of SEQ ID NO: 167 and the second sequence encoding a second ITR comprises the nucleic acid sequence of SEQ ID NO: 5. In an aspect, the first sequence encoding a first ITR comprises the nucleic acid sequence of SEQ ID NO: 168 and the second sequence encoding a second ITR comprises the nucleic acid sequence of SEQ ID NO: 5. In an aspect, the first sequence encoding a first ITR comprises the nucleic acid sequence of SEQ ID NO: 169 and the second sequence encoding a second ITR comprises the nucleic acid sequence of SEQ ID NO: 5. In an aspect, the first sequence encoding a first ITR comprises the nucleic acid sequence of SEQ ID NO: 170 and the second sequence encoding a second ITR comprises the nucleic acid sequence of SEQ ID NO: 5. In an aspect, the first sequence encoding a first ITR comprises the nucleic acid sequence of SEQ ID NO: 171 and the second sequence encoding a second ITR comprises the nucleic acid sequence of SEQ ID NO: 5. In an aspect, the first sequence encoding a first ITR comprises the nucleic acid sequence of SEQ ID NO: 172 and the second sequence encoding a second ITR comprises the nucleic acid sequence of SEQ ID NO: 5. In an aspect, the first sequence encoding a first ITR comprises the nucleic acid sequence of SEQ ID NO: 173 and the second sequence encoding a second ITR comprises the nucleic acid sequence of SEQ ID NO: 5. In an aspect, the first sequence encoding a first ITR comprises the nucleic acid sequence of SEQ ID NO: 174 and the second sequence encoding a second ITR comprises the nucleic acid sequence of SEQ ID NO: 5. In an aspect, the first sequence encoding a first ITR comprises the nucleic acid sequence of SEQ ID NO: 175 and the second sequence encoding a second ITR comprises the nucleic acid sequence of SEQ ID NO: 5. In an aspect, the first sequence encoding a first ITR comprises the nucleic acid sequence of SEQ ID NO: 176 and the second sequence encoding a second ITR comprises the nucleic acid sequence of SEQ ID NO: 5. In an aspect, the first sequence encoding a first ITR comprises the nucleic acid sequence of SEQ ID NO: 177 and the second sequence encoding a second ITR comprises the nucleic acid sequence of SEQ ID NO: 5. In an aspect, the first sequence encoding a first ITR comprises the nucleic acid sequence of SEQ ID NO: 178 and the second sequence encoding a second ITR comprises the nucleic acid sequence of SEQ ID NO: 5. In an aspect, the first sequence encoding a first ITR comprises the nucleic acid sequence of SEQ ID NO: 179 and the second sequence encoding a second ITR comprises the nucleic acid sequence of SEQ ID NO: 5. In an aspect, the first sequence encoding a first ITR comprises the nucleic acid sequence of SEQ ID NO: 180 and the second sequence encoding a second ITR comprises the nucleic acid sequence of SEQ ID NO: 5. In an aspect, the first sequence encoding a first ITR comprises the nucleic acid sequence of SEQ ID NO: 181 and the second sequence encoding a second ITR comprises the nucleic acid sequence of SEQ ID NO: 5. In an aspect, the first sequence encoding a first ITR comprises the nucleic acid sequence of SEQ ID NO: 182 and the second sequence encoding a second ITR comprises the nucleic acid sequence of SEQ ID NO: 5. In an aspect, the first sequence encoding a first ITR comprises the nucleic acid sequence of SEQ ID NO: 183 and the second sequence encoding a second ITR comprises the nucleic acid sequence of SEQ ID NO: 5. In an aspect, the first sequence encoding a first ITR comprises the nucleic acid sequence of SEQ ID NO: 184 and the second sequence encoding a second ITR comprises the nucleic acid sequence of SEQ ID NO: 5. In an aspect, the first sequence encoding a first ITR comprises the nucleic acid sequence of SEQ ID NO: 185 and the second sequence encoding a second ITR comprises the nucleic acid sequence of SEQ ID NO: 5. In an aspect, the first sequence encoding a first ITR comprises the nucleic acid sequence of SEQ ID NO: 186 and the second sequence encoding a second ITR comprises the nucleic acid sequence of SEQ ID NO: 5. In an aspect, the first sequence encoding a first ITR comprises the nucleic acid sequence of SEQ ID NO: 187 and the second sequence encoding a second ITR comprises the nucleic acid sequence of SEQ ID NO: 5. In an aspect, the first sequence encoding a first ITR comprises the nucleic acid sequence of SEQ ID NO: 188 and the second sequence encoding a second ITR comprises the nucleic acid sequence of SEQ ID NO: 5. In an aspect, the first sequence encoding a first ITR comprises the nucleic acid sequence of SEQ ID NO: 189 and the second sequence encoding a second ITR comprises the nucleic acid sequence of SEQ ID NO: 5.

In an aspect, the first sequence encoding a first ITR comprises the nucleic acid sequence of SEQ ID NO: 4 and the second sequence encoding a second ITR comprises the nucleic acid sequence of SEQ ID NO: 11. In an aspect, the first sequence encoding a first ITR comprises the nucleic acid sequence of SEQ ID NO: 14 and the second sequence encoding a second ITR comprises the nucleic acid sequence of SEQ ID NO: 11. In an aspect, the first sequence encoding a first ITR comprises the nucleic acid sequence of SEQ ID NO: 15 and the second sequence encoding a second ITR comprises the nucleic acid sequence of SEQ ID NO: 11. In an aspect, the first sequence encoding a first ITR comprises the nucleic acid sequence of SEQ ID NO: 16 and the second sequence encoding a second ITR comprises the nucleic acid sequence of SEQ ID NO: 11. In an aspect, the first sequence encoding a first ITR comprises the nucleic acid sequence of SEQ ID NO: 160 and the second sequence encoding a second ITR comprises the nucleic acid sequence of SEQ ID NO: 11. In an aspect, the first sequence encoding a first ITR comprises the nucleic acid sequence of SEQ ID NO: 161 and the second sequence encoding a second ITR comprises the nucleic acid sequence of SEQ ID NO: 11. In an aspect, the first sequence encoding a first ITR comprises the nucleic acid sequence of SEQ ID NO: 162 and the second sequence encoding a second ITR comprises the nucleic acid sequence of SEQ ID NO: 11. In an aspect, the first sequence encoding a first ITR comprises the nucleic acid sequence of SEQ ID NO: 163 and the second sequence encoding a second ITR comprises the nucleic acid sequence of SEQ ID NO: 11. In an aspect, the first sequence encoding a first ITR comprises the nucleic acid sequence of SEQ ID NO: 165 and the second sequence encoding a second ITR comprises the nucleic acid sequence of SEQ ID NO: 11. In an aspect, the first sequence encoding a first ITR comprises the nucleic acid sequence of SEQ ID NO: 166 and the second sequence encoding a second ITR comprises the nucleic acid sequence of SEQ ID NO: 11. In an aspect, the first sequence encoding a first ITR comprises the nucleic acid sequence of SEQ ID NO: 167 and the second sequence encoding a second ITR comprises the nucleic acid sequence of SEQ ID NO: 11. In an aspect, the first sequence encoding a first ITR comprises the nucleic acid sequence of SEQ ID NO: 168 and the second sequence encoding a second ITR comprises the nucleic acid sequence of SEQ ID NO: 11. In an aspect, the first sequence encoding a first ITR comprises the nucleic acid sequence of SEQ ID NO: 169 and the second sequence encoding a second ITR comprises the nucleic acid sequence of SEQ ID NO: 11. In an aspect, the first sequence encoding a first ITR comprises the nucleic acid sequence of SEQ ID NO: 170 and the second sequence encoding a second ITR comprises the nucleic acid sequence of SEQ ID NO: 11. In an aspect, the first sequence encoding a first ITR comprises the nucleic acid sequence of SEQ ID NO: 171 and the second sequence encoding a second ITR comprises the nucleic acid sequence of SEQ ID NO: 11. In an aspect, the first sequence encoding a first ITR comprises the nucleic acid sequence of SEQ ID NO: 172 and the second sequence encoding a second ITR comprises the nucleic acid sequence of SEQ ID NO: 11. In an aspect, the first sequence encoding a first ITR comprises the nucleic acid sequence of SEQ ID NO: 173 and the second sequence encoding a second ITR comprises the nucleic acid sequence of SEQ ID NO: 11. In an aspect, the first sequence encoding a first ITR comprises the nucleic acid sequence of SEQ ID NO: 174 and the second sequence encoding a second ITR comprises the nucleic acid sequence of SEQ ID NO: 11. In an aspect, the first sequence encoding a first ITR comprises the nucleic acid sequence of SEQ ID NO: 175 and the second sequence encoding a second ITR comprises the nucleic acid sequence of SEQ ID NO: 11. In an aspect, the first sequence encoding a first ITR comprises the nucleic acid sequence of SEQ ID NO: 176 and the second sequence encoding a second ITR comprises the nucleic acid sequence of SEQ ID NO: 11. In an aspect, the first sequence encoding a first ITR comprises the nucleic acid sequence of SEQ ID NO: 177 and the second sequence encoding a second ITR comprises the nucleic acid sequence of SEQ ID NO: 11. In an aspect, the first sequence encoding a first ITR comprises the nucleic acid sequence of SEQ ID NO: 178 and the second sequence encoding a second ITR comprises the nucleic acid sequence of SEQ ID NO: 11. In an aspect, the first sequence encoding a first ITR comprises the nucleic acid sequence of SEQ ID NO: 179 and the second sequence encoding a second ITR comprises the nucleic acid sequence of SEQ ID NO: 11. In an aspect, the first sequence encoding a first ITR comprises the nucleic acid sequence of SEQ ID NO: 180 and the second sequence encoding a second ITR comprises the nucleic acid sequence of SEQ ID NO: 11. In an aspect, the first sequence encoding a first ITR comprises the nucleic acid sequence of SEQ ID NO: 181 and the second sequence encoding a second ITR comprises the nucleic acid sequence of SEQ ID NO: 11. In an aspect, the first sequence encoding a first ITR comprises the nucleic acid sequence of SEQ ID NO: 182 and the second sequence encoding a second ITR comprises the nucleic acid sequence of SEQ ID NO: 11. In an aspect, the first sequence encoding a first ITR comprises the nucleic acid sequence of SEQ ID NO: 183 and the second sequence encoding a second ITR comprises the nucleic acid sequence of SEQ ID NO: 11. In an aspect, the first sequence encoding a first ITR comprises the nucleic acid sequence of SEQ ID NO: 184 and the second sequence encoding a second ITR comprises the nucleic acid sequence of SEQ ID NO: 11. In an aspect, the first sequence encoding a first ITR comprises the nucleic acid sequence of SEQ ID NO: 185 and the second sequence encoding a second ITR comprises the nucleic acid sequence of SEQ ID NO: 11. In an aspect, the first sequence encoding a first ITR comprises the nucleic acid sequence of SEQ ID NO: 186 and the second sequence encoding a second ITR comprises the nucleic acid sequence of SEQ ID NO: 11. In an aspect, the first sequence encoding a first ITR comprises the nucleic acid sequence of SEQ ID NO: 187 and the second sequence encoding a second ITR comprises the nucleic acid sequence of SEQ ID NO: 11. In an aspect, the first sequence encoding a first ITR comprises the nucleic acid sequence of SEQ ID NO: 188 and the second sequence encoding a second ITR comprises the nucleic acid sequence of SEQ ID NO: 11. In an aspect, the first sequence encoding a first ITR comprises the nucleic acid sequence of SEQ ID NO: 189 and the second sequence encoding a second ITR comprises the nucleic acid sequence of SEQ ID NO: 11.

In an aspect, the first sequence encoding a first ITR comprises the nucleic acid sequence of SEQ ID NO: 4 and the second sequence encoding a second ITR comprises the nucleic acid sequence of SEQ ID NO: 267. In an aspect, the first sequence encoding a first ITR comprises the nucleic acid sequence of SEQ ID NO: 14 and the second sequence encoding a second ITR comprises the nucleic acid sequence of SEQ ID NO: 267. In an aspect, the first sequence encoding a first ITR comprises the nucleic acid sequence of SEQ ID NO: 15 and the second sequence encoding a second ITR comprises the nucleic acid sequence of SEQ ID NO: 267. In an aspect, the first sequence encoding a first ITR comprises the nucleic acid sequence of SEQ ID NO: 16 and the second sequence encoding a second ITR comprises the nucleic acid sequence of SEQ ID NO: 267. In an aspect, the first sequence encoding a first ITR comprises the nucleic acid sequence of SEQ ID NO: 160 and the second sequence encoding a second ITR comprises the nucleic acid sequence of SEQ ID NO: 267. In an aspect, the first sequence encoding a first ITR comprises the nucleic acid sequence of SEQ ID NO: 161 and the second sequence encoding a second ITR comprises the nucleic acid sequence of SEQ ID NO: 267. In an aspect, the first sequence encoding a first ITR comprises the nucleic acid sequence of SEQ ID NO: 162 and the second sequence encoding a second ITR comprises the nucleic acid sequence of SEQ ID NO: 267. In an aspect, the first sequence encoding a first ITR comprises the nucleic acid sequence of SEQ ID NO: 163 and the second sequence encoding a second ITR comprises the nucleic acid sequence of SEQ ID NO: 267. In an aspect, the first sequence encoding a first ITR comprises the nucleic acid sequence of SEQ ID NO: 165 and the second sequence encoding a second ITR comprises the nucleic acid sequence of SEQ ID NO: 267. In an aspect, the first sequence encoding a first ITR comprises the nucleic acid sequence of SEQ ID NO: 166 and the second sequence encoding a second ITR comprises the nucleic acid sequence of SEQ ID NO: 267. In an aspect, the first sequence encoding a first ITR comprises the nucleic acid sequence of SEQ ID NO: 167 and the second sequence encoding a second ITR comprises the nucleic acid sequence of SEQ ID NO: 267. In an aspect, the first sequence encoding a first ITR comprises the nucleic acid sequence of SEQ ID NO: 168 and the second sequence encoding a second ITR comprises the nucleic acid sequence of SEQ ID NO: 267. In an aspect, the first sequence encoding a first ITR comprises the nucleic acid sequence of SEQ ID NO: 169 and the second sequence encoding a second ITR comprises the nucleic acid sequence of SEQ ID NO: 267. In an aspect, the first sequence encoding a first ITR comprises the nucleic acid sequence of SEQ ID NO: 170 and the second sequence encoding a second ITR comprises the nucleic acid sequence of SEQ ID NO: 267. In an aspect, the first sequence encoding a first ITR comprises the nucleic acid sequence of SEQ ID NO: 171 and the second sequence encoding a second ITR comprises the nucleic acid sequence of SEQ ID NO: 267. In an aspect, the first sequence encoding a first ITR comprises the nucleic acid sequence of SEQ ID NO: 172 and the second sequence encoding a second ITR comprises the nucleic acid sequence of SEQ ID NO: 267. In an aspect, the first sequence encoding a first ITR comprises the nucleic acid sequence of SEQ ID NO: 173 and the second sequence encoding a second ITR comprises the nucleic acid sequence of SEQ ID NO: 267. In an aspect, the first sequence encoding a first ITR comprises the nucleic acid sequence of SEQ ID NO: 174 and the second sequence encoding a second ITR comprises the nucleic acid sequence of SEQ ID NO: 267. In an aspect, the first sequence encoding a first ITR comprises the nucleic acid sequence of SEQ ID NO: 175 and the second sequence encoding a second ITR comprises the nucleic acid sequence of SEQ ID NO: 267. In an aspect, the first sequence encoding a first ITR comprises the nucleic acid sequence of SEQ ID NO: 176 and the second sequence encoding a second ITR comprises the nucleic acid sequence of SEQ ID NO: 267. In an aspect, the first sequence encoding a first ITR comprises the nucleic acid sequence of SEQ ID NO: 177 and the second sequence encoding a second ITR comprises the nucleic acid sequence of SEQ ID NO: 267. In an aspect, the first sequence encoding a first ITR comprises the nucleic acid sequence of SEQ ID NO: 178 and the second sequence encoding a second ITR comprises the nucleic acid sequence of SEQ ID NO: 267. In an aspect, the first sequence encoding a first ITR comprises the nucleic acid sequence of SEQ ID NO: 179 and the second sequence encoding a second ITR comprises the nucleic acid sequence of SEQ ID NO: 267. In an aspect, the first sequence encoding a first ITR comprises the nucleic acid sequence of SEQ ID NO: 180 and the second sequence encoding a second ITR comprises the nucleic acid sequence of SEQ ID NO: 267. In an aspect, the first sequence encoding a first ITR comprises the nucleic acid sequence of SEQ ID NO: 181 and the second sequence encoding a second ITR comprises the nucleic acid sequence of SEQ ID NO: 267. In an aspect, the first sequence encoding a first ITR comprises the nucleic acid sequence of SEQ ID NO: 182 and the second sequence encoding a second ITR comprises the nucleic acid sequence of SEQ ID NO: 267. In an aspect, the first sequence encoding a first ITR comprises the nucleic acid sequence of SEQ ID NO: 183 and the second sequence encoding a second ITR comprises the nucleic acid sequence of SEQ ID NO: 267. In an aspect, the first sequence encoding a first ITR comprises the nucleic acid sequence of SEQ ID NO: 184 and the second sequence encoding a second ITR comprises the nucleic acid sequence of SEQ ID NO: 267. In an aspect, the first sequence encoding a first ITR comprises the nucleic acid sequence of SEQ ID NO: 185 and the second sequence encoding a second ITR comprises the nucleic acid sequence of SEQ ID NO: 267. In an aspect, the first sequence encoding a first ITR comprises the nucleic acid sequence of SEQ ID NO: 186 and the second sequence encoding a second ITR comprises the nucleic acid sequence of SEQ ID NO: 267. In an aspect, the first sequence encoding a first ITR comprises the nucleic acid sequence of SEQ ID NO: 187 and the second sequence encoding a second ITR comprises the nucleic acid sequence of SEQ ID NO: 267. In an aspect, the first sequence encoding a first ITR comprises the nucleic acid sequence of SEQ ID NO: 188 and the second sequence encoding a second ITR comprises the nucleic acid sequence of SEQ ID NO: 267. In an aspect, the first sequence encoding a first ITR comprises the nucleic acid sequence of SEQ ID NO: 189 and the second sequence encoding a second ITR comprises the nucleic acid sequence of SEQ ID NO: 267.

In an aspect, the first sequence encoding a first ITR comprises the nucleic acid sequence of SEQ ID NO: 4 and the second sequence encoding a second ITR comprises the nucleic acid sequence of SEQ ID NO: 268. In an aspect, the first sequence encoding a first ITR comprises the nucleic acid sequence of SEQ ID NO: 14 and the second sequence encoding a second ITR comprises the nucleic acid sequence of SEQ ID NO: 268. In an aspect, the first sequence encoding a first ITR comprises the nucleic acid sequence of SEQ ID NO: 15 and the second sequence encoding a second ITR comprises the nucleic acid sequence of SEQ ID NO: 268. In an aspect, the first sequence encoding a first ITR comprises the nucleic acid sequence of SEQ ID NO: 16 and the second sequence encoding a second ITR comprises the nucleic acid sequence of SEQ ID NO: 268. In an aspect, the first sequence encoding a first ITR comprises the nucleic acid sequence of SEQ ID NO: 160 and the second sequence encoding a second ITR comprises the nucleic acid sequence of SEQ ID NO: 268. In an aspect, the first sequence encoding a first ITR comprises the nucleic acid sequence of SEQ ID NO: 161 and the second sequence encoding a second ITR comprises the nucleic acid sequence of SEQ ID NO: 268. In an aspect, the first sequence encoding a first ITR comprises the nucleic acid sequence of SEQ ID NO: 162 and the second sequence encoding a second ITR comprises the nucleic acid sequence of SEQ ID NO: 268. In an aspect, the first sequence encoding a first ITR comprises the nucleic acid sequence of SEQ ID NO: 163 and the second sequence encoding a second ITR comprises the nucleic acid sequence of SEQ ID NO: 268. In an aspect, the first sequence encoding a first ITR comprises the nucleic acid sequence of SEQ ID NO: 165 and the second sequence encoding a second ITR comprises the nucleic acid sequence of SEQ ID NO: 268. In an aspect, the first sequence encoding a first ITR comprises the nucleic acid sequence of SEQ ID NO: 166 and the second sequence encoding a second ITR comprises the nucleic acid sequence of SEQ ID NO: 268. In an aspect, the first sequence encoding a first ITR comprises the nucleic acid sequence of SEQ ID NO: 167 and the second sequence encoding a second ITR comprises the nucleic acid sequence of SEQ ID NO: 268. In an aspect, the first sequence encoding a first ITR comprises the nucleic acid sequence of SEQ ID NO: 168 and the second sequence encoding a second ITR comprises the nucleic acid sequence of SEQ ID NO: 268. In an aspect, the first sequence encoding a first ITR comprises the nucleic acid sequence of SEQ ID NO: 169 and the second sequence encoding a second ITR comprises the nucleic acid sequence of SEQ ID NO: 268. In an aspect, the first sequence encoding a first ITR comprises the nucleic acid sequence of SEQ ID NO: 170 and the second sequence encoding a second ITR comprises the nucleic acid sequence of SEQ ID NO: 268. In an aspect, the first sequence encoding a first ITR comprises the nucleic acid sequence of SEQ ID NO: 171 and the second sequence encoding a second ITR comprises the nucleic acid sequence of SEQ ID NO: 268. In an aspect, the first sequence encoding a first ITR comprises the nucleic acid sequence of SEQ ID NO: 172 and the second sequence encoding a second ITR comprises the nucleic acid sequence of SEQ ID NO: 268. In an aspect, the first sequence encoding a first ITR comprises the nucleic acid sequence of SEQ ID NO: 173 and the second sequence encoding a second ITR comprises the nucleic acid sequence of SEQ ID NO: 268. In an aspect, the first sequence encoding a first ITR comprises the nucleic acid sequence of SEQ ID NO: 174 and the second sequence encoding a second ITR comprises the nucleic acid sequence of SEQ ID NO: 268. In an aspect, the first sequence encoding a first ITR comprises the nucleic acid sequence of SEQ ID NO: 175 and the second sequence encoding a second ITR comprises the nucleic acid sequence of SEQ ID NO: 268. In an aspect, the first sequence encoding a first ITR comprises the nucleic acid sequence of SEQ ID NO: 176 and the second sequence encoding a second ITR comprises the nucleic acid sequence of SEQ ID NO: 268. In an aspect, the first sequence encoding a first ITR comprises the nucleic acid sequence of SEQ ID NO: 177 and the second sequence encoding a second ITR comprises the nucleic acid sequence of SEQ ID NO: 268. In an aspect, the first sequence encoding a first ITR comprises the nucleic acid sequence of SEQ ID NO: 178 and the second sequence encoding a second ITR comprises the nucleic acid sequence of SEQ ID NO: 268. In an aspect, the first sequence encoding a first ITR comprises the nucleic acid sequence of SEQ ID NO: 179 and the second sequence encoding a second ITR comprises the nucleic acid sequence of SEQ ID NO: 268. In an aspect, the first sequence encoding a first ITR comprises the nucleic acid sequence of SEQ ID NO: 180 and the second sequence encoding a second ITR comprises the nucleic acid sequence of SEQ ID NO: 268. In an aspect, the first sequence encoding a first ITR comprises the nucleic acid sequence of SEQ ID NO: 181 and the second sequence encoding a second ITR comprises the nucleic acid sequence of SEQ ID NO: 268. In an aspect, the first sequence encoding a first ITR comprises the nucleic acid sequence of SEQ ID NO: 182 and the second sequence encoding a second ITR comprises the nucleic acid sequence of SEQ ID NO: 268. In an aspect, the first sequence encoding a first ITR comprises the nucleic acid sequence of SEQ ID NO: 183 and the second sequence encoding a second ITR comprises the nucleic acid sequence of SEQ ID NO: 268. In an aspect, the first sequence encoding a first ITR comprises the nucleic acid sequence of SEQ ID NO: 184 and the second sequence encoding a second ITR comprises the nucleic acid sequence of SEQ ID NO: 268. In an aspect, the first sequence encoding a first ITR comprises the nucleic acid sequence of SEQ ID NO: 185 and the second sequence encoding a second ITR comprises the nucleic acid sequence of SEQ ID NO: 268. In an aspect, the first sequence encoding a first ITR comprises the nucleic acid sequence of SEQ ID NO: 186 and the second sequence encoding a second ITR comprises the nucleic acid sequence of SEQ ID NO: 268. In an aspect, the first sequence encoding a first ITR comprises the nucleic acid sequence of SEQ ID NO: 187 and the second sequence encoding a second ITR comprises the nucleic acid sequence of SEQ ID NO: 268. In an aspect, the first sequence encoding a first ITR comprises the nucleic acid sequence of SEQ ID NO: 188 and the second sequence encoding a second ITR comprises the nucleic acid sequence of SEQ ID NO: 268. In an aspect, the first sequence encoding a first ITR comprises the nucleic acid sequence of SEQ ID NO: 189 and the second sequence encoding a second ITR comprises the nucleic acid sequence of SEQ ID NO: 268.

In an aspect, the first sequence encoding a first ITR comprises the nucleic acid sequence of SEQ ID NO: 4 and the second sequence encoding a second ITR comprises the nucleic acid sequence of SEQ ID NO: 269. In an aspect, the first sequence encoding a first ITR comprises the nucleic acid sequence of SEQ ID NO: 14 and the second sequence encoding a second ITR comprises the nucleic acid sequence of SEQ ID NO: 269. In an aspect, the first sequence encoding a first ITR comprises the nucleic acid sequence of SEQ ID NO: 15 and the second sequence encoding a second ITR comprises the nucleic acid sequence of SEQ ID NO: 269. In an aspect, the first sequence encoding a first ITR comprises the nucleic acid sequence of SEQ ID NO: 16 and the second sequence encoding a second ITR comprises the nucleic acid sequence of SEQ ID NO: 269. In an aspect, the first sequence encoding a first ITR comprises the nucleic acid sequence of SEQ ID NO: 160 and the second sequence encoding a second ITR comprises the nucleic acid sequence of SEQ ID NO: 269. In an aspect, the first sequence encoding a first ITR comprises the nucleic acid sequence of SEQ ID NO: 161 and the second sequence encoding a second ITR comprises the nucleic acid sequence of SEQ ID NO: 269. In an aspect, the first sequence encoding a first ITR comprises the nucleic acid sequence of SEQ ID NO: 162 and the second sequence encoding a second ITR comprises the nucleic acid sequence of SEQ ID NO: 269. In an aspect, the first sequence encoding a first ITR comprises the nucleic acid sequence of SEQ ID NO: 163 and the second sequence encoding a second ITR comprises the nucleic acid sequence of SEQ ID NO: 269. In an aspect, the first sequence encoding a first ITR comprises the nucleic acid sequence of SEQ ID NO: 165 and the second sequence encoding a second ITR comprises the nucleic acid sequence of SEQ ID NO: 269. In an aspect, the first sequence encoding a first ITR comprises the nucleic acid sequence of SEQ ID NO: 166 and the second sequence encoding a second ITR comprises the nucleic acid sequence of SEQ ID NO: 269. In an aspect, the first sequence encoding a first ITR comprises the nucleic acid sequence of SEQ ID NO: 167 and the second sequence encoding a second ITR comprises the nucleic acid sequence of SEQ ID NO: 269. In an aspect, the first sequence encoding a first ITR comprises the nucleic acid sequence of SEQ ID NO: 168 and the second sequence encoding a second ITR comprises the nucleic acid sequence of SEQ ID NO: 269. In an aspect, the first sequence encoding a first ITR comprises the nucleic acid sequence of SEQ ID NO: 169 and the second sequence encoding a second ITR comprises the nucleic acid sequence of SEQ ID NO: 269. In an aspect, the first sequence encoding a first ITR comprises the nucleic acid sequence of SEQ ID NO: 170 and the second sequence encoding a second ITR comprises the nucleic acid sequence of SEQ ID NO: 269. In an aspect, the first sequence encoding a first ITR comprises the nucleic acid sequence of SEQ ID NO: 171 and the second sequence encoding a second ITR comprises the nucleic acid sequence of SEQ ID NO: 269. In an aspect, the first sequence encoding a first ITR comprises the nucleic acid sequence of SEQ ID NO: 172 and the second sequence encoding a second ITR comprises the nucleic acid sequence of SEQ ID NO: 269. In an aspect, the first sequence encoding a first ITR comprises the nucleic acid sequence of SEQ ID NO: 173 and the second sequence encoding a second ITR comprises the nucleic acid sequence of SEQ ID NO: 269. In an aspect, the first sequence encoding a first ITR comprises the nucleic acid sequence of SEQ ID NO: 174 and the second sequence encoding a second ITR comprises the nucleic acid sequence of SEQ ID NO: 269. In an aspect, the first sequence encoding a first ITR comprises the nucleic acid sequence of SEQ ID NO: 175 and the second sequence encoding a second ITR comprises the nucleic acid sequence of SEQ ID NO: 269. In an aspect, the first sequence encoding a first ITR comprises the nucleic acid sequence of SEQ ID NO: 176 and the second sequence encoding a second ITR comprises the nucleic acid sequence of SEQ ID NO: 269. In an aspect, the first sequence encoding a first ITR comprises the nucleic acid sequence of SEQ ID NO: 177 and the second sequence encoding a second ITR comprises the nucleic acid sequence of SEQ ID NO: 269. In an aspect, the first sequence encoding a first ITR comprises the nucleic acid sequence of SEQ ID NO: 178 and the second sequence encoding a second ITR comprises the nucleic acid sequence of SEQ ID NO: 269. In an aspect, the first sequence encoding a first ITR comprises the nucleic acid sequence of SEQ ID NO: 179 and the second sequence encoding a second ITR comprises the nucleic acid sequence of SEQ ID NO: 269. In an aspect, the first sequence encoding a first ITR comprises the nucleic acid sequence of SEQ ID NO: 180 and the second sequence encoding a second ITR comprises the nucleic acid sequence of SEQ ID NO: 269. In an aspect, the first sequence encoding a first ITR comprises the nucleic acid sequence of SEQ ID NO: 181 and the second sequence encoding a second ITR comprises the nucleic acid sequence of SEQ ID NO: 269. In an aspect, the first sequence encoding a first ITR comprises the nucleic acid sequence of SEQ ID NO: 182 and the second sequence encoding a second ITR comprises the nucleic acid sequence of SEQ ID NO: 269. In an aspect, the first sequence encoding a first ITR comprises the nucleic acid sequence of SEQ ID NO: 183 and the second sequence encoding a second ITR comprises the nucleic acid sequence of SEQ ID NO: 269. In an aspect, the first sequence encoding a first ITR comprises the nucleic acid sequence of SEQ ID NO: 184 and the second sequence encoding a second ITR comprises the nucleic acid sequence of SEQ ID NO: 269. In an aspect, the first sequence encoding a first ITR comprises the nucleic acid sequence of SEQ ID NO: 185 and the second sequence encoding a second ITR comprises the nucleic acid sequence of SEQ ID NO: 269. In an aspect, the first sequence encoding a first ITR comprises the nucleic acid sequence of SEQ ID NO: 186 and the second sequence encoding a second ITR comprises the nucleic acid sequence of SEQ ID NO: 269. In an aspect, the first sequence encoding a first ITR comprises the nucleic acid sequence of SEQ ID NO: 187 and the second sequence encoding a second ITR comprises the nucleic acid sequence of SEQ ID NO: 269. In an aspect, the first sequence encoding a first ITR comprises the nucleic acid sequence of SEQ ID NO: 188 and the second sequence encoding a second ITR comprises the nucleic acid sequence of SEQ ID NO: 269. In an aspect, the first sequence encoding a first ITR comprises the nucleic acid sequence of SEQ ID NO: 189 and the second sequence encoding a second ITR comprises the nucleic acid sequence of SEQ ID NO: 269.

In an aspect, the first sequence encoding a first ITR comprises the nucleic acid sequence of SEQ ID NO: 4 and the second sequence encoding a second ITR comprises the nucleic acid sequence of SEQ ID NO: 270. In an aspect, the first sequence encoding a first ITR comprises the nucleic acid sequence of SEQ ID NO: 14 and the second sequence encoding a second ITR comprises the nucleic acid sequence of SEQ ID NO: 270. In an aspect, the first sequence encoding a first ITR comprises the nucleic acid sequence of SEQ ID NO: 15 and the second sequence encoding a second ITR comprises the nucleic acid sequence of SEQ ID NO: 270. In an aspect, the first sequence encoding a first ITR comprises the nucleic acid sequence of SEQ ID NO: 16 and the second sequence encoding a second ITR comprises the nucleic acid sequence of SEQ ID NO: 270. In an aspect, the first sequence encoding a first ITR comprises the nucleic acid sequence of SEQ ID NO: 160 and the second sequence encoding a second ITR comprises the nucleic acid sequence of SEQ ID NO: 270. In an aspect, the first sequence encoding a first ITR comprises the nucleic acid sequence of SEQ ID NO: 161 and the second sequence encoding a second ITR comprises the nucleic acid sequence of SEQ ID NO: 270. In an aspect, the first sequence encoding a first ITR comprises the nucleic acid sequence of SEQ ID NO: 162 and the second sequence encoding a second ITR comprises the nucleic acid sequence of SEQ ID NO: 270. In an aspect, the first sequence encoding a first ITR comprises the nucleic acid sequence of SEQ ID NO: 163 and the second sequence encoding a second ITR comprises the nucleic acid sequence of SEQ ID NO: 270. In an aspect, the first sequence encoding a first ITR comprises the nucleic acid sequence of SEQ ID NO: 165 and the second sequence encoding a second ITR comprises the nucleic acid sequence of SEQ ID NO: 270. In an aspect, the first sequence encoding a first ITR comprises the nucleic acid sequence of SEQ ID NO: 166 and the second sequence encoding a second ITR comprises the nucleic acid sequence of SEQ ID NO: 270. In an aspect, the first sequence encoding a first ITR comprises the nucleic acid sequence of SEQ ID NO: 167 and the second sequence encoding a second ITR comprises the nucleic acid sequence of SEQ ID NO: 270. In an aspect, the first sequence encoding a first ITR comprises the nucleic acid sequence of SEQ ID NO: 168 and the second sequence encoding a second ITR comprises the nucleic acid sequence of SEQ ID NO: 270. In an aspect, the first sequence encoding a first ITR comprises the nucleic acid sequence of SEQ ID NO: 169 and the second sequence encoding a second ITR comprises the nucleic acid sequence of SEQ ID NO: 270. In an aspect, the first sequence encoding a first ITR comprises the nucleic acid sequence of SEQ ID NO: 170 and the second sequence encoding a second ITR comprises the nucleic acid sequence of SEQ ID NO: 270. In an aspect, the first sequence encoding a first ITR comprises the nucleic acid sequence of SEQ ID NO: 171 and the second sequence encoding a second ITR comprises the nucleic acid sequence of SEQ ID NO: 270. In an aspect, the first sequence encoding a first ITR comprises the nucleic acid sequence of SEQ ID NO: 172 and the second sequence encoding a second ITR comprises the nucleic acid sequence of SEQ ID NO: 270. In an aspect, the first sequence encoding a first ITR comprises the nucleic acid sequence of SEQ ID NO: 173 and the second sequence encoding a second ITR comprises the nucleic acid sequence of SEQ ID NO: 270. In an aspect, the first sequence encoding a first ITR comprises the nucleic acid sequence of SEQ ID NO: 174 and the second sequence encoding a second ITR comprises the nucleic acid sequence of SEQ ID NO: 270. In an aspect, the first sequence encoding a first ITR comprises the nucleic acid sequence of SEQ ID NO: 175 and the second sequence encoding a second ITR comprises the nucleic acid sequence of SEQ ID NO: 270. In an aspect, the first sequence encoding a first ITR comprises the nucleic acid sequence of SEQ ID NO: 176 and the second sequence encoding a second ITR comprises the nucleic acid sequence of SEQ ID NO: 270. In an aspect, the first sequence encoding a first ITR comprises the nucleic acid sequence of SEQ ID NO: 177 and the second sequence encoding a second ITR comprises the nucleic acid sequence of SEQ ID NO: 270. In an aspect, the first sequence encoding a first ITR comprises the nucleic acid sequence of SEQ ID NO: 178 and the second sequence encoding a second ITR comprises the nucleic acid sequence of SEQ ID NO: 270. In an aspect, the first sequence encoding a first ITR comprises the nucleic acid sequence of SEQ ID NO: 179 and the second sequence encoding a second ITR comprises the nucleic acid sequence of SEQ ID NO: 270. In an aspect, the first sequence encoding a first ITR comprises the nucleic acid sequence of SEQ ID NO: 180 and the second sequence encoding a second ITR comprises the nucleic acid sequence of SEQ ID NO: 270. In an aspect, the first sequence encoding a first ITR comprises the nucleic acid sequence of SEQ ID NO: 181 and the second sequence encoding a second ITR comprises the nucleic acid sequence of SEQ ID NO: 270. In an aspect, the first sequence encoding a first ITR comprises the nucleic acid sequence of SEQ ID NO: 182 and the second sequence encoding a second ITR comprises the nucleic acid sequence of SEQ ID NO: 270. In an aspect, the first sequence encoding a first ITR comprises the nucleic acid sequence of SEQ ID NO: 183 and the second sequence encoding a second ITR comprises the nucleic acid sequence of SEQ ID NO: 270. In an aspect, the first sequence encoding a first ITR comprises the nucleic acid sequence of SEQ ID NO: 184 and the second sequence encoding a second ITR comprises the nucleic acid sequence of SEQ ID NO: 270. In an aspect, the first sequence encoding a first ITR comprises the nucleic acid sequence of SEQ ID NO: 185 and the second sequence encoding a second ITR comprises the nucleic acid sequence of SEQ ID NO: 270. In an aspect, the first sequence encoding a first ITR comprises the nucleic acid sequence of SEQ ID NO: 186 and the second sequence encoding a second ITR comprises the nucleic acid sequence of SEQ ID NO: 270. In an aspect, the first sequence encoding a first ITR comprises the nucleic acid sequence of SEQ ID NO: 187 and the second sequence encoding a second ITR comprises the nucleic acid sequence of SEQ ID NO: 270. In an aspect, the first sequence encoding a first ITR comprises the nucleic acid sequence of SEQ ID NO: 188 and the second sequence encoding a second ITR comprises the nucleic acid sequence of SEQ ID NO: 270. In an aspect, the first sequence encoding a first ITR comprises the nucleic acid sequence of SEQ ID NO: 189 and the second sequence encoding a second ITR comprises the nucleic acid sequence of SEQ ID NO: 270.

In an aspect, the first sequence encoding a first ITR comprises the nucleic acid sequence of SEQ ID NO: 4 and the second sequence encoding a second ITR comprises the nucleic acid sequence of SEQ ID NO: 271. In an aspect, the first sequence encoding a first ITR comprises the nucleic acid sequence of SEQ ID NO: 14 and the second sequence encoding a second ITR comprises the nucleic acid sequence of SEQ ID NO: 271. In an aspect, the first sequence encoding a first ITR comprises the nucleic acid sequence of SEQ ID NO: 15 and the second sequence encoding a second ITR comprises the nucleic acid sequence of SEQ ID NO: 271. In an aspect, the first sequence encoding a first ITR comprises the nucleic acid sequence of SEQ ID NO: 16 and the second sequence encoding a second ITR comprises the nucleic acid sequence of SEQ ID NO: 271. In an aspect, the first sequence encoding a first ITR comprises the nucleic acid sequence of SEQ ID NO: 160 and the second sequence encoding a second ITR comprises the nucleic acid sequence of SEQ ID NO: 271. In an aspect, the first sequence encoding a first ITR comprises the nucleic acid sequence of SEQ ID NO: 161 and the second sequence encoding a second ITR comprises the nucleic acid sequence of SEQ ID NO: 271. In an aspect, the first sequence encoding a first ITR comprises the nucleic acid sequence of SEQ ID NO: 162 and the second sequence encoding a second ITR comprises the nucleic acid sequence of SEQ ID NO: 271. In an aspect, the first sequence encoding a first ITR comprises the nucleic acid sequence of SEQ ID NO: 163 and the second sequence encoding a second ITR comprises the nucleic acid sequence of SEQ ID NO: 271. In an aspect, the first sequence encoding a first ITR comprises the nucleic acid sequence of SEQ ID NO: 165 and the second sequence encoding a second ITR comprises the nucleic acid sequence of SEQ ID NO: 271. In an aspect, the first sequence encoding a first ITR comprises the nucleic acid sequence of SEQ ID NO: 166 and the second sequence encoding a second ITR comprises the nucleic acid sequence of SEQ ID NO: 271. In an aspect, the first sequence encoding a first ITR comprises the nucleic acid sequence of SEQ ID NO: 167 and the second sequence encoding a second ITR comprises the nucleic acid sequence of SEQ ID NO: 271. In an aspect, the first sequence encoding a first ITR comprises the nucleic acid sequence of SEQ ID NO: 168 and the second sequence encoding a second ITR comprises the nucleic acid sequence of SEQ ID NO: 271. In an aspect, the first sequence encoding a first ITR comprises the nucleic acid sequence of SEQ ID NO: 169 and the second sequence encoding a second ITR comprises the nucleic acid sequence of SEQ ID NO: 271. In an aspect, the first sequence encoding a first ITR comprises the nucleic acid sequence of SEQ ID NO: 170 and the second sequence encoding a second ITR comprises the nucleic acid sequence of SEQ ID NO: 271. In an aspect, the first sequence encoding a first ITR comprises the nucleic acid sequence of SEQ ID NO: 171 and the second sequence encoding a second ITR comprises the nucleic acid sequence of SEQ ID NO: 271. In an aspect, the first sequence encoding a first ITR comprises the nucleic acid sequence of SEQ ID NO: 172 and the second sequence encoding a second ITR comprises the nucleic acid sequence of SEQ ID NO: 271. In an aspect, the first sequence encoding a first ITR comprises the nucleic acid sequence of SEQ ID NO: 173 and the second sequence encoding a second ITR comprises the nucleic acid sequence of SEQ ID NO: 271. In an aspect, the first sequence encoding a first ITR comprises the nucleic acid sequence of SEQ ID NO: 174 and the second sequence encoding a second ITR comprises the nucleic acid sequence of SEQ ID NO: 271. In an aspect, the first sequence encoding a first ITR comprises the nucleic acid sequence of SEQ ID NO: 175 and the second sequence encoding a second ITR comprises the nucleic acid sequence of SEQ ID NO: 271. In an aspect, the first sequence encoding a first ITR comprises the nucleic acid sequence of SEQ ID NO: 176 and the second sequence encoding a second ITR comprises the nucleic acid sequence of SEQ ID NO: 271. In an aspect, the first sequence encoding a first ITR comprises the nucleic acid sequence of SEQ ID NO: 177 and the second sequence encoding a second ITR comprises the nucleic acid sequence of SEQ ID NO: 271. In an aspect, the first sequence encoding a first ITR comprises the nucleic acid sequence of SEQ ID NO: 178 and the second sequence encoding a second ITR comprises the nucleic acid sequence of SEQ ID NO: 271. In an aspect, the first sequence encoding a first ITR comprises the nucleic acid sequence of SEQ ID NO: 179 and the second sequence encoding a second ITR comprises the nucleic acid sequence of SEQ ID NO: 271. In an aspect, the first sequence encoding a first ITR comprises the nucleic acid sequence of SEQ ID NO: 180 and the second sequence encoding a second ITR comprises the nucleic acid sequence of SEQ ID NO: 271. In an aspect, the first sequence encoding a first ITR comprises the nucleic acid sequence of SEQ ID NO: 181 and the second sequence encoding a second ITR comprises the nucleic acid sequence of SEQ ID NO: 271. In an aspect, the first sequence encoding a first ITR comprises the nucleic acid sequence of SEQ ID NO: 182 and the second sequence encoding a second ITR comprises the nucleic acid sequence of SEQ ID NO: 271. In an aspect, the first sequence encoding a first ITR comprises the nucleic acid sequence of SEQ ID NO: 183 and the second sequence encoding a second ITR comprises the nucleic acid sequence of SEQ ID NO: 271. In an aspect, the first sequence encoding a first ITR comprises the nucleic acid sequence of SEQ ID NO: 184 and the second sequence encoding a second ITR comprises the nucleic acid sequence of SEQ ID NO: 271. In an aspect, the first sequence encoding a first ITR comprises the nucleic acid sequence of SEQ ID NO: 185 and the second sequence encoding a second ITR comprises the nucleic acid sequence of SEQ ID NO: 271. In an aspect, the first sequence encoding a first ITR comprises the nucleic acid sequence of SEQ ID NO: 186 and the second sequence encoding a second ITR comprises the nucleic acid sequence of SEQ ID NO: 271. In an aspect, the first sequence encoding a first ITR comprises the nucleic acid sequence of SEQ ID NO: 187 and the second sequence encoding a second ITR comprises the nucleic acid sequence of SEQ ID NO: 271. In an aspect, the first sequence encoding a first ITR comprises the nucleic acid sequence of SEQ ID NO: 188 and the second sequence encoding a second ITR comprises the nucleic acid sequence of SEQ ID NO: 271. In an aspect, the first sequence encoding a first ITR comprises the nucleic acid sequence of SEQ ID NO: 189 and the second sequence encoding a second ITR comprises the nucleic acid sequence of SEQ ID NO: 271.

In an aspect, the first sequence encoding a first ITR comprises the nucleic acid sequence of SEQ ID NO: 4 and the second sequence encoding a second ITR comprises the nucleic acid sequence of SEQ ID NO: 272. In an aspect, the first sequence encoding a first ITR comprises the nucleic acid sequence of SEQ ID NO: 14 and the second sequence encoding a second ITR comprises the nucleic acid sequence of SEQ ID NO: 272. In an aspect, the first sequence encoding a first ITR comprises the nucleic acid sequence of SEQ ID NO: 15 and the second sequence encoding a second ITR comprises the nucleic acid sequence of SEQ ID NO: 272. In an aspect, the first sequence encoding a first ITR comprises the nucleic acid sequence of SEQ ID NO: 16 and the second sequence encoding a second ITR comprises the nucleic acid sequence of SEQ ID NO: 272. In an aspect, the first sequence encoding a first ITR comprises the nucleic acid sequence of SEQ ID NO: 160 and the second sequence encoding a second ITR comprises the nucleic acid sequence of SEQ ID NO: 272. In an aspect, the first sequence encoding a first ITR comprises the nucleic acid sequence of SEQ ID NO: 161 and the second sequence encoding a second ITR comprises the nucleic acid sequence of SEQ ID NO: 272. In an aspect, the first sequence encoding a first ITR comprises the nucleic acid sequence of SEQ ID NO: 162 and the second sequence encoding a second ITR comprises the nucleic acid sequence of SEQ ID NO: 272. In an aspect, the first sequence encoding a first ITR comprises the nucleic acid sequence of SEQ ID NO: 163 and the second sequence encoding a second ITR comprises the nucleic acid sequence of SEQ ID NO: 272. In an aspect, the first sequence encoding a first ITR comprises the nucleic acid sequence of SEQ ID NO: 165 and the second sequence encoding a second ITR comprises the nucleic acid sequence of SEQ ID NO: 272. In an aspect, the first sequence encoding a first ITR comprises the nucleic acid sequence of SEQ ID NO: 166 and the second sequence encoding a second ITR comprises the nucleic acid sequence of SEQ ID NO: 272. In an aspect, the first sequence encoding a first ITR comprises the nucleic acid sequence of SEQ ID NO: 167 and the second sequence encoding a second ITR comprises the nucleic acid sequence of SEQ ID NO: 272. In an aspect, the first sequence encoding a first ITR comprises the nucleic acid sequence of SEQ ID NO: 168 and the second sequence encoding a second ITR comprises the nucleic acid sequence of SEQ ID NO: 272. In an aspect, the first sequence encoding a first ITR comprises the nucleic acid sequence of SEQ ID NO: 169 and the second sequence encoding a second ITR comprises the nucleic acid sequence of SEQ ID NO: 272. In an aspect, the first sequence encoding a first ITR comprises the nucleic acid sequence of SEQ ID NO: 170 and the second sequence encoding a second ITR comprises the nucleic acid sequence of SEQ ID NO: 272. In an aspect, the first sequence encoding a first ITR comprises the nucleic acid sequence of SEQ ID NO: 171 and the second sequence encoding a second ITR comprises the nucleic acid sequence of SEQ ID NO: 272. In an aspect, the first sequence encoding a first ITR comprises the nucleic acid sequence of SEQ ID NO: 172 and the second sequence encoding a second ITR comprises the nucleic acid sequence of SEQ ID NO: 272. In an aspect, the first sequence encoding a first ITR comprises the nucleic acid sequence of SEQ ID NO: 173 and the second sequence encoding a second ITR comprises the nucleic acid sequence of SEQ ID NO: 272. In an aspect, the first sequence encoding a first ITR comprises the nucleic acid sequence of SEQ ID NO: 174 and the second sequence encoding a second ITR comprises the nucleic acid sequence of SEQ ID NO: 272. In an aspect, the first sequence encoding a first ITR comprises the nucleic acid sequence of SEQ ID NO: 175 and the second sequence encoding a second ITR comprises the nucleic acid sequence of SEQ ID NO: 272. In an aspect, the first sequence encoding a first ITR comprises the nucleic acid sequence of SEQ ID NO: 176 and the second sequence encoding a second ITR comprises the nucleic acid sequence of SEQ ID NO: 272. In an aspect, the first sequence encoding a first ITR comprises the nucleic acid sequence of SEQ ID NO: 177 and the second sequence encoding a second ITR comprises the nucleic acid sequence of SEQ ID NO: 272. In an aspect, the first sequence encoding a first ITR comprises the nucleic acid sequence of SEQ ID NO: 178 and the second sequence encoding a second ITR comprises the nucleic acid sequence of SEQ ID NO: 272. In an aspect, the first sequence encoding a first ITR comprises the nucleic acid sequence of SEQ ID NO: 179 and the second sequence encoding a second ITR comprises the nucleic acid sequence of SEQ ID NO: 272. In an aspect, the first sequence encoding a first ITR comprises the nucleic acid sequence of SEQ ID NO: 180 and the second sequence encoding a second ITR comprises the nucleic acid sequence of SEQ ID NO: 272. In an aspect, the first sequence encoding a first ITR comprises the nucleic acid sequence of SEQ ID NO: 181 and the second sequence encoding a second ITR comprises the nucleic acid sequence of SEQ ID NO: 272. In an aspect, the first sequence encoding a first ITR comprises the nucleic acid sequence of SEQ ID NO: 182 and the second sequence encoding a second ITR comprises the nucleic acid sequence of SEQ ID NO: 272. In an aspect, the first sequence encoding a first ITR comprises the nucleic acid sequence of SEQ ID NO: 183 and the second sequence encoding a second ITR comprises the nucleic acid sequence of SEQ ID NO: 272. In an aspect, the first sequence encoding a first ITR comprises the nucleic acid sequence of SEQ ID NO: 184 and the second sequence encoding a second ITR comprises the nucleic acid sequence of SEQ ID NO: 272. In an aspect, the first sequence encoding a first ITR comprises the nucleic acid sequence of SEQ ID NO: 185 and the second sequence encoding a second ITR comprises the nucleic acid sequence of SEQ ID NO: 272. In an aspect, the first sequence encoding a first ITR comprises the nucleic acid sequence of SEQ ID NO: 186 and the second sequence encoding a second ITR comprises the nucleic acid sequence of SEQ ID NO: 272. In an aspect, the first sequence encoding a first ITR comprises the nucleic acid sequence of SEQ ID NO: 187 and the second sequence encoding a second ITR comprises the nucleic acid sequence of SEQ ID NO: 272. In an aspect, the first sequence encoding a first ITR comprises the nucleic acid sequence of SEQ ID NO: 188 and the second sequence encoding a second ITR comprises the nucleic acid sequence of SEQ ID NO: 272. In an aspect, the first sequence encoding a first ITR comprises the nucleic acid sequence of SEQ ID NO: 189 and the second sequence encoding a second ITR comprises the nucleic acid sequence of SEQ ID NO: 272.

In an aspect, the first sequence encoding a first ITR comprises the nucleic acid sequence of SEQ ID NO: 4 and the second sequence encoding a second ITR comprises the nucleic acid sequence of SEQ ID NO: 273. In an aspect, the first sequence encoding a first ITR comprises the nucleic acid sequence of SEQ ID NO: 14 and the second sequence encoding a second ITR comprises the nucleic acid sequence of SEQ ID NO: 273. In an aspect, the first sequence encoding a first ITR comprises the nucleic acid sequence of SEQ ID NO: 15 and the second sequence encoding a second ITR comprises the nucleic acid sequence of SEQ ID NO: 273. In an aspect, the first sequence encoding a first ITR comprises the nucleic acid sequence of SEQ ID NO: 16 and the second sequence encoding a second ITR comprises the nucleic acid sequence of SEQ ID NO: 273. In an aspect, the first sequence encoding a first ITR comprises the nucleic acid sequence of SEQ ID NO: 160 and the second sequence encoding a second ITR comprises the nucleic acid sequence of SEQ ID NO: 273. In an aspect, the first sequence encoding a first ITR comprises the nucleic acid sequence of SEQ ID NO: 161 and the second sequence encoding a second ITR comprises the nucleic acid sequence of SEQ ID NO: 273. In an aspect, the first sequence encoding a first ITR comprises the nucleic acid sequence of SEQ ID NO: 162 and the second sequence encoding a second ITR comprises the nucleic acid sequence of SEQ ID NO: 273. In an aspect, the first sequence encoding a first ITR comprises the nucleic acid sequence of SEQ ID NO: 163 and the second sequence encoding a second ITR comprises the nucleic acid sequence of SEQ ID NO: 273. In an aspect, the first sequence encoding a first ITR comprises the nucleic acid sequence of SEQ ID NO: 165 and the second sequence encoding a second ITR comprises the nucleic acid sequence of SEQ ID NO: 273. In an aspect, the first sequence encoding a first ITR comprises the nucleic acid sequence of SEQ ID NO: 166 and the second sequence encoding a second ITR comprises the nucleic acid sequence of SEQ ID NO: 273. In an aspect, the first sequence encoding a first ITR comprises the nucleic acid sequence of SEQ ID NO: 167 and the second sequence encoding a second ITR comprises the nucleic acid sequence of SEQ ID NO: 273. In an aspect, the first sequence encoding a first ITR comprises the nucleic acid sequence of SEQ ID NO: 168 and the second sequence encoding a second ITR comprises the nucleic acid sequence of SEQ ID NO: 273. In an aspect, the first sequence encoding a first ITR comprises the nucleic acid sequence of SEQ ID NO: 169 and the second sequence encoding a second ITR comprises the nucleic acid sequence of SEQ ID NO: 273. In an aspect, the first sequence encoding a first ITR comprises the nucleic acid sequence of SEQ ID NO: 170 and the second sequence encoding a second ITR comprises the nucleic acid sequence of SEQ ID NO: 273. In an aspect, the first sequence encoding a first ITR comprises the nucleic acid sequence of SEQ ID NO: 171 and the second sequence encoding a second ITR comprises the nucleic acid sequence of SEQ ID NO: 273. In an aspect, the first sequence encoding a first ITR comprises the nucleic acid sequence of SEQ ID NO: 172 and the second sequence encoding a second ITR comprises the nucleic acid sequence of SEQ ID NO: 273. In an aspect, the first sequence encoding a first ITR comprises the nucleic acid sequence of SEQ ID NO: 173 and the second sequence encoding a second ITR comprises the nucleic acid sequence of SEQ ID NO: 273. In an aspect, the first sequence encoding a first ITR comprises the nucleic acid sequence of SEQ ID NO: 174 and the second sequence encoding a second ITR comprises the nucleic acid sequence of SEQ ID NO: 273. In an aspect, the first sequence encoding a first ITR comprises the nucleic acid sequence of SEQ ID NO: 175 and the second sequence encoding a second ITR comprises the nucleic acid sequence of SEQ ID NO: 273. In an aspect, the first sequence encoding a first ITR comprises the nucleic acid sequence of SEQ ID NO: 176 and the second sequence encoding a second ITR comprises the nucleic acid sequence of SEQ ID NO: 273. In an aspect, the first sequence encoding a first ITR comprises the nucleic acid sequence of SEQ ID NO: 177 and the second sequence encoding a second ITR comprises the nucleic acid sequence of SEQ ID NO: 273. In an aspect, the first sequence encoding a first ITR comprises the nucleic acid sequence of SEQ ID NO: 178 and the second sequence encoding a second ITR comprises the nucleic acid sequence of SEQ ID NO: 273. In an aspect, the first sequence encoding a first ITR comprises the nucleic acid sequence of SEQ ID NO: 179 and the second sequence encoding a second ITR comprises the nucleic acid sequence of SEQ ID NO: 273. In an aspect, the first sequence encoding a first ITR comprises the nucleic acid sequence of SEQ ID NO: 180 and the second sequence encoding a second ITR comprises the nucleic acid sequence of SEQ ID NO: 273. In an aspect, the first sequence encoding a first ITR comprises the nucleic acid sequence of SEQ ID NO: 181 and the second sequence encoding a second ITR comprises the nucleic acid sequence of SEQ ID NO: 273. In an aspect, the first sequence encoding a first ITR comprises the nucleic acid sequence of SEQ ID NO: 182 and the second sequence encoding a second ITR comprises the nucleic acid sequence of SEQ ID NO: 273. In an aspect, the first sequence encoding a first ITR comprises the nucleic acid sequence of SEQ ID NO: 183 and the second sequence encoding a second ITR comprises the nucleic acid sequence of SEQ ID NO: 273. In an aspect, the first sequence encoding a first ITR comprises the nucleic acid sequence of SEQ ID NO: 184 and the second sequence encoding a second ITR comprises the nucleic acid sequence of SEQ ID NO: 273. In an aspect, the first sequence encoding a first ITR comprises the nucleic acid sequence of SEQ ID NO: 185 and the second sequence encoding a second ITR comprises the nucleic acid sequence of SEQ ID NO: 273. In an aspect, the first sequence encoding a first ITR comprises the nucleic acid sequence of SEQ ID NO: 186 and the second sequence encoding a second ITR comprises the nucleic acid sequence of SEQ ID NO: 273. In an aspect, the first sequence encoding a first ITR comprises the nucleic acid sequence of SEQ ID NO: 187 and the second sequence encoding a second ITR comprises the nucleic acid sequence of SEQ ID NO: 273. In an aspect, the first sequence encoding a first ITR comprises the nucleic acid sequence of SEQ ID NO: 188 and the second sequence encoding a second ITR comprises the nucleic acid sequence of SEQ ID NO: 273. In an aspect, the first sequence encoding a first ITR comprises the nucleic acid sequence of SEQ ID NO: 189 and the second sequence encoding a second ITR comprises the nucleic acid sequence of SEQ ID NO: 273.

In an aspect, the first sequence encoding a first ITR comprises the nucleic acid sequence of SEQ ID NO: 4 and the second sequence encoding a second ITR comprises the nucleic acid sequence of SEQ ID NO: 274. In an aspect, the first sequence encoding a first ITR comprises the nucleic acid sequence of SEQ ID NO: 14 and the second sequence encoding a second ITR comprises the nucleic acid sequence of SEQ ID NO: 274. In an aspect, the first sequence encoding a first ITR comprises the nucleic acid sequence of SEQ ID NO: 15 and the second sequence encoding a second ITR comprises the nucleic acid sequence of SEQ ID NO: 274. In an aspect, the first sequence encoding a first ITR comprises the nucleic acid sequence of SEQ ID NO: 16 and the second sequence encoding a second ITR comprises the nucleic acid sequence of SEQ ID NO: 274. In an aspect, the first sequence encoding a first ITR comprises the nucleic acid sequence of SEQ ID NO: 160 and the second sequence encoding a second ITR comprises the nucleic acid sequence of SEQ ID NO: 274. In an aspect, the first sequence encoding a first ITR comprises the nucleic acid sequence of SEQ ID NO: 161 and the second sequence encoding a second ITR comprises the nucleic acid sequence of SEQ ID NO: 274. In an aspect, the first sequence encoding a first ITR comprises the nucleic acid sequence of SEQ ID NO: 162 and the second sequence encoding a second ITR comprises the nucleic acid sequence of SEQ ID NO: 274. In an aspect, the first sequence encoding a first ITR comprises the nucleic acid sequence of SEQ ID NO: 163 and the second sequence encoding a second ITR comprises the nucleic acid sequence of SEQ ID NO: 274. In an aspect, the first sequence encoding a first ITR comprises the nucleic acid sequence of SEQ ID NO: 165 and the second sequence encoding a second ITR comprises the nucleic acid sequence of SEQ ID NO: 274. In an aspect, the first sequence encoding a first ITR comprises the nucleic acid sequence of SEQ ID NO: 166 and the second sequence encoding a second ITR comprises the nucleic acid sequence of SEQ ID NO: 274. In an aspect, the first sequence encoding a first ITR comprises the nucleic acid sequence of SEQ ID NO: 167 and the second sequence encoding a second ITR comprises the nucleic acid sequence of SEQ ID NO: 274. In an aspect, the first sequence encoding a first ITR comprises the nucleic acid sequence of SEQ ID NO: 168 and the second sequence encoding a second ITR comprises the nucleic acid sequence of SEQ ID NO: 274. In an aspect, the first sequence encoding a first ITR comprises the nucleic acid sequence of SEQ ID NO: 169 and the second sequence encoding a second ITR comprises the nucleic acid sequence of SEQ ID NO: 274. In an aspect, the first sequence encoding a first ITR comprises the nucleic acid sequence of SEQ ID NO: 170 and the second sequence encoding a second ITR comprises the nucleic acid sequence of SEQ ID NO: 274. In an aspect, the first sequence encoding a first ITR comprises the nucleic acid sequence of SEQ ID NO: 171 and the second sequence encoding a second ITR comprises the nucleic acid sequence of SEQ ID NO: 274. In an aspect, the first sequence encoding a first ITR comprises the nucleic acid sequence of SEQ ID NO: 172 and the second sequence encoding a second ITR comprises the nucleic acid sequence of SEQ ID NO: 274. In an aspect, the first sequence encoding a first ITR comprises the nucleic acid sequence of SEQ ID NO: 173 and the second sequence encoding a second ITR comprises the nucleic acid sequence of SEQ ID NO: 274. In an aspect, the first sequence encoding a first ITR comprises the nucleic acid sequence of SEQ ID NO: 174 and the second sequence encoding a second ITR comprises the nucleic acid sequence of SEQ ID NO: 274. In an aspect, the first sequence encoding a first ITR comprises the nucleic acid sequence of SEQ ID NO: 175 and the second sequence encoding a second ITR comprises the nucleic acid sequence of SEQ ID NO: 274. In an aspect, the first sequence encoding a first ITR comprises the nucleic acid sequence of SEQ ID NO: 176 and the second sequence encoding a second ITR comprises the nucleic acid sequence of SEQ ID NO: 274. In an aspect, the first sequence encoding a first ITR comprises the nucleic acid sequence of SEQ ID NO: 177 and the second sequence encoding a second ITR comprises the nucleic acid sequence of SEQ ID NO: 274. In an aspect, the first sequence encoding a first ITR comprises the nucleic acid sequence of SEQ ID NO: 178 and the second sequence encoding a second ITR comprises the nucleic acid sequence of SEQ ID NO: 274. In an aspect, the first sequence encoding a first ITR comprises the nucleic acid sequence of SEQ ID NO: 179 and the second sequence encoding a second ITR comprises the nucleic acid sequence of SEQ ID NO: 274. In an aspect, the first sequence encoding a first ITR comprises the nucleic acid sequence of SEQ ID NO: 180 and the second sequence encoding a second ITR comprises the nucleic acid sequence of SEQ ID NO: 274. In an aspect, the first sequence encoding a first ITR comprises the nucleic acid sequence of SEQ ID NO: 181 and the second sequence encoding a second ITR comprises the nucleic acid sequence of SEQ ID NO: 274. In an aspect, the first sequence encoding a first ITR comprises the nucleic acid sequence of SEQ ID NO: 182 and the second sequence encoding a second ITR comprises the nucleic acid sequence of SEQ ID NO: 274. In an aspect, the first sequence encoding a first ITR comprises the nucleic acid sequence of SEQ ID NO: 183 and the second sequence encoding a second ITR comprises the nucleic acid sequence of SEQ ID NO: 274. In an aspect, the first sequence encoding a first ITR comprises the nucleic acid sequence of SEQ ID NO: 184 and the second sequence encoding a second ITR comprises the nucleic acid sequence of SEQ ID NO: 274. In an aspect, the first sequence encoding a first ITR comprises the nucleic acid sequence of SEQ ID NO: 185 and the second sequence encoding a second ITR comprises the nucleic acid sequence of SEQ ID NO: 274. In an aspect, the first sequence encoding a first ITR comprises the nucleic acid sequence of SEQ ID NO: 186 and the second sequence encoding a second ITR comprises the nucleic acid sequence of SEQ ID NO: 274. In an aspect, the first sequence encoding a first ITR comprises the nucleic acid sequence of SEQ ID NO: 187 and the second sequence encoding a second ITR comprises the nucleic acid sequence of SEQ ID NO: 274. In an aspect, the first sequence encoding a first ITR comprises the nucleic acid sequence of SEQ ID NO: 188 and the second sequence encoding a second ITR comprises the nucleic acid sequence of SEQ ID NO: 274. In an aspect, the first sequence encoding a first ITR comprises the nucleic acid sequence of SEQ ID NO: 189 and the second sequence encoding a second ITR comprises the nucleic acid sequence of SEQ ID NO: 274.

In an aspect, the first sequence encoding a first ITR comprises the nucleic acid sequence of SEQ ID NO: 4 and the second sequence encoding a second ITR comprises the nucleic acid sequence of SEQ ID NO: 275. In an aspect, the first sequence encoding a first ITR comprises the nucleic acid sequence of SEQ ID NO: 14 and the second sequence encoding a second ITR comprises the nucleic acid sequence of SEQ ID NO: 275. In an aspect, the first sequence encoding a first ITR comprises the nucleic acid sequence of SEQ ID NO: 15 and the second sequence encoding a second ITR comprises the nucleic acid sequence of SEQ ID NO: 275. In an aspect, the first sequence encoding a first ITR comprises the nucleic acid sequence of SEQ ID NO: 16 and the second sequence encoding a second ITR comprises the nucleic acid sequence of SEQ ID NO: 275. In an aspect, the first sequence encoding a first ITR comprises the nucleic acid sequence of SEQ ID NO: 160 and the second sequence encoding a second ITR comprises the nucleic acid sequence of SEQ ID NO: 275. In an aspect, the first sequence encoding a first ITR comprises the nucleic acid sequence of SEQ ID NO: 161 and the second sequence encoding a second ITR comprises the nucleic acid sequence of SEQ ID NO: 275. In an aspect, the first sequence encoding a first ITR comprises the nucleic acid sequence of SEQ ID NO: 162 and the second sequence encoding a second ITR comprises the nucleic acid sequence of SEQ ID NO: 275. In an aspect, the first sequence encoding a first ITR comprises the nucleic acid sequence of SEQ ID NO: 163 and the second sequence encoding a second ITR comprises the nucleic acid sequence of SEQ ID NO: 275. In an aspect, the first sequence encoding a first ITR comprises the nucleic acid sequence of SEQ ID NO: 165 and the second sequence encoding a second ITR comprises the nucleic acid sequence of SEQ ID NO: 275. In an aspect, the first sequence encoding a first ITR comprises the nucleic acid sequence of SEQ ID NO: 166 and the second sequence encoding a second ITR comprises the nucleic acid sequence of SEQ ID NO: 275. In an aspect, the first sequence encoding a first ITR comprises the nucleic acid sequence of SEQ ID NO: 167 and the second sequence encoding a second ITR comprises the nucleic acid sequence of SEQ ID NO: 275. In an aspect, the first sequence encoding a first ITR comprises the nucleic acid sequence of SEQ ID NO: 168 and the second sequence encoding a second ITR comprises the nucleic acid sequence of SEQ ID NO: 275. In an aspect, the first sequence encoding a first ITR comprises the nucleic acid sequence of SEQ ID NO: 169 and the second sequence encoding a second ITR comprises the nucleic acid sequence of SEQ ID NO: 275. In an aspect, the first sequence encoding a first ITR comprises the nucleic acid sequence of SEQ ID NO: 170 and the second sequence encoding a second ITR comprises the nucleic acid sequence of SEQ ID NO: 275. In an aspect, the first sequence encoding a first ITR comprises the nucleic acid sequence of SEQ ID NO: 171 and the second sequence encoding a second ITR comprises the nucleic acid sequence of SEQ ID NO: 275. In an aspect, the first sequence encoding a first ITR comprises the nucleic acid sequence of SEQ ID NO: 172 and the second sequence encoding a second ITR comprises the nucleic acid sequence of SEQ ID NO: 275. In an aspect, the first sequence encoding a first ITR comprises the nucleic acid sequence of SEQ ID NO: 173 and the second sequence encoding a second ITR comprises the nucleic acid sequence of SEQ ID NO: 275. In an aspect, the first sequence encoding a first ITR comprises the nucleic acid sequence of SEQ ID NO: 174 and the second sequence encoding a second ITR comprises the nucleic acid sequence of SEQ ID NO: 275. In an aspect, the first sequence encoding a first ITR comprises the nucleic acid sequence of SEQ ID NO: 175 and the second sequence encoding a second ITR comprises the nucleic acid sequence of SEQ ID NO: 275. In an aspect, the first sequence encoding a first ITR comprises the nucleic acid sequence of SEQ ID NO: 176 and the second sequence encoding a second ITR comprises the nucleic acid sequence of SEQ ID NO: 275. In an aspect, the first sequence encoding a first ITR comprises the nucleic acid sequence of SEQ ID NO: 177 and the second sequence encoding a second ITR comprises the nucleic acid sequence of SEQ ID NO: 275. In an aspect, the first sequence encoding a first ITR comprises the nucleic acid sequence of SEQ ID NO: 178 and the second sequence encoding a second ITR comprises the nucleic acid sequence of SEQ ID NO: 275. In an aspect, the first sequence encoding a first ITR comprises the nucleic acid sequence of SEQ ID NO: 179 and the second sequence encoding a second ITR comprises the nucleic acid sequence of SEQ ID NO: 275. In an aspect, the first sequence encoding a first ITR comprises the nucleic acid sequence of SEQ ID NO: 180 and the second sequence encoding a second ITR comprises the nucleic acid sequence of SEQ ID NO: 275. In an aspect, the first sequence encoding a first ITR comprises the nucleic acid sequence of SEQ ID NO: 181 and the second sequence encoding a second ITR comprises the nucleic acid sequence of SEQ ID NO: 275. In an aspect, the first sequence encoding a first ITR comprises the nucleic acid sequence of SEQ ID NO: 182 and the second sequence encoding a second ITR comprises the nucleic acid sequence of SEQ ID NO: 275. In an aspect, the first sequence encoding a first ITR comprises the nucleic acid sequence of SEQ ID NO: 183 and the second sequence encoding a second ITR comprises the nucleic acid sequence of SEQ ID NO: 275. In an aspect, the first sequence encoding a first ITR comprises the nucleic acid sequence of SEQ ID NO: 184 and the second sequence encoding a second ITR comprises the nucleic acid sequence of SEQ ID NO: 275. In an aspect, the first sequence encoding a first ITR comprises the nucleic acid sequence of SEQ ID NO: 185 and the second sequence encoding a second ITR comprises the nucleic acid sequence of SEQ ID NO: 275. In an aspect, the first sequence encoding a first ITR comprises the nucleic acid sequence of SEQ ID NO: 186 and the second sequence encoding a second ITR comprises the nucleic acid sequence of SEQ ID NO: 275. In an aspect, the first sequence encoding a first ITR comprises the nucleic acid sequence of SEQ ID NO: 187 and the second sequence encoding a second ITR comprises the nucleic acid sequence of SEQ ID NO: 275. In an aspect, the first sequence encoding a first ITR comprises the nucleic acid sequence of SEQ ID NO: 188 and the second sequence encoding a second ITR comprises the nucleic acid sequence of SEQ ID NO: 275. In an aspect, the first sequence encoding a first ITR comprises the nucleic acid sequence of SEQ ID NO: 189 and the second sequence encoding a second ITR comprises the nucleic acid sequence of SEQ ID NO: 275.

In an aspect, the first sequence encoding a first ITR comprises the nucleic acid sequence of SEQ ID NO: 4 and the second sequence encoding a second ITR comprises the nucleic acid sequence of SEQ ID NO: 276. In an aspect, the first sequence encoding a first ITR comprises the nucleic acid sequence of SEQ ID NO: 14 and the second sequence encoding a second ITR comprises the nucleic acid sequence of SEQ ID NO: 276. In an aspect, the first sequence encoding a first ITR comprises the nucleic acid sequence of SEQ ID NO: 15 and the second sequence encoding a second ITR comprises the nucleic acid sequence of SEQ ID NO: 276. In an aspect, the first sequence encoding a first ITR comprises the nucleic acid sequence of SEQ ID NO: 16 and the second sequence encoding a second ITR comprises the nucleic acid sequence of SEQ ID NO: 276. In an aspect, the first sequence encoding a first ITR comprises the nucleic acid sequence of SEQ ID NO: 160 and the second sequence encoding a second ITR comprises the nucleic acid sequence of SEQ ID NO: 276. In an aspect, the first sequence encoding a first ITR comprises the nucleic acid sequence of SEQ ID NO: 161 and the second sequence encoding a second ITR comprises the nucleic acid sequence of SEQ ID NO: 276. In an aspect, the first sequence encoding a first ITR comprises the nucleic acid sequence of SEQ ID NO: 162 and the second sequence encoding a second ITR comprises the nucleic acid sequence of SEQ ID NO: 276. In an aspect, the first sequence encoding a first ITR comprises the nucleic acid sequence of SEQ ID NO: 163 and the second sequence encoding a second ITR comprises the nucleic acid sequence of SEQ ID NO: 276. In an aspect, the first sequence encoding a first ITR comprises the nucleic acid sequence of SEQ ID NO: 165 and the second sequence encoding a second ITR comprises the nucleic acid sequence of SEQ ID NO: 276. In an aspect, the first sequence encoding a first ITR comprises the nucleic acid sequence of SEQ ID NO: 166 and the second sequence encoding a second ITR comprises the nucleic acid sequence of SEQ ID NO: 276. In an aspect, the first sequence encoding a first ITR comprises the nucleic acid sequence of SEQ ID NO: 167 and the second sequence encoding a second ITR comprises the nucleic acid sequence of SEQ ID NO: 276. In an aspect, the first sequence encoding a first ITR comprises the nucleic acid sequence of SEQ ID NO: 168 and the second sequence encoding a second ITR comprises the nucleic acid sequence of SEQ ID NO: 276. In an aspect, the first sequence encoding a first ITR comprises the nucleic acid sequence of SEQ ID NO: 169 and the second sequence encoding a second ITR comprises the nucleic acid sequence of SEQ ID NO: 276. In an aspect, the first sequence encoding a first ITR comprises the nucleic acid sequence of SEQ ID NO: 170 and the second sequence encoding a second ITR comprises the nucleic acid sequence of SEQ ID NO: 276. In an aspect, the first sequence encoding a first ITR comprises the nucleic acid sequence of SEQ ID NO: 171 and the second sequence encoding a second ITR comprises the nucleic acid sequence of SEQ ID NO: 276. In an aspect, the first sequence encoding a first ITR comprises the nucleic acid sequence of SEQ ID NO: 172 and the second sequence encoding a second ITR comprises the nucleic acid sequence of SEQ ID NO: 276. In an aspect, the first sequence encoding a first ITR comprises the nucleic acid sequence of SEQ ID NO: 173 and the second sequence encoding a second ITR comprises the nucleic acid sequence of SEQ ID NO: 276. In an aspect, the first sequence encoding a first ITR comprises the nucleic acid sequence of SEQ ID NO: 174 and the second sequence encoding a second ITR comprises the nucleic acid sequence of SEQ ID NO: 276. In an aspect, the first sequence encoding a first ITR comprises the nucleic acid sequence of SEQ ID NO: 175 and the second sequence encoding a second ITR comprises the nucleic acid sequence of SEQ ID NO: 276. In an aspect, the first sequence encoding a first ITR comprises the nucleic acid sequence of SEQ ID NO: 176 and the second sequence encoding a second ITR comprises the nucleic acid sequence of SEQ ID NO: 276. In an aspect, the first sequence encoding a first ITR comprises the nucleic acid sequence of SEQ ID NO: 177 and the second sequence encoding a second ITR comprises the nucleic acid sequence of SEQ ID NO: 276. In an aspect, the first sequence encoding a first ITR comprises the nucleic acid sequence of SEQ ID NO: 178 and the second sequence encoding a second ITR comprises the nucleic acid sequence of SEQ ID NO: 276. In an aspect, the first sequence encoding a first ITR comprises the nucleic acid sequence of SEQ ID NO: 179 and the second sequence encoding a second ITR comprises the nucleic acid sequence of SEQ ID NO: 276. In an aspect, the first sequence encoding a first ITR comprises the nucleic acid sequence of SEQ ID NO: 180 and the second sequence encoding a second ITR comprises the nucleic acid sequence of SEQ ID NO: 276. In an aspect, the first sequence encoding a first ITR comprises the nucleic acid sequence of SEQ ID NO: 181 and the second sequence encoding a second ITR comprises the nucleic acid sequence of SEQ ID NO: 276. In an aspect, the first sequence encoding a first ITR comprises the nucleic acid sequence of SEQ ID NO: 182 and the second sequence encoding a second ITR comprises the nucleic acid sequence of SEQ ID NO: 276. In an aspect, the first sequence encoding a first ITR comprises the nucleic acid sequence of SEQ ID NO: 183 and the second sequence encoding a second ITR comprises the nucleic acid sequence of SEQ ID NO: 276. In an aspect, the first sequence encoding a first ITR comprises the nucleic acid sequence of SEQ ID NO: 184 and the second sequence encoding a second ITR comprises the nucleic acid sequence of SEQ ID NO: 276. In an aspect, the first sequence encoding a first ITR comprises the nucleic acid sequence of SEQ ID NO: 185 and the second sequence encoding a second ITR comprises the nucleic acid sequence of SEQ ID NO: 276. In an aspect, the first sequence encoding a first ITR comprises the nucleic acid sequence of SEQ ID NO: 186 and the second sequence encoding a second ITR comprises the nucleic acid sequence of SEQ ID NO: 276. In an aspect, the first sequence encoding a first ITR comprises the nucleic acid sequence of SEQ ID NO: 187 and the second sequence encoding a second ITR comprises the nucleic acid sequence of SEQ ID NO: 276. In an aspect, the first sequence encoding a first ITR comprises the nucleic acid sequence of SEQ ID NO: 188 and the second sequence encoding a second ITR comprises the nucleic acid sequence of SEQ ID NO: 276. In an aspect, the first sequence encoding a first ITR comprises the nucleic acid sequence of SEQ ID NO: 189 and the second sequence encoding a second ITR comprises the nucleic acid sequence of SEQ ID NO: 276.

The composition can further comprise an origin of replication sequence or a sequence encoding an replication sequence. The first nucleic acid sequence or the second nucleic acid sequence can further comprise an origin of replication sequence or a sequence encoding an replication sequence. Preferably, the first nucleic acid sequence comprises an origin of replication sequence or a sequence encoding an replication sequence.

The origin of replication sequence can comprise an R6K origin of replication. The R6K origin of replication can comprise an R6K gamma origin of replication. The origin of replication sequence can comprise a mini origin of replication. The mini origin of replication can comprise an R6K mini origin of replication. The R6K mini origin of replication can comprise an R6K gamma mini origin of replication. The length of the R6K gamma mini origin of replication is 281 nucleotides (281 base pairs) and comprises, consists essentially of, or consists of the nucleic acid sequence of SEQ ID NO: 17.

The composition can further comprise a selectable marker or a sequence encoding a selectable marker. The nucleic acid sequence or the second nucleic acid sequence can further comprise a selectable marker or a sequence encoding a selectable marker. Preferably, the nucleic acid sequence comprises a selectable marker or a sequence encoding a selectable marker.

The nucleic acid sequence can further comprise at least one exogenous sequence and at least one promoter capable of expressing an exogenous sequence in a mammalian cell. In one aspect, the promoter is capable of expressing an exogenous sequence in a human cell. In one aspect, the transposon sequence of the composition comprises the at least one exogenous sequence and at least one promoter capable of expressing an exogenous sequence in a mammalian cell.

In some aspects, a transposon of the present disclosure can comprise at least one promoter sequence. In some aspects, a promoter sequence can comprise any promoter sequence known in the art. The promoter can be a constitutive promoter. The promoter can be an inducible promoter. The promoter can be a cell-type or tissue-type specific promoter. The promoter can be a EF1a promoter (SEQ ID NO: 18), a CMV promoter, an MND promoter, an SV40 promoter, a PGK1 promoter, a Ubc promoter, a CAG promoter, an H1 promoter, or a U6 promoter. In one aspect, the promoter is a EF1a promoter.

In some aspects, a promoter sequence can comprise a hybrid liver promoter (HLP). In some aspects, a promoter sequence can comprise an LP1 promoter. In some aspects, a promoter sequence can comprise a leukocyte-specific expression of the pp52 (LSP1) long promoter. In some aspects, a promoter sequence can comprise a thyroxine binding globulin (TBG) promoter.

In some aspects, a promoter sequence can comprise a wTBG promoter. In some aspects, a promoter sequence can comprise a hepatic combinatorial bundle (HCB) promoter. In some aspects, a promoter sequence can comprise an ApoE-hAAT promoter. In some aspects, a promoter sequence can comprise a 2×ApoE-hAAT promoter. In some aspects, a promoter sequence can comprise a leukocyte-specific expression of the pp52 (LSP1) plus chimeric intron promoter. In some aspects, a promoter sequence can comprise a cytomegalovirus (CMV) promoter.

In some aspects, a promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOS: 19-28, or a reverse complement thereof.

In some aspects, a promoter sequence can comprise a hybrid liver promoter (HLP). An HLP can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 19 or 27, or a reverse complement thereof.

In some aspects, a promoter sequence can comprise an LP1 promoter. An LP1 promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 20, or a reverse complement thereof.

In some aspects, a promoter sequence can comprise a leukocyte-specific expression of the pp52 (LSP1) long promoter. An LSP1 long promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 21, or a reverse complement thereof.

In some aspects, a promoter sequence can comprise a thyroxine binding globulin (TBG) promoter. A TBG promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 22, or a reverse complement thereof.

In some aspects, a promoter sequence can comprise a wTBG promoter. A wTBG promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 23, or a reverse complement thereof.

In some aspects, a promoter sequence can comprise a hepatic combinatorial bundle (HCB) promoter. An HCB promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 24, or a reverse complement thereof.

In some aspects, a promoter sequence can comprise a 2×ApoE-hAAT promoter. An 2×ApoE-hAAT promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 25, or a reverse complement thereof.

In some aspects, a promoter sequence can comprise a leukocyte-specific expression of the pp52 (LSP1) plus chimeric intron promoter. An LSP1 plus chimeric intron promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 26, or a reverse complement thereof.

In some aspects, a promoter sequence can comprise a cytomegalovirus (CMV) promoter. A CMV promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 28, or a reverse complement thereof.

The at least one exogenous sequence comprises, consists essentially of, or consists of a sequence encoding a non-naturally occurring antigen receptor, a sequence encoding a therapeutic polypeptide, or a combination thereof. The non-naturally occurring antigen receptor can comprise a chimeric antigen receptor (CAR), a T cell Receptor (TCR), a chimeric stimulatory receptor (CSR), an HLA class I histocompatibility antigen, alpha chain E recombinant polypeptide (HLA-E), Beta-2-Microglobulin (B2M) recombinant polypeptide, or a combination thereof. TCRs, CSRs, HLA-Es and B2Ms are described in detail herein. In one aspect, the non-naturally occurring antigen receptor comprises a CAR.

In some aspects, the at least one exogenous sequence comprises, consists essentially of, or consists of a sequence encoding a therapeutic polypeptide, a sequence encoding a therapeutic polypeptide, or a combination thereof.

In some aspects, a therapeutic polypeptide can comprise, consist essentially of, or consist of a methylmalonyl-CoA mutase (MUT1) polypeptide. In some aspects, nucleic acid sequence encoding a protein and/or peptide can comprise a nucleic acid sequence that encodes for a methylmalonyl-CoA mutase (MUT1) polypeptide. In some aspects, a nucleic acid sequence encoding a protein and/or peptide can comprise a nucleic acid sequence that encodes for a MUT1 polypeptide, wherein the MUT1 polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 29-32. In some aspects, a nucleic acid sequence that encodes for a MUT1 polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 33-44, or reverse complement thereof.

In some aspects, a therapeutic polypeptide can comprise, consist essentially of, or consist of an ornithine transcarbamylase (OTC) polypeptide. In some aspects, a nucleic acid sequence encoding a protein and/or peptide can comprise a nucleic acid sequence that encodes for an ornithine transcarbamylase (OTC) polypeptide. In some aspects, a nucleic acid sequence encoding a protein and/or peptide can comprise a nucleic acid sequence that encodes for an OTC polypeptide, wherein the OTC polypeptide comprises, consists essentially of or consist of an amino acid sequence at least 65%, 70%, 75%, 80%, 85%, 90%, 95%, 96%, 97%, 98%, 99% or 100% (or any percentage in between) identical to SEQ ID NO: 45 or 46. In some aspects, a nucleic acid sequence that encodes for an OTC polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 47-50, or a reverse complement thereof.

In some aspects, a therapeutic polypeptide can comprise, consist essentially of, or consist of a Factor VIII (FVIII) polypeptide. In some aspects, a FVIII polypeptide can be a FVIII polypeptide that is lacking the B-domain (hereafter referred to as a FVIII-BDD polypeptide). As would be appreciated by the skilled artisan, a Factor VIII-BDD polypeptide retains biological activity in vitro and in vivo (see Kessler et al. Haemophilia, 2005, 11 (2): 84-91). An FVIII-BDD polypeptide can comprise, consist essentially of consist of an amino acid sequence at least 65%, 70%, 75%, 80%, 85%, 90%, 95%, 96%, 97%, 98%, 99% or 100% (or any percentage in between) identical to SEQ ID NO: 51. In some aspects, a nucleic acid sequence that encodes for an FVIII-BDD polypeptide can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 52, or a reverse complement thereof.

In some aspects, a therapeutic polypeptide can comprise, consist essentially of, or consist of a Factor IX (FIX) polypeptide. In some aspects, a FIX polypeptide can comprise a R338L mutation. As would be appreciated by the skilled artisan, the R338L mutation can be referred to as the Padua mutation (see VandenDriessche and Chuah, Molecular Therapy, 2018, Vol. 26, Issue 1, P14-16, the contents of which are incorporated herein by reference in their entireties).

In some aspects, a transposon of the present disclosure can comprise at least one poly A sequence, or a reverse complement thereof. In some aspects, a polyA sequence can comprise any polyA sequence known in the art. In some aspects, a poly-A sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any of the sequences put forth in SEQ ID NOs: 53-57, or a reverse complement thereof.

In some aspects, a transposon of the present disclosure can comprise at least one 5′ UTR sequence.

Accordingly, a transposon of the present disclosure can comprise can comprise any combination of: i) at least one insulator sequence, or a reverse complement thereof: ii) at least one promoter sequence, or a reverse complement thereof: iii) at least one nucleic acid sequence encoding a protein and/or peptide, or a reverse complement thereof: iv) at least one polyA sequence, or a reverse complement thereof; at least one 5′ UTR sequence, or a reverse complement thereof, and/or vi) at least one 3′ UTR sequence, or a reverse complement thereof.

Accordingly, a transposon of the present disclosure can comprise any combination of: i) at least one insulator sequence, or a reverse complement thereof; ii) at least one promoter sequence, or a reverse complement thereof; iii) at least one nucleic acid sequence encoding a protein and/or peptide, or a reverse complement thereof; and/or iv) at least one polyA sequence, or a reverse complement thereof.

In a non-limiting example, a transposon of the present disclosure can comprise: i) a first insulator sequence, or a reverse complement thereof, followed by at least one promoter sequence, or a reverse complement thereof, followed by at least one nucleic acid sequence encoding a protein and/or peptide, or a reverse complement thereof, followed by at least on poly A sequence, or a reverse complement thereof, followed by a second insulator sequence, or a reverse complement thereof.

In some aspects, a transposon of the present disclosure can comprise at least one sequence encoding a therapeutic polypeptide and a 3′ UTR sequence. In some aspects, a transposon of the present disclosure can comprise, in the 5′ to 3′ direction, at least one sequence encoding a therapeutic polypeptide and a 3′ UTR sequence. In some aspects, a transposon of the present disclosure can comprise at least one sequence encoding a therapeutic polypeptide, followed by a 3′ UTR sequence.

In some aspects, a transposon of the present disclosure can comprise a first insulator sequence, at least one promoter sequence, at least one sequence encoding at least one therapeutic polypeptide, a 3′ UTR sequence, a polyA sequence and a second insulator sequence. In some aspects a transposon of the present disclosure can comprise, in the 5′ to 3′ direction, a first insulator sequence, at least one promoter sequence, at least one sequence encoding at least one therapeutic polypeptide, a 3′ UTR sequence, a polyA sequence, and a second insulator sequence. In some aspects, a transposon of the present disclosure can comprise, a first insulator sequence, followed by at least one promoter sequence, followed by at least one promoter sequence, followed by at least one sequence encoding at least one therapeutic polypeptide, followed by a 3′ UTR sequence, followed by a polyA sequence, followed by a second insulator sequence.

In some aspects of the preceding transposons, an at least one sequence encoding at least one therapeutic polypeptide can be a sequence encoding a FVIII-BDD polypeptide, wherein the FVIII-BDD polypeptide comprises the amino acid sequence of SEQ ID NO: 51. In some aspects, the sequence encoding a FVIII-BDD polypeptide can comprise the nucleic acid sequence of SEQ ID NO: 52, or a reverse complement thereof.

In some aspects of the preceding transposons, a 3′ UTR sequence can comprise the nucleic acid sequence of SEQ ID NO: 58, or a reverse complement thereof.

Accordingly, in a non-limiting example, a transposon of the present disclosure can comprise at least one sequence encoding a therapeutic polypeptide and a 3′ UTR sequence, wherein the at least one sequence encoding a therapeutic polypeptide is a sequence encoding a FVIII-BDD polypeptide, wherein the FVIII-BDD polypeptide comprises the amino acid sequence of SEQ ID NO: 51, and wherein the 3′ UTR sequence comprises the nucleic acid sequence of SEQ ID NO: 58, or a reverse complement thereof.

In some aspects, a transposon of the present disclosure can comprise at least one sequence encoding a therapeutic polypeptide, a first 3′ UTR sequence and a second 3′ UTR sequence. In some aspects, a transposon of the present disclosure can comprise, in the 5′ to 3′ sequence, at least one sequence encoding a therapeutic polypeptide, a first 3′ UTR sequence and a second 3′ UTR sequence. In some aspects, a transposon of the present disclosure can comprise at least one sequence encoding a therapeutic polypeptide, followed by a first 3′ UTR, followed by a second 3′ UTR sequence.

In some aspects, a transposon of the present disclosure can comprise a first insulator sequence, at least one promoter sequence, at least one sequence encoding at least one therapeutic polypeptide, a first 3′ UTR sequence, a second 3′ UTR sequence, a polyA sequence and a second insulator sequence. In some aspects, a transposon of the present disclosure can comprise, in the 5′ to 3′ direction, a first insulator sequence, at least one promoter sequence, at least one sequence encoding at least one therapeutic polypeptide, a first 3′ UTR sequence, a second 3′ UTR sequence, a polyA sequence, and a second insulator sequence. In some aspects, a transposon of the present disclosure can comprise a first insulator sequence, followed by at least one promoter sequence, followed by at least one promoter sequence, followed by at least one sequence encoding at least one therapeutic polypeptide, followed by a first 3′ UTR sequence, followed by a second 3′ UTR sequence, followed by a poly A sequence, followed by a second insulator sequence.

In some aspects of the preceding transposons, an at least one sequence encoding at least one therapeutic polypeptide can be a sequence encoding a FVIII-BDD polypeptide, wherein the FVIII-BDD polypeptide comprises the amino acid sequence of SEQ ID NO: 51. In some aspects, the sequence encoding a FVIII-BDD polypeptide can comprise the nucleic acid sequence of SEQ ID NO: 52, or a reverse complement thereof.

In some aspects of the preceding transposons, a first 3′ UTR sequence can be an AES 3′ UTR sequence, wherein the AES 3′ UTR sequence comprises the nucleic acid sequence of SEQ ID NO: 59.

In some aspects of the preceding transposons, a second 3′ UTR sequence can be a mtRNR1 3′ UTR sequence, wherein the mtRNR1 3′ UTR sequence comprises the nucleic acid sequence of SEQ ID NO: 60, or a reverse complement thereof.

Accordingly, in a non-limiting example, a transposon of the present disclosure can comprise at least one sequence encoding a therapeutic polypeptide, a first 3′ UTR sequence and a second 3′ UTR sequence, wherein the at least one sequence encoding a therapeutic polypeptide is a sequence encoding a FVIII-BDD polypeptide, wherein the FVIII-BDD polypeptide comprises the amino acid sequence of SEQ ID NO: 51, and wherein the first 3′ UTR sequence comprises the nucleic acid sequence of SEQ ID NO: 59, or a reverse complement thereof and the second 3′ UTR sequence comprises the nucleic acid sequence of SEQ ID NO: 60, or a reverse complement thereof.

The at least one exogenous sequence can further comprise, consist essential of, or consist of a sequence encoding an inducible proapoptotic polypeptide. Inducible proapoptotic polypeptides are described in detail herein.

The at least one exogenous sequence can further comprise, consist essential of, or consist of a sequence encoding a second selectable marker. The second selectable marker can encode a gene product essential for cell viability and survival. The second selectable marker can encode a gene product essential for cell viability and survival when challenged by selective cell culture conditions. Selective cell culture conditions may comprise a compound harmful to cell viability or survival and wherein the gene product confers resistance to the compound. Non-limiting examples of selection genes include neo (conferring resistance to neomycin), DHFR (encoding Dihydrofolate Reductase and conferring resistance to Methotrexate), TYMS (encoding Thymidylate Synthetase), MGMT (encoding O(6)-methylguanine-DNA methyltransferase), multidrug resistance gene (MDR1), ALDH1 (encoding Aldehyde dehydrogenase 1 family, member A1), FRANCF, RAD51C (encoding RAD51 Paralog C), GCS (encoding glucosylceramide synthase), NKX2.2 (encoding NK2 Homeobox 2), or any combination thereof.

The second selectable marker can be a detectable marker. The detectable marker can be a fluorescent marker, a cell-surface marker or a metabolic marker. In a preferred aspect, the second selectable marker comprises a sequence encoding a dihydrofolate reductase (DHFR) mutein enzyme. The DHFR mutein enzyme comprises, consists essentially of, or consists of the amino acid sequence of SEQ ID NO: 61. The DHFR mutein enzyme is encoded by a polynucleotide comprising, consisting essential of, or consisting of the nucleic acid sequence of SEQ ID NO: 62. The amino acid sequence of the DHFR mutein enzyme can further comprise a mutation at one or more of positions 80, 113, or 153. The amino acid sequence of the DHFR mutein enzyme can comprise one or more of a substitution of a Phenylalanine (F) or a Leucine (L) at position 80, a substitution of a Leucine (L) or a Valine (V) at position 113, and a substitution of a Valine (V) or an Aspartic Acid (D) at position 153.

The at least one exogenous sequence can further comprise, consist essential of, or consist of a sequence encoding at least one self-cleaving peptide. For example, a self-cleaving peptide can be located between a CAR and an inducible proapoptotic polypeptide; or, a self-cleaving peptide can be located between a CAR and second selectable marker.

The at least one exogenous sequence can further comprise, consist essential of, or consist of a sequence encoding at least two self-cleaving peptides. For example, a first self-cleaving peptide is located upstream or immediately upstream of a CAR and a second self-cleaving peptide is located downstream or immediately downstream of a CAR; or, the first self-cleaving peptide and the second self-cleaving peptide flank a CAR. For example, a first self-cleaving peptide is located upstream or immediately upstream of an inducible proapoptotic polypeptide and a second self-cleaving peptide is located downstream or immediately downstream of an inducible proapoptotic polypeptide; or, the first self-cleaving peptide and the second self-cleaving peptide flank an inducible proapoptotic polypeptide. For example, a first self-cleaving peptide is located upstream or immediately upstream of a second selectable marker and a second self-cleaving peptide is located downstream or immediately downstream of a second selectable marker; or, the first self-cleaving peptide and the second self-cleaving peptide flank a second selectable marker.

Non-limiting examples of self-cleaving peptides include a T2A peptide, GSG-T2A peptide, an E2A peptide, a GSG-E2A peptide, an F2A peptide, a GSG-F2A peptide, a P2A peptide, or a GSG-P2A peptide. Non-limiting examples of self cleaving peptides are described in PCT Publication No. WO 2020/132396.

The polynucleotide encoding a transposon comprising at least one exogenous sequence and at least one promoter capable of expressing an exogenous sequence in a mammalian cell can further comprise at least one sequence encoding an insulator. In an aspect, the polynucleotide can comprise a first sequence encoding a first insulator and a second sequence encoding a second insulator. In some embodiments the sequence encoding a first or second insulator comprises, consists essential of, or consists of, the nucleic acid sequence at least 75%, 80%, 85%, 90%, 95%, 96%, 97%, 98%, 99% or 100% (or any percentage in between) identical to SEQ ID NO: 64 or SEQ ID NO: 65.

The polynucleotide encoding a transposon comprising at least one exogenous sequence and at least one promoter capable of expressing an exogenous sequence in a mammalian cell can further comprise a polyadenosine (polyA) sequence. The polynucleotide comprising at least one exogenous sequence, at least one promoter capable of expressing an exogenous sequence in a mammalian cell and at least one sequence encoding an insulator can further comprise a polyadenosine (polyA) sequence. The polyA sequence can be isolated or derived from a viral poly A sequence. The poly A sequence can be isolated or derived from an (SV40) polyA sequence. In some embodiments the sequence encoding a first or second insulator comprises, consists essential of, or consists of, the nucleic acid sequence at least 75%, 80%, 85%, 90%, 95%, 96%, 97%, 98%, 99% or 100% (or any percentage in between) identical to SEQ ID NO: 68.

In an aspect, the composition does not comprise a sequence encoding foreign DNA. In an aspect, the polynucleotide encoding a transposon does not comprise a sequence encoding foreign DNA. In an aspect, the composition comprises a sequence encoding foreign DNA. In an aspect, the polynucleotide encoding a transposon comprises a sequence encoding foreign DNA. Foreign DNA is an DNA sequence which is not derived or obtained from the same organism as the mammalian cell in which the exogenous sequence will be expressed. For example, foreign DNA could be DNA from a virus, rather than a mammal; or the foreign DNA could be DNA from a reptile, rather than a mammal. In another aspect, the foreign DNA could be from one mammal but that mammal is different from the mammal in which the exogenous sequence will be expressed. For example, the foreign DNA is from a rat rather than a human.

In an aspect, the composition does not comprise a recombination site, an excision site, a ligation site, or a combination thereof. In an aspect, the composition does not comprise a product of a recombination event, an excision event, a ligation event, or a combination thereof. In an aspect, the composition is not derived from a recombination event, an excision event, a ligation event, or a combination thereof.

In an aspect, the first nucleic acid sequence does not comprise a recombination site, an excision site, a ligation site, or a combination thereof. In an aspect, the first nucleic acid sequence does not comprise a product of a recombination event, an excision event, a ligation event, or a combination thereof. In an aspect, the first nucleic acid sequence is not derived from a recombination event, an excision event, a ligation event, or a combination thereof.

A recombination site can comprise a sequence resulting from a recombination event, can comprise a sequence that is a product of a recombination event, or can comprise an activity of a recombinase (e.g., a recombinase site).

### Chimeric Antigen Receptor (CAR)

The present disclosure also provides a polynucleotide encoding a transposon comprising a nucleic acid sequence encoding a CAR, wherein the CAR comprises an ectodomain comprising antigen recognition region; a transmembrane domain, and an endodomain comprising at least one costimulatory domain. The CAR can further comprise a hinge region between the antigen recognition domain and the transmembrane domain.

The antigen recognition region can comprise at least one single chain variable fragment (scFv), Centyrin, single domain antibody, or a combination thereof. In an aspect, the at least one single domain antibody is a VHH. In an aspect, the at least one single domain antibody is a VH.

scFv

The compositions of the disclosure (e.g., transposons or nanotransposons) can comprise a CAR; and in some aspects, the antigen recognition region of the CAR can comprise one or more scFv compositions to recognize and bind to a specific target protein/antigen. The antigen recognition region can comprise at least two scFvs. The antigen recognition region can comprise at least three scFvs. In an aspect, a CAR of the disclosure is a bi-specific CAR comprising at least two scFvs that specifically bind two distinct antigens.

The scFv compositions comprise a heavy chain variable region and a light chain variable region of an antibody. An scFv is a fusion protein of the variable regions of the heavy (VH) and light (VL) chains of immunoglobulins, and the VH and VL domains are connected with a short peptide linker. An scFv can retain the specificity of the original immunoglobulin, despite removal of the constant regions and the introduction of the linker. In some aspects, the linker polypeptide comprises, consists essentially of, or consists of the amino acid sequence of SEQ ID NO: 69. The linker polypeptide can be encoded by a polynucleotide comprising, consisting essentially of, or consists of the nucleic acid sequence of SEQ ID NO: 70.

### Centyrin

The compositions of the disclosure (e.g., transposons or nanotransposons) can comprise a CAR; and in some aspects, the antigen recognition region of the CAR can comprise one or more Centyrin compositions to recognize and bind to a specific target protein/antigen. Centyrins that specifically bind an antigen may be used to direct the specificity of a cell, (e.g., a cytotoxic immune cell) towards the specific antigen. A CAR comprising a Centyrin is referred to herein as a CARTyrin.

Centyrins of the disclosure may comprise a protein scaffold, wherein the scaffold is capable of specifically binding an antigen. Centyrins of the disclosure may comprise a protein scaffold comprising a consensus sequence of at least one fibronectin type III (FN3) domain, wherein the scaffold is capable of specifically binding an antigen. The at least one fibronectin type III (FN3) domain may be derived from a human protein. The human protein may be Tenascin-C. Non-limiting examples of Centyrins are described in PCT Publication No. WO 2020/132396.

The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Antibody mimetics may provide superior properties over antibodies including, but not limited to, superior solubility, tissue penetration, stability towards heat and enzymes (e.g., resistance to enzymatic degradation), and lower production costs. Exemplary antibody mimetics include, but are not limited to, an affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody. Non-limiting examples of affibody, an afflilin, an affimer, an affitin, an alphabody, an anticalin, and avimer (also known as avidity multimer), a DARPin (Designed Ankyrin Repeat Protein), a Fynomer, a Kunitz domain peptide, and a monobody are described in PCT Publication No. WO 2020/132396.

### VHH

The polynucleotide encoding a transposon in the disclosure can comprise a nucleic acid sequence encoding a CAR. In some aspects, the antigen recognition region of the CAR can comprise at least one single domain antibodies (SdAb) to recognize and bind to a specific target protein/antigen. In an aspect, the single domain antibody is a VHH. A VHH is a heavy chain antibody found in camelids. A VHH that specifically binds an antigen may be used to direct the specificity of a cell, (e.g., a cytotoxic immune cell) towards the specific antigen. The antigen recognition region can comprise at least two VHHs. The antigen recognition region can comprise at least three VHHs. In an aspect, a CAR of the disclosure is a bi-specific CAR comprising at least two VHHs that specifically bind two distinct antigens. A CAR comprising a VHH is referred to herein as a VCAR.

At least one VHH protein or VCAR of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a VHH protein can be altered, added and/or deleted to reduce immunogenicity or reduce, enhance or modify binding, affinity, on-rate, off-rate, avidity, specificity, half-life, stability, solubility or any other suitable characteristic, as known in the art.

### VH

The polynucleotide encoding a transposon of the disclosure can comprise a nucleic acid sequence encoding a CAR; and in some aspects, the antigen recognition region of the CAR can comprise at least one single domain antibodies (SdAb) to recognize and bind to a specific target protein/antigen. In an aspect, the single domain antibody is a VH. A VH is a single domain binder derived from common IgG. A VH that specifically binds an antigen may be used to direct the specificity of a cell, (e.g., a cytotoxic immune cell) towards the specific antigen. The antigen recognition region can comprise at least two VHs. The antigen recognition region can comprise at least three VHs. In an aspect, a CAR of the disclosure is a bi-specific CAR comprising at least two VHs that specifically bind two distinct antigens. Methods of VH isolation and VH engineering are described in PCT Publication No. WO 2020/132396.

A CAR of the present disclosure may bind human antigen with at least one affinity selected from a K_{D} of less than or equal to 10⁻⁹M, less than or equal to 10⁻¹⁰M, less than or equal to 10⁻¹¹M, less than or equal to 10⁻¹²M, less than or equal to 10⁻¹³M, less than or equal to 10⁻¹⁴M, and less than or equal to 10⁻¹⁵M. The K_{D} may be determined by any means, including, but not limited to, surface plasmon resonance.

In an aspect, the antigen recognition region of the disclosed CAR comprises at least one anti-BCMA Centyrin. The anti-BCMA Centyrin comprises, consists essentially of, or consists of the amino acid sequence at least 95%, 96%, 97%, 98%, 99% or 100% (or any percentage in between) identical to SEQ ID NO: 71. The anti-BCMA Centyrin is encoded by a polynucleotide comprising, consisting essentially of, or consisting of the nucleic acid sequence at least 95%, 96%, 97%, 98%, 99% or 100% (or any percentage in between) identical to SEQ ID NO: 72.

A CAR comprising the anti-BCMA Centyrin is referred to as a BCMA CARTyrin herein. In a preferred aspect, the BCMA CARTyrin comprises, consists essentially of, or consists of the amino acid sequence at least 95%, 96%, 97%, 98%, 99% or 100% (or any percentage in between) identical to SEQ ID NO: 73. The BCMA CARTyrin is encoded by a polynucleotide comprising, consisting essentially of, or consisting of the nucleic acid sequence at least 95%, 96%, 97%, 98%, 99% or 100% (or any percentage in between) identical to SEQ ID NO: 74.

A composition of the disclosure (e.g., transposon) comprising a BCMA CARTyrin comprises, consists essentially of, or consists of the amino acid sequence at least 95%, 96%, 97%, 98%, 99% or 100% (or any percentage in between) identical to SEQ ID NO: 75. A composition of the disclosure (e.g., transposon) comprising a BCMA CARTyrin is encoded by a polynucleotide comprising, consisting essentially of, or consisting of the nucleic acid sequence at least 95%, 96%, 97%, 98%, 99% or 100% (or any percentage in between) identical to SEQ ID NO: 76.

In an aspect, the antigen recognition region of the disclosed CAR comprises at least one anti-PSMA Centyrin. The anti-PSMA Centyrin comprises, consists essentially of, or consists of the amino acid sequence at least 95%, 96%, 97%, 98%, 99% or 100% (or any percentage in between) identical to SEQ ID NO: 77. The anti-PSMA Centyrin is encoded by a polynucleotide comprising, consisting essentially of, or consisting of the nucleic acid sequence at least 95%, 96%, 97%, 98%, 99% or 100% (or any percentage in between) identical to SEQ ID NO: 78.

A CAR comprising the anti-PSMA Centyrin is referred to as a PSMA CARTyrin herein. In a preferred aspect, the PSMA CARTyrin comprises, consists essentially of, or consists of the amino acid sequence at least 95%, 96%, 97%, 98%, 99% or 100% (or any percentage in between) identical to SEQ ID NO: 79. The PSMA CARTyrin is encoded by a polynucleotide comprising, consisting essentially of, or consisting of the nucleic acid sequence at least 95%, 96%, 97%, 98%, 99% or 100% (or any percentage in between) identical to SEQ ID NO: 80.

A composition of the disclosure (e.g., transposon) comprising a PSMA CARTyrin comprises, consists essentially of, or consists of the amino acid sequence at least 95%, 96%, 97%, 98%, 99% or 100% (or any percentage in between) identical to SEQ ID NO: 81. A composition of the disclosure (e.g., transposon) comprising a PSMA CARTyrin is encoded by a polynucleotide comprising, consisting essentially of, or consisting of the nucleic acid sequence at least 95%, 96%, 97%, 98%, 99% or 100% (or any percentage in between) identical to SEQ ID NO: 82.

In an aspect, the antigen recognition region of the disclosed CAR comprises at least one anti-BCMA VH. The anti-BCMA VH comprises, consists essentially of, or consists of the amino acid sequence at least 95%, 96%, 97%, 98%, 99% or 100% (or any percentage in between) identical to SEQ ID NO: 83. The anti-BCMA VH is encoded by a polynucleotide comprising, consisting essentially of, or consisting of the nucleic acid sequence at least 95%, 96%, 97%, 98%, 99% or 100% (or any percentage in between) identical to SEQ ID NO: 84.

A CAR comprising the anti-BCMA VH is referred to as a BCMA VCAR herein. In a preferred aspect, the BCMA VCAR comprises, consists essentially of, or consists of the amino acid sequence at least 95%, 96%, 97%, 98%, 99% or 100% (or any percentage in between) identical to SEQ ID NO: 85. The BCMA VCAR is encoded by a polynucleotide comprising, consisting essentially of, or consisting of the nucleic acid sequence at least 95%, 96%, 97%, 98%, 99% or 100% (or any percentage in between) identical to SEQ ID NO: 86.

A composition of the disclosure (e.g., transposon) comprising a BCMA VCAR comprises, consists essentially of, or consists of the amino acid sequence at least 95%, 96%, 97%, 98%, 99% or 100% (or any percentage in between) identical to SEQ ID NO: 87. A composition of the disclosure (e.g., transposon) comprising a BCMA VCAR is encoded by a polynucleotide comprising, consisting essentially of, or consisting of the nucleic acid sequence at least 95%, 96%, 97%, 98%, 99% or 100% (or any percentage in between) identical to SEQ ID NO: 88.

In an aspect, the antigen recognition region of the disclosed CAR comprises at least one anti-MUC1-C ScFv. The anti-MUC1-C ScFv comprises, consists essentially of, or consists of the amino acid sequence at least 95%, 96%, 97%, 98%, 99% or 100% (or any percentage in between) identical to SEQ ID NO: 89. The anti-MUC1-C ScFv is encoded by a polynucleotide comprising, consisting essentially of, or consisting of the nucleic acid sequence at least 95%, 96%, 97%, 98%, 99% or 100% (or any percentage in between) identical to SEQ ID NO: 90.

A CAR comprising the anti-MUC1-C ScFv is referred to as a MUC1-C CAR herein. In a preferred aspect, the MUC1-C CAR comprises, consists essentially of, or consists of the amino acid sequence at least 95%, 96%, 97%, 98%, 99% or 100% (or any percentage in between) identical to SEQ ID NO: 91. The MUC1-C CAR is encoded by a polynucleotide comprising, consisting essentially of, or consisting of the nucleic acid sequence at least 95%, 96%, 97%, 98%, 99% or 100% (or any percentage in between) identical to SEQ ID NO: 92.

A composition of the disclosure (e.g., transposon) comprising a MUC1-C CAR comprises, consists essentially of, or consists of the amino acid sequence at least 95%, 96%, 97%, 98%, 99% or 100% (or any percentage in between) identical to SEQ ID NO: 93. A composition of the disclosure (e.g., transposon) comprising a MUC1-C CAR is encoded by a polynucleotide comprising, consisting essentially of, or consisting of the nucleic acid sequence at least 95%, 96%, 97%, 98%, 99% or 100% (or any percentage in between) identical to SEQ ID NO: 94.

The ectodomain can comprise a signal peptide. The signal peptide can comprise a sequence encoding a human CD2, CD3δ, CD3ϵ, CD3γ, CD3ζ, CD4, CD8α, CD19, CD28, 4-1BB or GM-CSFR signal peptide. In a preferred aspect, the signal peptide comprises, consists essentially of, or consists of: a human CD8 alpha (CD8α) signal peptide (SP) or a portion thereof. The human CD8α SP comprises, consists essentially of, or consists of an amino acid sequence at least 75%, 80%, 85%, 90%, 95%, 96%, 97%, 98%, 99% or 100% (or any percentage in between) identical to SEQ ID NO: 95. Preferably, the human CD8α SP comprises, consists essentially of, or consists of the amino acid sequence of SEQ ID NO: 95.

The human CD8α SP is encoded by a polynucleotide comprising, consisting essentially of or consisting of a nucleic acid sequence at least 75%, 80%, 85%, 90%, 95%, 96%, 97%, 98%, 99% or 100% (or any percentage in between) identical to SEQ ID NO: 96. Preferably, the human CD8α SP is encoded by a polynucleotide comprising, consisting essentially of, or consisting of the amino acid sequence of SEQ ID NO: 96.

The hinge domain or hinge region can comprise a human CD8α, IgG4, CD4 sequence, or a combination thereof. In a preferred aspect, the hinge can comprise, consist essentially of, or consist of a human CD8 alpha (CD8α) hinge or a portion thereof. The human CD8α hinge comprises, consists essentially, of or consists of an amino acid sequence at least 75%, 80%, 85%, 90%, 95%, 96%, 97%, 98%, 99% or 100% (or any percentage in between) identical to SEQ ID NO: 97. Preferably, the human CD8α hinge domain comprises, consists essentially of, or consists of the amino acid sequence of SEQ ID NO: 97.

The human CD8α hinge is encoded by a polynucleotide comprising, consisting essentially of or consisting of a nucleic acid sequence at least 75%, 80%, 85%, 90%, 95%, 96%, 97%, 98%, 99% or 100% (or any percentage in between) identical to SEQ ID NO: 98. Preferably, the human CD8α hinge domain is encoded by a polynucleotide comprising, consisting essentially of or consisting of the nucleic acid sequence of SEQ ID NO: 98.

The transmembrane domain can comprise, consist essentially of, or consist of a sequence encoding a human CD2, CD3δ, CD3ϵ, CD3γ, CD3ζ, CD4, CD8α, CD19, CD28, 4-1BB or GM-CSFR transmembrane domain. Preferably, the transmembrane domain can comprise, consist essentially of, or consist of a human CD8 alpha (CD8α) transmembrane domain, or a portion thereof. The CD8α transmembrane domain comprises, consists essentially of or consists of an amino acid sequence at least 75%, 80%, 85%, 90%, 95%, 96%, 97%, 98%, 99% or 100% (or any percentage in between) identical to SEQ ID NO: 99. Preferably, the human CD8α transmembrane domain comprises, consists essentially of, or consists of the amino acid sequence of SEQ ID NO: 99.

The CD8α transmembrane domain is encoded by a polynucleotide comprising, consisting essentially of, or consisting of a nucleic acid sequence at least 75%, 80%, 85%, 90%, 95%, 96%, 97%, 98%, 99% or 100% (or any percentage in between) identical to SEQ ID NO: 100. Preferably, the CD8α transmembrane domain is encoded by a polynucleotide comprising, consisting essentially of, or consisting of the nucleic acid sequence of SEQ ID NO: 100.

The at least one costimulatory domain can comprise, consist essentially of, or consist of a human 4-1BB, CD28, CD3 zeta (CD3ζ), CD40, ICOS, MyD88, OX-40 intracellular domain, or any combination thereof. Preferably, the at least one costimulatory domain comprises a CD3ζ, a 4-1BB costimulatory domain, or a combination thereof.

The 4-1BB intracellular domain comprises, consists essentially of, or consists of an amino acid sequence at least 75%, 80%, 85%, 90%, 95%, 96%, 97%, 98%, 99% or 100% (or any percentage in between) identical to SEQ ID NO: 101. Preferably, the 4-1BB intracellular domain comprises, consists essentially of, or consists of the amino acid sequence of SEQ ID NO: 101.

The 4-1BB intracellular domain is encoded by a polynucleotide comprising, consisting essentially of, or consisting of a nucleic acid sequence at least 75%, 80%, 85%, 90%, 95%, 96%, 97%, 98%, 99% or 100% (or any percentage in between) identical to SEQ ID NO: 102. Preferably, the 4-1BB intracellular domain is encoded by a polynucleotide comprising, consisting essentially of or consisting of the nucleic acid sequence of SEQ ID NO: 102.

The CD3ζ intracellular domain comprises, consists essentially of, or consists of an amino acid sequence at least 75%, 80%, 85%, 90%, 95%, 96%, 97%, 98%, 99% or 100% (or any percentage in between) identical to SEQ ID NO: 103. Preferably, the CD3ζ intracellular domain comprises, consists essentially of, or consists of the amino acid sequence of SEQ ID NO: 103.

The CD3ζ intracellular domain is encoded by a polynucleotide comprising, consisting essentially of, or consisting of a nucleic acid sequence at least 75%, 80%, 85%, 90%, 95%, 96%, 97%, 98%, 99% or 100% (or any percentage in between) identical to SEQ ID NO: 104. Preferably, the CD3ζ intracellular domain is encoded by a polynucleotide comprising, consisting essentially of, or consisting of the nucleic acid sequence of SEQ ID NO: 104.

### Transposon and Vector Compositions

### Transposition Systems

The present disclosure provides a polynucleotide encoding a transposon comprising: a) a first inverted terminal repeat (ITR) and b) a second ITR, wherein the first ITR and/or the second ITR comprises at least one nucleic acid substitution relative to a wildtype ITR of a transposon. In some aspects, the transposon is a nanotransposon. In some aspects, the first ITR is the left end ITR. In some aspects, the at least one nucleic acid substitution relative to a wildtype ITR of the transposon provides an increase in transposition activity. In some aspects, the increase in transposition activity is an increase in excision of a transposon in a cell. In some aspects, the increase in transposition activity is an increase in integration of a transposon in a cell. In some aspects, the increase in transposition activity is an increase in excision and an increase in integration of a transposon in a cell.

The transposon or nanotransposon of the disclosure comprises a protein scaffold (e.g., a CAR comprising at least one scFv, single domain antibody or Centyrin). The transposon or nanotransposon can be a plasmid DNA transposon comprising a sequence encoding a protein scaffold (e.g., a CAR comprising at least one scFv, single domain antibody or Centyrin) flanked by two cis-regulatory insulator elements. The transposon or nanotransposon can further comprises a plasmid comprising a sequence encoding a transposase. The sequence encoding the transposase may be a DNA sequence or an RNA sequence. Preferably, the sequence encoding the transposase is an mRNA sequence.

The transposon or nanotransposon of the present disclosure can be a piggyBac™ (PB) transposon. In some aspects when the transposon is a PB transposon, the transposase is a piggyBac™ (PB) transposase a piggyBac-like (PBL) transposase or a Super piggyBac™ (SPB) transposase. Preferably, the sequence encoding the SPB transposase is an mRNA sequence.

Non-limiting examples of PB transposons and PB, PBL and SPB transposases are described in detail in U.S. Pat. Nos. 6,218,182; 6,962,810; 8,399,643 and PCT Publication No. WO 2010/099296.

The PB, PBL and SPB transposases recognize transposon-specific inverted terminal repeat sequences (ITRs) on the ends of the transposon, and inserts the contents between the ITRs at the sequence 5′-TTAT-3′ within a chromosomal site (a TTAT target sequence) or at the sequence 5′-TTAA-3′ within a chromosomal site (a TTAA target sequence). The target sequence of the PB or PBL transposon can comprise or consist of 5′-CTAA-3′, 5′-TTAG-3′, 5′-ATAA-3″, 5′-TCAA-3′, 5′AGTT-3′, 5′-ATTA-3′, 5′-GTTA-3′, 5′-TTGA-3′, 5′-TTTA-3′, 5′-TTAC-3″, 5′-ACTA-3′, 5′-AGGG-3′, 5′-CTAG-3′, 5′-TGAA-3′, 5′-AGGT-3′, 5′-ATCA-3′, 5′-CTCC-3″, 5′-TAAA-3′, 5′-TCTC-3′, 5′TGAA-3′, 5′-AAAT-3′, 5′-AATC-3′, 5′-ACAA-3′, 5′-ACAT-3″, 5′-ACTC-3′, 5′-AGTG-3′, 5′-ATAG-3′, 5′-CAAA-3′, 5′-CACA-3′, 5′-CATA-3′, 5′-CCAG-3″, 5′-CCCA-3′, 5′-CGTA-3′, 5′-GTCC-3′, 5′-TAAG-3′, 5′-TCTA-3′, 5′-TGAG-3′, 5′-TGTT-3″, 5′-TTCA-3′5′-TTCT-3′ and 5′-TTTT-3′. The PB or PBL transposon system has no payload limit for the genes of interest that can be included between the ITRs.

Exemplary amino acid sequence for one or more PB, PBL and SPB transposases are disclosed in U.S. Pat. Nos. 6,218,185; 6,962,810 and 8,399,643. In an aspect, the PB transposase comprises or consists of an amino acid sequence at least 75%, 80%, 85%, 90%, 95%, 96%, 97%, 98%, 99% or 100% (or any percentage in between) identical to SEQ ID NO: 105.

In one aspect, the SPB transposase (“wildtype SPB”) comprises, consists essentially of, or consists of the amino acid sequence at least 95%, 96%, 97%, 98%, 99% or 100% (or any percentage in between) identical to SEQ ID NO: 107.

(SEQ ID NO: 107)
MAPKKKRKVGGGGSSLDDEHILSALLQSDDELVGEDSDSEVSDHVSEDD
VQSDTEEAFIDEVHEVQPTSSGSEILDEQNVIEQPGSSLASNRILTLPQ
RTIRGKNKHCWSTSKSTRRSRVSALNIVRSQRGPTRMCRNIYDPLLCFK
LFFTDEIISEIVKWTNAEISLKRRESMTSATERDTNEDEIYAFFGILVM
TAVRKDNHMSTDDLFDRSLSMVYVSVMSRDREDFLIRCLRMDDKSIRPT
LRENDVFTPVRKIWDLFIHQCIQNYTPGAHLTIDEQLLGFRGRCPFRVY
IPNKPSKYGIKILMMCDSGTKYMINGMPYLGRGTQTNGVPLGEYYVKEL
SKPVHGSCRNITCDNWFTSIPLAKNLLQEPYKLTIVGTVRSNKREIPEV
LKNSRSRPVGTSMFCFDGPLTLVSYKPKPAKMVYLLSSCDEDASINEST
GKPQMVMYYNQTKGGVDTLDQMCSVMTCSRKTNRWPMALLYGMINIACI
NSFIIYSHNVSSKGEKVQSRKKFMRNLYMSLTSSFMRKRLEAPTLKRYL
RDNISNILPKEVPGTSDDSTEEPVMKKRTYCTYCPSKIRRKANASCKKC
KKVICREHNIDMCQSCF

In one aspect, the SPB transposase (“wildtype SPB”) is encoded by a polynucleotide comprising, consisting essentially of, or consisting of the nucleic acid sequence at least 95%, 96%, 97%, 98%, 99% or 100% (or any percentage in between) identical to SEQ ID NO: 108.

(SEQ ID NO: 108)
ATGGCTCCCAAGAAGAAGCGGAAAGTTGGCGGCGGAGGCAGCAGCCTGGATGATGAGCATATTC
TGAGCGCCCTGCTGCAGAGCGACGATGAACTCGTGGGCGAAGATAGCGACAGCGAGGTGTCCGA
TCACGTGTCCGAGGATGACGTGCAGTCCGATACCGAGGAAGCCTTCATCGACGAGGTGCACGAA
GTGCAGCCTACAAGCAGCGGCAGCGAGATCCTGGACGAGCAGAATGTGATCGAGCAGCCAGGAT
CTAGCCTGGCCAGCAACAGAATCCTGACACTGCCCCAGAGAACCATCCGGGGCAAGAACAAGCA
CTGCTGGTCCACCAGCAAGAGCACCAGACGGTCTAGAGTGTCTGCCCTGAACATCGTGCGAAGC
CAGAGGGGCCCTACCAGAATGTGCCGGAACATCTACGACCCTCTGCTGTGCTTCAAGCTGTTCT
TCACCGACGAGATCATCTCCGAGATCGTGAAGTGGACCAACGCCGAGATCAGCCTGAAGCGGAG
AGAATCCATGACCAGCGCCACCTTCAGAGACACCAACGAGGACGAGATCTACGCCTTCTTCGGC
ATCCTGGTCATGACAGCCGTGCGGAAGGACAACCACATGAGCACCGACGACCTGTTCGACCGCA
GCCTGTCTATGGTGTACGTGTCCGTGATGAGCCGGGACAGATTCGACTTCCTGATCCGGTGCCT
GCGGATGGACGACAAGTCCATCAGACCCACACTGCGCGAGAACGACGTGTTCACACCTGTGCGG
AAGATCTGGGACCTGTTCATCCACCAGTGCATCCAGAACTACACCCCTGGCGCTCACCTGACCA
TCGACGAACAGCTGCTGGGCTTCAGAGGCAGATGCCCCTTCAGAGTGTACATCCCCAACAAGCC
CTCTAAGTACGGCATCAAGATCCTGATGATGTGCGACAGCGGCACCAAGTACATGATCAACGGC
ATGCCCTACCTCGGCAGAGGCACCCAAACAAATGGCGTGCCACTGGGCGAGTACTACGTGAAAG
AACTGAGCAAGCCTGTGCACGGCAGCTGCAGAAACATCACCTGTGACAACTGGTTTACCAGCAT
TCCCCTGGCCAAGAACCTGCTGCAAGAACCCTACAAGCTGACAATCGTGGGCACCGTGCGGAGC
AACAAGAGGGAAATTCCCGAGGTGCTGAAGAACTCTCGGAGCAGACCTGTGGGCACCAGCATGT
TCTGCTTCGACGGACCTCTGACACTGGTGTCCTACAAGCCCAAGCCTGCCAAGATGGTGTACCT
GCTGAGCAGCTGTGACGAGGACGCCAGCATCAATGAGAGCACCGGCAAGCCCCAGATGGTCATG
TACTACAACCAGACCAAAGGCGGCGTGGACACCCTGGATCAGATGTGCAGCGTGATGACCTGCA
GCAGAAAGACCAACAGATGGCCCATGGCTCTGCTGTACGGCATGATCAATATCGCCTGCATCAA
CAGCTTCATCATCTACAGCCACAACGTGTCCAGCAAGGGCGAGAAGGTGCAGAGCCGGAAGAAA
TTCATGCGGAACCTGTACATGAGCCTGACCAGCAGCTTCATGAGAAAGCGGCTGGAAGCCCCTA
CACTGAAGAGATACCTGCGGGACAACATCAGCAACATCCTGCCTAAAGAGGTGCCCGGCACCAG
CGACGATAGCACAGAGGAACCCGTGATGAAGAAGAGGACCTACTGCACCTACTGTCCCAGCAAG
ATCCGGCGGAAGGCCAACGCCAGCTGCAAAAAGTGCAAGAAAGTGATCTGCCGCGAGCACAACA
TCGATATGTGCCAGAGCTGCTTCTGA

The PB or PBL transposase can comprise or consist of an amino acid sequence having an amino acid substitution at two or more, at three or more or at each of positions 30, 165, 282, or 538 of the sequence of SEQ ID NO: 105. The transposase can be a SPB transposase that comprises or consists of the amino acid sequence of the sequence of SEQ ID NO: 105 wherein the amino acid substitution at position 30 can be a substitution of a valine (V) for an isoleucine (I), the amino acid substitution at position 165 can be a substitution of a serine(S) for a glycine (G), the amino acid substitution at position 282 can be a substitution of a valine (V) for a methionine (M), and the amino acid substitution at position 538 can be a substitution of a lysine (K) for an asparagine (N). In a preferred aspect, the SPB transposase comprises or consists of an amino acid sequence at least 75%, 80%, 85%, 90%, 95%, 96%, 97%, 98%, 99% or 100% (or any percentage in between) identical to SEQ ID NO: 106.

In certain aspects wherein the transposase comprises the above-described mutations at positions 30, 165, 282 and/or 538, the PB, PBL and SPB transposases can further comprise an amino acid substitution at one or more of positions 3, 46, 82, 103,119, 125, 177, 180, 185, 187, 200, 207, 209, 226, 235, 240, 241, 243, 258, 296, 298, 311, 315, 319, 327, 328, 340, 421, 436, 456, 470, 486, 503, 552, 570 and 591 of the sequence of SEQ ID NO: 105 or SEQ ID NO: 106 are described in more detail in PCT Publication No. WO 2019/173636 and PCT/US2019/049816.

In one aspect, the SPB transposase (“M226F SPB” or “mutant M226F SPB”) comprises, consists essentially of, or consists of the amino acid sequence at least 95%, 96%, 97%, 98%, 99% or 100% (or any percentage in between) identical to SEQ ID NO: 109. The M226F mutation is shown in bolded and underlined font.

(SEQ ID NO: 109)
MAPKKKRKVGGGGSSLDDEHILSALLQSDDELVGEDSDSEVSDHVSEDD
VQSDTEEAFIDEVHEVQPTSSGSEILDEQNVIEQPGSSLASNRILTLPQ
RTIRGKNKHCWSTSKSTRRSRVSALNIVRSQRGPTRMCRNIYDPLLCFK
LFFTDEIISEIVKWTNAEISLKRRESMTSATERDTNEDEIYAFFGILVM
TAVRKDNHMSTDDLFDRSLSMVYVSVMSRDRFDFLIRCLRFDDKSIRPT
LRENDVFTPVRKIWDLFIHQCIQNYTPGAHLTIDEQLLGFRGRCPFRVY
IPNKPSKYGIKILMMCDSGTKYMINGMPYLGRGTQTNGVPLGEYYVKEL
SKPVHGSCRNITCDNWFTSIPLAKNLLQEPYKLTIVGTVRSNKREIPEV
LKNSRSRPVGTSMFCFDGPLTLVSYKPKPAKMVYLLSSCDEDASINEST
GKPQMVMYYNQTKGGVDTLDQMCSVMTCSRKTNRWPMALLYGMINIACI
NSFIIYSHNVSSKGEKVQSRKKEMRNLYMSLTSSFMRKRLEAPTLKRYL
RDNISNILPKEVPGTSDDSTEEPVMKKRTYCTYCPSKIRRKANASCKKC
KKVICREHNIDMCQSCF

In one aspect, the SPB transposase (“M226F SPB” or “mutant M226F SPB”) is encoded by a polynucleotide comprising, consisting essentially of, or consisting of the nucleic acid sequence at least 95%, 96%, 97%, 98%, 99% or 100% (or any percentage in between) identical to SEQ ID NO: 110. The nucleic acids encoding the M226F mutation are shown in bolded and underlined font.

(SEQ ID NO: 110)
ATGGCTCCCAAGAAGAAGCGGAAAGTTGGCGGCGGAGGCAGCAGCCTGGATGATGAGCATATTC
TGAGCGCCCTGCTGCAGAGCGACGATGAACTCGTGGGCGAAGATAGCGACAGCGAGGTGTCCGA
TCACGTGTCCGAGGATGACGTGCAGTCCGATACCGAGGAAGCCTTCATCGACGAGGTGCACGAA
GTGCAGCCTACAAGCAGCGGCAGCGAGATCCTGGACGAGCAGAATGTGATCGAGCAGCCAGGAT
CTAGCCTGGCCAGCAACAGAATCCTGACACTGCCCCAGAGAACCATCCGGGGCAAGAACAAGCA
CTGCTGGTCCACCAGCAAGAGCACCAGACGGTCTAGAGTGTCTGCCCTGAACATCGTGCGAAGC
CAGAGGGGCCCTACCAGAATGTGCCGGAACATCTACGACCCTCTGCTGTGCTTCAAGCTGTTCT
TCACCGACGAGATCATCTCCGAGATCGTGAAGTGGACCAACGCCGAGATCAGCCTGAAGCGGAG
AGAATCCATGACCAGCGCCACCTTCAGAGACACCAACGAGGACGAGATCTACGCCTTCTTCGGC
ATCCTGGTCATGACAGCCGTGCGGAAGGACAACCACATGAGCACCGACGACCTGTTCGACCGCA
GCCTGTCTATGGTGTACGTGTCCGTGATGAGCCGGGACAGATTCGACTTCCTGATCCGGTGCCT
GCGGTTCGACGACAAGTCCATCAGACCCACACTGCGCGAGAACGACGTGTTCACACCTGTGCGG
AAGATCTGGGACCTGTTCATCCACCAGTGCATCCAGAACTACACCCCTGGCGCTCACCTGACCA
TCGACGAACAGCTGCTGGGCTTCAGAGGCAGATGCCCCTTCAGAGTGTACATCCCCAACAAGCC
CTCTAAGTACGGCATCAAGATCCTGATGATGTGCGACAGCGGCACCAAGTACATGATCAACGGC
ATGCCCTACCTCGGCAGAGGCACCCAAACAAATGGCGTGCCACTGGGCGAGTACTACGTGAAAG
AACTGAGCAAGCCTGTGCACGGCAGCTGCAGAAACATCACCTGTGACAACTGGTTTACCAGCAT
TCCCCTGGCCAAGAACCTGCTGCAAGAACCCTACAAGCTGACAATCGTGGGCACCGTGCGGAGC
AACAAGAGGGAAATTCCCGAGGTGCTGAAGAACTCTCGGAGCAGACCTGTGGGCACCAGCATGT
TCTGCTTCGACGGACCTCTGACACTGGTGTCCTACAAGCCCAAGCCTGCCAAGATGGTGTACCT
GCTGAGCAGCTGTGACGAGGACGCCAGCATCAATGAGAGCACCGGCAAGCCCCAGATGGTCATG
TACTACAACCAGACCAAAGGCGGCGTGGACACCCTGGATCAGATGTGCAGCGTGATGACCTGCA
GCAGAAAGACCAACAGATGGCCCATGGCTCTGCTGTACGGCATGATCAATATCGCCTGCATCAA
CAGCTTCATCATCTACAGCCACAACGTGTCCAGCAAGGGCGAGAAGGTGCAGAGCCGGAAGAAA
TTCATGCGGAACCTGTACATGAGCCTGACCAGCAGCTTCATGAGAAAGCGGCTGGAAGCCCCTA
CACTGAAGAGATACCTGCGGGACAACATCAGCAACATCCTGCCTAAAGAGGTGCCCGGCACCAG
CGACGATAGCACAGAGGAACCCGTGATGAAGAAGAGGACCTACTGCACCTACTGTCCCAGCAAG
ATCCGGCGGAAGGCCAACGCCAGCTGCAAAAAGTGCAAGAAAGTGATCTGCCGCGAGCACAACA
TCGATATGTGCCAGAGCTGCTTCTGA

The PB, PBL or SPB transposases can be isolated or derived from an insect, vertebrate, crustacean or urochordate as described in more detail in PCT Publication No. WO 2019/173636 and PCT/US2019/049816. In preferred aspects, the PB, PBL or SPB transposases is be isolated or derived from the insect Trichoplusia ni (GenBank Accession No. AAA87375) or Bombyx mori (GenBank Accession No. BAD11135).

A hyperactive PB or PBL transposase is a transposase that is more active than the naturally occurring variant from which it is derived. In a preferred aspect, a hyperactive PB or PBL transposase is isolated or derived from Bombyx mori or Xenopus tropicalis. Examples of hyperactive PB or PBL transposases are disclosed in U.S. Pat. Nos. 6,218,185; 6,962,810, 8,399,643 and WO 2019/173636. A list of hyperactive amino acid substitutions is disclosed in U.S. Pat. No. 10,041,077.

In some aspects, the PB or PBL transposase is integration deficient. An integration deficient PB or PBL transposase is a transposase that can excise its corresponding transposon, but that integrates the excised transposon at a lower frequency than a corresponding wild type transposase. Examples of integration deficient PB or PBL transposases are disclosed in U.S. Pat. Nos. 6,218,185; 6,962,810, 8,399,643 and WO 2019/173636. A list of integration deficient amino acid substitutions is disclosed in U.S. Pat. No. 10,041,077.

In some aspects, the PB or PBL transposase is fused to a nuclear localization signal. Examples of PB or PBL transposases fused to a nuclear localization signal are disclosed in U.S. Pat. Nos. 6,218,185; 6,962,810, 8,399,643 and WO 2019/173636.

A transposon or nanotransposon of the present disclosure can be a Sleeping Beauty transposon. In some aspects, when the transposon is a Sleeping Beauty transposon, the transposase is a Sleeping Beauty transposase (for example as disclosed in U.S. Pat. No. 9,228,180) or a hyperactive Sleeping Beauty (SB100X) transposase. In a preferred aspect, the Sleeping Beauty transposase comprises or consists of an amino acid sequence at least 75%, 80%, 85%, 90%, 95%, 96%, 97%, 98%, 99% or 100% (or any percentage in between) identical to SEQ ID NO: 111. In a preferred aspect, hyperactive Sleeping Beauty (SB100X) transposase comprises or consists of an amino acid sequence at least 75%, 80%, 85%, 90%, 95%, 96%, 97%, 98%, 99% or 100% (or any percentage in between) identical to SEQ ID NO: 112.

A transposon or nanotransposon of the present disclosure can be a piggyBac transposon which comprises or consists of a nucleic acid sequence at least 75%, 80%, 85%, 90%, 95%, 96%, 97%, 98%, 99% or 100% (or any percentage in between) identical to any of the nucleic acids set forth in SEQ ID NOs: 144-156 and 158. A transposon or nanotransposon of the present disclosure can be a piggyBac transposon which comprises or consists of a nucleic acid sequence at least 75%, 80%, 85%, 90%, 95%, 96%, 97%, 98%, 99% or 100% (or any percentage in between) identical to SEQ ID NO: 114. A transposon or nanotransposon of the present disclosure can be a piggyBac transposon which comprises or consists of a nucleic acid sequence at least 75%, 80%, 85%, 90%, 95%, 96%, 97%, 98%, 99% or 100% (or any percentage in between) identical to SEQ ID NO: 115. In some aspects, when the transposon is a piggyBac transposon, the transposase is a piggyBac transposase (for example, as disclosed in U.S. Pat. Nos. 6,218,182; 6,962,810; 8,399,643 and PCT Publication No. WO 2010/099296), a piggyBac-like transposase, a Super piggyBac transposase or a mutant Super piggyBac transposase. In one aspect, the piggyBac transposase comprises or consists of an amino acid sequence at least 75%, 80%, 85%, 90%, 95%, 96%, 97%, 98%, 99% or 100% (or any percentage in between) identical to SEQ ID NO: 105. In one aspect, Super piggyBac transposase comprises or consists of an amino acid sequence at least 75%, 80%, 85%, 90%, 95%, 96%, 97%, 98%, 99% or 100% (or any percentage in between) identical to SEQ ID NO: 107. In one aspect, Super piggyBac transposase comprises or consists of an amino acid sequence at least 75%, 80%, 85%, 90%, 95%, 96%, 97%, 98%, 99% or 100% (or any percentage in between) identical to SEQ ID NO: 109.

A transposon or nanotransposon of the present disclosure can be a Helraiser transposon. An exemplary Helraiser transposon includes Helibat1, which comprises or consists of a nucleic acid sequence at least 75%, 80%, 85%, 90%, 95%, 96%, 97%, 98%, 99% or 100% (or any percentage in between) identical to SEQ ID NO: 116. In some aspects, when the transposon is a Helraiser transposon, the transposase is a Helitron transposase (for example, as disclosed in WO 2019/173636). In a preferred aspect, Helitron transposase comprises or consists of an amino acid sequence at least 75%, 80%, 85%, 90%, 95%, 96%, 97%, 98%, 99% or 100% (or any percentage in between) identical to SEQ ID NO: 117.

A transposon or nanotransposon of the present disclosure can be a Tol2 transposon. An exemplary Tol2 transposon, including inverted repeats, subterminal sequences and the Tol2 transposase, comprises or consists of a nucleic acid sequence at least 75%, 80%, 85%, 90%, 95%, 96%, 97%, 98%, 99% or 100% (or any percentage in between) identical to SEQ ID NO: 118. In some aspects, when the transposon is a Tol2 transposon, the transposase is a Tol2 transposase (for example, as disclosed in WO 2019/173636). In a preferred aspect, Tol2 transposase comprises or consists of an amino acid sequence at least 75%, 80%, 85%, 90%, 95%, 96%, 97%, 98%, 99% or 100% (or any percentage in between) identical to SEQ ID NO: 119.

A transposon or nanotransposon of the present disclosure can be a TcBuster transposon. In some aspects, when the transposon is a TcBuster transposon, the transposase is a TcBuster transposase or a hyperactive TcBuster transposase (for example, as disclosed in WO 2019/173636). The TcBuster transposase can comprise or consist of a naturally occurring amino acid sequence or a non-naturally occurring amino acid sequence. In a preferred aspect, a TcBuster transposase comprises or consists of an amino acid sequence at least 75%, 80%, 85%, 90%, 95%, 96%, 97%, 98%, 99% or 100% (or any percentage in between) identical to SEQ ID NO: 120. The polynucleotide encoding a TcBuster transposase can comprise or consist of a naturally occurring nucleic acid sequence or a non-naturally occurring nucleic acid sequence. In a preferred aspect, a TcBuster transposase is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 121.

In some aspects, a mutant TcBuster transposase comprises one or more sequence variations when compared to a wild type TcBuster transposase as described in more detail in PCT Publication No. WO 2019/173636 and PCT/US2019/049816.

The cell delivery compositions (e.g., transposons) disclosed herein can comprise a nucleic acid encoding a therapeutic protein or therapeutic agent. Examples of therapeutic proteins include those disclosed in PCT Publication No. WO 2019/173636 and PCT/US2019/049816.

### Vector Systems

In some aspects, a polynucleotide of the present disclosure (e.g., transposon) can be utilized with in combination with another transposon or nanotransposon or with vector. A vector of the present disclose can be a viral vector or a recombinant vector. Viral vectors can comprise a sequence isolated or derived from a retrovirus, a lentivirus, an adenovirus, an adeno-associated virus or any combination thereof. The viral vector may comprise a sequence isolated or derived from an adeno-associated virus (AAV). The viral vector may comprise a recombinant AAV (rAAV). Exemplary adeno-associated viruses and recombinant adeno-associated viruses comprise two or more inverted terminal repeat (ITR) sequences located in cis next to a sequence encoding an scFv or a CAR of the disclosure. Exemplary adeno-associated viruses and recombinant adeno-associated viruses include, but are not limited to all serotypes (e.g., AAV1, AAV2, AAV3, AAV4, AAV5, AAV6, AAV7, AAV8, and AAV9). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03.

A vector of the present disclose can be a nanoparticle. Non-limiting examples of nanoparticle vectors include nucleic acids (e.g., RNA, DNA, synthetic nucleotides, modified nucleotides or any combination thereof), amino acids (L-amino acids, D-amino acids, synthetic amino acids, modified amino acids, or any combination thereof), polymers (e.g., polymersomes), micelles, lipids (e.g., liposomes), organic molecules (e.g., carbon atoms, sheets, fibers, tubes), inorganic molecules (e.g., calcium phosphate or gold) or any combination thereof. A nanoparticle vector can be passively or actively transported across a cell membrane.

The cell delivery compositions (e.g., transposons, vectors) disclosed herein can comprise a nucleic acid encoding a therapeutic protein or therapeutic agent. Examples of therapeutic proteins include those disclosed in PCT Publication No. WO 2019/173636 and PCT/US2019/049816.

### Cells and Modified Cells of the Disclosure

Cells and modified cells of the disclosure can be mammalian cells. Preferably, the cells and modified cells are human cells. Cells and modified cells of the disclosure can be immune cells. The immune cells of the disclosure can comprise lymphoid progenitor cells, natural killer (NK) cells, T lymphocytes (T-cell), stem memory T cells (T_{SCM} cells), central memory T cells (T_{CM}), stem cell-like T cells, B lymphocytes (B-cells), antigen presenting cells (APCs), cytokine induced killer (CIK) cells, myeloid progenitor cells, neutrophils, basophils, eosinophils, monocytes, macrophages, platelets, erythrocytes, red blood cells (RBCs), megakaryocytes or osteoclasts.

The immune precursor cells can comprise any cells which can differentiate into one or more types of immune cells. The immune precursor cells can comprise multipotent stem cells that can self-renew and develop into immune cells. The immune precursor cells can comprise hematopoietic stem cells (HSCs) or descendants thereof. The immune precursor cells can comprise precursor cells that can develop into immune cells. The immune precursor cells can comprise hematopoietic progenitor cells (HPCs).

Hematopoietic stem cells (HSCs) are multipotent, self-renewing cells. All differentiated blood cells from the lymphoid and myeloid lineages arise from HSCs. HSCs can be found in adult bone marrow, peripheral blood, mobilized peripheral blood, peritoneal dialysis effluent and umbilical cord blood.

HSCs can be isolated or derived from a primary or cultured stem cell. HSCs can be isolated or derived from an embryonic stem cell, a multipotent stem cell, a pluripotent stem cell, an adult stem cell, or an induced pluripotent stem cell (iPSC).

Immune precursor cells can comprise an HSC or an HSC descendent cell. Non-limiting examples of HSC descendent cells include multipotent stem cells, lymphoid progenitor cells, natural killer (NK) cells, T lymphocyte cells (T-cells), B lymphocyte cells (B-cells), myeloid progenitor cells, neutrophils, basophils, eosinophils, monocytes and macrophages.

HSCs produced by the disclosed methods can retain features of “primitive” stem cells that, while isolated or derived from an adult stem cell and while committed to a single lineage, share characteristics of embryonic stem cells. For example, the “primitive” HSCs produced by the disclosed methods retain their “stemness” following division and do not differentiate. Consequently, as an adoptive cell therapy, the “primitive” HSCs produced by the disclosed methods not only replenish their numbers, but expand in vivo. “Primitive” HSCs produced by disclosed the methods can be therapeutically-effective when administered as a single dose.

Primitive HSCs can be CD34+. Primitive HSCs can be CD34+ and CD38−. Primitive HSCs can be CD34+, CD38− and CD90+. Primitive HSCs can be CD34+, CD38−, CD90+ and CD45RA−. Primitive HSCs can be CD34+, CD38−, CD90+, CD45RA−, and CD49f+. Primitive HSCs can be CD34+, CD38−, CD90+, CD45RA−, and CD49f+.

Primitive HSCs, HSCs, and/or HSC descendent cells can be modified according to the disclosed methods to express an exogenous sequence (e.g., a chimeric antigen receptor or therapeutic protein). Modified primitive HSCs, modified HSCs, and/or modified HSC descendent cells can be forward differentiated to produce a modified immune cell including, but not limited to, a modified T cell, a modified natural killer cell and/or a modified B-cell.

The modified immune or immune precursor cells can be NK cells. The NK cells can be cytotoxic lymphocytes that differentiate from lymphoid progenitor cells. Modified NK cells can be derived from modified hematopoietic stem and progenitor cells (HSPCs) or modified HSCs. In some aspects, non-activated NK cells are derived from CD3-depleted leukapheresis (containing CD14/CD19/CD56+ cells).

The modified immune or immune precursor cells can be B cells. B cells are a type of lymphocyte that express B cell receptors on the cell surface. B cell receptors bind to specific antigens. Modified B cells can be derived from modified hematopoietic stem and progenitor cells (HSPCs) or modified HSCs.

Modified T cells of the disclosure may be derived from modified hematopoietic stem and progenitor cells (HSPCs) or modified HSCs. Unlike traditional biologics and chemotherapeutics, the disclosed modified-T cells the capacity to rapidly reproduce upon antigen recognition, thereby potentially obviating the need for repeat treatments. To achieve this, in some aspects, modified-T cells not only drive an initial response, but also persist in the patient as a stable population of viable memory T cells to prevent potential relapses. Alternatively, in some aspects, when it is not desired, the modified-T cells do not persist in the patient.

Intensive efforts have been focused on the development of antigen receptor molecules that do not cause T cell exhaustion through antigen-independent (tonic) signaling, as well as of a modified-T cell product containing early memory T cells, especially stem cell memory (T_{SCM}) or stem cell-like T cells. Stem cell-like modified-T cells of the disclosure exhibit the greatest capacity for self-renewal and multipotent capacity to derive central memory (T_{CM}) T cells or T_{CM} like cells, effector memory (T_{EM}) and effector T cells (T_{E}), thereby producing better tumor eradication and long-term modified-T cell engraftment. A linear pathway of differentiation may be responsible for generating these cells: Naïve T cells (T_{N})>T_{SCM}>T_{CM}>T_{EM}>T_{E}>T_{TE}, whereby TN is the parent precursor cell that directly gives rise to T_{SCM}, which then, in turn, directly gives rise to T_{CM}, etc. Compositions of T cells of the disclosure can comprise one or more of each parental T cell subset with T_{SCM} cells being the most abundant (e.g., T_{SCM}>T_{CM}>T_{EM}>T_{E}>T_{TE}).

The immune cell precursor can be differentiated into or is capable of differentiating into an early memory T cell, a stem cell like T-cell, a Naïve T cells (T_{N}), a T_{SCM}, a T_{CM}, a T_{EM}, a T_{E}, or a T_{TE}. The immune cell precursor can be a primitive HSC, an HSC, or a HSC descendent cell of the disclosure. The immune cell can be an early memory T cell, a stem cell like T-cell, a Naïve T cells (T_{N}), a T_{SCM}, a T_{CM}, a T_{EM}, a T_{E}, or a T_{TE}.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of a plurality of modified T cells in the population expresses one or more cell-surface marker(s) of an early memory T cell. The population of modified early memory T cells comprises a plurality of modified stem cell-like T cells. The population of modified early memory T cells comprises a plurality of modified T_{SCM} cells. The population of modified early memory T cells comprises a plurality of modified T_{CM} cells.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a stem cell-like T cell. The population of modified stem cell-like T cells comprises a plurality of modified T_{SCM} cells. The population of modified stem cell-like T cells comprises a plurality of modified T_{CM} cells.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% or any percentage in between of the plurality of modified T cells in the population expresses one or more cell-surface marker(s) of a stem memory T cell (T_{SCM}) or a T_{SCM}-like cell; and wherein the one or more cell-surface marker(s) comprise CD45RA and CD62L. The cell-surface markers can comprise one or more of CD62L, CD45RA, CD28, CCR7, CD127, CD45RO, CD95, CD95 and IL-2RB. The cell-surface markers can comprise one or more of CD45RA, CD95, IL-2RB, CCR7, and CD62L.

In some aspects, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the plurality of modified T cells in the population expresses one or more cell-surface marker(s) of a central memory T cell (T_{CM}) or a T_{CM}-like cell; and wherein the one or more cell-surface marker(s) comprise CD45RO and CD62L. The cell-surface markers can comprise one or more of CD45RO, CD95, IL-2Rβ, CCR7, and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of a naïve T cell (T_{N}). The cell-surface markers can comprise one or more of CD45RA, CCR7 and CD62L.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells in the population expresses one or more cell-surface marker(s) of an effector T-cell (modified T_{EFF}). The cell-surface markers can comprise one or more of CD45RA, CD95, and IL-2Rβ.

The methods of the disclosure can modify and/or produce a population of modified T cells, wherein at least 2%, 5%, 10%, 15%, 20%, 25%, 30%, 35%, 40%, 45%, 50%, 60%, 65%, 70%, 75%, 80%, 85%, 90%, 95%, 99% or any percentage in between of the plurality of modified T cells of the population expresses one or more cell-surface marker(s) of a stem cell-like T cell, a stem memory T cell (T_{SCM}) or a central memory T cell (T_{CM}).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 or wherein at least about 70% to about 99%, about 75% to about 95% or about 85% to about 95% of the population of modified cells express one or more cell-surface marker(s) comprising CD34 (e.g., comprise the cell-surface marker phenotype CD34+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and do not express one or more cell-surface marker(s) comprising CD38, or wherein at least about 45% to about 90%, about 50% to about 80% or about 65% to about 75% of the population of modified cells express one or more cell-surface marker(s) comprising CD34 and do not express one or more cell-surface marker(s) comprising CD38 (e.g., comprise the cell-surface marker phenotype CD34+ and CD38−).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38, or wherein at least about 0.2% to about 40%, about 0.2% to about 30%, about 0.2% to about 2% or 0.5% to about 1.5% of the population of modified cells express one or more cell-surface marker(s) comprising CD34 and CD90) and do not express one or more cell-surface marker(s) comprising CD38 (e.g., comprise the cell-surface marker phenotype CD34+, CD38− and CD90+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD38 and CD45RA, or wherein at least about 0.2% to about 40%, about 0.2% to about 30%, about 0.2% to about 2% or 0.5% to about 1.5% of the population of modified cells express one or more cell-surface marker(s) comprising CD34 and CD90 and do not express one or more cell-surface marker(s) comprising CD38 and CD45RA (e.g., comprise the cell-surface marker phenotype CD34+, CD38−, CD90+, CD45RA−).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34, CD90 and CD49f and do not express one or more cell-surface marker(s) comprising CD38 and CD45RA, or wherein at least about 0.02% to about 30%, about 0.02% to about 2%, about 0.04% to about 2% or about 0.04% to about 1% of the population of modified cells express one or more cell-surface marker(s) comprising CD34, CD90 and CD49f and do not express one or more cell-surface marker(s) comprising CD38 and CD45RA (e.g., comprise the cell-surface marker phenotype CD34+, CD38−, CD90+, CD45RA− and CD49f+).

A plurality of modified cells of the population comprise a transgene or a sequence encoding the transgene (e.g., a CAR), wherein at least 75%, at least 85%, at least 90%, at least 91%, at least 92%, at least 93%, at least 94%, at least 95%, at least 96%, at least 97%, at least 98%, at least 99%, at least 99.5%, at least 99.9% or 100% of the plurality of cells of the population comprise the transgene or the sequence encoding the transgene, wherein at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 1.5%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% or 100% of the population of modified cells express one or more cell-surface marker(s) comprising CD34 and CD90 and do not express one or more cell-surface marker(s) comprising CD45RA, or wherein at least about 0.2% to about 5%, about 0.2% to about 3% or about 0.4% to about 3% of the population of modified cells express one or more cell-surface marker(s) comprising CD34 and CD90 and do not express one or more cell-surface marker(s) comprising CD45RA (e.g., comprise the cell-surface marker phenotype CD34+, CD90+ and CD45RA−)

Compositions and methods of producing and/or expanding the immune cells or immune precursor cells (e.g., the disclosed modified T-cells) and buffers for maintaining or enhancing a level of cell viability and/or a stem-like phenotype of the immune cells or immune precursor cells (e.g., the disclosed modified T-cells) are disclosed elsewhere herein and are disclosed in more detail in U.S. Pat. No. 10,329,543 and PCT Publication No. WO 2019/173636.

Cells and modified cells of the disclosure can be somatic cells. Cells and modified cells of the disclosure can be differentiated cells. Cells and modified cells of the disclosure can be autologous cells or allogenic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

### Methods of Expressing a Chimeric Antigen Receptor or a Therapeutic Polypeptide

The disclosure provides methods of producing a population of modified cells. In an aspect, the disclosure provides methods of expressing a CAR on the surface of a cell. In an aspect, the disclosure provides methods of expressing a therapeutic polypeptide in a cell. The method comprises delivering to a population of cells a) the polynucleotide encoding a transposon of the disclosure (e.g. transposon encoding a sequence for a CAR or a therapeutic polypeptide) and b) a nucleic acid or amino acid sequence comprising a sequence encoding a transposase enzyme. The method may further comprise (c) culturing the modified cell population under conditions suitable for integration of the sequence encoding the CAR or the therapeutic polypeptide; and (d) expanding and/or selecting at least one cell from the modified cell population that express the CAR on the cell surface or at least one cell from the modified cell population that express the therapeutic protein in the cell.

In some aspects, the cell population can comprise leukocytes and/or CD4+ and CD8+ leukocytes. The cell population can comprise CD4+ and CD8+ leukocytes in an optimized ratio. The optimized ratio of CD4+ to CD8+ leukocytes does not naturally occur in vivo. The cell population can comprise a tumor cell.

In some aspects, the conditions sufficient to transfer the CAR or the sequence encoding the CAR, transposon, or vector across a cell membrane of at least one cell in the cell population comprises at least one of an application of one or more pulses of electricity at a specified voltage, a buffer, and one or more supplemental factor(s). In some aspects, the conditions suitable for integration of the sequence encoding the CAR comprise at least one of a buffer and one or more supplemental factor(s).

The buffer can comprise PBS, HBSS, OptiMEM, BTXpress, Amaxa Nucleofector, Human T cell nucleofection buffer or any combination thereof. The one or more supplemental factor(s) can comprise (a) a recombinant human cytokine, a chemokine, an interleukin or any combination thereof; (b) a salt, a mineral, a metabolite or any combination thereof; (c) a cell medium; (d) an inhibitor of cellular DNA sensing, metabolism, differentiation, signal transduction, one or more apoptotic pathway(s) or combinations thereof; and (e) a reagent that modifies or stabilizes one or more nucleic acids. The recombinant human cytokine, the chemokine, the interleukin or any combination thereof can comprise IL2, IL7, IL12, IL15, IL21, IL1, IL3, IL4, IL5, IL6, IL8, CXCL8, IL9, IL10, IL11, IL13, IL14, IL16, IL17, IL18, IL19, IL20, IL22, IL23, IL25, IL26, IL27, IL28, IL29, IL30, IL31, IL32, IL33, IL35, IL36, GM-CSF, IFN-gamma, IL-1 alpha/IL-1F1, IL-1 beta/IL-1F2, IL-12 p70, IL-12/IL-35 p35, IL-13, IL-17/IL-17A, IL-17A/F Heterodimer, IL-17F, IL-18/IL-1F4, IL-23, IL-24, IL-32, IL-32 beta, IL-32 gamma, IL-33. LAP (TGF-beta 1), Lymphotoxin-alpha/TNF-beta, TGF-beta, TNF-alpha, TRANCE/TNFSF11/RANK L or any combination thereof. The salt, the mineral, the metabolite or any combination thereof can comprise HEPES, Nicotinamide, Heparin, Sodium Pyruvate, L-Glutamine, MEM Non-Essential Amino Acid Solution, Ascorbic Acid, Nucleosides, FBS/FCS, Human serum, serum-substitute, antibiotics, pH adjusters, Earle's Salts, 2-Mercaptoethanol, Human transferrin, Recombinant human insulin, Human serum albumin, Nucleofector PLUS Supplement, KCL, MgCl₂, Na₂HPO₄, NAH₂PO₄, Sodium lactobionate, Mannitol, Sodium succinate, Sodium Chloride, CINa, Glucose, Ca(NO₃)₂, Tris/HCl, K₂HPO₄, KH₂PO₄, Polyethylenimine, Poly-ethylene-glycol, Poloxamer 188, Poloxamer 181, Poloxamer 407, Poly-vinylpyrrolidone. Pop313, Crown-5, or any combination thereof. The cell medium can comprise PBS, HBSS, OptiMEM, DMEM, RPMI 1640, AIM-V, X-VIVO 15, CellGro DC Medium, CTS OpTimizer T Cell Expansion SFM, TexMACS Medium, PRIME-XV T Cell Expansion Medium, ImmunoCult-XF T Cell Expansion Medium or any combination thereof. The inhibitor of cellular DNA sensing, metabolism, differentiation, signal transduction, one or more apoptotic pathway(s) or combinations thereof comprise inhibitors of TLR9, MyD88, IRAK, TRAF6, TRAF3, IRF-7, NF-KB, Type 1 Interferons, pro-inflammatory cytokines, cGAS, STING, Sec5, TBK1, IRF-3, RNA pol III, RIG-1, IPS-1, FADD, RIP1, TRAF3, AIM2, ASC, Caspase1, Pro-IL1B, PI3K, Akt, Wnt3A, inhibitors of glycogen synthase kinase-3β (GSK-3β) (e.g. TWS119), or any combination thereof. Examples of such inhibitors can include Bafilomycin, Chloroquine, Quinacrine, AC-YVAD-CMK, Z-VAD-FMK, Z-IETD-FMK or any combination thereof. The reagent that modifies or stabilizes one or more nucleic acids comprises a pH modifier, a DNA-binding protein, a lipid, a phospholipid, CaPO4, a net neutral charge DNA binding peptide with or without a NLS sequence, a TREX1 enzyme or any combination thereof.

The expansion and selection steps can occur concurrently or sequentially. The expansion can occur prior to selection. The expansion can occur following selection, and, optionally, a further (i.e. second) selection can occur following expansion. Concurrent expansion and selection can be simultaneous. The expansion and/or selection steps can proceed for a period of 10 to 14 days, inclusive of the endpoints.

The expansion can comprise contacting at least one cell of the modified cell population with an antigen to stimulate the at least one cell through the CAR, thereby generating an expanded cell population. The antigen can be presented on the surface of a substrate. The substrate can have any form, including, but not limited to a surface, a well, a bead or a plurality thereof, and a matrix. The substrate can further comprise a paramagnetic or magnetic component. The antigen can be presented on the surface of a substrate, wherein the substrate is a magnetic bead, and wherein a magnet can be used to remove or separate the magnetic beads from the modified and expanded cell population. The antigen can be presented on the surface of a cell or an artificial antigen presenting cell. Artificial antigen presenting cells can include, but are not limited to, tumor cells and stem cells.

In some aspects wherein the transposon or vector comprises a selection gene, the selection step comprises contacting at least one cell of the modified cell population with a compound to which the selection gene confers resistance, thereby identifying a cell expressing the selection gene as surviving the selection and identifying a cell failing to express the selection gene as failing to survive the selection step.

The disclosure provides a composition comprising the modified, expanded and selected cell population of the methods described herein.

A more detailed description of methods for expressing a CAR on the surface of a cell is disclosed in PCT Publication No. WO 2019/049816 and PCT/US2019/049816.

The present disclosure provides a cell or a population of cells wherein the cell comprises a transposon of the disclosure comprising (a) an inducible transgene construct, comprising a sequence encoding an inducible promoter and a sequence encoding a transgene, such as a therapeutic polypeptide, and (b) a receptor construct, comprising a sequence encoding a constitutive promoter and a sequence encoding an exogenous receptor, such as a CAR, wherein, upon integration of the construct of (a) and the construct of (b) into a genomic sequence of a cell, the exogenous receptor is expressed, and wherein the exogenous receptor, upon binding a ligand or antigen, transduces an intracellular signal that targets directly or indirectly the inducible promoter regulating expression of the inducible transgene (a) to modify gene expression.

The composition can modify gene expression by decreasing gene expression. The composition can modify gene expression by transiently modifying gene expression (e.g., for the duration of binding of the ligand to the exogenous receptor). The composition can modify gene expression acutely (e.g., the ligand reversibly binds to the exogenous receptor). The composition can modify gene expression chronically (e.g., the ligand irreversibly binds to the exogenous receptor).

The exogenous receptor can comprise an endogenous receptor with respect to the genomic sequence of the cell. Exemplary receptors include, but are not limited to, intracellular receptors, cell-surface receptors, transmembrane receptors, ligand-gated ion channels, and G-protein coupled receptors.

The exogenous receptor can comprise a non-naturally occurring receptor. The non-naturally occurring receptor can be a synthetic, modified, recombinant, mutant or chimeric receptor. The non-naturally occurring receptor can comprise one or more sequences isolated or derived from a T-cell receptor (TCR). The non-naturally occurring receptor can comprise one or more sequences isolated or derived from a scaffold protein. In some aspects, including those wherein the non-naturally occurring receptor does not comprise a transmembrane domain, the non-naturally occurring receptor interacts with a second transmembrane, membrane-bound and/or an intracellular receptor that, following contact with the non-naturally occurring receptor, transduces an intracellular signal. The non-naturally occurring receptor can comprise a transmembrane domain. The non-naturally occurring receptor can interact with an intracellular receptor that transduces an intracellular signal. The non-naturally occurring receptor can comprise an intracellular signaling domain. The non-naturally occurring receptor can be a chimeric ligand receptor (CLR). The CLR can be a chimeric antigen receptor (CAR).

The sequence encoding the inducible promoter of comprises a sequence encoding an NF_{K}B promoter, a sequence encoding an interferon (IFN) promoter or a sequence encoding an interleukin-2 promoter. In some aspects, the IFN promoter is an IFNγ promoter. The inducible promoter can be isolated or derived from the promoter of a cytokine or a chemokine. The cytokine or chemokine can comprise IL2, IL3, IL4, IL5, IL6, IL10, IL12, IL13, IL17A/F, IL21, IL22, IL23, transforming growth factor beta (TGFβ), colony stimulating factor 2 (GM-CSF), interferon gamma (IFNγ), Tumor necrosis factor alpha (TNFα), LTα, perforin, Granzyme C (Gzmc), Granzyme B (Gzmb), C—C motif chemokine ligand 5 (CCL5), C—C motif chemokine ligand 4 (Ccl4), C—C motif chemokine ligand 3 (Ccl3), X—C motif chemokine ligand 1 (Xcl1) or LIF interleukin 6 family cytokine (Lif).

The inducible promoter can be isolated or derived from the promoter of a gene comprising a surface protein involved in cell differentiation, activation, exhaustion and function. In some aspects, the gene comprises CD69, CD71, CTLA4, PD-1, TIGIT, LAG3, TIM-3, GITR, MHCII, COX-2, FASL or 4-1BB.

The inducible promoter can be isolated or derived from the promoter of a gene involved in CD metabolism and differentiation. The inducible promoter can be isolated or derived from the promoter of Nr4a1, Nr4a3, Tnfrsf) (4-1BB), Sema7a, Zfp36l2, Gadd45b, Dusp5, Dusp6 and Neto2.

In some aspects, the inducible transgene construct comprises or drives expression of a signaling component downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of which are disclosed in PCT Publication No. WO 2019/173636 and PCT Application No. PCT/US2019/049816.

### Armored Cells

The modified cells of disclosure (e.g., CAR T-cells) can be further modified to enhance their therapeutic potential. Alternatively, or in addition, the modified cells may be further modified to render them less sensitive to immunologic and/or metabolic checkpoints. Modifications of this type “armor” the cells, which, following the modification, may be referred to here as “armored” cells (e.g., armored T-cells). Armored cells may be produced by, for example, blocking and/or diluting specific checkpoint signals delivered to the cells (e.g., checkpoint inhibition) naturally, within the tumor immunosuppressive microenvironment.

An armored cell of the disclosure can be derived from any cell, for example, a T cell, a NK cell, a hematopoietic progenitor cell, a peripheral blood (PB) derived T cell (including a T cell isolated or derived from G-CSF-mobilized peripheral blood), or an umbilical cord blood (UCB) derived T cell. An armored cell (e.g., armored T-cell) can comprise one or more of a chimeric ligand receptor (CLR comprising a protein scaffold, an antibody, an ScFv, or an antibody mimetic)/chimeric antigen receptor (CAR comprising a protein scaffold, an antibody, an ScFv, or an antibody mimetic), a CARTyrin (a CAR comprising a Centyrin), and/or a VCAR (a CAR comprising a camelid VHH or a single domain VH). An armored cell (e.g., armored T-cell) can comprise an inducible proapoptotic polypeptide as disclosed herein. An armored cell (e.g., armored T-cell) can comprise an exogenous sequence. The exogenous sequence can comprise a sequence encoding a therapeutic protein. Exemplary therapeutic proteins may be nuclear, cytoplasmic, intracellular, transmembrane, cell-surface bound, or secreted proteins. Exemplary therapeutic proteins expressed by the armored cell (e.g., armored T-cell) may modify an activity of the armored cell or may modify an activity of a second cell. An armored cell (e.g., armored T-cell) can comprise a selection gene or a selection marker. An armored cell (e.g., armored T-cell) can comprise a synthetic gene expression cassette (also referred to herein as an inducible transgene construct).

The modified cells of disclosure (e.g., CAR T-cells) can be further modified to silence or reduce expression one or more gene(s) encoding receptor(s) of inhibitory checkpoint signals to produce an armored cell (e.g., armored CAR T-cell). Receptors of inhibitory checkpoint signals are expressed on the cell surface or within the cytoplasm of a cell. Silencing or reducing expressing of the gene encoding the receptor of the inhibitory checkpoint signal results a loss of protein expression of the inhibitory checkpoint receptors on the surface or within the cytoplasm of an armored cell. Thus, armored cells having silenced or reduced expression of one or more genes encoding an inhibitory checkpoint receptor is resistant, non-receptive or insensitive to checkpoint signals. The resistance or decreased sensitivity of the armored cell to inhibitory checkpoint signals enhances the therapeutic potential of the armored cell in the presence of these inhibitory checkpoint signals. Non-limiting examples of inhibitory checkpoint signals (and proteins that induce immunosuppression) are disclosed in PCT Publication No. WO 2019/173636. Preferred examples of inhibitory checkpoint signals that may be silenced include, but are not limited to, PD-1 and TGFβRII.

The modified cells of disclosure (e.g., CAR T-cells) can be further modified to silence or reduce expression of one or more gene(s) encoding intracellular proteins involved in checkpoint signaling to produce an armored cell (e.g., armored CAR T-cell). The activity of the modified cells may be enhanced by targeting any intracellular signaling protein involved in a checkpoint signaling pathway, thereby achieving checkpoint inhibition or interference to one or more checkpoint pathways. Non-limiting examples of intracellular signaling proteins involved in checkpoint signaling are disclosed in PCT Publication No. WO 2019/173636.

The modified cells of disclosure (e.g., CAR T-cells) can be further modified to silence or reduce expression of one or more gene(s) encoding a transcription factor that hinders the efficacy of a therapy to produce an armored cell (e.g., armored CAR T-cell). The activity of modified cells may be enhanced or modulated by silencing or reducing expression (or repressing a function) of a transcription factor that hinders the efficacy of a therapy. Non-limiting examples of transcription factors that may be modified to silence or reduce expression or to repress a function thereof include, but are not limited to, the exemplary transcription factors are disclosed in PCT Publication No. WO 2019/173636.

The modified cells of disclosure (e.g., CAR T-cells) can be further modified to silence or reduce expression of one or more gene(s) encoding a cell death or cell apoptosis receptor to produce an armored cell (e.g., armored CAR T-cell). Interaction of a death receptor and its endogenous ligand results in the initiation of apoptosis. Disruption of an expression, an activity, or an interaction of a cell death and/or cell apoptosis receptor and/or ligand render a modified cell less receptive to death signals, consequently, making the armored cell more efficacious in a tumor environment. Non-limiting examples of cell death and/or cell apoptosis receptors and ligands are disclosed in PCT Publication No. WO 2019/173636. A preferred example of cell death receptor which may be modified is Fas (CD95).

The modified cells of disclosure (e.g., CAR T-cells) can be further modified to silence or reduce expression of one or more gene(s) encoding a metabolic sensing protein to produce an armored cell (e.g., armored CAR T-cell). Disruption to the metabolic sensing of the immunosuppressive tumor microenvironment (characterized by low levels of oxygen, pH, glucose and other molecules) by a modified cell leads to extended retention of T-cell function and, consequently, more tumor cells killed per cell. Non-limiting examples of metabolic sensing genes and proteins are disclosed in PCT Publication No. WO 2019/173636. A preferred example, HIF1a and VHL play a role in T-cell function while in a hypoxic environment. An armored T-cell may have silenced or reduced expression of one or more genes encoding HIF1a or VHL.

The modified cells of disclosure (e.g., CAR T-cells) can be further modified to silence or reduce expression of one or more gene(s) encoding proteins that that confer sensitivity to a cancer therapy, including a monoclonal antibody, to produce an armored cell (e.g., armored CAR T-cell). Thus, an armored cell can function and may demonstrate superior function or efficacy whilst in the presence of a cancer therapy (e.g., a chemotherapy, a monoclonal antibody therapy, or another anti-tumor treatment). Non-limiting examples of proteins involved in conferring sensitivity to a cancer therapy are disclosed in PCT Publication No. WO 2019/173636.

The modified cells of disclosure (e.g., CAR T-cells) can be further modified to silence or reduce expression of one or more gene(s) encoding a growth advantage factor to produce an armored cell (e.g., armored CAR T-cell). Silencing or reducing expression of an oncogene can confer a growth advantage for the cell. For example, silencing or reducing expression (e.g., disrupting expression) of a TET2 gene during a CAR T-cell manufacturing process results in the generation of an armored CAR T-cell with a significant capacity for expansion and subsequent eradication of a tumor when compared to a non-armored CAR T-cell lacking this capacity for expansion. This strategy may be coupled to a safety switch (e.g., an iC9 safety switch described herein), which permits the targeted disruption of an armored CAR T-cell in the event of an adverse reaction from a subject or uncontrolled growth of the armored CAR T-cell. Non-limiting examples of growth advantage factors are disclosed in PCT Publication No. WO 2019/173636.

The modified cells of disclosure (e.g., CAR T-cells) can be further modified to express a modified/chimeric checkpoint receptor to produce an armored T-cell of the disclosure.

The modified/chimeric checkpoint receptor can comprise a null receptor, decoy receptor or dominant negative receptor. A null receptor, decoy receptor or dominant negative receptor can be modified/chimeric receptor/protein. A null receptor, decoy receptor or dominant negative receptor can be truncated for expression of the intracellular signaling domain. Alternatively, or in addition, a null receptor, decoy receptor or dominant negative receptor can be mutated within an intracellular signaling domain at one or more amino acid positions that are determinative or required for effective signaling. Truncation or mutation of null receptor, decoy receptor or dominant negative receptor can result in loss of the receptor's capacity to convey or transduce a checkpoint signal to the cell or within the cell.

For example, a dilution or a blockage of an immunosuppressive checkpoint signal from a PD-LI receptor expressed on the surface of a tumor cell may be achieved by expressing a modified/chimeric PD-1 null receptor on the surface of an armored cell (e.g., armored CAR T-cell), which effectively competes with the endogenous (non-modified) PD-1 receptors also expressed on the surface of the armored cell to reduce or inhibit the transduction of the immunosuppressive checkpoint signal through endogenous PD-1 receptors of the armored cell. In this non-limiting example, competition between the two different receptors for binding to PD-LI expressed on the tumor cell reduces or diminishes a level of effective checkpoint signaling, thereby enhancing a therapeutic potential of the armored cell expressing the PD-1 null receptor.

The modified/chimeric checkpoint receptor can comprise a null receptor, decoy receptor or dominant negative receptor that is a transmembrane receptor, a membrane-associated or membrane-linked receptor/protein or an intracellular receptor/protein. Exemplary null, decoy, or dominant negative intracellular receptors/proteins include, but are not limited to, signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene. Non-limiting examples of cytokines, cytokine receptors, chemokines and chemokine receptors are disclosed in PCT Publication No. WO 2019/173636.

The modified/chimeric checkpoint receptor can comprise a switch receptor. Exemplary switch receptors comprise a modified/chimeric receptor/protein wherein a native or wild type intracellular signaling domain is switched or replaced with a different intracellular signaling domain that is either non-native to the protein and/or not a wild-type domain. For example, replacement of an inhibitory signaling domain with a stimulatory signaling domain would switch an immunosuppressive signal into an immunostimulatory signal. Alternatively, replacement of an inhibitory signaling domain with a different inhibitory domain can reduce or enhance the level of inhibitory signaling. Expression or overexpression, of a switch receptor can result in the dilution and/or blockage of a cognate checkpoint signal via competition with an endogenous wild-type checkpoint receptor (not a switch receptor) for binding to the cognate checkpoint receptor expressed within the immunosuppressive tumor microenvironment. Armored cells (e.g., armored CAR T-cells) can comprise a sequence encoding a switch receptor, leading to the expression of one or more switch receptors, and consequently, altering an activity of an armored cell. Armored cells (e.g., armored CAR T-cells) can express a switch receptor that targets an intracellularly expressed protein downstream of a checkpoint receptor, a transcription factor, a cytokine receptor, a death receptor, a metabolic sensing molecule, a cancer therapy, an oncogene, and/or a tumor suppressor protein or gene.

Exemplary switch receptors can comprise or can be derived from a protein including, but are not limited to, the signaling components downstream of an inhibitory checkpoint signal, a transcription factor, a cytokine or a cytokine receptor, a chemokine or a chemokine receptor, a cell death or apoptosis receptor/ligand, a metabolic sensing molecule, a protein conferring sensitivity to a cancer therapy, and an oncogene or a tumor suppressor gene.

The modified cells of disclosure (e.g., CAR T-cells) can be further modified to express a CLR/CAR that mediates conditional gene expression to produce an armored T-cell. The combination of the CLR/CAR and the condition gene expression system in the nucleus of the armored T-cell constitutes a synthetic gene expression system that is conditionally activated upon binding of cognate ligand(s) with CLR or cognate antigen(s) with CAR. This system may help to ‘armor’ or enhance therapeutic potential of modified T-cells by reducing or limiting synthetic gene expression at the site of ligand or antigen binding, at or within the tumor environment for example.

### Gene Editing Compositions and Methods

A modified cell be produced by introducing a transgene into the cell. The introducing step may comprise delivery of a nucleic acid sequence, a transgene, and/or a genomic editing construct via a non-transposition delivery system.

Introducing a nucleic acid sequence, transgene and/or a genomic editing construct into a cell ex vivo, in vivo, in vitro or in situ can comprise one or more of topical delivery, adsorption, absorption, electroporation, spin-fection, co-culture, transfection, mechanical delivery, sonic delivery, vibrational delivery, magnetofection or by nanoparticle-mediated delivery. Introducing a nucleic acid sequence, a transgene and/or a genomic editing construct into a cell ex vivo, in vivo, in vitro or in situ can comprise liposomal transfection, calcium phosphate transfection, fugene transfection, and dendrimer-mediated transfection. Introducing a nucleic acid sequence, a transgene, and/or a genomic editing construct into a cell ex vivo, in vivo, in vitro or in situ by mechanical transfection can comprise cell squeezing, cell bombardment, or gene gun techniques. Introducing a nucleic acid sequence, transgene and/or a genomic editing construct into a cell ex vivo, in vivo, in vitro or in situ by nanoparticle-mediated transfection can comprise liposomal delivery, delivery by micelles, and delivery by polymerosomes.

Introducing a nucleic acid sequence, transgene and/or a genomic editing construct into a cell ex vivo, in vivo, in vitro or in situ can comprise a non-viral vector. The non-viral vector can comprise a nucleic acid. The non-viral vector can comprise plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). The non-viral vector can comprise a transposon as described herein.

Introducing a nucleic acid sequence, transgene and/or a genomic editing construct into a cell ex vivo, in vivo, in vitro or in situ can comprise a viral vector. The viral vector can be a non-integrating non-chromosomal vector. Non-limiting examples of non-integrating non-chromosomal vectors include adeno-associated virus (AAV), adenovirus, and herpes viruses. The viral vector can be an integrating chromosomal vector. Non-limiting examples of integrating chromosomal vectors include adeno-associated vectors (AAV), Lentiviruses, and gamma-retroviruses.

Introducing a nucleic acid sequence, transgene and/or a genomic editing construct into a cell ex vivo, in vivo, in vitro or in situ can comprise a combination of vectors. Non-limiting examples of vector combinations include viral and non-viral vectors, a plurality of non-viral vectors, or a plurality of viral vectors. Non-limiting examples of vector combinations include a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

Genome modification can comprise introducing a nucleic acid sequence, transgene and/or a genomic editing construct into a cell ex vivo, in vivo, in vitro or in situ to stably integrate a nucleic acid sequence, transiently integrate a nucleic acid sequence, produce site-specific integration of a nucleic acid sequence, or produce a biased integration of a nucleic acid sequence. The nucleic acid sequence can be a transgene.

Genome modification can comprise introducing a nucleic acid sequence, transgene and/or a genomic editing construct into a cell ex vivo, in vivo, in vitro or in situ to stably integrate a nucleic acid sequence. The stable chromosomal integration can be a random integration, a site-specific integration, or a biased integration. The site-specific integration can be non-assisted or assisted. The assisted site-specific integration is co-delivered with a site-directed nuclease. The site-directed nuclease comprises a transgene with 5′ and 3′ nucleotide sequence extensions that contain a percentage homology to upstream and downstream regions of the site of genomic integration. The transgene with homologous nucleotide extensions enable genomic integration by homologous recombination, microhomology-mediated end joining, or nonhomologous end-joining. The site-specific integration can occur at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Non-limiting examples of potential genomic safe harbors include intronic sequences of the human albumin gene, the adeno-associated virus site 1 (AAVS1), a naturally occurring site of integration of AAV virus on chromosome 19, the site of the chemokine (C—C motif) receptor 5 (CCR5) gene and the site of the human ortholog of the mouse Rosa26 locus.

The site-specific transgene integration can occur at a site that disrupts expression of a target gene. Disruption of target gene expression can occur by site-specific integration at introns, exons, promoters, genetic elements, enhancers, suppressors, start codons, stop codons, and response elements. Non-limiting examples of target genes targeted by site-specific integration include TRAC, TRAB, PDI, any immunosuppressive gene, and genes involved in allo-rejection.

The site-specific transgene integration can occur at a site that results in enhanced expression of a target gene. Enhancement of target gene expression can occur by site-specific integration at introns, exons, promoters, genetic elements, enhancers, suppressors, start codons, stop codons, and response elements.

Enzymes can be used to create strand breaks in the host genome to facilitate delivery or integration of the transgene. Enzymes can create single-strand breaks or double-strand breaks. Non-limiting examples of break-inducing enzymes include transposases, integrases, endonucleases, CRISPR-Cas9, transcription activator-like effector nucleases (TALEN), zinc finger nucleases (ZFN), Cas-CLOVER™, and CPF1. Break-inducing enzymes can be delivered to the cell encoded in DNA, encoded in mRNA, as a protein, or as a nucleoprotein complex with a guide RNA (gRNA).

The site-specific transgene integration can be controlled by a vector-mediated integration site bias. Vector-mediated integration site bias can controlled by the chosen lentiviral vector or by the chosen gamma-retroviral vector.

The site-specific transgene integration site can be a non-stable chromosomal insertion. The integrated transgene can be become silenced, removed, excised, or further modified. The genome modification can be a non-stable integration of a transgene. The non-stable integration can be a transient non-chromosomal integration, a semi-stable non chromosomal integration, a semi-persistent non-chromosomal insertion, or a non-stable chromosomal insertion. The transient non-chromosomal insertion can be epi-chromosomal or cytoplasmic. In an aspect, the transient non-chromosomal insertion of a transgene does not integrate into a chromosome and the modified genetic material is not replicated during cell division.

The genome modification can be a semi-stable or persistent non-chromosomal integration of a transgene. A DNA vector encodes a Scaffold/matrix attachment region (S-MAR) module that binds to nuclear matrix proteins for episomal retention of a non-viral vector allowing for autonomous replication in the nucleus of dividing cells.

The genome modification can be a non-stable chromosomal integration of a transgene. The integrated transgene can become silenced, removed, excised, or further modified.

The modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. The modification to the genome by transgene insertion can occur via CRISPR-Cas9, TALEN, ZFNs, Cas-CLOVER™ and cpf1.

In gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. An example recombinase that may be used as a cutting enzyme is the CRE recombinase. Non-limiting examples of integrases that may be used in insertion tools include viral based enzymes taken from any of a number of viruses including AAV, gamma retrovirus, and lentivirus. Examples transposons/retrotransposons that may be used in insertion tools are described in more detail herein.

A cell with an ex vivo, in vivo, in vitro or in situ genomic modification can be a germline cell or a somatic cell. The modified cell can be a human, non-human, mammalian, rat, mouse, or dog cell. The modified cell can be differentiated, undifferentiated, or immortalized. The modified undifferentiated cell can be a stem cell. The modified undifferentiated cell can be an induced pluripotent stem cell. The modified cell can be an immune cell. The modified cell can be a T cell, a hematopoietic stem cell, a natural killer cell, a macrophage, a dendritic cell, a monocyte, a megakaryocyte, or an osteoclast. The modified cell can be modified while the cell is quiescent, in an activated state, resting, in interphase, in prophase, in metaphase, in anaphase, or in telophase. The modified cell can be fresh, cryopreserved, bulk, sorted into sub-populations, from whole blood, from leukapheresis, or from an immortalized cell line. A detailed description for isolating cells from a leukapheresis product or blood is disclosed in in PCT Publication No. WO 2019/173636 and PCT/US2019/049816.

The present disclosure provides a gene editing composition and/or a cell comprising the gene editing composition. The gene editing composition can comprise a sequence encoding a DNA binding domain and a sequence encoding a nuclease protein or a nuclease domain thereof. The sequence encoding a nuclease protein or the sequence encoding a nuclease domain thereof can comprise a DNA sequence, an RNA sequence, or a combination thereof. The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein. The fusion protein can comprise a nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a small, Cas) (Cas9) operatively-linked to an effector. The disclosure provides a fusion protein comprising, consisting essentially of or consisting of a DNA localization component and an effector molecule, wherein the effector comprises a small, Cas9 (Cas9). A small Cas9 construct of the disclosure can comprise an effector comprising a type IIS endonuclease. A Staphylococcus aureus Cas9 with an active catalytic site comprises the amino acid sequence of SEQ ID NO: 122.

The disclosure provides compositions comprising an inactivated, small, Cas9 (dSaCas9) operatively-linked to an effector. The disclosure provides a fusion protein comprising, consisting essentially of or consisting of a DNA localization component and an effector molecule, wherein the effector comprises a small, inactivated Cas9 (dSaCas9). A small, inactivated Cas9 (dSaCas9) construct of the disclosure can comprise an effector comprising a type IIS endonuclease. A dSaCas9 comprises the amino acid sequence of SEQ ID NO: 123, which includes a D10A and a N580A mutation to inactivate the catalytic site.

The disclosure provides compositions comprising an inactivated Cas9 (dCas9) operatively-linked to an effector. The disclosure provides a fusion protein comprising, consisting essentially of or consisting of a DNA localization component and an effector molecule, wherein the effector comprises an inactivated Cas9 (dCas9). An inactivated Cas9 (dCas9) construct of the disclosure can comprise an effector comprising a type IIS endonuclease.

The dCas9 can be isolated or derived from Streptococcus pyogenes. The dCas9 can comprise a dCas9 with substitutions at amino acid positions 10 and 840, which inactivate the catalytic site. In some aspects, these substitutions are D10A and H840A. The dCas9 can comprise the amino acid sequence of SEQ ID NO: 124 or SEQ ID NO: 125.

An exemplary Clo051 nuclease domain comprises, consists essentially of or consists of, the amino acid sequence of SEQ ID NO: 126.

An exemplary dCas9-Clo051 (Cas-CLOVER) fusion protein can comprise, consist essentially of, or consist of, the amino acid sequence of SEQ ID NO: 127. The exemplary dCas9-Clo051 fusion protein can be encoded by a polynucleotide which comprises, consists essentially of, or consists of, the nucleic acid sequence of SEQ ID NO: 128. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 129. The exemplary dCas9-Clo051 fusion protein can be encoded by a polynucleotide which comprises, consists essentially of, or consists of, the nucleic acid sequence of SEQ ID NO: 130. The nucleic acid encoding the dCas9-Clo051 fusion protein can be DNA or RNA.

A cell comprising the gene editing composition can express the gene editing composition stably or transiently. Preferably, the gene editing composition is expressed transiently. The guide RNA can comprise a sequence complementary to a target sequence within a genomic DNA sequence. The target sequence within a genomic DNA sequence can be a target sequence within a safe harbor site of a genomic DNA sequence.

Gene editing compositions, including Cas-CLOVER, and methods of using these compositions for gene editing are described in detail in U.S. Patent Publication Nos. 2017/0107541, 2017/0114149, 2018/0187185 and U.S. Pat. No. 10,415,024.

Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)-containing triblock copolymers may assemble into a micelle with positively charged poly(histidine) units on the surface, thereby enabling complexing with the negatively-charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a nuclease using the poly(histidine)-based micelles. Without wishing to be bound by a particular theory, it is believed that believed that in the micelles that are formed by the various triblock copolymers, the hydrophobic blocks aggregate to form a core, leaving the hydrophilic blocks and poly(histidine) blocks on the ends to form one or more surrounding layer.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example triblock copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ϵ-caprolactone) (PCL), and poly(trimethylene carbonate) (PTMC). Polymeric micelles comprised of 100% PEGylated surfaces possess improved in vitro chemical stability, augmented in vivo bioavailability, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589.

### Inducible Proapoptotic Polypeptides

The inducible proapoptotic polypeptides disclosed herein are superior to existing inducible polypeptides because the inducible proapoptotic polypeptides of the disclosure are far less immunogenic. The inducible proapoptotic polypeptides are recombinant polypeptides, and, therefore, non-naturally occurring. Further, the sequences that are recombined to produce inducible proapoptotic polypeptides that do not comprise non-human sequences that the host human immune system could recognize as “non-self” and, consequently, induce an immune response in the subject receiving the inducible proapoptotic polypeptide, a cell comprising the inducible proapoptotic polypeptide or a composition comprising the inducible proapoptotic polypeptide or the cell comprising the inducible proapoptotic polypeptide.

The disclosure provides inducible proapoptotic polypeptides comprising a ligand binding region, a linker, and a proapoptotic peptide, wherein the inducible proapoptotic polypeptide does not comprise a non-human sequence. In certain aspects, the non-human sequence comprises a restriction site. In certain aspects, the ligand binding region can be a multimeric ligand binding region. In certain aspects, the proapoptotic peptide is a caspase polypeptide. Non-limiting examples of caspase polypeptides include caspase 1, caspase 2, caspase 3, caspase 4, caspase 5, caspase 6, caspase 7, caspase 8, caspase 9, caspase 10, caspase 11, caspase 12, and caspase 14. Preferably, the caspase polypeptide is a caspase 9 polypeptide. The caspase 9 polypeptide can be a truncated caspase 9 polypeptide. Inducible proapoptotic polypeptides can be non-naturally occurring. When the caspase is caspase 9) or a truncated caspase 9, the inducible proapoptotic polypeptides can also be referred to as an “iC9 safety switch”.

An inducible caspase polypeptide can comprise (a) a ligand binding region, (b) a linker, and (c) a caspase polypeptide, wherein the inducible proapoptotic polypeptide does not comprise a non-human sequence. In certain aspects, an inducible caspase polypeptide comprises (a) a ligand binding region, (b) a linker, and (c) a truncated caspase 9) polypeptide, wherein the inducible proapoptotic polypeptide does not comprise a non-human sequence.

The ligand binding region can comprise a FK506 binding protein 12 (FKBP12) polypeptide. The amino acid sequence of the ligand binding region that comprises a FK506 binding protein 12 (FKBP12) polypeptide can comprise a modification at position 36 of the sequence. The modification can be a substitution of valine (V) for phenylalanine (F) at position 36 (F36V). The FKBP12 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 131. The FKBP12 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 132.

The linker region can comprise, consist essential of, or consist of, the amino acid sequence at least 75%, 80%, 85%, 90%, 95%, 96%, 97%, 98%, 99% or 100% (or any percentage in between) identical to SEQ ID NO: 133 or the linker region can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 134. In some aspects, the nucleic acid sequence encoding the linker does not comprise a restriction site.

The truncated caspase 9) polypeptide can comprise an amino acid sequence that does not comprise an arginine (R) at position 87 of the sequence. Alternatively, or in addition, the truncated caspase 9 polypeptide can comprise an amino acid sequence that does not comprise an alanine (A) at position 282 the sequence. The truncated caspase 9 polypeptide can comprise, consist essential of, or consist of, the amino acid sequence at least 75%, 80%, 85%, 90%, 95%, 96%, 97%, 98%, 99% or 100% (or any percentage in between) identical to SEQ ID NO: 135 or the truncated caspase 9 polypeptide can be encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 136.

In certain aspects when the polypeptide comprises a truncated caspase 9) polypeptide, the inducible proapoptotic polypeptide comprises, consists essential of, or consists of, the amino acid sequence at least 75%, 80%, 85%, 90%, 95%, 96%, 97%, 98%, 99% or 100% (or any percentage in between) identical to SEQ ID NO: 137 or the inducible proapoptotic polypeptide is encoded by a polynucleotide comprising or consisting of an nucleic acid sequence at least 75%, 80%, 85%, 90%, 95%, 96%, 97%, 98%, 99% or 100% (or any percentage in between) identical to SEQ ID NO: 138, SEQ ID NO: 139, or SEQ ID NO: 140.

Inducible proapoptotic polypeptides can be expressed in a cell under the transcriptional regulation of any promoter known in the art that is capable of initiating and/or regulating the expression of an inducible proapoptotic polypeptide in that cell.

Activation of inducible proapoptotic polypeptides can be accomplished through, for example, chemically induced dimerization (CID) mediated by an induction agent to produce a conditionally controlled protein or polypeptide. Proapoptotic polypeptides not only inducible, but the induction of these polypeptides is also reversible, due to the degradation of the labile dimerizing agent or administration of a monomeric competitive inhibitor.

In certain aspects when the ligand binding region comprises a FKBP12 polypeptide having a substitution of valine (V) for phenylalanine (F) at position 36 (F36V), the induction agent can comprise AP1903, a synthetic drug (CAS Index Name: 2-Piperidinecarboxylic acid, 1-[(2S)-1-oxo-2-(3,4,5-trimethoxyphenyl)butyl]-, 1,2-ethanediylbis[imino (2-oxo-2,1-ethanediyl)oxy-3,1-phenylene[(1R)-3-(3,4-dimethoxyphenyl) propylidene]]ester. [2S-[1(R*),2R*[S*[S*[1(R*),2R*]]]]]-(9Cl) CAS Registry Number: 195514-63-7; Molecular Formula: C78H98N4O20; Molecular Weight: 1411.65)); AP20187 (CAS Registry Number: 195514-80-8 and Molecular Formula: C82H107N5O20) or an AP20187 analog, such as, for example, AP1510. As used herein, the induction agents AP20187, AP1903 and AP1510 can be used interchangeably.

Inducible proapoptotic peptides and methods of inducing these peptides are described in detail in U.S. Patent Publication No. WO 2019/0225667 and PCT Publication No. WO 2018/068022.

### Chimeric Stimulator Receptors and Recombinant HLA-E Polypeptides

Adoptive cell compositions that are “universally” safe for administration to any patient requires a significant reduction or elimination of alloreactivity. Towards this end, cells of the disclosure (e.g., allogenic cells) can be modified to interrupt expression or function of a T-cell Receptor (TCR) and/or a class of Major Histocompatibility Complex (MHC). The TCR mediates graft vs host (GvH) reactions whereas the MHC mediates host vs graft (HvG) reactions. In preferred aspects, any expression and/or function of the TCR is eliminated to prevent T-cell mediated GvH that could cause death to the subject. Thus, in a preferred aspect, the disclosure provides a pure TCR-negative allogeneic T-cell composition (e.g., each cell of the composition expresses at a level so low as to either be undetectable or non-existent).

Expression and/or function of MHC class I (MHC-I, specifically, HLA-A, HLA-B, and HLA-C) is reduced or eliminated to prevent HvG and, consequently, to improve engraftment of cells in a subject. Improved engraftment results in longer persistence of the cells, and, therefore, a larger therapeutic window for the subject. Specifically, expression and/or function of a structural element of MHC-I. Beta-2-Microglobulin (B2M), is reduced or eliminated.

The above strategies induce further challenges. T Cell Receptor (TCR) knockout (KO) in T cells results in loss of expression of CD3-zeta (CD3% or CD3ζ), which is part of the TCR complex. The loss of CD3ζ in TCR-KO T-cells dramatically reduces the ability of optimally activating and expanding these cells using standard stimulation/activation reagents, including, but not limited to, agonist anti-CD3 mAb. When the expression or function of any one component of the TCR complex is interrupted, all components of the complex are lost, including TCR-alpha (TCRα), TCR-beta (TCRβ), CD3-gamma (CD3γ), CD3-epsilon (CD3ϵ), CD3-delta (CD3δ), and CD3-zeta (CD3ζ). Both CD3ϵ and CD3ζ are required for T cell activation and expansion. Agonist anti-CD3 mAbs typically recognize CD3ϵ and possibly another protein within the complex which, in turn, signals to CD3ζ. CD3ζ provides the primary stimulus for T cell activation (along with a secondary co-stimulatory signal) for optimal activation and expansion. Under normal conditions, full T-cell activation depends on the engagement of the TCR in conjunction with a second signal mediated by one or more co-stimulatory receptors (e.g., CD28, CD2, 4-1BBL) that boost the immune response. However, when the TCR is not present, T cell expansion is severely reduced when stimulated using standard activation/stimulation reagents, including agonist anti-CD3 mAb. In fact, T cell expansion is reduced to only 20-40% of the normal level of expansion when stimulated using standard activation/stimulation reagents, including agonist anti-CD3 mAb.

Thus, the present disclosure provides a non-naturally occurring chimeric stimulatory receptor (CSR) comprising: (a) an ectodomain comprising a activation component, wherein the activation component is isolated or derived from a first protein; (b) a transmembrane domain; and (c) an endodomain comprising at least one signal transduction domain, wherein the at least one signal transduction domain is isolated or derived from a second protein; wherein the first protein and the second protein are not identical.

The activation component can comprise a portion of one or more of a component of a T-cell Receptor (TCR), a component of a TCR complex, a component of a TCR co-receptor, a component of a TCR co-stimulatory protein, a component of a TCR inhibitory protein, a cytokine receptor, and a chemokine receptor to which an agonist of the activation component binds. The activation component can comprise a CD2 extracellular domain or a portion thereof to which an agonist binds.

The signal transduction domain can comprise one or more of a component of a human signal transduction domain, T-cell Receptor (TCR), a component of a TCR complex, a component of a TCR co-receptor, a component of a TCR co-stimulatory protein, a component of a TCR inhibitory protein, a cytokine receptor, and a chemokine receptor. The signal transduction domain can comprise a CD3 protein or a portion thereof. The CD3 protein can comprise a CD3% protein or a portion thereof.

The endodomain can further comprise a cytoplasmic domain. The cytoplasmic domain can be isolated or derived from a third protein. The first protein and the third protein can be identical. The ectodomain can further comprise a signal peptide. The signal peptide can be derived from a fourth protein. The first protein and the fourth protein can be identical. The transmembrane domain can be isolated or derived from a fifth protein. The first protein and the fifth protein can be identical.

In some aspects, the activation component does not bind a naturally-occurring molecule. In some aspects, the activation component binds a naturally-occurring molecule but the CSR does not transduce a signal upon binding of the activation component to a naturally-occurring molecule. In some aspects, the activation component binds to a non-naturally occurring molecule. In some aspects, the activation component does not bind a naturally-occurring molecule but binds a non-naturally occurring molecule. The CSR can selectively transduces a signal upon binding of the activation component to a non-naturally occurring molecule.

In a preferred aspect, the present disclosure provides a non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof to which an agonist binds; (b) a transmembrane domain, wherein the transmembrane domain comprises a CD2 transmembrane domain or a portion thereof; and (c) an endodomain comprising a cytoplasmic domain and at least one signal transduction domain, wherein the cytoplasmic domain comprises a CD2 cytoplasmic domain or a portion thereof and wherein the at least one signal transduction domain comprises a CD3L protein or a portion thereof. In some aspects, the non-naturally CSR comprises an amino acid sequence at least 75%, 80%, 85%, 90%, 95%, 96%, 97%, 98%, 99% or 100% (or any percentage in between) identical to SEQ ID NO: 141. In a preferred aspect, the non-naturally occurring CSR comprises an amino acid sequence of SEQ ID NO: 141.

The present disclosure also provides a non-naturally occurring chimeric stimulatory receptor (CSR) wherein the ectodomain comprises a modification. The modification can comprise a mutation or a truncation of the amino acid sequence of the activation component or the first protein when compared to a wild type sequence of the activation component or the first protein. The mutation or a truncation of the amino acid sequence of the activation component can comprise a mutation or truncation of a CD2 extracellular domain or a portion thereof to which an agonist binds. The mutation or truncation of the CD2 extracellular domain can reduce or eliminate binding with naturally occurring CD58. In some aspects, the CD2 extracellular domain comprising the mutation or truncation comprises an amino acid sequence at least 75%, 80%, 85%, 90%, 95%, 96%, 97%, 98%, 99% or 100% (or any percentage in between) identical to SEQ ID NO: 142. In a preferred aspect, the CD2 extracellular domain comprising the mutation or truncation comprises an amino acid sequence of SEQ ID NO: 142.

In a preferred aspect, the present disclosure provides non-naturally occurring chimeric stimulatory receptor (CSR) comprising: (a) an ectodomain comprising a signal peptide and an activation component, wherein the signal peptide comprises a CD2 signal peptide or a portion thereof and wherein the activation component comprises a CD2 extracellular domain or a portion thereof to which an agonist binds and wherein the CD2 extracellular domain or a portion thereof to which an agonist binds comprises a mutation or truncation; (b) a transmembrane domain, wherein the transmembrane domain comprises a CD2 transmembrane domain or a portion thereof; and (c) an endodomain comprising a cytoplasmic domain and at least one signal transduction domain, wherein the cytoplasmic domain comprises a CD2 cytoplasmic domain or a portion thereof and wherein the at least one signal transduction domain comprises a CD35 protein or a portion thereof. In some aspects, the non-naturally CSR comprises an amino acid sequence at least 75%, 80%, 85%, 90%, 95%, 96%, 97%, 98%, 99% or 100% (or any percentage in between) identical to SEQ ID NO: 143. In a preferred aspect, the non-naturally occurring CSR comprises an amino acid sequence of SEQ ID NO: 143.

The present disclosure provides a nucleic acid sequence encoding any CSR disclosed herein. The present disclosure provides a transposon or a vector comprising a nucleic acid sequence encoding any CSR disclosed herein.

The present disclosure provides a cell comprising any CSR disclosed herein. The present disclosure provides a cell comprising a nucleic acid sequence encoding any CSR disclosed herein. The present disclosure provides a cell comprising a vector comprising a nucleic acid sequence encoding any CSR disclosed herein. The present disclosure provides a cell comprising a transposon comprising a nucleic acid sequence encoding any CSR disclosed herein.

A modified cell disclosed herein can be an allogeneic cell or an autologous cell. In some preferred aspects, the modified cell is an allogeneic cell. In some aspects, the modified cell is an autologous T-cell or a modified autologous CAR T-cell. In some preferred aspects, the modified cell is an allogeneic T-cell or a modified allogeneic CAR T-cell.

The present disclosure provides a composition comprising any CSR disclosed herein. The present disclosure provides a composition comprising a nucleic acid sequence encoding any CSR disclosed herein. The present disclosure provides a composition comprising a vector comprising a nucleic acid sequence encoding any CSR disclosed herein. The present disclosure provides a composition comprising a transposon comprising a nucleic acid sequence encoding any CSR disclosed herein. The present disclosure provides a composition comprising a modified cell disclosed herein or a composition comprising a plurality of modified cells disclosed herein.

The present disclosure provides a modified T lymphocyte (T-cell), comprising: (a) a modification of an endogenous sequence encoding a T-cell Receptor (TCR), wherein the modification reduces or eliminates a level of expression or activity of the TCR; and (b) a chimeric stimulatory receptor (CSR) comprising: (i) an ectodomain comprising an activation component, wherein the activation component is isolated or derived from a first protein; (ii) a transmembrane domain; and (iii) an endodomain comprising at least one signal transduction domain, wherein the at least one signal transduction domain is isolated or derived from a second protein; wherein the first protein and the second protein are not identical.

The modified T-cell can further comprise an inducible proapoptotic polypeptide. The modified T-cell can further comprise a modification of an endogenous sequence encoding Beta-2-Microglobulin (B2M), wherein the modification reduces or eliminates a level of expression or activity of a major histocompatibility complex (MHC) class I (MHC-I).

The modified T-cell can further comprise a non-naturally occurring polypeptide comprising an HLA class I histocompatibility antigen, alpha chain E (HLA-E) polypeptide. The non-naturally occurring polypeptide comprising a HLA-E polypeptide can further comprise a B2M signal peptide. The non-naturally occurring polypeptide comprising a HLA-E polypeptide can further comprise a B2M polypeptide. The non-naturally occurring polypeptide comprising an HLA-E polypeptide can further comprise a linker, wherein the linker is positioned between the B2M polypeptide and the HLA-E polypeptide. The non-naturally occurring polypeptide comprising an HLA-E polypeptide can further comprise a peptide and a B2M polypeptide. The non-naturally occurring polypeptide comprising an HLA-E can further comprise a first linker positioned between the B2M signal peptide and the peptide, and a second linker positioned between the B2M polypeptide and the peptide encoding the HLA-E.

The modified T-cell can further comprise a non-naturally occurring antigen receptor, a sequence encoding a therapeutic polypeptide, or a combination thereof. The non-naturally occurring antigen receptor can comprise a chimeric antigen receptor (CAR).

The CSR can be transiently expressed in the modified T-cell. The CSR can be stably expressed in the modified T-cell. The polypeptide comprising the HLA-E polypeptide can be transiently expressed in the modified T-cell. The polypeptide comprising the HLA-E polypeptide can be stably expressed in the modified T-cell. The inducible proapoptotic polypeptide can be transiently expressed in the modified T-cell. The inducible proapoptotic polypeptide can be stably expressed in the modified T-cell. The non-naturally occurring antigen receptor or a sequence encoding a therapeutic protein can be transiently expressed in the modified T-cell. The non-naturally occurring antigen receptor or a sequence encoding a therapeutic protein can be stably expressed in the modified T-cell.

Gene editing compositions, including but not limited to, RNA-guided fusion proteins comprising dCas9-Clo051, as described in detail herein, can be used to target and decrease or eliminate expression of an endogenous T-cell receptor. In preferred aspects, the gene editing compositions target and delete a gene, a portion of a gene, or a regulatory element of a gene (such as a promoter) encoding an endogenous T-cell receptor. Non-limiting examples of primers (including a T7 promoter, genome target sequence, and gRNA scaffold) for the generation of guide RNA (gRNA) templates for targeting and deleting TCR-alpha (TCR-α), targeting and deleting TCR-beta (TCR-β), and targeting and deleting beta-2-microglobulin (β2M) are disclosed in PCT Application No. PCT/US2019/049816.

Gene editing compositions, including but not limited to, RNA-guided fusion proteins comprising dCas9-Clo051, can be used to target and decrease or eliminate expression of an endogenous MHCI, MHCII, or MHC activator. In preferred aspects, the gene editing compositions target and delete a gene, a portion of a gene, or a regulatory element of a gene (such as a promoter) encoding one or more components of an endogenous MHCI, MHCII, or MHC activator. Non-limiting examples of guide RNAs (gRNAs) for targeting and deleting MHC activators are disclosed in PCT Application No. PCT/US2019/049816.

A detailed description of non-naturally occurring chimeric stimulatory receptors, genetic modifications of endogenous sequences encoding TCR-alpha (TCR-α), TCR-beta (TCR-β), and/or Beta-2-Microglobulin (B2M), and non-naturally occurring polypeptides comprising an HLA class I histocompatibility antigen, alpha chain E (HLA-E) polypeptide is disclosed in PCT Application No. PCT/US2019/049816.

### Formulations, Dosages and Modes of Administration

The present disclosure provides formulations, dosages and methods for administration of the compositions described herein.

The disclosed compositions and pharmaceutical compositions can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, Pa.) 1990 and in the “Physician's Desk Reference”, 52nd ed., Medical Economics (Montvale, N.J.) 1998. Pharmaceutically acceptable carriers can be routinely selected that are suitable for the mode of administration, solubility and/or stability of the protein scaffold, fragment or variant composition as well known in the art or as described herein.

Non-limiting examples of pharmaceutical excipients and additives suitable for use include proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Non-limiting examples of protein excipients include serum albumin, such as human serum albumin (HSA), recombinant human albumin (rHA), gelatin, casein, and the like. Representative amino acid/protein components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Non-limiting examples of carbohydrate excipients suitable for use include monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferably, the carbohydrate excipients are mannitol, trehalose, and/or raffinose.

The compositions can also include a buffer or a pH-adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers are organic acid salts, such as citrate.

Additionally, the disclosed compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20)” and “TWEEN 80”), lipids (e.g., phospholipids, fatty acids), steroids (e.g., cholesterol), and chelating agents (e.g., EDTA).

Many known and developed modes can be used for administering therapeutically effective amounts of the compositions or pharmaceutical compositions disclosed herein. Non-limiting examples of modes of administration include bolus, buccal, infusion, intrarticular, intrabronchial, intraabdominal, intracapsular, intracartilaginous, intracavitary, intracelial, intracerebellar, intracerebroventricular, intracolic, intracervical, intragastric, intrahepatic, intralesional, intramuscular, intramyocardial, intranasal, intraocular, intraosseous, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intratumoral, intravenous, intravesical, oral, parenteral, rectal, sublingual, subcutaneous, transdermal or vaginal means.

A composition of the disclosure can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

For parenteral administration, any composition disclosed herein can be formulated as a solution, suspension, emulsion, particle, powder, or lyophilized powder in association, or separately provided, with a pharmaceutically acceptable parenteral vehicle. Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

Formulations for oral administration rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. Formulations for delivery of hydrophilic agents including proteins and protein scaffolds and a combination of at least two surfactants intended for oral, buccal, mucosal, nasal, pulmonary, vaginal transmembrane, or rectal administration are described in U.S. Pat. No. 6,309,663. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations can contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 and used to deliver biologically active agents orally are known in the art.

For pulmonary administration, preferably, a composition or pharmaceutical composition described herein is delivered in a particle size effective for reaching the lower airways of the lung or sinuses. The composition or pharmaceutical composition can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers (e.g., jet nebulizer, ultrasonic nebulizer), dry powder generators, sprayers, and the like. All such devices can use formulations suitable for the administration for the dispensing of a composition or pharmaceutical composition described herein in an aerosol. Such aerosols can be comprised of either solutions (both aqueous and non-aqueous) or solid particles. Additionally, a spray including a composition or pharmaceutical composition described herein can be produced by forcing a suspension or solution of at least one protein scaffold through a nozzle under pressure. In a metered dose inhaler (MDI), a propellant, a composition or pharmaceutical composition described herein, and any excipients or other additives are contained in a canister as a mixture including a liquefied compressed gas. Actuation of the metering valve releases the mixture as an aerosol, preferably containing particles in the size range of less than about 10 μm, preferably, about 1 μm to about 5 μm, and, most preferably, about 2 μm to about 3 μm. A more detailed description of pulmonary administration, formulations and related devices is disclosed in PCT Publication No. WO 2019/049816.

For absorption through mucosal surfaces, compositions include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucous surfaces suitable for application of the emulsions of the disclosure can include corneal, conjunctival, buccal, sublingual, nasal, vaginal, pulmonary, stomachic, intestinal, and rectal routes of administration. Formulations for vaginal or rectal administration, e.g., suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695). A more detailed description of mucosal administration and formulations is disclosed in PCT Publication No. WO 2019/049816.

For transdermal administration, a composition or pharmaceutical composition disclosed herein is encapsulated in a delivery device, such as a liposome or polymeric nanoparticles, microparticle, microcapsule, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599). A more detailed description of transdermal administration, formulations and suitable devices is disclosed in PCT Publication No. WO 2019/049816.

It can be desirable to deliver the disclosed compounds to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b), e.g., a zinc tannate salt. Additionally, the disclosed compounds or, preferably, a relatively insoluble salt, such as those just described, can be formulated in a gel, for example, an aluminum monostearate gel with, e.g., sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer for example as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and “Sustained and Controlled Release Drug Delivery Systems”, J. R. Robinson ed., Marcel Dekker, Inc., N.Y., 1978).

Suitable dosages are well known in the art. See, e.g. Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000); Nursing 2001 Handbook of Drugs, 21st edition, Springhouse Corp., Springhouse, Pa., 2001; Health Professional's Drug Guide 2001, ed., Shannon, Wilson, Stang, Prentice-Hall, Inc, Upper Saddle River, N.J. Preferred doses can optionally include about 0.1-99 and/or 100-500 mg/kg/administration, or any range, value or fraction thereof, or to achieve a serum concentration of about 0.1-5000 μg/ml serum concentration per single or multiple administration, or any range, value or fraction thereof. A preferred dosage range for the compositions or pharmaceutical compositions disclosed herein is from about 1 mg/kg, up to about 3, about 6 or about 12 mg/kg of body weight of the subject.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably, 0.1 to 10 milligrams per kilogram per administration or in sustained release form is effective to obtain desired results.

As a non-limiting example, treatment of humans or animals can be provided as a one-time or periodic dosage of the compositions or pharmaceutical compositions disclosed herein about 0.1 to 100 mg/kg or any range, value or fraction thereof per day, on at least one of day 1-40, or, alternatively or additionally, at least one of week 1-52, or, alternatively or additionally, at least one of 1-20 years, or any combination thereof, using single, infusion or repeated doses.

Dosage forms suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

An effective amount can comprise an amount of about 0.001 to about 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

In aspects where the compositions to be administered to a subject in need thereof are modified cells as disclosed herein, the cells can be administered between about 1×10³ and 1×10¹⁵ cells; about 1×10⁴ and 1×10¹² cells; about 1×10⁵ and 1×10¹⁰ cells; about 1×10⁶ and 1×10⁹ cells; about 1×10⁶ and 1×10⁸ cells; about 1×10⁶ and 1×10⁷ cells; or about 1×10⁶ and 25×10⁶ cells. In an aspect the cells are administered between about 5×10⁶ and 25×10⁶ cells.

A more detailed description of pharmaceutically acceptable excipients, formulations, dosages and methods of administration of the disclosed compositions and pharmaceutical compositions is disclosed in PCT Publication No. WO 2019/049816.

### Methods of Using the Compositions of the Disclosure

The disclosure provides the use of a disclosed polynucleotide for improving transposition activity. Transposition activity may include excision, integration or a combination thereof. Specifically, the method comprising contacting a cell or a plurality of cells with: a) a polynucleotide sequence encoding a transposon comprising a first ITR and a second ITR, wherein the first ITR and/or the second ITR comprises a nucleic acid substitution; and b) a nucleic acid sequence or an amino acid sequence comprising a sequence encoding a transposase enzyme. In some aspects the first ITR corresponds to the LE ITR. In some aspects, the second ITR correspond to the RE ITR.

In some aspects, the nucleic acid substitution is located in the first ITR. In some aspects, the first ITR comprises a nucleic acid substitution in at least one of nucleic acid position 31 and position 33. In some aspects, the first ITR comprises the nucleic acid substitution of 31G>T. In some aspects, the first ITR comprises the nucleic acid substitution of 33A>C. In some aspects, the first ITR comprises the nucleic acid substitution of 31G>T and 33A>C. In some aspects, the first ITR comprises the nucleic acid sequence of SEQ ID NO: 14. In some aspects, the first ITR comprises the nucleic acid sequence of SEQ ID NO: 15. In some aspects, the first ITR comprises the nucleic acid sequence of SEQ ID NO: 16.

In an aspect, the polynucleotide encoding the transposon of the disclosure increases excision by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a polynucleotide encoding a transposon having a wildtype first ITR (i.e. LE ITR) and a wildtype second ITR (i.e. RE ITR).

In an aspect, the polynucleotide encoding the transposon of the disclosure increases integration by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a polynucleotide encoding a transposon having a wildtype first ITR (i.e. LE ITR) and a wildtype second ITR (i.e. RE ITR).

In an aspect, the polynucleotide encoding the transposon of the disclosure increases transposition activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a polynucleotide encoding a transposon having a wildtype first ITR (i.e. LE ITR) and a wildtype second ITR (i.e. RE ITR).

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In an aspect, the subject is a mammal. Preferably, the subject is human. The terms “subject” and “patient” are used interchangeably herein.

The disclosure provides a method for modulating or treating at least one malignant disease or disorder in a cell, tissue, organ, animal or subject. Preferably, the malignant disease is cancer. Non-limiting examples of a malignant disease or disorder include leukemia, acute leukemia, acute lymphoblastic leukemia (ALL), acute lymphocytic leukemia, B-cell, T-cell or FAB ALL, acute myeloid leukemia (AML), acute myelogenous leukemia, chronic myelocytic leukemia (CML), chronic lymphocytic leukemia (CLL), hairy cell leukemia, myelodyplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, bladder cancer, breast cancer, colorectal cancer, endometrial cancer, head cancer, neck cancer, hereditary nonpolyposis cancer, Hodgkin's lymphoma, liver cancer, lung cancer, non-small cell lung cancer, ovarian cancer, pancreatic cancer, prostate cancer, renal cell carcinoma, testicular cancer, adenocarcinomas, sarcomas, malignant melanoma, hemangioma, metastatic disease, cancer related bone resorption, cancer related bone pain, and the like.

In some aspects, the treatment of a malignant disease or disorder comprises adoptive cell therapy. For example, in an aspect, the disclosure provides modified cells that express at least one disclosed protein scaffold and/or CAR comprising a protein scaffold (e.g., scFv, single domain antibody, Centyrin, delivered to the cell with a composition of the disclosure) that have been selected and/or expanded for administration to a subject in need thereof. Modified cells can be formulated for storage at any temperature including room temperature and body temperature. Modified cells can be formulated for cryopreservation and subsequent thawing. Modified cells can be formulated in a pharmaceutically acceptable carrier for direct administration to a subject from sterile packaging. Modified cells can be formulated in a pharmaceutically acceptable carrier with an indicator of cell viability and/or CAR expression level to ensure a minimal level of cell function and CAR expression. Modified cells can be formulated in a pharmaceutically acceptable carrier at a prescribed density with one or more reagents to inhibit further expansion and/or prevent cell death.

In a non-limiting example, the present disclosure provides methods of treating a metabolic liver disorder in a subject, the methods comprising administering to the subject: a) at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the metabolic liver disorder can be Ornithine Transcarbamylase (OTC) Deficiency and the at least one therapeutic protein can comprise ornithine transcarbamylase (OTC) polypeptide. In some aspects, the metabolic liver disorder can be methylmalonic acidemia (MMA) and the at least one therapeutic protein can comprise a methylmalonyl-CoA mutase (MUT1) polypeptide.

In a non-limiting example, the present disclosure provides methods of treating a hemophilia disease in a subject, the methods comprising administering to the subject: at least one therapeutically effective amount of at least one composition comprising a transposon of the present disclosure comprising a sequence encoding a therapeutic polypeptide; and b) at least one therapeutically effective amount of a composition comprising a nucleic acid sequence encoding at least one transposase. In some aspects, the hemophilia disease can be hemophilia A and the at least one therapeutic protein can comprise Factor VIII. In some aspects, the hemophilia disease can be hemophilia B and the at least one therapeutic protein can comprise Factor IX.

Any can comprise administering an effective amount of any composition or pharmaceutical composition disclosed herein to a cell, tissue, organ, animal or subject in need of such modulation, treatment or therapy. Such a method can optionally further comprise co-administration or combination therapy for treating such diseases or disorders, wherein the administering of any composition or pharmaceutical composition disclosed herein, further comprises administering, before concurrently, and/or after, at least one chemotherapeutic agent (e.g., an alkylating agent, an a mitotic inhibitor, a radiopharmaceutical).

In some aspects, the subject does not develop graft vs. host (GvH) and/or host vs. graft (HvG) following administration. In an aspect, the administration is systemic. Systemic administration can be any means known in the art and described in detail herein. Preferably, systemic administration is by an intravenous injection or an intravenous infusion. In an aspect, the administration is local. Local administration can be any means known in the art and described in detail herein. Preferably, local administration is by intra-tumoral injection or infusion, intraspinal injection or infusion, intracerebroventricular injection or infusion, intraocular injection or infusion, or intraosseous injection or infusion.

In some aspects, the therapeutically effective dose is a single dose. In some aspects, the single dose is one of at least 2, 5, 10, 15, 20, 25, 30, 35, 40, 45, 50, 55, 60, 65, 70, 75, 80, 85, 90, 95, 100 or any number of doses in between that are manufactured simultaneously. In some aspects, where the composition is autologous cells or allogeneic cells, the dose is an amount sufficient for the cells to engraft and/or persist for a sufficient time to treat the disease or disorder.

In one example, the disclosure provides a method of treating cancer in a subject in need thereof, comprising administering to the subject a composition comprising a protein scaffold or a CAR comprising a protein scaffold (e.g., scFv, single domain antibody. Centyrin) the antibody or CAR specifically binds to an antigen on a tumor cell. In aspects where the composition comprises a modified cell or cell population, the cell or cell population may be autologous or allogeneic.

In some aspects of the methods of treatment described herein, the treatment can be modified or terminated. Specifically, in aspects where the composition used for treatment comprises an inducible proapoptotic polypeptide, apoptosis may be selectively induced in the cell by contacting the cell with an induction agent. A treatment may be modified or terminated in response to, for example, a sign of recovery or a sign of decreasing disease severity/progression, a sign of disease remission/cessation, and/or the occurrence of an adverse event. In some aspects, the method comprises the step of administering an inhibitor of the induction agent to inhibit modification of the cell therapy, thereby restoring the function and/or efficacy of the cell therapy (for example, when a sign or symptom of the disease reappear or increase in severity and/or an adverse event is resolved).

### Protein Scaffold Production, Screening and Purification

At least one protein scaffold (e.g., monoclonal antibody, a chimeric antibody, a single domain antibody, a VHH, a VH, a single chain variable fragment (scFv), a Centyrin, an antigen-binding fragment (Fab) or a Fab fragment) of the disclosure can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

Amino acids from a protein scaffold can be altered, added and/or deleted to reduce immunogenicity or reduce, enhance or modify binding, affinity, on-rate, off-rate, avidity, specificity, half-life, stability, solubility or any other suitable characteristic, as known in the art.

Optionally, a protein scaffold can be engineered with retention of high affinity for the antigen and other favorable biological properties. To achieve this goal, the scaffold proteins can be optionally prepared by a process of analysis of the parental sequences and various conceptual engineered products using three-dimensional models of the parental and engineered sequences. Three-dimensional models are commonly available and are familiar to those skilled in the art. Computer programs are available which illustrate and display probable three-dimensional conformational structures of selected candidate sequences and can measure possible immunogenicity (e.g., Immunofilter program of Xencor, Inc. of Monrovia, Calif.). Inspection of these displays permits analysis of the likely role of the residues in the functioning of the candidate sequence, i.e., the analysis of residues that influence the ability of the candidate protein scaffold to bind its antigen. In this way, residues can be selected and combined from the parent and reference sequences so that the desired characteristic, such as affinity for the target antigen(s), is achieved. Alternatively, or in addition to, the above procedures, other suitable methods of engineering can be used.

Screening of a protein scaffold for specific binding to similar proteins or fragments can be conveniently achieved using nucleotide (DNA or RNA display) or peptide display libraries, for example, in vitro display. This method involves the screening of large collections of peptides for individual members having the desired function or structure. The displayed nucleotide or peptide sequences can be from 3 to 5000 or more nucleotides or amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. WO 91/17271, WO 91/18980, WO 91/19818, and WO 93/08278.

Other systems for generating libraries of peptides have aspects of both in vitro chemical synthesis and recombinant methods. See, PCT Patent Publication Nos. WO 92/05258, WO 92/14843, and WO 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge Antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra.

A protein scaffold of the disclosure can bind human or other mammalian proteins with a wide range of affinities (KD). In a preferred aspect, at least one protein scaffold of the present disclosure can optionally bind to a target protein with high affinity, for example, with a KD equal to or less than about 10⁻⁷ M, such as but not limited to, 0.1-9.9 (or any range or value therein)×10⁻⁸, 10⁻⁹, 10⁻¹⁰, 10⁻¹¹, 10⁻¹², 10⁻¹³, 10⁻¹⁴, 10⁻¹⁵ or any range or value therein, as determined by surface plasmon resonance or the Kinexa method, as practiced by those of skill in the art.

The affinity or avidity of a protein scaffold for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W.H. Freeman and Company: New York, N.Y. (1992); and methods described herein). The measured affinity of a particular protein scaffold-antigen interaction can vary if measured under different conditions (e.g., salt concentration, pH). Thus, measurements of affinity and other antigen-binding parameters (e.g., KD, Kon, Koff) are preferably made with standardized solutions of protein scaffold and antigen, and a standardized buffer, such as the buffer described herein.

Competitive assays can be performed with a protein scaffold in order to determine what proteins, antibodies, and other antagonists compete for binding to a target protein with the protein scaffold and/or share the epitope region. These assays as readily known to those of ordinary skill in the art evaluate competition between antagonists or ligands for a limited number of binding sites on a protein. The protein and/or antibody is immobilized or insolubilized before or after the competition and the sample bound to the target protein is separated from the unbound sample, for example, by decanting (where the protein/antibody was pre-insolubilized) or by centrifuging (where the protein/antibody was precipitated after the competitive reaction). Also, the competitive binding may be determined by whether function is altered by the binding or lack of binding of the protein scaffold to the target protein, e.g., whether the protein scaffold inhibits or potentiates the enzymatic activity of, for example, a label. ELISA and other functional assays may be used, as well known in the art.

### Nucleic Acid Molecules

Nucleic acid molecules of the disclosure encoding a protein scaffold can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the disclosure can include nucleic acid molecules comprising an open reading frame (ORF), optionally, with one or more introns, e.g., but not limited to, at least one specified portion of at least one protein scaffold; nucleic acid molecules comprising the coding sequence for a protein scaffold or loop region that binds to the target protein; and nucleic acid molecules which comprise a nucleotide sequence substantially different from those described above but which, due to the degeneracy of the genetic code, still encode the protein scaffold as described herein and/or as known in the art. Of course, the genetic code is well known in the art. Thus, it would be routine for one skilled in the art to generate such degenerate nucleic acid variants that code for a specific protein scaffold of the present disclosure. See, e.g., Ausubel, et al., supra, and such nucleic acid variants are included in the present disclosure.

As indicated herein, nucleic acid molecules of the disclosure which comprise a nucleic acid encoding a protein scaffold can include, but are not limited to, those encoding the amino acid sequence of a protein scaffold fragment, by itself; the coding sequence for the entire protein scaffold or a portion thereof: the coding sequence for a protein scaffold, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding a protein scaffold can be fused to a marker sequence, such as a sequence encoding a peptide that facilitates purification of the fused protein scaffold comprising a protein scaffold fragment or portion.

### Polynucleotides Selectively Hybridizing to a Polynucleotide as Described Herein

The disclosure provides isolated nucleic acids that hybridize under selective hybridization conditions to a polynucleotide disclosed herein. Thus, the polynucleotides can be used for isolating, detecting, and/or quantifying nucleic acids comprising such polynucleotides. For example, polynucleotides of the present disclosure can be used to identify, isolate, or amplify partial or full-length clones in a deposited library. The polynucleotides can be genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of a protein scaffold encoded by the polynucleotides described herein. The polynucleotides embrace nucleic acid sequences that can be employed for selective hybridization to a polynucleotide encoding a protein scaffold of the present disclosure. See, e.g., Ausubel, supra; Colligan, supra, each entirely incorporated herein by reference.

### Construction of Nucleic Acids

The isolated nucleic acids of the disclosure can be made using (a) recombinant methods, (b) synthetic techniques, (c) purification techniques, and/or (d) combinations thereof, as well-known in the art.

The nucleic acids can conveniently comprise sequences in addition to a polynucleotide of the present disclosure. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the disclosure. For example, a hexa-histidine marker sequence provides a convenient means to purify the proteins of the disclosure. The nucleic acid of the disclosure, excluding the coding sequence, is optionally a vector, adapter, or linker for cloning and/or expression of a polynucleotide of the disclosure.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra).

### Recombinant Methods for Constructing Nucleic Acids

The isolated nucleic acid compositions of this disclosure, such as RNA, cDNA, genomic DNA, or any combination thereof, can be obtained from biological sources using any number of cloning methodologies known to those of skill in the art. In some aspects, oligonucleotide probes that selectively hybridize, under stringent conditions, to the polynucleotides of the present disclosure are used to identify the desired sequence in a cDNA or genomic DNA library. The isolation of RNA, and construction of cDNA and genomic libraries are well known to those of ordinary skill in the art. (See, e.g., Ausubel, supra; or Sambrook, supra).

### Nucleic Acid Screening and Isolation Methods

A cDNA or genomic library can be screened using a probe based upon the sequence of a polynucleotide of the disclosure. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the disclosure without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the disclosure and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids of the disclosure can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The disclosure further provides recombinant expression cassettes comprising a nucleic acid of the disclosure. A nucleic acid sequence of the disclosure, for example, a cDNA or a genomic sequence encoding a protein scaffold of the disclosure, can be used to construct a recombinant expression cassette that can be introduced into at least one desired host cell. A recombinant expression cassette will typically comprise a polynucleotide of the disclosure operably linked to transcriptional initiation regulatory sequences that will direct the transcription of the polynucleotide in the intended host cell. Both heterologous and non-heterologous (i.e., endogenous) promoters can be employed to direct expression of the nucleic acids of the disclosure.

In some aspects, isolated nucleic acids that serve as promoter, enhancer, or other elements can be introduced in the appropriate position (upstream, downstream or in the intron) of a non-heterologous form of a polynucleotide of the disclosure so as to up or down regulate expression of a polynucleotide of the disclosure. For example, endogenous promoters can be altered in vivo or in vitro by mutation, deletion and/or substitution.

### Expression Vectors and Host Cells

The disclosure also relates to vectors that include isolated nucleic acid molecules of the disclosure, host cells that are genetically engineered with the recombinant vectors, and the production of at least one protein scaffold by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, ampicillin, zeocin (Sh bla gene), puromycin (pac gene), hygromycin B (hygB gene), G418/Geneticin (neo gene), DHFR (encoding Dihydrofolate Reductase and conferring resistance to Methotrexate), mycophenolic acid, or glutamine synthetase (GS, U.S. Pat. Nos. 5,122,464:5,770,359:5,827,739), blasticidin (bsd gene), resistance genes for eukaryotic cell culture as well as ampicillin, zeocin (Sh bla gene), puromycin (pac gene), hygromycin B (hygB gene), G418/Geneticin (neo gene), kanamycin, spectinomycin, streptomycin, carbenicillin, bleomycin, erythromycin, polymyxin B, or tetracycline resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

Expression vectors will preferably but optionally include at least one selectable cell surface marker for isolation of cells modified by the compositions and methods of the disclosure. Selectable cell surface markers of the disclosure comprise surface proteins, glycoproteins, or group of proteins that distinguish a cell or subset of cells from another defined subset of cells. Preferably the selectable cell surface marker distinguishes those cells modified by a composition or method of the disclosure from those cells that are not modified by a composition or method of the disclosure. Such cell surface markers include, e.g., but are not limited to, “cluster of designation” or “classification determinant” proteins (often abbreviated as “CD”) such as a truncated or full length form of CD19, CD271, CD34, CD22, CD20, CD33, CD52, or any combination thereof. Cell surface markers further include the suicide gene marker RQR8 (Philip B et al. Blood. 2014 Aug. 21; 124 (8): 1277-87).

Expression vectors will preferably but optionally include at least one selectable drug resistance marker for isolation of cells modified by the compositions and methods of the disclosure. Selectable drug resistance markers of the disclosure may comprise wild-type or mutant Neo, DHFR, TYMS, FRANCF, RAD51C, GCS, MDR1, ALDH1, NKX2.2, or any combination thereof.

At least one protein scaffold of the disclosure can be expressed in a modified form, such as a fusion protein, and can include not only secretion signals, but also additional heterologous functional regions. For instance, a region of additional amino acids, particularly charged amino acids, can be added to the N-terminus of a protein scaffold to improve stability and persistence in the host cell, during purification, or during subsequent handling and storage. Also, peptide moieties can be added to a protein scaffold of the disclosure to facilitate purification. Such regions can be removed prior to final preparation of a protein scaffold or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the disclosure. Alternatively, nucleic acids of the disclosure can be expressed in a host cell by turning on (by manipulation) in a host cell that contains endogenous DNA encoding a protein scaffold of the disclosure. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the protein scaffolds, specified portions or variants thereof, are bacterial, yeast, and mammalian cells as known in the art. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0)-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0)-Ag14 cells (ATCC Accession Number CRL-1851). In a preferred aspect, the recombinant cell is a P3X63Ab8.653 or an SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present disclosure are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Protein Scaffold Purification

A protein scaffold can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

A protein scaffold of the disclosure include purified products, products of chemical synthetic procedures, and products produced by recombinant techniques from a prokaryotic or eukaryotic host, including, for example, E. coli, yeast, higher plant, insect and mammalian cells. Depending upon the host employed in a recombinant production procedure, the protein scaffold of the disclosure can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Amino Acid Codes

The amino acids that make up protein scaffolds of the disclosure are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994). A protein scaffold of the disclosure can include one or more amino acid substitutions, deletions or additions, from spontaneous or mutations and/or human manipulation, as specified herein. Amino acids in a protein scaffold of the disclosure that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one neutralizing activity. Sites that are critical for protein scaffold binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

As those of skill will appreciate, the disclosure includes at least one biologically active protein scaffold of the disclosure. Biologically active protein scaffolds have a specific activity at least 20%, 30%, or 40%, and, preferably, at least 50%, 60%, or 70%, and, most preferably, at least 80%, 90%, or 95%-99% or more of the specific activity of the native (non-synthetic), endogenous or related and known protein scaffold. Methods of assaying and quantifying measures of enzymatic activity and substrate specificity are well known to those of skill in the art.

In another aspect, the disclosure relates to protein scaffolds and fragments, as described herein, which are modified by the covalent attachment of an organic moiety. Such modification can produce a protein scaffold fragment with improved pharmacokinetic properties (e.g., increased in vivo serum half-life). The organic moiety can be a linear or branched hydrophilic polymeric group, fatty acid group, or fatty acid ester group. In particular aspect, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified protein scaffolds and fragments of the disclosure can comprise one or more organic moieties that are covalently bonded, directly or indirectly, to the antibody. Each organic moiety that is bonded to a protein scaffold or fragment of the disclosure can independently be a hydrophilic polymeric group, a fatty acid group or a fatty acid ester group. As used herein, the term “fatty acid” encompasses mono-carboxylic acids and di-carboxylic acids. A “hydrophilic polymeric group.” as the term is used herein, refers to an organic polymer that is more soluble in water than in octane. For example, polylysine is more soluble in water than in octane. Thus, a protein scaffold modified by the covalent attachment of polylysine is encompassed by the disclosure. Hydrophilic polymers suitable for modifying protein scaffolds of the disclosure can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the protein scaffold of the disclosure has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying protein scaffolds of the disclosure can be saturated or can contain one or more units of unsaturation. Fatty acids that are suitable for modifying protein scaffolds of the disclosure include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40)), cis-49-octadecanoate (C18, oleate), all cis-Δ5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified protein scaffolds and fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified protein scaffolds of the disclosure can be produced by reacting a protein scaffold or fragment with a modifying agent. For example, the organic moieties can be bonded to the protein scaffold in a non-site specific manner by employing an amine-reactive modifying agent, for example, an NHS ester of PEG. Modified protein scaffolds and fragments comprising an organic moiety that is bonded to specific sites of a protein scaffold of the disclosure can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6 (10): 2233-2241 (1997); Itoh et al., Bioorg. Chem., 24 (1): 59-68 (1996); Capellas et al., Biotechnol. Bioeng., 56 (4): 456-463 (1997)), and the methods described in Hermanson, G. T., Bioconjugate Techniques, Academic Press: San Diego, Calif. (1996).

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

The disclosure provides fragments and variants of the disclosed DNA sequences and proteins encoded by these DNA sequences. As used throughout the disclosure, the term “fragment” refers to a portion of the DNA sequence or a portion of the amino acid sequence and hence protein encoded thereby. Fragments of a DNA sequence comprising coding sequences may encode protein fragments that retain biological activity of the native protein and hence DNA recognition or binding activity to a target DNA sequence as herein described. Alternatively, fragments of a DNA sequence that are useful as hybridization probes generally do not encode proteins that retain biological activity or do not retain promoter activity. Thus, fragments of a DNA sequence may range from at least about 20 nucleotides, about 50 nucleotides, about 100 nucleotides, and up to the full-length polynucleotide of the disclosure.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptide produced by this method as well nucleic acid sequences encoding these polypeptides. The disclosure provides host organisms and cells comprising nucleic acid sequences encoding polypeptides produced this modular approach.

The term “antibody” is used in the broadest sense and specifically covers single monoclonal antibodies (including agonist and antagonist antibodies) and antibody compositions with polyepitopic specificity. It is also within the scope hereof to use natural or synthetic analogs, mutants, variants, alleles, homologs and orthologs (herein collectively referred to as “analogs”) of the antibodies hereof as defined herein. Thus, according to an aspect hereof, the term “antibody hereof” in its broadest sense also covers such analogs. Generally, in such analogs, one or more amino acid residues may have been replaced, deleted and/or added, compared to the antibodies hereof as defined herein.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CHI in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). The term further includes single domain antibodies (“sdAB”) which generally refers to an antibody fragment having a single monomeric variable antibody domain, (for example, from camelids). Such antibody fragment types will be readily understood by a person having ordinary skill in the art.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

The term “epitope” refers to an antigenic determinant of a polypeptide. An epitope could comprise three amino acids in a spatial conformation, which is unique to the epitope. Generally, an epitope consists of at least 4, 5, 6, or 7 such amino acids, and more usually, consists of at least 8, 9, or 10 such amino acids. Methods of determining the spatial conformation of amino acids are known in the art, and include, for example, x-ray crystallography and two-dimensional nuclear magnetic resonance.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

“Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

Non-covalently linked components and methods of making and using non-covalently linked components, are disclosed. The various components may take a variety of different forms as described herein. For example, non-covalently linked (i.e., operatively linked) proteins may be used to allow temporary interactions that avoid one or more problems in the art. The ability of non-covalently linked components, such as proteins, to associate and dissociate enables a functional association only or primarily under circumstances where such association is needed for the desired activity. The linkage may be of duration sufficient to allow the desired effect.

A method for directing proteins to a specific locus in a genome of an organism is disclosed. The method may comprise the steps of providing a DNA localization component and providing an effector molecule, wherein the DNA localization component and the effector molecule are capable of operatively linking via a non-covalent linkage.

The term “scFv” refers to a single-chain variable fragment. scFv is a fusion protein of the variable regions of the heavy (VH) and light chains (VL) of immunoglobulins, connected with a linker peptide. The linker peptide may be from about 5 to 40 amino acids or from about 10 to 30 amino acids or about 5, 10, 15, 20, 25, 30, 35, or 40 amino acids in length. Single-chain variable fragments lack the constant Fc region found in complete antibody molecules, and, thus, the common binding sites (e.g., Protein G) used to purify antibodies. The term further includes a scFv that is an intrabody, an antibody that is stable in the cytoplasm of the cell, and which may bind to an intracellular protein.

The term “single domain antibody” means an antibody fragment having a single monomeric variable antibody domain which is able to bind selectively to a specific antigen. A single-domain antibody generally is a peptide chain of about 110 amino acids long, comprising one variable domain (VH) of a heavy-chain antibody, or of a common IgG, which generally have similar affinity to antigens as whole antibodies, but are more heat-resistant and stable towards detergents and high concentrations of urea. Examples are those derived from camelid or fish antibodies. Alternatively, single-domain antibodies can be made from common murine or human IgG with four chains.

The terms “specifically bind” and “specific binding” as used herein refer to the ability of an antibody, an antibody fragment or a nanobody to preferentially bind to a particular antigen that is present in a homogeneous mixture of different antigens. In some aspects, a specific binding interaction will discriminate between desirable and undesirable antigens in a sample. In some aspects, more than about ten- to 100-fold or more (e.g., more than about 1000- or 10,000-fold). “Specificity” refers to the ability of an immunoglobulin or an immunoglobulin fragment, such as a nanobody, to bind preferentially to one antigenic target versus a different antigenic target and does not necessarily imply high affinity.

A “target site” or “target sequence” is a nucleic acid sequence that defines a portion of a nucleic acid to which a binding molecule will bind, provided sufficient conditions for binding exist.

The terms “nucleic acid” or “oligonucleotide” or “polynucleotide” refer to at least two nucleotides covalently linked together. The depiction of a single strand also defines the sequence of the complementary strand. Thus, a nucleic acid may also encompass the complementary strand of a depicted single strand. A nucleic acid of the disclosure also encompasses substantially identical nucleic acids and complements thereof that retain the same structure or encode for the same protein.

Probes of the disclosure may comprise a single stranded nucleic acid that can hybridize to a target sequence under stringent hybridization conditions. Thus, nucleic acids of the disclosure may refer to a probe that hybridizes under stringent hybridization conditions.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally-occur, rendering the entire nucleic acid sequence non-naturally occurring. Nucleic acids of the disclosure may contain one or more duplicated, inverted or repeated sequences, the resultant sequence of which does not naturally-occur, rendering the entire nucleic acid sequence non-naturally occurring. Nucleic acids of the disclosure may contain modified, artificial, or synthetic nucleotides that do not naturally-occur, rendering the entire nucleic acid sequence non-naturally occurring.

Given the redundancy in the genetic code, a plurality of nucleotide sequences may encode any particular protein. All such nucleotides sequences are contemplated herein.

As used throughout the disclosure, the term “operably linked” refers to the expression of a gene that is under the control of a promoter with which it is spatially connected. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

As used throughout the disclosure, the term “promoter” refers to a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, EF-1 Alpha promoter, CAG promoter, SV40 early promoter or SV40 late promoter and the CMV IE promoter.

As used throughout the disclosure, the term “substantially complementary” refers to a first sequence that is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, or more nucleotides or amino acids, or that the two sequences hybridize under stringent hybridization conditions.

As used throughout the disclosure, the term “substantially identical” refers to a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540 or more nucleotides or amino acids, or with respect to nucleic acids, if the first sequence is substantially complementary to the complement of the second sequence.

As used throughout the disclosure, the term “variant” when used to describe a nucleic acid, refers to (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

As used throughout the disclosure, the term “vector” refers to a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. A vector may comprise a combination of an amino acid with a DNA sequence, an RNA sequence, or both a DNA and an RNA sequence.

As used throughout the disclosure, the term “variant” when used to describe a peptide or polypeptide, refers to a peptide or polypeptide that differs in amino acid sequence by the insertion, deletion, or conservative substitution of amino acids, but retain at least one biological activity. Variant can also mean a protein with an amino acid sequence that is substantially identical to a referenced protein with an amino acid sequence that retains at least one biological activity.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example immunogenicity. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table A.

TABLE A
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger. (Biochemistry. Second Edition; Worth Publishers. Inc. NY. N.Y. (1975). pp. 71-77) as set forth in Table B.

TABLE B
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged- | Hydroxyl: | S T Y |
| polar | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table C.

TABLE C
Conservative Substitutions III

| Original | Exemplary |
|---|---|
| Residue | Substitution |
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitution.

As used throughout the disclosure, the term “more than one” of the aforementioned amino acid substitutions refers to 2, 3, 4, 5, 6, 7, 8, 9, 10, 11, 12, 13, 14, 15, 16, 17, 18, 19, or 20 or more of the recited amino acid substitutions. The term “more than one” may refer to 2, 3, 4, or 5 of the recited amino acid substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally-occur, rendering the entire amino acid sequence non-naturally occurring. Polypeptides and proteins of the disclosure may contain one or more duplicated, inverted or repeated sequences, the resultant sequence of which does not naturally-occur, rendering the entire amino acid sequence non-naturally occurring. Polypeptides and proteins of the disclosure may contain modified, artificial, or synthetic amino acids that do not naturally-occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequence naturally associated with a target gene or a host cell into which it is introduced.

As used throughout the disclosure, the term “exogenous” refers to nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended presenting to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

### EXAMPLES

### Example 1: Identification of Mutations within the ITR Regions of Transposons which Improve PiggyBac Mediated Transposition

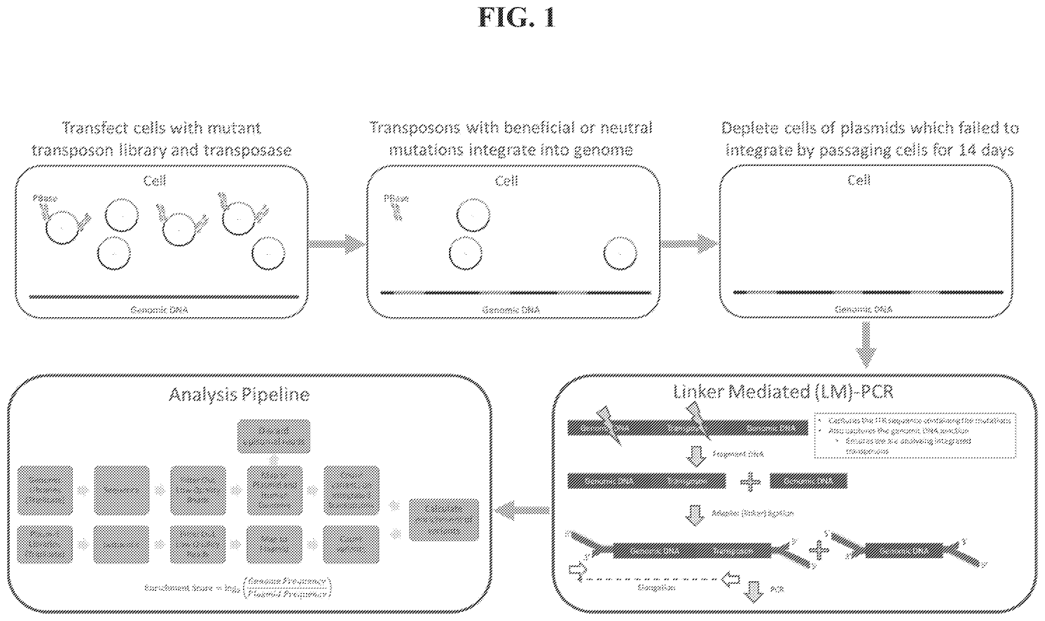

Mutant PiggyBac (PB) transposon libraries were designed with the goal of identifying mutations within the ITR regions of the transposons which confer an increase in PB transposase or Super PiggyBac (SPB) transposase mediated transposition. To screen for mutants with increased transposition activity, cells were co-transfected with a PB transposase and a pool of PB transposons containing mutant ITR sequences from various mutational libraries disclosed herein. All mutants were cloned into a PB transposon as shown in FIG. 1. PuroR was used for selection and GFP was used for visualization and quantification. PB transposons with beneficial or neutral mutations integrate into the genome were selected for and cells with plasmids which failed to integrate were depleted by passaging the cells for 14 days. Linker mediated (LM)-PCR was used to capture and identify the ITR sequence containing the mutations. Genomic junctions were included in the LM-PCR analysis in order to confirm that analysis only included integrated transposons.

### DDBD Binding Site Library of the LE ITR

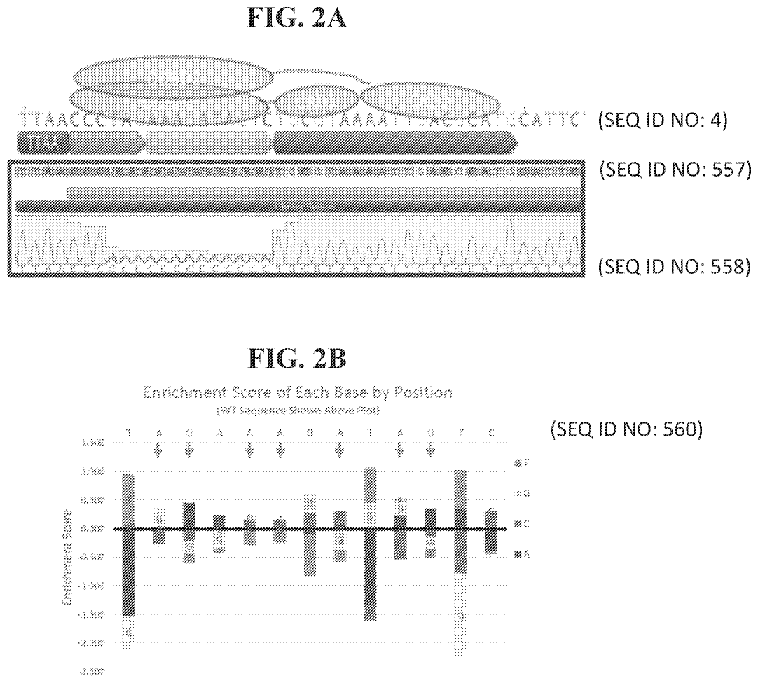

Single base pair substitution mutations of a 13 bp window around the Dimerization and DNA Binding Domain (DDBD) binding site of the LE ITR were investigated in the first library design (i.e. “DDBD library”) (FIG. 2A). Each position of the 13 bp window can be mutated alone or in combination to create 67 million possible mutants. A random subset of 1 million mutants out of the 67 million possible mutants was generated to produce the DDBD library. Analysis of the enrichment score of each of the 13 base pairs around the DDBD binding site of the LE ITR shows that the wildtype nucleotide is not always the most enriched (FIG. 2B).

### Substitution, Insertion, Deletion and Combination Mutational Libraries of the LE ITR and RE ITR

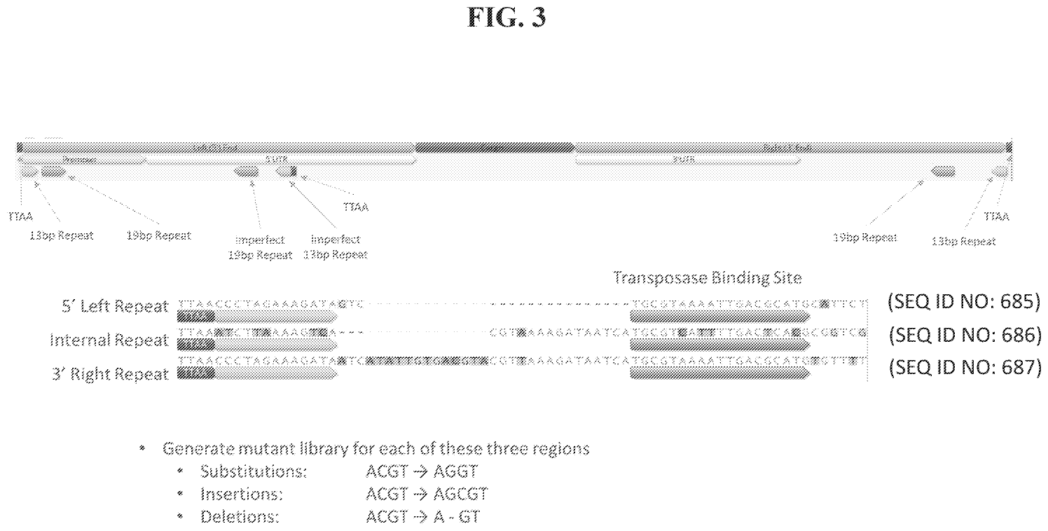

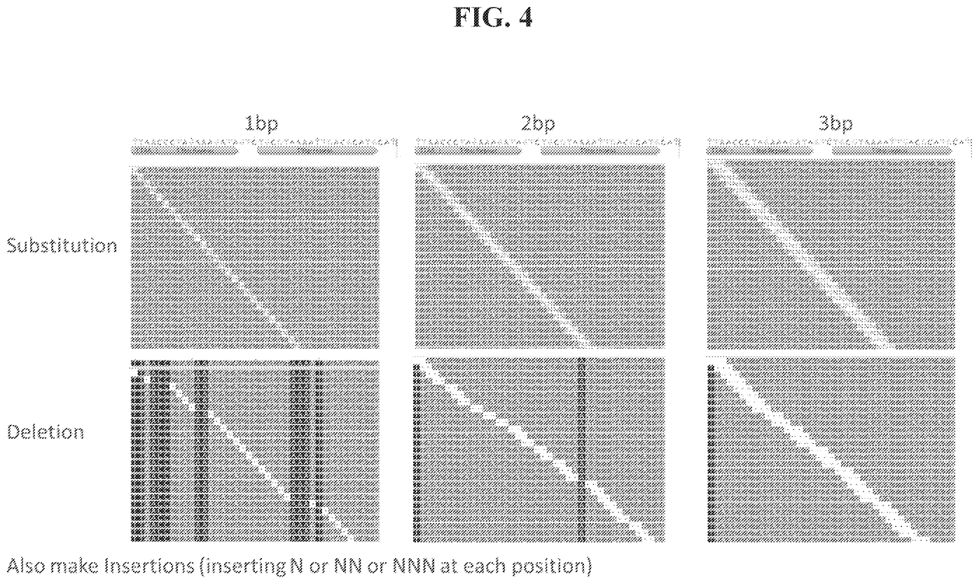

Substitution, insertion, deletion mutations of the 5′ left end ITR (i.e. “5′ end library” or “5′ left repeat library”), the 5′ internal repeated region (i.e. “internal repeat library” or “5′ internal library”) and the 3′ right end ITR (i.e. 3′ end library” or 3′ left repeat library”) of a transposon was investigated in the next library designs (FIG. 3). Mutant libraries were prepared for each of these three regions by generating 1 bp, 2 bp or 3 bp substitution, deletion or insertion mutations at each position (FIG. 4). The composition of each library and the diversity and number of mutations are shown in Tables 3, 4 and 5 for the 5′ left end repeated region, the 5′ and the 3′ right end repeated region mutational library, respectively.

TABLE 3
Composition and Diversity of
the 5′ End Mutational Library

| | # | Diversity |
|---|---|---|
| 5′ End | Oligos | per Oligo |
| 1 bp Substitution | 45 | 4 |
| 2 bp Substitution | 44 | 16 |
| 3 bp Substitution | 43 | 64 |
| 1 bp Insertion | 45 | 4 |
| 2 bp Insertion | 45 | 16 |
| 3 bp Insertion | 45 | 64 |
| 1 bp Deletion | 34 | 1 |
| 2 bp Deletion | 33 | 1 |
| 3 bp Deletion | 36 | 1 |

TABLE 4
Composition and Diversity of
the 5′ Internal Mutational Library

| | # | Diversity |
|---|---|---|
| 5′ Internal | Oligos | per Oligo |
| 1 bp Substitution | 57 | 4 |
| 2 bp Substitution | 56 | 16 |
| 3 bp Substitution | 55 | 64 |
| 1 bp Insertion | 57 | 4 |
| 2 bp Insertion | 57 | 16 |
| 3 bp Insertion | 57 | 64 |
| 1 bp Deletion | 42 | 1 |
| 2 bp Deletion | 40 | 1 |
| 3 bp Deletion | 42 | 1 |

TABLE 5
Composition and Diversity of
the 3′ End Mutational Library

| | # | Diversity |
|---|---|---|
| 3′ End | Oligos | per Oligo |
| 1 bp Substitution | 73 | 4 |
| 2 bp Substitution | 72 | 16 |
| 3 bp Substitution | 71 | 64 |
| 1 bp Insertion | 73 | 4 |
| 2 bp Insertion | 73 | 16 |
| 3 bp Insertion | 73 | 64 |
| 1 bp Deletion | 54 | 1 |
| 2 bp Deletion | 51 | 1 |
| 3 bp Deletion | 53 | 1 |

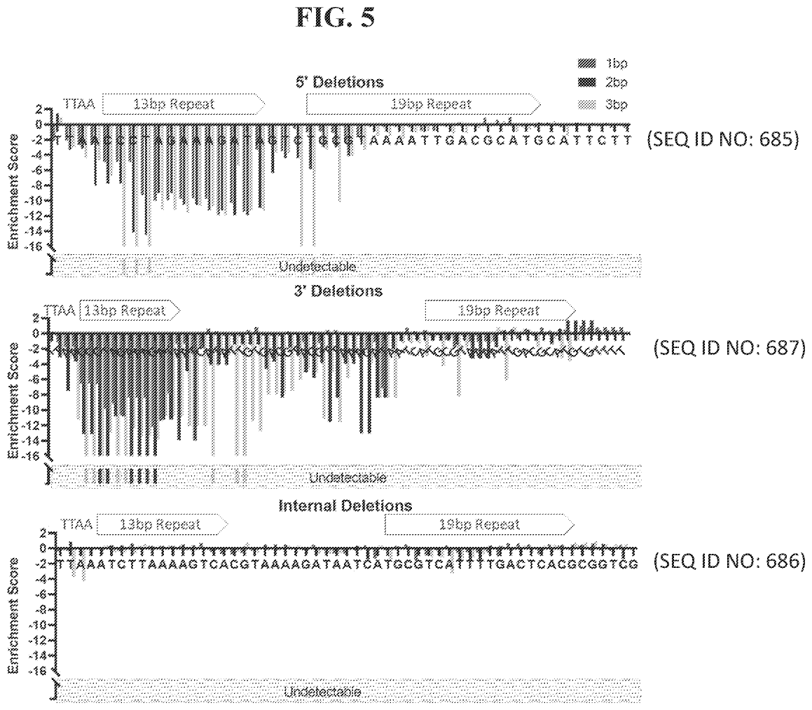

The 1 bp, 2 bp and 3 bp deletion mutants from the 5′ end, 5′ internal and 3′ end libraries were tested for transposition activity (FIG. 5). Deletions within the 13 bp repeat region and linker between repeats are poorly tolerated. The 19 bp repeat region tolerates many types of deletions. However few deletions appear to be beneficial for transposition. Deletions within the internal “ITR-like” region are neutral.

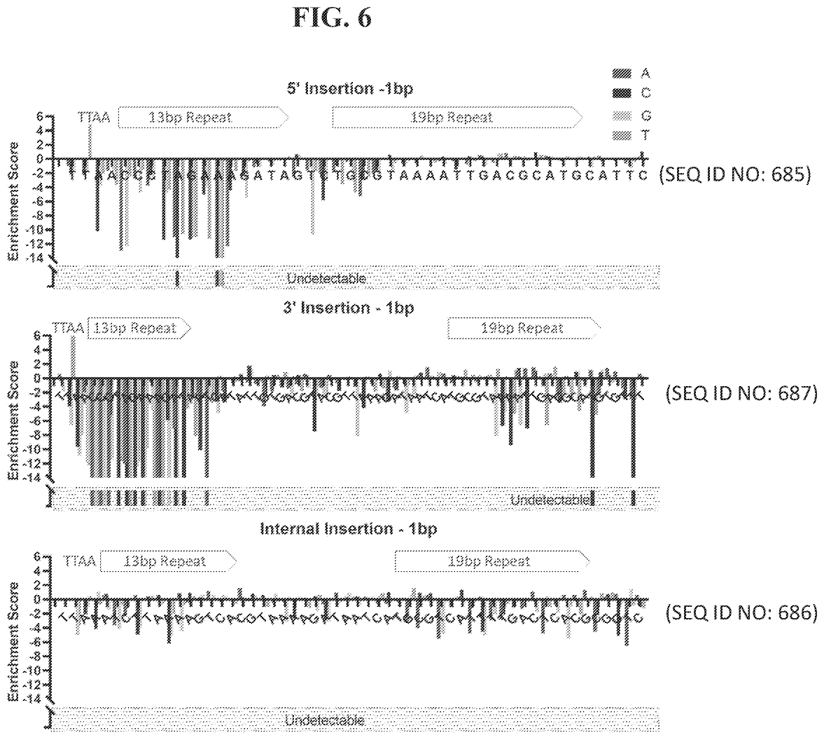

The 1 bp insertion mutants from the 5′ end, 5′ internal and 3′ end libraries were tested for transposition activity (FIG. 6). Overall, the insertions were detrimental and poorly tolerated within the 13 bp repeat region. Insertions in the 19 bp repeat region and the internal ITR-like region had more neutral effects. However, few deletions appear to be beneficial for transposition. Overall, insertions and deletions within the LE ITR or RE ITR regions of a transposon are mostly detrimental and unlikely improve transposition activity.

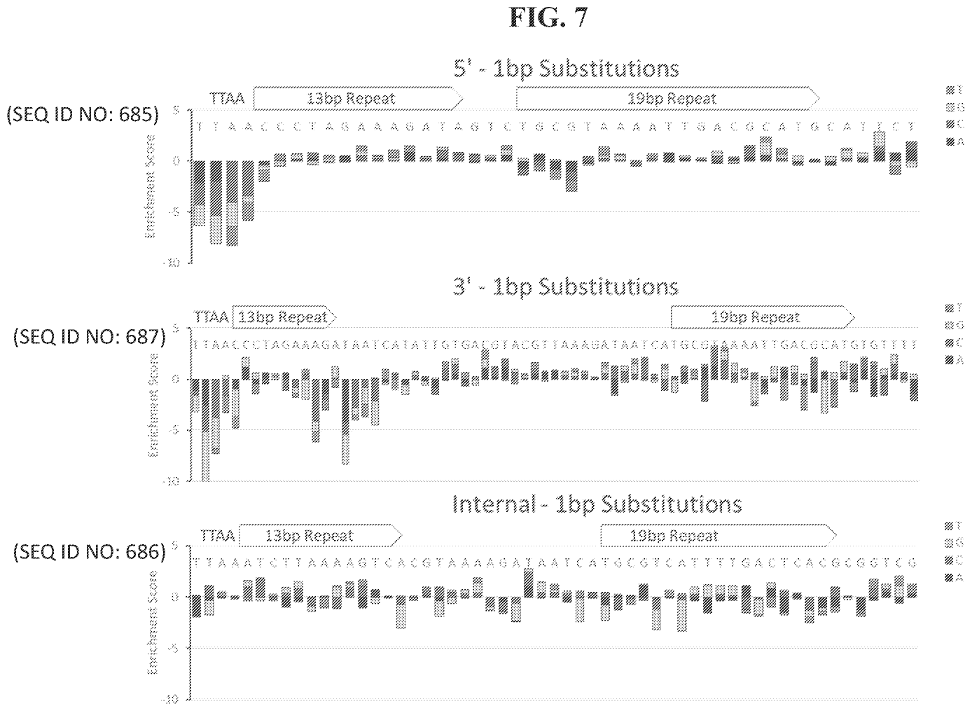

The 1 bp substitution mutants from the 5′ end, 5′ internal and 3′ end libraries were tested for transposition activity (FIG. 7). Substitutions were tolerated within the 13 bp repeat region of the 5′ end and a portion of the 3′ end. Certain substitutions were enriched within the 13 bp repeat region of the 5′ end. Overall, 1 bp substitutions were well tolerated. However, a difference of enrichment was observed in the 5′ and 3′ ends. Overall, mutations have more extreme effects in the 5′ and 3′ ITR regions than the internal “ITR-like” region. Mutations within the 13 bp repeat region have a stronger effect than mutations within the 19 bp repeat region.

### Mutant ITR Libraries

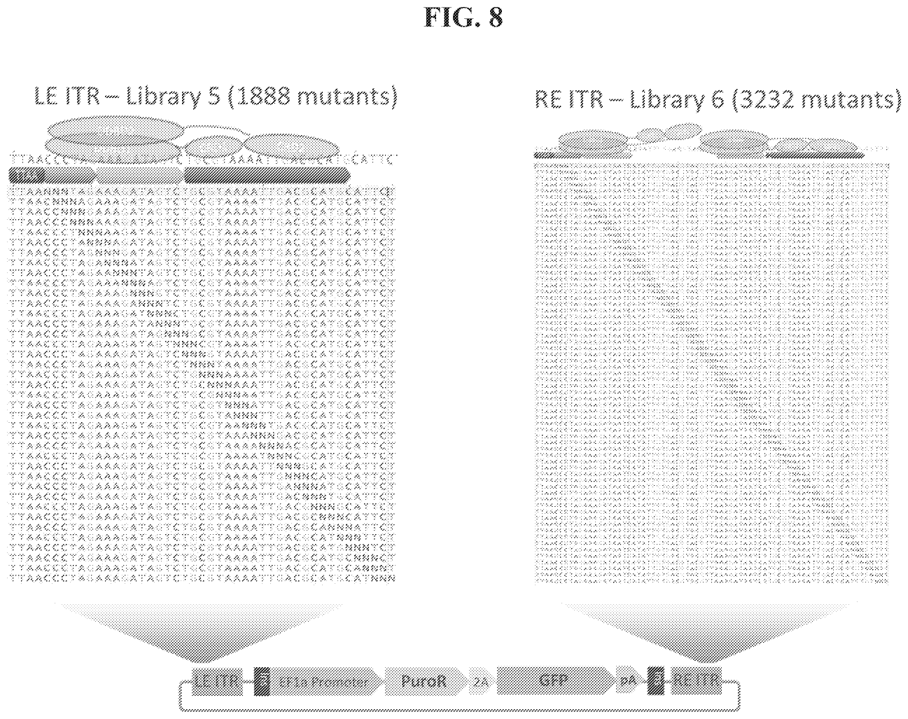

To further investigate substitution mutations and combinational substitution mutations which improve transposition activity, the 3 bp substitution mutants from the 5′ end and 3′ end libraries were tested. Two libraries were created corresponding to a left end ITR mutational library (LE ITR) (Library 5-1888 mutants) and a right end ITR (RE ITR) mutational library (Library 6-3232 mutants) (FIG. 8). To create the mutational libraries, 64 different combinations of trimer substitutions were generated and substituted at each position of the LE ITR and the RE ITR. The libraries excluded mutations in the TTAA position of the ITRs.

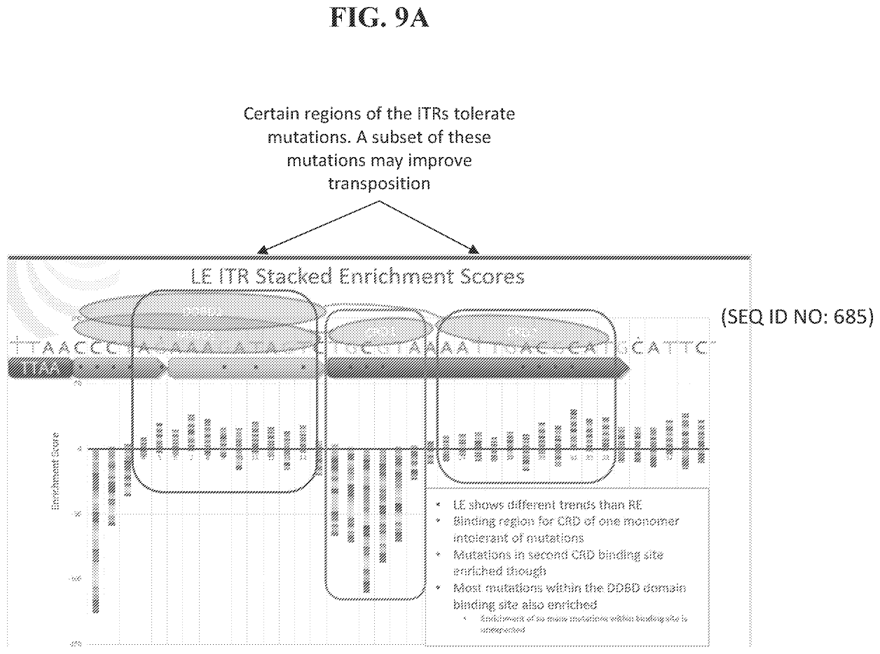

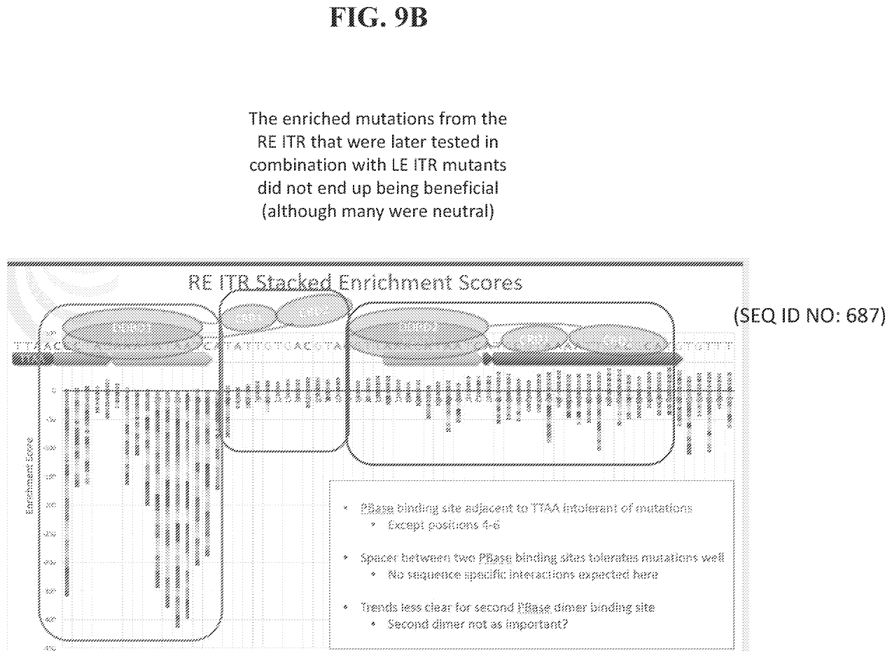

The stacked enrichment scores were analyzed for each position of the ITRs and many positions were enriched in the screen at both the LE ITR and RE ITR (FIGS. 9A and 9B). But, different trends were observed between LE ITRs and RE ITRs.

In the LE ITR, the predicted binding positions of the first CRD monomer (e.g. CRD1) of PB transposase were intolerant to mutations. However, predicted binding positions for the second CRD monomer (e.g. CRD2) were tolerant to mutations. Unexpectedly, most mutations within the predicted binding sites of DDBD domains (e.g. DDBD1 and DDBD2) were enriched. This suggests that mutations within certain positions of the LE ITR contribute to higher transposition activity, while most other positions are detrimental to transposition activity.

In the RE LTR, predicted binding positions of PB transposase adjacent to the TTAA position of the ITR were not tolerant to mutations except for at positions 4 to 6. There are no sequence specific interactions expected within the spacer regions of the ITR between the two PB transposase binding sites, and this spacer region tolerated mutations. The trends were less clear for the second PB transposase dimer binding site.

### Secondary Mutant ITR Libraries

The top three hits with the highest enrichment score for each three bp window of the LE ITRs and RE ITRs libraries described above were used to construct a more focused secondary library, shown in Table 6 and Table 7, respectively. The secondary mutant ITR libraries were screened as described above. The enrichment score of each mutant listed from highest to lowest is in Tables 6-7. A subset of the mutant LE ITR sequences resulted in an increased pooled enrichment score relative to (SEQ ID NOS: 16, 160-163, 165-204, 214, 230 and 237) the WT LE ITR sequence (SEQ ID NO: 4). A subset of the mutant RE ITR sequences resulted in an increased pooled enrichment score relative to (SEQ ID NOS: 267-277 and 279-329) the WT RE ITR sequence (SEQ ID NO: 5).

TABLE 6
LE ITR Pooled and Individual Mutant Library Enrichment Scores and
Integration Efficiency

| | | | Individual |
|---|---|---|---|
| | | Pooled | Test |
| SEQ ID | | Enrichment | Integration |
| NO: | Mutant LE ITR Sequence | Score | (%GFP) |
| 160 | CCCTAGAAAGATAGTCTGCGTAAAATTGACTTCTG | 0.494494304 | 50.2 |
| 161 | CCCTAGAAAAACAGTCTGCGTAAAATTGACGCATG | 0.481917949 | 55.0 |
| 162 | CCCTAGAAAGATAGCATGCGTAAAATTGACGCATG | 0.44526056 | 50.0 |
| 163 | CCCTAGTACGATAGTCTGCGTAAAATTGACGCATG | 0.437641928 | |
| 16 | CCCTAGAAAGATAGTCTGCGTAAAATTGACTCCTG | 0.426780979 | 55.0 |
| 165 | CCCTAGAAAGATAGTCTGCGTAAAATTGACCTCTG | 0.422667267 | 53.5 |
| 166 | CCCTAGTCCGATAGTCTGCGTAAAATTGACGCATG | 0.40295593 | |
| 167 | CCCTAGAAAGATAGTCTGCGTAAAATTGATCTATG | 0.349092814 | |
| 168 | CCCTAGAAAGATAGTCTGCGTAAAATTGACTTATG | 0.335467672 | |
| 169 | CCCTAGAAAGATAGTCTGCGTAAAATTGGCTCATG | 0.332261837 | 51.3 |
| 170 | CCCTAGAAAGATAGTCTGCGTAAAATTGACGCATC | 0.316002875 | |
| 171 | CCCTAGCATGATAGTCTGCGTAAAATTGACGCATG | 0.281243357 | |
| 172 | CCCTAGAAAGATAGTCTGCGTAAAATTGACGCGCC | 0.27403919 | |
| 173 | CCCTAGAAAGACCATCTGCGTAAAATTGACGCATG | 0.263425198 | |
| 174 | CCCTAGAAAGATCAACTGCGTAAAATTGACGCATG | 0.259316679 | 54.4 |
| 175 | CCCTAGAAAGATCTTCTGCGTAAAATTGACGCATG | 0.251227637 | 52.2 |
| 176 | CCCTAGTCAGATAGTCTGCGTAAAATTGACGCATG | 0.243414035 | |
| 177 | CCCTAGAAAGATAGTCTGCGTAAAATTGGTGCATG | 0.232302162 | 51.4 |
| 178 | CCCTAGAAAGATAGTCTGCGTAAAATTGTCACATG | 0.216069187 | 49.9 |
| 179 | CCCTAGAAAGATAGTCTGCGTAAAATTGACGCCCT | 0.213339606 | 49.7 |
| 180 | CCCTAGAAAGGGGGTCTGCGTAAAATTGACGCATG | 0.211727688 | 53.9 |
| 181 | CCCTCAGAAGATAGTCTGCGTAAAATTGACGCATG | 0.207330642 | |
| 182 | CCCTAGAAAGATTTACTGCGTAAAATTGACGCATG | 0.201703311 | |
| 183 | CCCTAGAAAGATACAATGCGTAAAATTGACGCATG | 0.201424097 | 51.7 |
| 184 | CCCTAGAGCAATAGTCTGCGTAAAATTGACGCATG | 0.197047935 | |
| 185 | CCCTAGAAAGATAGTCTGCGTAAAATTGTACCATG | 0.191692289 | 53.7 |
| 186 | CCCTAGAAAGATAGTCTGCGTAAAATTGACGCATC | 0.179747284 | |
| 187 | CCCTAGAAAGATAGTATACGTAAAATTGACGCATG | 0.162937375 | 48.8 |
| 188 | CCCTAGAAAGATAGTGTACGTAAAATTGACGCATG | 0.155810402 | |
| 189 | CCCTAGAAACCCAGTCTGCGTAAAATTGACGCATG | 0.154770509 | 50.7 |
| 190 | CCCTAGAAATACAGTCTGCGTAAAATTGACGCATG | 0.151407622 | |
| 191 | CCCTAGAAAGATAGTCTGCGTAAAATTGACGTCGG | 0.149752024 | 51.4 |
| 192 | CCCTAGAAAGATAGTCTGCGTAAAATTGACGCACC | 0.145568859 | |
| 193 | CCCTAGAAAGATAGTCTGCGTAAATCTGACGCATG | 0.126683453 | 52.1 |
| 194 | CCCTAGCTAGATAGTCTGCGTAAAATTGACGCATG | 0.126148846 | |
| 195 | CCCTAGAACGTTAGTCTGCGTAAAATTGACGCATG | 0.099745914 | |
| 196 | CCCTAGAAAGATAGTCTGCGTAATCTTGACGCATG | 0.094001908 | 52.9 |
| 197 | CCCTAGAAAGATAGTCTGCGTAAAATTGACGGGCG | 0.088618841 | |
| 198 | CCCTAGAAAGATAGTCTGCGTAAAATTGACGGGGG | 0.08492759 | |
| 199 | CCCTTAAAAGATAGTCTGCGTAAAATTGACGCATG | 0.080360203 | |
| 200 | CCCTAGAAAGATACAGTGCGTAAAATTGACGCATG | 0.036747438 | 50.3 |
| 201 | CCCTCGCAAGATAGTCTGCGTAAAATTGACGCATG | 0.034141573 | |
| 202 | CCCTAGAAAGATAGTCTGCGTAAAATCTGCGCATG | 0.034068457 | |
| 203 | CCCTAGAAAGATAGTCTGCGTAATTGTGACGCATG | 0.027665506 | 53.1 |
| 204 | CCCTAGAAAGAAGATCTGCGTAAAATTGACGCATG | 0.015777316 | 48.8 |
| 4 | CCCTAGAAAGATAGTCTGCGTAAAATTGACGCATG | 0.01088247 | 50.8 |
| 206 | CCCTAGAAAGATAGTCTGCGTAAAGCGGACGCATG | 0.010834679 | 47.2 |
| 207 | CCCTAGAAAGATAGTCTGCGTAAAATTGACGCAGA | 0.009189589 | |
| 208 | CCCTAGAAAGTAGGTCTGCGTAAAATTGACGCATG | 0.008835633 | 50.1 |
| 209 | CCCCTGAAAGATAGTCTGCGTAAAATTGACGCATG | 0.006769454 | |
| 210 | CCCTAGAGGTATAGTCTGCGTAAAATTGACGCATG | −0.009124959 | |
| 211 | CCCTAGAAAGATAGTCTGCGTAAAACTCACGCATG | −0.010071054 | |
| 212 | CCCTAAGCAGATAGTCTGCGTAAAATTGACGCATG | −0.012245064 | |
| 213 | CCCTCCAAAGATAGTCTGCGTAAAATTGACGCATG | −0.012713063 | |
| 214 | CCCTAGAAAGATAGTCTGCGTAAATTCGACGCATG | −0.017425699 | 52.6 |
| 215 | CCCCAGAAAGATAGTCTGCGTAAAATTGACGCATG | −0.035645229 | |
| 216 | CCCTAGAAAGATAGTCTGCGTAAAAGCCACGCATG | −0.043428664 | |
| 217 | CCCTGCGAAGATAGTCTGCGTAAAATTGACGCATG | −0.047164991 | |
| 218 | CCCTAGAAAGATAGTCTGCGTAAAATTGACGCATT | −0.047321011 | |
| 219 | CCCTAGATCCATAGTCTGCGTAAAATTGACGCATG | −0.059039967 | |
| 220 | CCCTAGAAAGATAGTCTGCGTAACGCTGACGCATG | −0.05949643 | 50.0 |
| 221 | CCCTAGAAAGATAGTCTGCGTAAAATGGGCGCATG | −0.059906714 | |
| 222 | CCCTAGAAAGATAGTCTGCGTAGAATTGACGCATG | −0.077724163 | |
| 223 | CCCTAGAAAGTCGGTCTGCGTAAAATTGACGCATG | −0.08890708 | |
| 224 | CCCCTCAAAGATAGTCTGCGTAAAATTGACGCATG | −0.094828782 | |
| 225 | CCCTAGAACTCTAGTCTGCGTAAAATTGACGCATG | −0.09730042 | |
| 226 | CCCAAGAAAGATAGTCTGCGTAAAATTGACGCATG | −0.097366634 | |
| 227 | CCCTAGAAAGATAGTACGCGTAAAATTGACGCATG | −0.102754554 | 45.7 |
| 228 | CCCTAGAAAGATAGTCTGCGTGAAATTGACGCATG | −0.109198653 | 50.2 |
| 229 | CCCTAGAAAGATAGTCTGCGTACGGTTGACGCATG | −0.115394803 | 48.7 |
| 230 | CCCTAGAAAGATAGTCTGCGTATCCTTGACGCATG | −0.116141571 | 53.1 |
| 231 | CCCTAGAAAGATGGACTGCGTAAAATTGACGCATG | −0.119259608 | 50.4 |
| 232 | CCCTAGAAAGATAGTCTGCGTCAGATTGACGCATG | −0.134623149 | 49.1 |
| 233 | CCCTAGAAAGATAGTCTGCGTAAAATTGACGCAGT | −0.142624125 | |
| 234 | CCCTAGAAAGATAGTCTGCGTCTAATTGACGCATG | −0.160826877 | |
| 235 | CCCTAGAAAGATAGTCTGCGTAAAATCTCCGCATG | −0.165231407 | |
| 236 | CCCAGGAAAGATAGTCTGCGTAAAATTGACGCATG | −0.16667335 | |
| 237 | CCCTAGAAAGATAGTCTGCGTAAAATATACGCATG | −0.187380792 | 51.0 |
| 238 | CCCTAGAAAGATAGTCTGCGTCGTATTGACGCATG | −0.188276151 | |
| 239 | CCCTAGAAAGATAGTCTGCGTAAAATTGATTAATG | −0.207047026 | 48.4 |
| 240 | CCCTAGAAAGATAGTCTGCGTAAACTGGACGCATG | −0.213499793 | |
| 241 | CCCTAGAAAGATAGTCTGCGCTAAATTGACGCATG | −0.215901388 | 48.7 |
| 242 | CCCGAGAAAGATAGTCTGCGTAAAATTGACGCATG | −0.217109502 | |
| 243 | CCCTAGAAGGTTAGTCTGCGTAAAATTGACGCATG | −0.230096771 | |
| 244 | CCCTAGAAAGATAGTCTGCGTAAAATTGACGCAGA | −0.242577702 | |
| 245 | CCCTAGAAAGATAGTCTGCGGAAAATTGACGCATG | −0.259961385 | 45.8 |
| 246 | CCTTAGAAAGATAGTCTGCGTAAAATTGACGCATG | −0.275245168 | |
| 247 | CCGTGGAAAGATAGTCTGCGTAAAATTGACGCATG | −0.314294941 | |
| 248 | CCCTAGAAAGATAGTCTGCGTAAAATTCTAGCATG | −0.322440526 | |
| 249 | CCCTAGAAAGATAGTCCACGTAAAATTGACGCATG | −0.32357052 | 43.1 |
| 250 | CCCTAGAAAGATAGTCTGCGCCAAATTGACGCATG | −0.325987356 | 47.0 |
| 251 | CCCTAGAAAGATAGTCCGCGTAAAATTGACGCATG | −0.35063157 | 43.6 |
| 252 | CGGTAGAAAGATAGTCTGCGTAAAATTGACGCATG | −0.366687092 | |
| 253 | CCCTAGAAAGATAGACCGCGTAAAATTGACGCATG | −0.445942658 | 45.1 |
| 254 | CCCTAGAAAGATAGTCACAGTAAAATTGACGCATG | −0.465108418 | |
| 255 | CTGTAGAAAGATAGTCTGCGTAAAATTGACGCATG | −0.473171776 | |
| 256 | CCCTAGAAAGATAGTCTGCGTCCGATTGACGCATG | −0.482436736 | 46.3 |
| 257 | CCCTAGAAAGATAGTCTGCGTAAAATTTTAGCATG | −0.668482946 | |
| 258 | CCCTAGAAAGATAGTCCGTGTAAAATTGACGCATG | −0.734087241 | |
| 259 | CCCTAGAAAGATAGTCTGCGGTGAATTGACGCATG | −0.844507596 | |
| 260 | CCCTAGAAAGATAGTCAGTGTAAAATTGACGCATG | −0.994792436 | 35.4 |
| 261 | CCCTAGAAAGATAGTCTGGGCAAAATTGACGCATG | −1.032569579 | 34.7 |
| 262 | CCCTAGAAAGATAGTCTGGAGAAAATTGACGCATG | −1.751354607 | |
| 263 | CCCTAGAAAGATAGTCTAGTTAAAATTGACGCATG | −1.778438232 | 31.0 |
| 264 | CCCTAGAAAGATAGTCTTGATAAAATTGACGCATG | −1.822553789 | |
| 265 | CCCTAGAAAGATAGTCTAGATAAAATTGACGCATG | −1.867973975 | 30.7 |

TABLE 7
RE ITR Pooled Mutant Library Enrichment Scores

| SEQ | | Pooled Enrichment |
|---|---|---|
| ID NO: | Mutant ITR Sequence | Score |
| 267 | CCCTAGAAAGATAATCATATTGTGACGTATGCTAAAGATAATCA | 0.771450697 |
| | TGCGTAAAATTGACGCATG | |
| 268 | CCCTAGAAAGATAATCATATTGTGACGTACGTTAAAGATAATCA | 0.655952089 |
| | TGCGTTTTATTGACGCATG | |
| 269 | CCCTAGAAAGATAATCATATTGTGACGTACGTTAAAGATAATCA | 0.652902935 |
| | TGCGTAATTTTGACGCATG | |
| 270 | CCCTAGAAAGATAATCATATTGTGACGTACGTTAAAGATAATCA | 0.63805728 |
| | TGCGTAAGCCTGACGCATG | |
| 271 | CCCTAGAAAGATAATCATATTGTGACGTACGTTAAAGATAATCA | 0.605480089 |
| | TGCGTAAAATTGACTGGTG | |
| 272 | CCCTAGAAAGATAATCATATTGTGACGTACGTTAAAGATAATCA | 0.59773679 |
| | TGCGTAAAATATACGCATG | |
| 273 | CCCTAGAAAGATAATCATATTGTGACGTACGTTAAAGATAATCA | 0.564613475 |
| | TGCGCCGAATTGACGCATG | |
| 274 | CCCTAGAAAGATAATCATATTGTGACGTACGTTAAAGATAATCA | 0.561410846 |
| | TGCGTAAAATTGCTACATG | |
| 275 | CCCTAGAAAGATAATCATATTGTGACGTACGTTAAAGATAATCA | 0.553862346 |
| | TGCGTAAAATGAACGCATG | |
| 276 | CCCTAGAAAGATAATCATATTGTGACGTACGTTAAAGATAATCA | 0.5533987 |
| | TGCGTAAAATTGGTCCATG | |
| 277 | CCCTAGAAAGATAATCATATTGTGATCGACGTTAAAGATAATCA | 0.51672722 |
| | TGCGTAAAATTGACGCATG | |
| 279 | CCCTAGAAAGATAATCATATTGTGACGTACGTTAAAGATAAACT | 0.511305895 |
| | TGCGTAAAATTGACGCATG | |
| 280 | CCCTAGAAAGATAATCATATTGTGACGTACGTTAAAGATAATCA | 0.506952684 |
| | GTAGTAAAATTGACGCATG | |
| 281 | CCCTAGAAAGATAATCATATTGTGACGTACGTTAAAGATAATCA | 0.49864359 |
| | TGCGTAATGATGACGCATG | |
| 282 | CCCTAGAAAGATAATCATATTGTGACGTACGTTAAAGATAATCA | 0.492197692 |
| | TGCGTAAAATTGACTACTG | |
| 283 | CCCTAGAAAGATAATCATATTGTGACGTACGTTAAAGATAATCA | 0.46993074 |
| | TGCGTAAAGGCGACGCATG | |
| 284 | CCCTAGAAAGATAATCATATTGTGTGCTACGTTAAAGATAATCA | 0.459024754 |
| | TGCGTAAAATTGACGCATG | |
| 285 | CCCTAGAAAGATAATCATATTGTGACGTACGTTAAAGATAATCA | 0.450261394 |
| | TGCGAGTAATTGACGCATG | |
| 286 | CCCTCCAAAGATAATCATATTGTGACGTACGTTAAAGATAATCA | 0.431316901 |
| | TGCGTAAAATTGACGCATG | |
| 287 | CCCTAGAAAGATAATCATATTGTGACGTACGTTAAAGATAATCA | 0.426027797 |
| | GTCGTAAAATTGACGCATG | |
| 288 | CCCTAGAAAGATAATCATATTGTGACGTACGTTAAAGATAATCA | 0.389293167 |
| | TGCGTAAAATTGACGCGGT | |
| 289 | CCCTAGAAAGATAATCATATTGTGACGTACGCAGAAGATAATCA | 0.376264622 |
| | TGCGTAAAATTGACGCATG | |
| 290 | CCCTAGAAAGATAATCATATTGTGGGCTACGTTAAAGATAATCA | 0.367534939 |
| | TGCGTAAAATTGACGCATG | |
| 291 | CCCTAGAAAGATAATCATATTTGCACGTACGTTAAAGATAATCA | 0.355449001 |
| | TGCGTAAAATTGACGCATG | |
| 292 | CCCTAGAAAGATAATCATATTGTGACGTACGTTAAAGATAATCA | 0.352064434 |
| | TGCCCTAAATTGACGCATG | |
| 293 | CCCTAGAAAGATAATCATATTGTGACGTACGCTCAAGATAATCA | 0.337825254 |
| | TGCGTAAAATTGACGCATG | |
| 294 | CCCTAGAAAGATAATCATATTGTGACGTACGTTAAAGATAGCCA | 0.317594261 |
| | TGCGTAAAATTGACGCATG | |
| 295 | CCCTAGAAAGATAATCATATTGTGACGTACGTTAAAGATAATCA | 0.314688283 |
| | TGCGTAAAATCTCCGCATG | |
| 296 | CCCTAGAAAGATAATCATATTGTGACGTACGTTAAAGCTGATCA | 0.307643993 |
| | TGCGTAAAATTGACGCATG | |
| 297 | CCCTAGAAAGATAATCATATTGTGACGTACGTTAAAGATACCGA | 0.304721869 |
| | TGCGTAAAATTGACGCATG | |
| 298 | CCCTAGAAAGATAATCATATTGTGACGTACGTTAAAGATAATCA | 0.302346332 |
| | TGCGTAAAATTGGTGCATG | |
| 299 | CCCTAGAAAGATAATCATATGGCGACGTACGTTAAAGATAATCA | 0.300625083 |
| | TGCGTAAAATTGACGCATG | |
| 300 | CCCTAGAAAGATAATCATATTGTGACGTACGTTAAAGATGGACA | 0.296688004 |
| | TGCGTAAAATTGACGCATG | |
| 301 | CCCTAGAAAGATAATCATATTGTGACGCTTGTTAAAGATAATCA | 0.296190267 |
| | TGCGTAAAATTGACGCATG | |
| 302 | CCCTAGAAAGATAATCATATTGTGACGTACGTTGCCGATAATCA | 0.291831049 |
| | TGCGTAAAATTGACGCATG | |
| 303 | CCCTAGAAAGATAATCATATTGTGACGTACGTTAAAGATAATCA | 0.287610119 |
| | TGCGTGCTATTGACGCATG | |
| 304 | CCCTAGAAAGATAATCATATTGTGACGTACGTTAAAGCCAATCA | 0.287233992 |
| | TGCGTAAAATTGACGCATG | |
| 305 | CCCTAGAAAGATAATCATATTGTGACGTTAATTAAAGATAATCA | 0.283261981 |
| | TGCGTAAAATTGACGCATG | |
| 306 | CCCTAGAAAGATAATCATATTGTGACGTACGTTAAAGATAATCA | 0.282978252 |
| | TGCCCCAAATTGACGCATG | |
| 307 | CCCTAGAAAGATAATCATATTGTGACGTACGTTAAAGATAATCT | 0.282121311 |
| | AACGTAAAATTGACGCATG | |
| 308 | CCCTAGAAAGATAATCATATTTTAACGTACGTTAAAGATAATCA | 0.276566686 |
| | TGCGTAAAATTGACGCATG | |
| 309 | CCCTAGAAAGATAATCATATTGTGACGTACGTTAAAGATAATCA | 0.273585432 |
| | TAGCTAAAATTGACGCATG | |
| 310 | CCCTAGAAAGATAATCATATTGTGACGTACGTTAAAGATAATCA | 0.260959353 |
| | TGCGTAAAATTGCGGCATG | |
| 311 | CCCTAGAAAGATAATCATATTGTGACGTACGTTAAAGGTTATCA | 0.255202507 |
| | TGCGTAAAATTGACGCATG | |
| 312 | CCCTAGAAAGATAATCATATTGTGACCACCGTTAAAGATAATCA | 0.249969774 |
| | TGCGTAAAATTGACGCATG | |
| 313 | CCCTAGAAAGATAATCATATTGTGACGTACGTTAAAGATAATCA | 0.248245353 |
| | TGCGTAAAATGCCCGCATG | |
| 314 | CCCTAGAAAGATAATCATATTGTGACGTACGTTAATCATAATCA | 0.236439935 |
| | TGCGTAAAATTGACGCATG | |
| 315 | CCCTAGAAAGATAATCATATTGTGACGTACGTTAAAGATAATCA | 0.231546781 |
| | TGAGGAAAATTGACGCATG | |
| 316 | CCCTAGAAAGATAATCATATTGTGACGTACGTTAAAGATAATCA | 0.222951698 |
| | TGCGTATTGTTGACGCATG | |
| 317 | CCCTAGAAAGATAATCATATTGTGACGTACGTGTGAGATAATCA | 0.217931291 |
| | TGCGTAAAATTGACGCATG | |
| 318 | CCCTAGAAAGATAATCATATTGTGACGTACGTTAAAGATAATCA | 0.216795324 |
| | TGCGTAAAATTGACGCCGG | |
| 319 | CCCTAGAAAGATAATCATATTGTGAGGCACGTTAAAGATAATCA | 0.212870727 |
| | TGCGTAAAATTGACGCATG | |
| 320 | CCCTAGAAAGATAATCATATTGTGACGTACGTTAAAGATAATCA | 0.202641822 |
| | TGCGTAAAATTGACGAGCG | |
| 321 | CCCTAGAAAGATAATCATATTGTGAAGCACGTTAAAGATAATCA | 0.201683149 |
| | TGCGTAAAATTGACGCATG | |
| 322 | CCCTAGAAAGATAATCATATTGTGACGTACGTTAAAGATAATCA | 0.185336208 |
| | TGCGTAATTGTGACGCATG | |
| 323 | CCCTAGAAAGATAATCATATTGTGACGTCCTTTAAAGATAATCA | 0.183968505 |
| | TGCGTAAAATTGACGCATG | |
| 324 | CCCTAGAAAGATAATCATATTGTGACGTACGTTAGCCATAATCA | 0.181313886 |
| | TGCGTAAAATTGACGCATG | |
| 325 | CCCTAGAAAGATAATCATATTGTGACGTACTCCAAAGATAATCA | 0.179550084 |
| | TGCGTAAAATTGACGCATG | |
| 326 | CCCTAGAAAGATAATCATATTGTGACAACCGTTAAAGATAATCA | 0.175515506 |
| | TGCGTAAAATTGACGCATG | |
| 327 | CCCTAGAAAGATAATCATATTGTGACGTACGTTGGAGATAATCA | 0.173485007 |
| | TGCGTAAAATTGACGCATG | |
| 328 | CCCTAGAAAGATAATCATACTTTGACGTACGTTAAAGATAATCA | 0.171953804 |
| | TGCGTAAAATTGACGCATG | |
| 329 | CCCTAGAAAGATAATCATATTGTGACGTACGTTAAAGATAATCA | 0.167055238 |
| | TGCGTAAAATTGCTTCATG | |
| 5 | CCCTAGAAAGATAATCATATTGTGACGTACGTTAAAGATAAT | 0.155312147 |
| | CATGCGTAAAATTGACGCATG | |
| 331 | CCCTAGAAAGATAATCATATTGTGACGTACGTTAAAGATAATCA | 0.153756441 |
| | TGCGTAAAATTGATCAATG | |
| 332 | CCCCGGAAAGATAATCATATTGTGACGTACGTTAAAGATAATCA | 0.153153715 |
| | TGCGTAAAATTGACGCATG | |
| 333 | CCCTAGAAAGATAATCATATTGTGACGTACGTTAAAGTGAATCA | 0.144061363 |
| | TGCGTAAAATTGACGCATG | |
| 334 | CCCTAGAAAGATAATCATATTGGTCCGTACGTTAAAGATAATCA | 0.127264428 |
| | TGCGTAAAATTGACGCATG | |
| 335 | CCCTAGAAAGATAATCATATTGTGCATTACGTTAAAGATAATCA | 0.123853103 |
| | TGCGTAAAATTGACGCATG | |
| 336 | CCCTAGAAAGATAATCATATTGTGACGTACGTTAAAGATAATCA | 0.120618537 |
| | TGCGTAAAATGGGCGCATG | |
| 337 | CCCTAGAAAGATAATCATATTGTGACGTAAACTAAAGATAATCA | 0.115999925 |
| | TGCGTAAAATTGACGCATG | |
| 338 | CCCTAGAAAGATAATCATATTGTGACGTACGTTAAAGATAATCA | 0.113583902 |
| | TGCGTCCAATTGACGCATG | |
| 339 | CCCTAGAAAGATAATCATATTGTGACGTACGTTAAAGATAATCA | 0.112145734 |
| | TGCGTAAAATTAATGCATG | |
| 340 | CCCTAGAAAGATAATCATATTGTGACGTACGTTAAAGATAATTA | 0.10793313 |
| | CGCGTAAAATTGACGCATG | |
| 341 | CCCTAGAAAGATAATCATATTGTGACGTACGTTTGCGATAATCA | 0.106370615 |
| | TGCGTAAAATTGACGCATG | |
| 342 | CCCTAGAAAGATAATCATATTGTGACGTACATCAAAGATAATCA | 0.106209144 |
| | TGCGTAAAATTGACGCATG | |
| 343 | CCCTAGAAAGATAATCATATTGTGACGTACGTTAAAGATAATCA | 0.103730318 |
| | GAGGTAAAATTGACGCATG | |
| 344 | CCCTAGAAAGATAATCATATTGTGACGTACGTTAAAGATAATCA | 0.100318999 |
| | TGCGTAAAATTGAGAAATG | |
| 345 | CCCTCAGAAGATAATCATATTGTGACGTACGTTAAAGATAATCA | 0.100158661 |
| | TGCGTAAAATTGACGCATG | |
| 346 | CCCTAGAAAGATAATCATATTGTGACGTACGTATGAGATAATCA | 0.096457752 |
| | TGCGTAAAATTGACGCATG | |
| 347 | CCCTAGAAAGATAATCATATTGTGACGCGTGTTAAAGATAATCA | 0.092132482 |
| | TGCGTAAAATTGACGCATG | |
| 348 | CCCTAGAAAGATAATCATATTGTGGTGTACGTTAAAGATAATCA | 0.089684356 |
| | TGCGTAAAATTGACGCATG | |
| 349 | CCCTAGAAAGATAATCATAGCCTGACGTACGTTAAAGATAATCA | 0.089262164 |
| | TGCGTAAAATTGACGCATG | |
| 350 | CCCTAGAAAGATAATCATATTGTGACGTACGTTAAAGATAACTC | 0.088586478 |
| | TGCGTAAAATTGACGCATG | |
| 351 | CCCTAGAAAGATAATCATATTGTGACGTACGTTAAAGATAATCA | 0.087805082 |
| | TGCGTAGCCTTGACGCATG | |
| 352 | CCCTAGAAAGATAATCATATTGTGACGTACGTTAAAGATAATCA | 0.084294727 |
| | TGCGTAAAAACCACGCATG | |
| 353 | CCCTAGAAAGATAATCATATTGTGACGTACGTTAAAGATAATCA | 0.080286185 |
| | ACAGTAAAATTGACGCATG | |
| 354 | CCCTAGAAAGATAATCATATTGTGACGTACGTTAAAGATAATCT | 0.080035412 |
| | CGCGTAAAATTGACGCATG | |
| 355 | CCCTAGAAAGATAATCATATTGTGACGTACGTTAAAGATAACCC | 0.07831462 |
| | TGCGTAAAATTGACGCATG | |
| 356 | CCCTAGAAAGATAATCATATTGTGACGTACGTTAAAGATAATCA | 0.075681953 |
| | TGCGTAAAATTGACAGCTG | |
| 357 | CCCTAGAAAGATAATCATATTGTGACGCTCGTTAAAGATAATCA | 0.074317167 |
| | TGCGTAAAATTGACGCATG | |
| 358 | CCCTAGAAAGATAATCATATTGTGACGTACGTTTTCGATAATCAT | 0.067269516 |
| | GCGTAAAATTGACGCATG | |
| 359 | CCCTAGAAAGATAATCATATTGTGACGTACGTTAAAGAGATTCA | 0.064262734 |
| | TGCGTAAAATTGACGCATG | |
| 360 | CCCTAGAAAGATAATCATATTGTGACGTACGTTAAAGATAATCA | 0.056897235 |
| | TGCGTGACATTGACGCATG | |
| 361 | CCCTAGAAAGATAATCATATTGTGACGTACGTTAAAGATAATCA | 0.038150093 |
| | TGCGTAAAATTGACGCCCA | |
| 362 | CCCTAGAAAGATAATCATATTGTGACGTACGTTAAGCGTAATCA | 0.018137924 |
| | TGCGTAAAATTGACGCATG | |
| 363 | CCCTAGAAAGATAATCATATTGTACCGTACGTTAAAGATAATCA | 0.001731914 |
| | TGCGTAAAATTGACGCATG | |
| 364 | CCCTAGAAAGATAATCATATTGTGACGTACGTTAAAGATAATCA | −0.008219511 |
| | TTGCTAAAATTGACGCATG | |
| 365 | CCCTAGAAAGATAATCATATTGTGACGTACGTTAAAGATAATCA | −0.008807087 |
| | TGCGTAAAATTGACGCCCG | |
| 366 | CCCTAGAAAGATAATCATATTGTGACGTACGTTAAAGATAATCA | −0.011374651 |
| | TGCTTGAAATTGACGCATG | |
| 367 | CCCTAGAAAGATAATCATATTCACACGTACGTTAAAGATAATCA | −0.013006461 |
| | TGCGTAAAATTGACGCATG | |
| 368 | CCCTAGAAAGATAATCATATTGTGACGTAGGATAAAGATAATCA | −0.014308316 |
| | TGCGTAAAATTGACGCATG | |
| 369 | CCCTAGAAAGATAATCTCATTGTGACGTACGTTAAAGATAATCA | −0.016303299 |
| | TGCGTAAAATTGACGCATG | |
| 370 | CCCTGGAAAGATAATCATATTGTGACGTACGTTAAAGATAATCA | −0.01929024 |
| | TGCGTAAAATTGACGCATG | |
| 371 | CCCTAGAAAGATAATCATATTGTGACGTACGTTAAAGAGGGTCA | −0.021156879 |
| | TGCGTAAAATTGACGCATG | |
| 372 | CCCTAGAAAGATAATCATATTGTGACGTACGTTAAACGGAATCA | −0.021374464 |
| | TGCGTAAAATTGACGCATG | |
| 373 | CCCTTACAAGATAATCATATTGTGACGTACGTTAAAGATAATCA | −0.021690733 |
| | TGCGTAAAATTGACGCATG | |
| 374 | CCCTAGAAAGATAATCATATTGTGACGTACTAGAAAGATAATCA | −0.022434016 |
| | TGCGTAAAATTGACGCATG | |
| 375 | CCCTAGAAAGATAATCTAGTTGTGACGTACGTTAAAGATAATCA | −0.023579275 |
| | TGCGTAAAATTGACGCATG | |
| 376 | CCCTAGAAAGATAATCATATACGGACGTACGTTAAAGATAATCA | −0.024625488 |
| | TGCGTAAAATTGACGCATG | |
| 377 | CCCTAGAAAGATAATCATATTGTGACGTACGTTAAAGATAATCA | −0.031602167 |
| | TGCGTAAATACGACGCATG | |
| 378 | CCCTAGAAAGATAATCATATTGTGACGTACGTTAAAGATAATCT | −0.043521282 |
| | GGCGTAAAATTGACGCATG | |
| 379 | CCCTGCAAAGATAATCATATTGTGACGTACGTTAAAGATAATCA | −0.045048866 |
| | TGCGTAAAATTGACGCATG | |
| 380 | CCCTAGAAAGATAATCATATTGTGACGTACGTTAAAGATAATCA | −0.049252135 |
| | TGCGTAAAATTGAAGAATG | |
| 381 | CCCTAGAAAGATAATCATATTGTGACGTACGTTAAAGATGCTCA | −0.049379563 |
| | TGCGTAAAATTGACGCATG | |
| 382 | CCCTAGAAAGATAATCATATTGTGACGTACGTTAAAGATAATCA | −0.069043976 |
| | TGATTAAAATTGACGCATG | |
| 383 | CCCTAGAAAGATAATCATATTGTGACGTACGTTAAAGGGGATCA | −0.073195604 |
| | TGCGTAAAATTGACGCATG | |
| 384 | CCCTAGAAAGATAATCATATTGTGACGTACGTTAAAGATAATCA | −0.088083572 |
| | TGGCAAAAATTGACGCATG | |
| 385 | CCCTAGAAAGATAATCATATTGCCTCGTACGTTAAAGATAATCA | −0.110739039 |
| | TGCGTAAAATTGACGCATG | |
| 386 | CCCCAGAAAGATAATCATATTGTGACGTACGTTAAAGATAATCA | −0.121743511 |
| | TGCGTAAAATTGACGCATG | |
| 387 | CCCTAGAAAGATAATCATATTGTGACGTACGTTAAAGATAATCA | −0.130702773 |
| | TGCGTAAAATTGACGCGTC | |
| 388 | CCCTAGAAAGATAATCTATTTGTGACGTACGTTAAAGATAATCA | −0.149455179 |
| | TGCGTAAAATTGACGCATG | |
| 389 | CCCTAGAAAGATAATCATATTGTGACGTACGCATAAGATAATCA | −0.150580621 |
| | TGCGTAAAATTGACGCATG | |
| 390 | CCCTCGAAAGATAATCATATTGTGACGTACGTTAAAGATAATCA | −0.159888931 |
| | TGCGTAAAATTGACGCATG | |
| 391 | CCCTAGTGAGATAATCATATTGTGACGTACGTTAAAGATAATCA | −0.161611146 |
| | TGCGTAAAATTGACGCATG | |
| 392 | CCCTAGAAAGATAATCATATTGTGACGTACGTTAAAGATAATCC | −0.172057338 |
| | TTCGTAAAATTGACGCATG | |
| 393 | CCCTAGAAAGATAATCATAGTTTGACGTACGTTAAAGATAATCA | −0.177018851 |
| | TGCGTAAAATTGACGCATG | |
| 394 | CCCTAGAAAGATAATCCTATTGTGACGTACGTTAAAGATAATCA | −0.179211201 |
| | TGCGTAAAATTGACGCATG | |
| 395 | CCCTAGAAAGATAATCATATTGTGACGTACGTTAAAGATAATCA | −0.181883318 |
| | TGATGAAAATTGACGCATG | |
| 396 | CCCTAGAAAGATAATCATATTGTGACGCAGGTTAAAGATAATCA | −0.183314506 |
| | TGCGTAAAATTGACGCATG | |
| 397 | CCCTAGAAAGATAATCTTATTGTGACGTACGTTAAAGATAATCA | −0.183945265 |
| | TGCGTAAAATTGACGCATG | |
| 398 | CCCTAGAAAGATAATCTTTTTGTGACGTACGTTAAAGATAATCA | −0.185714848 |
| | TGCGTAAAATTGACGCATG | |
| 399 | CCCTAGAAAGATAATCATATTGTGACGTTTCTTAAAGATAATCA | −0.19264517 |
| | TGCGTAAAATTGACGCATG | |
| 400 | CCCTAGAAAGATAATCATATTGTCATGTACGTTAAAGATAATCA | −0.211560649 |
| | TGCGTAAAATTGACGCATG | |
| 401 | CCCAAGAAAGATAATCATATTGTGACGTACGTTAAAGATAATCA | −0.215774463 |
| | TGCGTAAAATTGACGCATG | |
| 402 | CCCTAGAAAGATAATCATATTGTGACGTACGTTAAAGATACTTA | −0.217583083 |
| | TGCGTAAAATTGACGCATG | |
| 403 | CCCTAGAAAGATAATCATTAGGTGACGTACGTTAAAGATAATCA | −0.227561614 |
| | TGCGTAAAATTGACGCATG | |
| 404 | CCCTAGAAAGATAATCAATGTGTGACGTACGTTAAAGATAATCA | −0.235073196 |
| | TGCGTAAAATTGACGCATG | |
| 405 | CCCTAGAAAGATAATCATATTGTGACGTACGTTAAAGATAATCA | −0.25166169 |
| | TGCGTAAATTAGACGCATG | |
| 406 | CCGTAGAAAGATAATCATATTGTGACGTACGTTAAAGATAATCA | −0.254837217 |
| | TGCGTAAAATTGACGCATG | |
| 407 | CCCTTTAAAGATAATCATATTGTGACGTACGTTAAAGATAATCA | −0.268304835 |
| | TGCGTAAAATTGACGCATG | |
| 408 | CCCTAGATGGATAATCATATTGTGACGTACGTTAAAGATAATCA | −0.276650368 |
| | TGCGTAAAATTGACGCATG | |
| 409 | CCCTAGAAAGATAATCACTCTGTGACGTACGTTAAAGATAATCA | −0.296864483 |
| | TGCGTAAAATTGACGCATG | |
| 410 | CCCTAGAAAGATAATCATATTGTGACGTACGTTAAAGATATAGA | −0.310189861 |
| | TGCGTAAAATTGACGCATG | |
| 411 | CCCTAGAAAGATAATCATATTGTGACGTACGTTAAGTGTAATCA | −0.313408827 |
| | TGCGTAAAATTGACGCATG | |
| 412 | CCCTAGAAAGATAATCATGCTGTGACGTACGTTAAAGATAATCA | −0.351400581 |
| | TGCGTAAAATTGACGCATG | |
| 413 | CCCTAGAAAGATAATCATATTGTTGAGTACGTTAAAGATAATCA | −0.355661604 |
| | TGCGTAAAATTGACGCATG | |
| 414 | CCCTACCAAGATAATCATATTGTGACGTACGTTAAAGATAATCA | −0.359588817 |
| | TGCGTAAAATTGACGCATG | |
| 415 | CCCTAGAAAGATAATCATAGGGTGACGTACGTTAAAGATAATCA | −0.365988993 |
| | TGCGTAAAATTGACGCATG | |
| 416 | CCCTAGAAAGATAATCATCAGGTGACGTACGTTAAAGATAATCA | −0.375646068 |
| | TGCGTAAAATTGACGCATG | |
| 417 | CCCTAGTCGGATAATCATATTGTGACGTACGTTAAAGATAATCA | −0.383811414 |
| | TGCGTAAAATTGACGCATG | |
| 418 | CCTTAGAAAGATAATCATATTGTGACGTACGTTAAAGATAATCA | −0.417920073 |
| | TGCGTAAAATTGACGCATG | |
| 419 | CCCTACATAGATAATCATATTGTGACGTACGTTAAAGATAATCA | −0.422742183 |
| | TGCGTAAAATTGACGCATG | |
| 420 | CCCTAGAAAGATAAACATATTGTGACGTACGTTAAAGATAATCA | −0.423935602 |
| | TGCGTAAAATTGACGCATG | |
| 421 | CCCTAGAAAGATAATCATATGTAGACGTACGTTAAAGATAATCA | −0.439287507 |
| | TGCGTAAAATTGACGCATG | |
| 422 | CCCTAGAAAGATAATCTAATTGTGACGTACGTTAAAGATAATCA | −0.460648345 |
| | TGCGTAAAATTGACGCATG | |
| 423 | CCCTAGCTAGATAATCATATTGTGACGTACGTTAAAGATAATCA | −0.47907597 |
| | TGCGTAAAATTGACGCATG | |
| 424 | CCCTAGAAAGATTATCATATTGTGACGTACGTTAAAGATAATCA | −0.491295297 |
| | TGCGTAAAATTGACGCATG | |
| 425 | CCCTAGAAAGATAATTATATTGTGACGTACGTTAAAGATAATCA | −0.496589293 |
| | TGCGTAAAATTGACGCATG | |
| 426 | CCCTAGAAAGTTAATCATATTGTGACGTACGTTAAAGATAATCA | −0.506706731 |
| | TGCGTAAAATTGACGCATG | |
| 427 | CCCTAGCGGGATAATCATATTGTGACGTACGTTAAAGATAATCA | −0.526460366 |
| | TGCGTAAAATTGACGCATG | |
| 428 | CCCTAGAAAGGTAATCATATTGTGACGTACGTTAAAGATAATCA | −0.545843847 |
| | TGCGTAAAATTGACGCATG | |
| 429 | CACCAGAAAGATAATCATATTGTGACGTACGTTAAAGATAATCA | −0.554684616 |
| | TGCGTAAAATTGACGCATG | |
| 430 | CCCTAGAAAGATATTCATATTGTGACGTACGTTAAAGATAATCA | −0.55512004 |
| | TGCGTAAAATTGACGCATG | |
| 431 | CCCTAGAAAGATAATGCTATTGTGACGTACGTTAAAGATAATCA | −0.622893065 |
| | TGCGTAAAATTGACGCATG | |
| 432 | CGCTAGAAAGATAATCATATTGTGACGTACGTTAAAGATAATCA | −0.651675005 |
| | TGCGTAAAATTGACGCATG | |
| 433 | CCCTAGAAAGATAATGATATTGTGACGTACGTTAAAGATAATCA | −0.69266863 |
| | TGCGTAAAATTGACGCATG | |
| 434 | CCCTAGAAAGCTAATCATATTGTGACGTACGTTAAAGATAATCA | −0.692700884 |
| | TGCGTAAAATTGACGCATG | |
| 435 | CCCTAGACTCATAATCATATTGTGACGTACGTTAAAGATAATCA | −0.725600686 |
| | TGCGTAAAATTGACGCATG | |
| 436 | CCCTAGAAAGATAATCATATTGTGACGTACGTTAACCTTAATCA | −0.744617749 |
| | TGCGTAAAATTGACGCATG | |
| 437 | CCCTAGAAAGATAATCATATTGTGACGTACGTTACCTATAATCA | −0.748174696 |
| | TGCGTAAAATTGACGCATG | |
| 438 | CCCTAGAAAGATAATCCAATTGTGACGTACGTTAAAGATAATCA | −0.807181379 |
| | TGCGTAAAATTGACGCATG | |
| 439 | CCCTAGAAAGAGAATCATATTGTGACGTACGTTAAAGATAATCA | −0.83693573 |
| | TGCGTAAAATTGACGCATG | |
| 440 | CCCTAGAAAGATAATCATATTGTGACGTACGTTAAAGATCGACA | −0.85076733 |
| | TGCGTAAAATTGACGCATG | |
| 441 | CCCTAGAAAGATGATCATATTGTGACGTACGTTAAAGATAATCA | −0.962504956 |
| | TGCGTAAAATTGACGCATG | |
| 442 | CCCTAGAAAGGGAATCATATTGTGACGTACGTTAAAGATAATCA | −0.964223108 |
| | TGCGTAAAATTGACGCATG | |
| 443 | CCCTAGAAAGTGAATCATATTGTGACGTACGTTAAAGATAATCA | −1.398792793 |
| | TGCGTAAAATTGACGCATG | |
| 444 | CCCTAGAGATATAATCATATTGTGACGTACGTTAAAGATAATCA | −1.501359031 |
| | TGCGTAAAATTGACGCATG | |
| 445 | CCCTAGAAGTATAATCATATTGTGACGTACGTTAAAGATAATCA | −1.586549107 |
| | TGCGTAAAATTGACGCATG | |
| 446 | CCCTAGAAAGATAAATATATTGTGACGTACGTTAAAGATAATCA | −1.703479875 |
| | TGCGTAAAATTGACGCATG | |
| 447 | CCCTAGAAAGAGTATCATATTGTGACGTACGTTAAAGATAATCA | −1.719235723 |
| | TGCGTAAAATTGACGCATG | |
| 448 | CCCTAGAAAGATAGTCATATTGTGACGTACGTTAAAGATAATCA | −1.775207464 |
| | TGCGTAAAATTGACGCATG | |
| 449 | CCCTAGAAAGATGAACATATTGTGACGTACGTTAAAGATAATCA | −2.116419062 |
| | TGCGTAAAATTGACGCATG | |
| 450 | CCCTAGAAAGAGATTCATATTGTGACGTACGTTAAAGATAATCA | −2.340327764 |
| | TGCGTAAAATTGACGCATG | |
| 451 | CCCTAGAAAGATAAGCATATTGTGACGTACGTTAAAGATAATCA | −2.530648073 |
| | TGCGTAAAATTGACGCATG | |

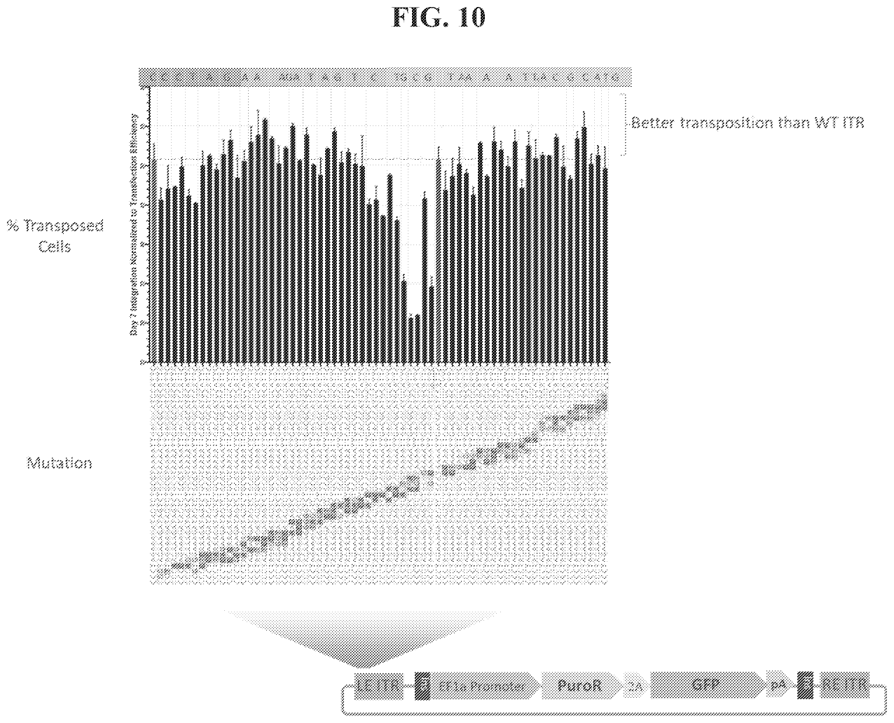

Next, a subset of mutant LE ITRs were co-transfected individually in HEK 293T cells with SPB transposase in order to confirm the best mutational hits from the screen. The percentage of GFP positive cells (a proxy for transposition activity) was measured by flow cytometry after 7 days. A subset of LE ITR mutants were tested individually. The results are shown in the Individual Test Integration (% GFP) column of Table 6. Within this subset, some LE ITR mutants were determined to have increased transposition activity (i.e. percentage of transposed cells with integration) relative to a transposon with a wildtype LE ITR (Table 6 and FIG. 10).

### Example 2: Improved Transposition with Mutant ITR Transposons and Super PiggyBac Transposases in K562 Cells, HEK 293T Cells and Primary T-Cells

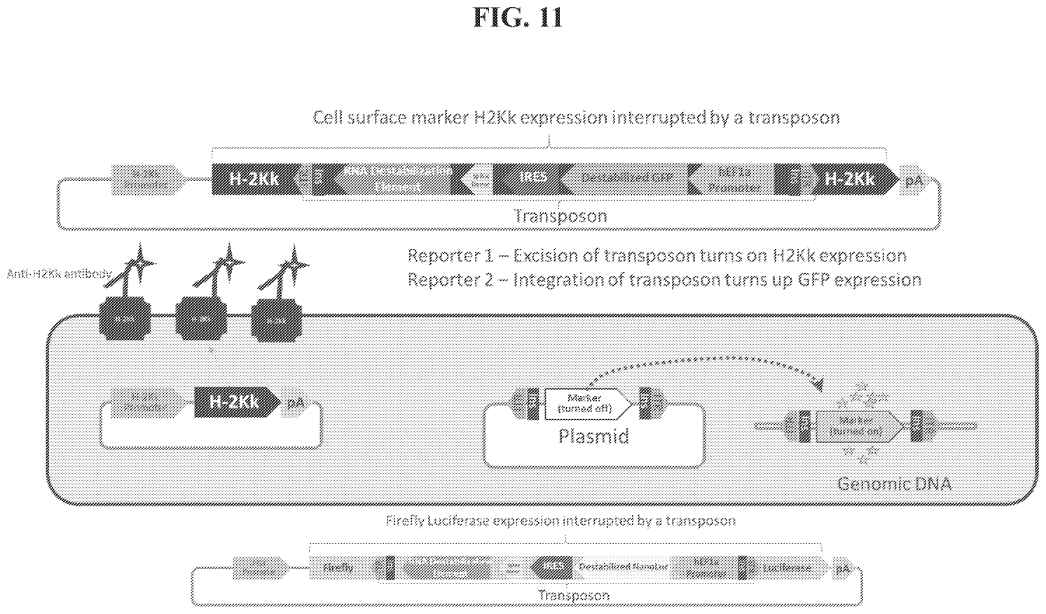

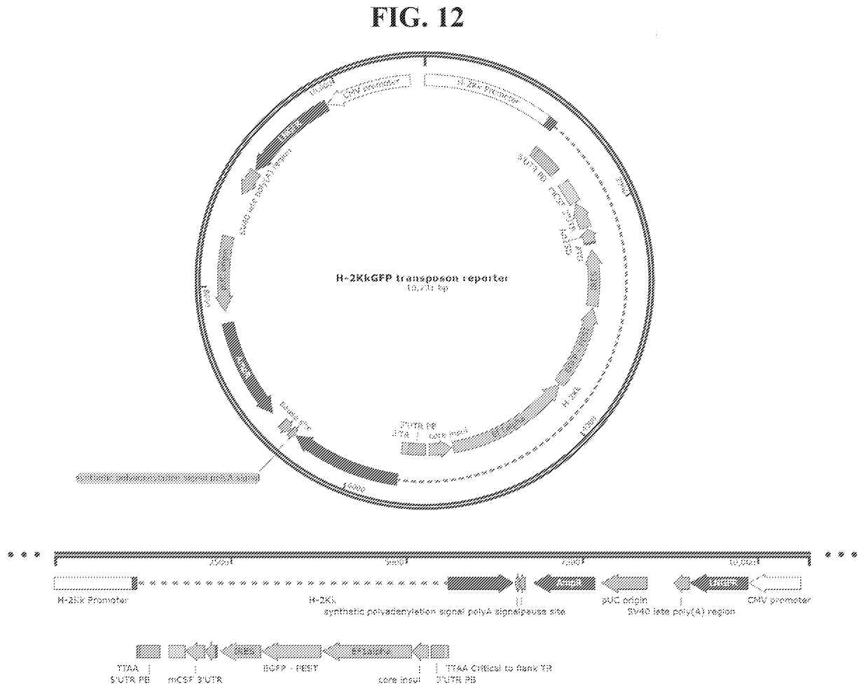

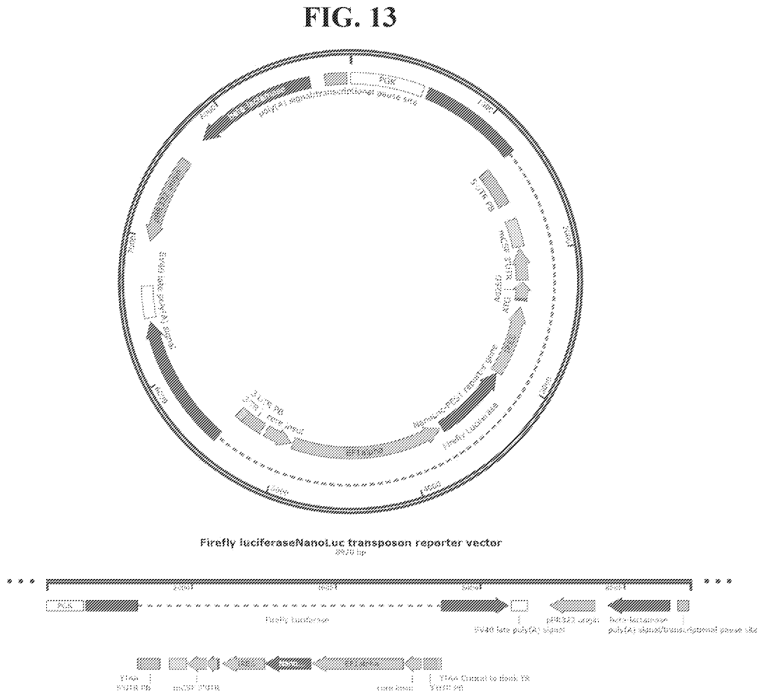

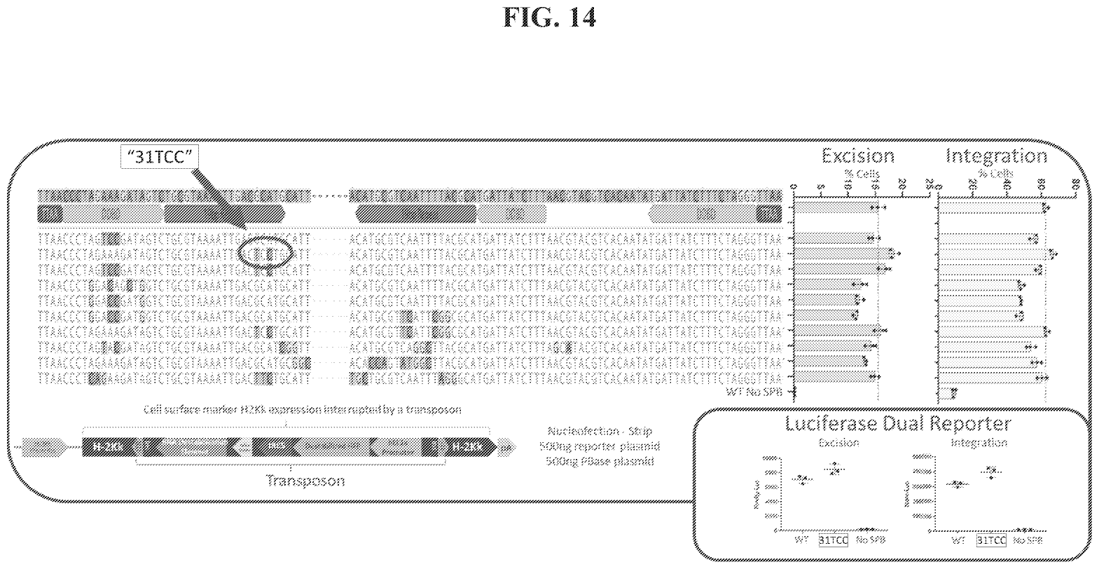

In order to determine the transposition activity of the top LE ITR mutants from the screens, the mutants were cloned into a dual excision and integration reporter system and nucleofected in K562 cells along with SPB transposase (FIG. 11). All mutants (SEQ ID NOs: 16, 166 and 548-556) were active, but only one showed an improvement over a transposon with wildtype LE ITR. Use of the 31TCC mutation of the LE ITR (SEQ ID NO: 16) (i.e. substitutions 31G>T and 33A>C) resulted in the highest percentage of cells with transposition activity (i.e. excision and/or integration) (FIG. 14). The 31TCC mutation also increased transposition activity using an alternative luciferase dual reporter, which confirms that this is a phenomenon associated with the 31TCC mutation in the LE ITR of the PB transposon.

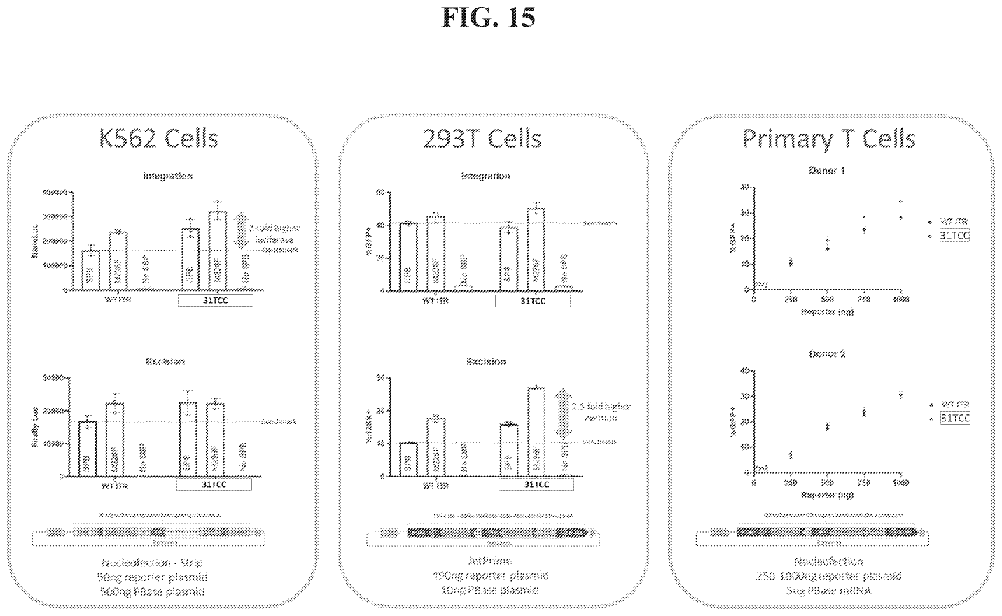

This experiment was repeated in K562 cells, HEK293T cells and primary T cells (FIG. 15). In K562 cells, transposition with a wildtype SPB and a mutant transposon with a 31TCC mutation in the LE ITR resulted in about a 1.5-fold increase in integration (i.e. transposition activity) relative to transposition with a wildtype SPB and transposon with a wildtype ITR. In HEK293T cells, transposition with a wildtype SPB and a mutant transposon with a 31TCC mutation in the LE ITR resulted in about a 1.6-fold increase in excision (i.e. transposition activity) relative to transposition with a wildtype SPB and transposon with a wildtype ITR. Transposition activity was also tested in primary T cells isolated from two donors. Transposons with the 31TCC mutation in the LE ITR resulted in about a 5% increase in integration relative to a transposon with a wildtype LE ITR.

Furthermore, the 31TCC mutant on the LE ITR further improves transposition when used in combination with SPB transposase with additional mutations (FIG. 15). Two SPB transposase mutants were tested (i.e. wildtype “SPB” and “M226F SPB”). In K562 cells, transposition with a mutant M226F SPB and a mutant transposon with a 31TCC mutation in the LE ITR resulted in about a 2-fold increase in integration (i.e. transposition activity) relative to transposition with a wildtype SPB and transposon with a wildtype ITR. In HEK293T cells, transposition with a mutant M226F SPB and a mutant transposon with a 31TCC mutation in the LE ITR resulted in about a 2.5-fold increase in excision (i.e. transposition activity) relative to transposition with a wildtype SPB and transposon with a wildtype ITR. Co-transfection of a cell with mutant SPB and a mutant transposon with a 31TCC mutation in the LE ITR results in increased in transposition activity relative to co-transfection of a cell with the same transposon and a wildtype SPB.

What is claimed is:

1. A polynucleotide encoding a transposon comprising:
a) a first inverted terminal repeat (ITR) comprising a nucleic acid sequence having at least 99% identity to SEQ ID NO: 4; and
b) a second ITR comprising a nucleic acid sequence having at least 99% identity to SEQ ID NO: 5; and
wherein the first ITR comprises a nucleic acid substitution in at least one of nucleic acid position 31 and position 33 of SEQ ID NO: 4.

2. The polynucleotide of claim 1, wherein the first ITR comprises the nucleic acid substitution of 31G>T.

3. The polynucleotide of claim 1, wherein the first ITR comprises the nucleic acid substitution of 33A>C.

4. The polynucleotide of claim 1, wherein the first ITR comprises the nucleic acid substitution of 31G>T and 33A>C.

5. The polynucleotide of claim 1, wherein the first ITR comprises the nucleic acid sequence of SEQ ID NO: 16.

6. The polynucleotide of any one of claims 1-5, wherein the transposon is a piggyBac transposon or a piggyBac-like transposon.

7. The polynucleotide of claim 6, wherein the piggyBac transposon comprises any of the nucleic acid sequences set forth in of SEQ ID NOs: 144-156 and 158.

8. The polynucleotide of any one of the preceding claims, wherein the polynucleotide further comprises at least one exogenous nucleic acid sequence.

9. The polynucleotide of claim 8, wherein the at least one exogenous nucleic acid sequence encodes a non-naturally occurring antigen receptor, a sequence encoding a therapeutic polypeptide, or a combination thereof.

10. The polynucleotide of claim 8, wherein at least one exogenous nucleic acid sequence encodes a non-naturally occurring antigen receptor comprising a chimeric antigen receptor.

11. The polynucleotide of claim 8, wherein the at least one exogenous nucleic acid sequence encodes a therapeutic polypeptide.

12. A vector comprising the polynucleotide of any one of claims 1-11.

13. A cell comprising the polynucleotide of any one of claims 1-11.

14. A pharmaceutical composition comprising the cell of claim 13 and a pharmaceutically acceptable carrier.

15. A method of producing a population of modified cells comprising delivering to a population of cells,
a) the polynucleotide of any one of claims 1-11, and
b) a nucleic acid or amino acid sequence comprising a sequence encoding a transposase enzyme, thereby inducing transposition activity and producing a population of modified cells, and
wherein the transposition activity of the polynucleotide is increased by at least 2.0 fold, at least 2.1 fold, at least 2.2 fold, at least 2.3 fold, at least 2.4 fold, at least 2.5 fold, at least 2.6 fold, at least 2.7 fold, at least 2.8 fold at least 2.9 fold or at least 3.0 fold in comparison to a polynucleotide comprising a first ITR comprising a nucleic acid sequence of SEQ ID NO: 4 and a second ITR comprising a nucleic acid sequence of SEQ ID NO: 5.

16. The method of claim 15, wherein the transposase enzyme is a piggyBac transposase or a piggy-Bac like transposase.

17. The method of claim 15, wherein the transposase enzyme is a Super piggyBac™ (SPB) transposase enzyme.

18. The method of claim 15, wherein the transposase enzyme comprises an amino acid sequence comprising SEQ ID NO: 107 or 109.

19. A composition comprising the population of modified cells produced according to the method of any one of claims 15-18.

20. A method of treating a disease or disorder in a subject in need thereof comprising administering to the subject at least one therapeutically effective dose of a polynucleotide, a vector, a cell and/or a composition of any of the preceding claims.

21. The method of claim 20, wherein the disease or disorder is cancer, a liver disease or disorder, a urea cycle disorder, a metabolic liver disorder or a hemophilia disease.


SEQUENCE LISTING (687 sequences)

SEQ ID NO: 1 - DNA, 303 bp, synthetic construct
  source 1..303: mol_type = other DNA
tataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 2 - DNA, 238 bp, synthetic construct
  source 1..238: mol_type = other DNA
ccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 3 - DNA, 309 bp, synthetic construct
  source 1..309: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 4 - DNA, 35 bp, synthetic construct
  source 1..35: mol_type = other DNA
ccctagaaagatagtctgcgtaaaattgacgcatg

SEQ ID NO: 5 - DNA, 63 bp, synthetic construct
  source 1..63: mol_type = other DNA
ccctagaaagataatcatattgtgacgtacgttaaagataatcatgcgtaaaattgacgcatg

SEQ ID NO: 6-10 - skipped (5 entries)

SEQ ID NO: 11 - DNA, 241 bp, synthetic construct
  source 1..241: mol_type = other DNA
ttaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaactttta

SEQ ID NO: 12-13 - skipped (2 entries)

SEQ ID NO: 14 - DNA, 35 bp, synthetic construct
  source 1..35: mol_type = other DNA
ccctagaaagatagtctgcgtaaaattgactcatg

SEQ ID NO: 15 - DNA, 35 bp, synthetic construct
  source 1..35: mol_type = other DNA
ccctagaaagatagtctgcgtaaaattgacgcctg

SEQ ID NO: 16 - DNA, 35 bp, synthetic construct
  source 1..35: mol_type = other DNA
ccctagaaagatagtctgcgtaaaattgactcctg

SEQ ID NO: 17 - DNA, 281 bp, synthetic construct
  source 1..281: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 18 - DNA, 1264 bp, synthetic construct
  source 1..1264: mol_type = other DNA
agctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 19 - DNA, 252 bp, synthetic construct
  source 1..252: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 20 - DNA, 449 bp, synthetic construct
  source 1..449: mol_type = other DNA
cccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatc

SEQ ID NO: 21 - DNA, 1214 bp, synthetic construct
  source 1..1214: mol_type = other DNA
gctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcggtaagtatcaaggttacaagacaggtttaaggagaccaatagaaactgggcttgtcgagacagagaagactcttgcgtttctgataggcacctattggtcttactgacatccactttgcctttctctccacagacctgggacagtgaat

SEQ ID NO: 22 - skipped

SEQ ID NO: 23 - DNA, 861 bp, synthetic construct
  source 1..861: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagactgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 24 - DNA, 146 bp, synthetic construct
  source 1..146: mol_type = other DNA
gttaatcattaagtcgttaatttttgtggcccttgcgatgtttgctctggttaataatctcaggacaaacagaggttaataattttccagatctctctgagcaatagtataaaaggccagcagcagcctgaccacatctcatcctc

SEQ ID NO: 25 - DNA, 716 bp, synthetic construct
  source 1..716: mol_type = other DNA
gctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgagatctgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 26 - DNA, 1224 bp, synthetic construct
  source 1..1224: mol_type = other DNA
tcgtcgacgggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgattaactgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccggcgaattaatgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaattgcggtaagtatcaaggttacaagacaggtttaaggagaccaatagaaactgggcttgtcgagacagagaagactcttgcgtttctgataggcacctattggtcttactgacatccactttgcctttctctccacagacctgggacagtgaat

SEQ ID NO: 27 - DNA, 256 bp, synthetic construct
  source 1..256: mol_type = other DNA
gcgcgtgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggatatccgaggacagggccctgtctcctcagcttcaggcaccaccactgacctgggacagt

SEQ ID NO: 28 - DNA, 869 bp, synthetic construct
  source 1..869: mol_type = other DNA
tgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcactagtagctttattgcggtagtttatcacagttaaattgctaacgcagtcagtgctcgactgatcacaggtaagtatcaaggttacaagacaggtttaaggaggccaatagaaactgggcttgtcgagacagagaagattcttgcgtttctgataggcacctattggtcttactgacatccactttgcctttctctccacaggggtaccgaagccgctagc

SEQ ID NO: 29 - protein, 750 aa, synthetic construct
MLRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 30 - protein, 749 aa, synthetic construct
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGISTLAAGHKTLVPELIKELNSLGRPDILVMCGGVIPPQDYEFLFEVGVSNVFGPGTRIPKAAVQVLDDIEKCLEKKQQSV

SEQ ID NO: 31 - protein, 750 aa, synthetic construct [sequence omitted]

SEQ ID NO: 32 - protein, 749 aa, synthetic construct
LRAKNQLFLLSPHYLRQVKESSGSRLIQQRLLHQQQPLHPEWAALAKKQLKGKNPEDLIWHTPEGISIKPLYSKRDTMDLPEELPGVKPFTRGPYPTMYTFRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLANFIVTGEEQGVPKEKLTGTIQNDILKEFMVRNTYIFPPEPSMKIIADIFEYTAKHMPKFNSISISGYHMQEAGADAILELAYTLADGLEYSRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMECLTNDVYDAALKLINEIEEMGGMAKAVAEGIPKLRIEECAARRQARIDSGSEVIVGVNKYQLEKEDAVEVLAIDNTSVRNRQIEKLKKIKSSRDQALAERCLAALTECAASGDGNILALAVDASRARCTVGEITDALKKVFGEHKANDRMVSGAYRQEFGESKEITSAIKRVHKFMEREGRRPRLLVAKMGQDGHDRGAKVIATGFADLGFDVDIGPLFQTPREVAQQAVDADVHAVGVSTLAAGHKTLVPELIKELNSLGRPDILVMCGGVIPPQDYEFLFEVGVSNVFGPGTRIPKAAVQVLDDIEKCLEKKQQSV

SEQ ID NO: 33 - DNA, 2250 bp, synthetic construct
  source 1..2250: mol_type = other DNA
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 34 - DNA, 2247 bp, synthetic construct
  source 1..2247: mol_type = other DNA
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcatctctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtg

SEQ ID NO: 35 - DNA, 2256 bp, synthetic construct
  source 1..2256: mol_type = other DNA
atgctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 36 - DNA, 2253 bp, synthetic construct
  source 1..2253: mol_type = other DNA
ctgcgggccaagaatcaactgttcctgctgtcccctcactacctgcggcaagtgaaagagagcagcggcagcagactgatccagcagagactgctgcatcagcagcagccactgcaccctgaatgggccgctctggctaagaagcagctcaagggcaagaaccccgaggacctgatctggcacacaccagagggcatcagcatcaagcccctgtactccaagcgggacacaatggatctgcccgaggaactgcctggcgtgaagccttttacaagaggcccctatcctaccatgtataccttcagaccctggaccatccggcagtacgccggcttttctaccgtggaagagagcaacaagttctacaaggacaacatcaaggccggccagcagggactgagcgtggcatttgatctggctacccacaggggctacgacagcgacaaccctagagtgcggggagatgttggaatggccggcgtggcaatcgacacagtggaagataccaagatcctgttcgacggcatccctctggaaaagatgagcgtgtccatgaccatgaacggcgctgtgatccccgtgctggctaactttattgtgaccggcgaggaacagggcgtgcccaaagaaaagctgaccggcaccatccagaacgacatcctgaaagagttcatggttcgaaacacctacatcttcccacctgagccgagcatgaagatcattgccgacatcttcgagtacaccgccaagcacatgcccaagttcaacagcatctccatcagcggctaccacatgcaagaggctggcgccgatgccatcctggaactggcttatacactggccgacggcctggaatactccagaacaggactgcaagccggcctgaccatcgatgagtttgcccctagactgagcttcttctggggcatcggcatgaacttctacatggaaatcgccaagatgagagccggcagacggctgtgggctcacctgatcgagaagatgttccagcctaagaacagcaagagcctgctcctgagagcccactgtcagacaagtggctggtccctgactgagcaggacccctacaacaacatcgtgcgcacagccatcgaagctatggccgccgtgtttggcggaacacagagcctgcacaccaacagctttgacgaggctctgggcctgcctaccgtgaagtctgccagaatcgcccggaacacccagatcatcatccaagaggaaagcggcatccccaaggtggcagatccttggggcggcagctacatgatggaatgcctgaccaacgacgtgtacgacgccgctctgaagctgatcaacgagatcgaagagatgggcggcatggctaaggctgtggccgagggaatccccaagctgagaatcgaggaatgcgccgccagacggcaggccagaattgatagcggaagcgaagtgatcgtgggcgtgaacaagtaccagctcgaaaaagaggacgccgtcgaggtcctggctatcgacaataccagcgtgcggaaccggcagattgagaagctgaagaagatcaagagcagccgcgatcaggccctggccgaaagatgtcttgctgccctgacagagtgtgccgccagcggcgacggaaatattctggctctggccgtggatgccagccgggctagatgtaccgtgggcgagattacagacgccctgaagaaggtgttcggcgagcacaaggccaacgacagaatggtgtctggcgcctacagacaagagtttggcgagagcaaagagatcaccagcgccatcaagcgggtccacaagttcatggaaagagaaggcaggcggcccagactgctggtggctaagatgggacaagacggccatgacagaggcgccaaagtgatcgccacaggctttgccgatctgggcttcgacgtggacatcggccctctgtttcagacccctagagaggtggcacagcaggccgttgatgccgatgttcacgctgtgggcgtgtctacactggctgccggacacaagacactggtgcccgaactgatcaaagagctgaacagcctgggcagacccgacatccttgtgatgtgtggcggagtgatcccaccgcaggactacgagttcctgtttgaagtgggcgtgtccaacgtgttcggccctggcacaagaatccctaaagccgccgtgcaggttctggacgacatcgagaagtgcctggaaaaaaagcagcagagcgtgggatcc

SEQ ID NO: 37-38 - skipped (2 entries)

SEQ ID NO: 39 - DNA, 2253 bp, synthetic construct
  source 1..2253: mol_type = other DNA
atgctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 40 - DNA, 2250 bp, synthetic construct
  source 1..2250: mol_type = other DNA
ctgagagccaaaaaccagctgttcctgctgagcccccactatctgagacaggtcaaagaaagttccgggagtagactgatccagcagagactgctgcaccagcagcagccactgcatcctgagtgggccgctctggccaagaaacagctgaagggcaaaaacccagaagacctgatctggcacactccagaggggatttcaatcaagcccctgtacagcaaaagggacactatggatctgccagaggaactgccaggagtgaagcctttcacccgcggaccttacccaactatgtatacctttcgaccctggacaattcggcagtacgccggcttcagtactgtggaggaatcaaacaagttttataaggacaacatcaaggctggacagcagggcctgagtgtggcattcgatctggccacacatcgcggctatgactcagataatcccagagtcaggggggacgtgggaatggcaggagtcgctatcgacacagtggaagatactaagattctgttcgatggaatccctctggagaaaatgtctgtgagtatgacaatgaacggcgctgtcattcccgtgctggcaaacttcatcgtcactggcgaggaacagggggtgcctaaggaaaaactgaccggcacaattcagaacgacatcctgaaggagttcatggtgcggaatacttacatttttccccctgaaccatccatgaaaatcattgccgatatcttcgagtacaccgctaagcacatgcccaagttcaactcaattagcatctccgggtatcatatgcaggaagcaggagccgacgctattctggagctggcttacaccctggcagatggcctggaatattctcgaaccggactgcaggcaggcctgacaatcgacgagttcgctcctagactgagtttcttttggggaattggcatgaacttttacatggagatcgccaagatgagggctggccggagactgtgggcacacctgatcgagaagatgttccagcctaagaactctaagagtctgctgctgcgggcccattgccagacatccggctggtctctgactgaacaggacccatataacaatattgtcagaaccgcaatcgaggcaatggcagccgtgttcggaggaacccagagcctgcacacaaactcctttgatgaggccctggggctgcctaccgtgaagtctgctaggattgcacgcaatacacagatcattatccaggaggaatccggaatcccaaaggtggccgatccctggggaggctcttacatgatggagtgcctgacaaacgacgtgtatgatgctgcactgaagctgattaatgaaatcgaggaaatggggggaatggcaaaggccgtggctgagggcattccaaaactgaggatcgaggaatgtgcagctaggcgccaggcacgaattgactcaggaagcgaagtgatcgtcggggtgaataagtaccagctggagaaagaagacgcagtcgaagtgctggccatcgataacacaagcgtgcgcaatcgacagattgagaagctgaagaaaatcaaaagctcccgcgatcaggcactggccgaacgatgcctggcagccctgactgagtgtgctgcaagcggggacggaaacattctggctctggcagtcgatgcctcccgggctagatgcactgtgggggaaatcaccgacgccctgaagaaagtcttcggagagcacaaggccaatgatcggatggtgagcggcgcttatagacaggagttcggggaatctaaagagattaccagtgccatcaagagggtgcacaagttcatggagagagaagggcgacggcccaggctgctggtggcaaagatgggacaggacggacatgatcgcggagcaaaagtcattgccaccgggttcgctgacctgggatttgacgtggatatcggccctctgttccagacaccacgagaggtcgcacagcaggcagtcgacgctgatgtgcacgcagtcggagtgtccactctggcagctggccataagaccctggtgcctgaactgatcaaagagctgaactctctgggcagaccagacatcctggtcatgtgcggcggcgtgatcccaccccaggattacgaattcctgtttgaggtcggggtgagcaacgtgttcggaccaggaaccaggatccctaaggccgcagtgcaggtcctggatgatattgaaaagtgtctggaaaagaaacagcagtcagtgtaa

SEQ ID NO: 41 - DNA, 2253 bp, synthetic construct
  source 1..2253: mol_type = other DNA
atgttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 42 - DNA, 2250 bp, synthetic construct
  source 1..2250: mol_type = other DNA
ttaagagctaagaatcagctttttttactttcacctcattacctgaggcaggtaaaagaatcatcaggctccaggctcatacagcaacgacttctacaccagcaacagccccttcacccagaatgggctgccctggctaaaaagcagctgaaaggcaaaaacccagaagacctaatatggcacaccccggaagggatctctataaaacccttgtattccaagagagatactatggacttacctgaagaacttccaggagtgaagccattcacacgtggaccatatcctaccatgtatacctttaggccctggaccatccgccagtatgctggttttagtactgtggaagaaagcaataagttctataaggacaacattaaggctggtcagcagggattatcagttgcctttgatctggcgacacatcgtggctatgattcagacaaccctcgagttcgtggtgatgttggaatggctggagttgctattgacactgtggaagataccaaaattctttttgatggaattcctttagaaaaaatgtcagtttccatgactatgaatggagcagttattccagttcttgcaaattttatagtaactggagaagaacaaggtgtacctaaagagaagcttactggtaccatccaaaatgatatactaaaggaatttatggttcgaaatacatacatttttcctccagaaccatccatgaaaattattgctgacatatttgaatatacagcaaagcacatgccaaaatttaattcaatttcaattagtggataccatatgcaggaagcaggggctgatgccattctggagctggcctatactttagcagatggattggagtactctagaactggactccaggctggcctgacaattgatgaatttgcaccaaggttgtctttcttctggggaattggaatgaatttctatatggaaatagcaaagatgagagctggtagaagactctgggctcacttaatagagaaaatgtttcagcctaaaaactcaaaatctcttcttctaagagcacactgtcagacatctggatggtcacttactgagcaggatccctacaataatattgtccgtactgcaatagaagcaatggcagcagtatttggagggactcagtctttgcacacaaattcttttgatgaagctttgggtttgccaactgtgaaaagtgctcgaattgccaggaacacacaaatcatcattcaagaagaatctgggattcccaaagtggctgatccttggggaggttcttacatgatggaatgtctcacaaatgatgtttatgatgctgctttaaagctcattaatgaaattgaagaaatgggtggaatggccaaagctgtagctgagggaatacctaaacttcgaattgaagaatgtgctgcccgaagacaagctagaatagattctggttctgaagtaattgttggagtaaataagtaccagttggaaaaagaagacgctgtagaagttctggcaattgataatacttcagtgcgaaacaggcagattgaaaaacttaagaagatcaaatccagcagggatcaagctttggctgaacgttgtcttgctgcactaaccgaatgtgctgctagcggagatggaaatatcctggctcttgcagtggatgcatctcgggcaagatgtacagtgggagaaatcacagatgccctgaaaaaggtatttggtgaacataaagcgaatgatcgaatggtgagtggagcatatcgccaggaatttggagaaagtaaagagataacatctgctatcaagagggttcataaattcatggaacgtgaaggtcgcagacctcgtcttcttgtagcaaaaatgggacaagatggccatgacagaggagcaaaagttattgctacaggatttgctgatcttggttttgatgtggacataggccctcttttccagactcctcgtgaagtggcccagcaggctgtggatgcggatgtgcatgctgtgggcataagcaccctcgctgctggtcataaaaccctagttcctgaactcatcaaagaacttaactcccttggacggccagatattcttgtcatgtgtggaggggtgataccacctcaggattatgaatttctgtttgaagttggtgtttccaatgtatttggtcctgggactcgaattccaaaggctgccgttcaggtgcttgatgatattgagaagtgtttggaaaagaagcagcaatctgtataa

SEQ ID NO: 43 - DNA, 2250 bp, synthetic construct
  source 1..2250: mol_type = other DNA
atgctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 44 - DNA, 2247 bp, synthetic construct
  source 1..2247: mol_type = other DNA
ctgcgggccaagaaccagctgttcctgctgagccctcactacctgcggcaggtgaaggagagcagcggcagccggctgatccagcagcggctgctgcaccagcagcagcccctgcaccccgagtgggccgccctggccaagaagcagctgaagggcaagaaccccgaggacctgatctggcacacgcccgagggcatcagcatcaagcccctgtacagcaagcgggacaccatggacctgcccgaggagctgcccggcgtgaagcccttcacccggggcccctaccccaccatgtacaccttccggccctggaccatccggcagtacgccggcttcagcaccgtggaggagagcaacaagttctacaaggacaacatcaaggccggccagcagggcctgagcgtggccttcgacctggccacccaccggggctacgacagcgacaacccacgggtgcggggcgacgtgggcatggccggcgtggccatcgacaccgtggaggacaccaagatcctgttcgacggcatccctctggagaagatgagcgtgagcatgaccatgaacggcgccgtgatccccgtgctggccaacttcatcgtgaccggcgaggagcagggcgtgcccaaggagaagctgaccggcaccatccagaacgacatcctgaaggagttcatggtgcggaacacctacatcttccctcccgagcccagcatgaagatcatcgccgacatcttcgagtacaccgccaagcacatgcccaagttcaacagcatcagcatcagcggctaccacatgcaggaggccggcgccgacgccatcctggagctggcctacaccctggccgacggcctggagtacagccggaccggcctgcaggccggcctgaccatcgacgagttcgcgccccggctgagcttcttctggggcatcggcatgaacttctacatggagatcgccaagatgcgggccggccggcggctgtgggcccacctgatcgagaagatgttccagcccaagaacagcaagagcctgctgctgcgggcccactgccagaccagcggctggagcctgaccgagcaggacccctacaacaacatcgtgcggaccgccatcgaggccatggccgccgtgttcggcggcacccagagcctgcacaccaacagcttcgacgaggccctgggcctgcccaccgtgaagagcgcccggatcgcccggaacacccagatcatcatccaggaggagagcggcatccccaaggtggccgacccctggggcggcagctacatgatggagtgcctgaccaacgacgtgtacgacgccgccctgaagctgatcaacgagatcgaggagatgggcggcatggccaaggccgtggccgagggcatccccaagctgcggatcgaggagtgcgccgcccggcggcaggcccggatcgacagcggcagcgaggtgatcgtgggcgtgaacaagtaccagctggagaaggaggacgccgtggaggtgctggccatcgacaacaccagcgtgcggaaccggcagatcgagaagctgaagaagatcaagagcagccgggaccaggccctggccgagcggtgcctggccgccctgaccgagtgcgccgccagcggcgacggcaacatcctggccctggccgtggacgccagccgggcccggtgcaccgtgggcgagatcaccgacgccctgaagaaggtgttcggcgagcacaaggccaacgaccggatggtgagcggcgcctaccggcaggagttcggcgagagcaaggagatcaccagcgccatcaagcgggtgcacaagttcatggagcgggagggccggcggccccggctgctggtggccaagatgggccaggacggccacgaccggggcgccaaggtgatcgccaccggcttcgccgacctgggcttcgacgtggacatcggcccactgttccagacgccccgggaggtggcccagcaggccgtggacgccgacgtgcacgccgtgggcgtgagcaccctggccgccggccacaagaccctggtgcccgagctgatcaaggagctgaacagcctgggccggcccgacatcctggtgatgtgcggcggcgtgatcccgccccaggactacgagttcctgttcgaggtgggcgtgagcaacgtgttcggccccggcacccggatccccaaggccgccgtgcaggtgctggacgacatcgagaagtgcctggagaagaagcagcagagcgtg

SEQ ID NO: 45 - protein, 354 aa, synthetic construct
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 46 - protein, 353 aa, synthetic construct
LFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 47 - DNA, 1062 bp, synthetic construct
  source 1..1062: mol_type = other DNA
atgctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 48 - DNA, 1062 bp, synthetic construct
  source 1..1062: mol_type = other DNA
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 49 - DNA, 1059 bp, synthetic construct
  source 1..1059: mol_type = other DNA
ctgttcaacctgcgcatcctgctgaacaacgccgccttcagaaacggccacaacttcatggttcgaaacttcagatgcggccagcctctccagaacaaggtgcagctgaaaggcagggacctgctgaccctgaagaacttcaccggcgaagagatcaagtacatgctgtggctgtccgccgacctgaagttcagaatcaagcagaagggcgagtacctgcctctgctccagggaaagtctctgggcatgatcttcgagaagcggagcaccagaaccagactgagcaccgagacaggctttgccctgctcggaggacacccctgctttctgacaacccaggacatccacctgggcgtgaacgagagcctgaccgatacagccagagtgctgtcctctatggccgatgccgtgctggctagagtgtataagcagagcgacctggacaccctggctaaagaggccagcattcccatcatcaacggcctgtccgacctgtatcaccccatccagatcctggccgactacctgacactgcaagagcactacagcagcctgaagggactgaccctgtcttggatcggcgacggcaacaacatcctgcacagcattatgatgagcgccgccaagttcggaatgcacctccaggccgctacacccaagggctatgaacctgatgccagcgtgacaaagctggccgagcagtacgccaaagagaacggcacaaagctgctgctgaccaacgatcccctggaagctgctcacggcggcaatgtgctgatcaccgatacctggatcagcatgggccaagaggaagagaagaagaagcggctgcaagccttccagggctaccaagtgaccatgaagacagccaaggtggccgccagcgattggacctttctgcactgcctgcctcggaagcctgaagaggtggacgacgaggtgttctacagccctagaagcctggtgttccccgaggccgagaacagaaagtggaccatcatggctgtgatggtgtctctgctgaccgactactcccctcagctccagaagcctaagttc

SEQ ID NO: 50 - DNA, 1059 bp, synthetic construct
  source 1..1059: mol_type = other DNA
ctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctcaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttc

SEQ ID NO: 51 - protein, 1457 aa, synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 52 - DNA, 4371 bp, synthetic construct
  source 1..4371: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtac

SEQ ID NO: 53 - DNA, 139 bp, synthetic construct
  source 1..139: mol_type = other DNA
attccgataacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 54 - DNA, 192 bp, synthetic construct
  source 1..192: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 55 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 56 - DNA, 139 bp, synthetic construct
  source 1..139: mol_type = other DNA
tgtttattgcagcttataatggttacaaataaagcaatagcatcacaaatttcacaaataaagcatttttttcactgcattctagttgtggtttgtccaaactcatcaatgtatcttatcatgtctggcgctagcagca

SEQ ID NO: 57 - skipped

SEQ ID NO: 58 - DNA, 278 bp, synthetic construct
  source 1..278: mol_type = other DNA
ctggtactgcatgcacgcaatgctagctgcccctttcccgtcctgggcaccccgagtctcccccgaccccgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 59 - DNA, 136 bp, synthetic construct
  source 1..136: mol_type = other DNA
ctggtactgcatgcacgcaatgctagctgcccctttcccgtcctgggcaccccgagtctcccccgaccccgggtcccaggtatgctcccacctccacctgccccactcaccacctctgctagttccagacacctcc

SEQ ID NO: 60 - DNA, 139 bp, synthetic construct
  source 1..139: mol_type = other DNA
caagcacgcagcaatgcagctcaaaacgcttagcctagccacacccccacgggaaacagcagtgattaacctttagcaataaacgaaagtttaactaagctatactaaccccagggttggtcaatttcgtgccagccac

SEQ ID NO: 61 - protein, 187 aa, synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 62 - DNA, 561 bp, synthetic construct
  source 1..561: mol_type = other DNA
atggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 63 - skipped

SEQ ID NO: 64 - DNA, 232 bp, synthetic construct
  source 1..232: mol_type = other DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 65 - DNA, 231 bp, synthetic construct
  source 1..231: mol_type = other DNA
ttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccct

SEQ ID NO: 66-67 - skipped (2 entries)

SEQ ID NO: 68 - DNA, 127 bp, synthetic construct
  source 1..127: mol_type = other DNA
cagacatgataagatacattgatgagtttggacaaaccacaactagaatgcagtgaaaaaaatgctttatttgtgaaatttgtgatgctattgctttatttgtaaccattataagctgcaataaaca

SEQ ID NO: 69 - protein, 15 aa, synthetic construct
GGGGSGGGGSGGGGS

SEQ ID NO: 70 - DNA, 45 bp, synthetic construct
  source 1..45: mol_type = other DNA
ggcggaggcggtagcggtggcggaggtagcggaggtggtggatct

SEQ ID NO: 71 - protein, 90 aa, synthetic construct
MLPAPKNLVVSRITEDSARLSWTAPDAAFDSFPIRYIETLIWGEAIWLDVPGSERSYDLTGLKPGTEYAVVITGVKGGRFSSPLVASFTT

SEQ ID NO: 72 - DNA, 270 bp, synthetic construct
  source 1..270: mol_type = other DNA
atgctgcctgcaccaaagaacctggtggtgagccggatcacagaggactccgccagactgtcttggaccgcccctgacgccgccttcgattcctttccaatccggtacatcgagacactgatctggggcgaggccatctggctggacgtgcccggctctgagaggagctacgatctgacaggcctgaagcctggcaccgagtatgcagtggtcatcacaggagtgaagggcggcaggttcagctcccctctggtggcctcttttaccaca

SEQ ID NO: 73 - protein, 334 aa, synthetic construct
MALPVTALLLPLALLLHAARPMLPAPKNLVVSRITEDSARLSWTAPDAAFDSFPIRYIETLIWGEAIWLDVPGSERSYDLTGLKPGTEYAVVITGVKGGRFSSPLVASFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1002 bp, synthetic construct
  source 1..1002: mol_type = other DNA
atggcactgccagtcaccgccctgctgctgcctctggctctgctgctgcacgcagctagaccaatgctgcctgcaccaaagaacctggtggtgagccggatcacagaggactccgccagactgtcttggaccgcccctgacgccgccttcgattcctttccaatccggtacatcgagacactgatctggggcgaggccatctggctggacgtgcccggctctgagaggagctacgatctgacaggcctgaagcctggcaccgagtatgcagtggtcatcacaggagtgaagggcggcaggttcagctcccctctggtggcctcttttaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 75 - protein, 958 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGEGRGSLLTCGDVEENPGPMALPVTALLLPLALLLHAARPMLPAPKNLVVSRITEDSARLSWTAPDAAFDSFPIRYIETLIWGEAIWLDVPGSERSYDLTGLKPGTEYAVVITGVKGGRFSSPLVASFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 76 - DNA, 6198 bp, synthetic construct
  source 1..6198: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatggacagttagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattctaatacgactcactatagggtgtgctgtctcatcattttggcaaagattggccaccaagcttgccaccatgggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttccggatctggagagggaaggggaagcctgctgacctgtggagacgtggaggaaaacccaggaccaatggcactgccagtcaccgccctgctgctgcctctggctctgctgctgcacgcagctagaccaatgctgcctgcaccaaagaacctggtggtgagccggatcacagaggactccgccagactgtcttggaccgcccctgacgccgccttcgattcctttccaatccggtacatcgagacactgatctggggcgaggccatctggctggacgtgcccggctctgagaggagctacgatctgacaggcctgaagcctggcaccgagtatgcagtggtcatcacaggagtgaagggcggcaggttcagctcccctctggtggcctcttttaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaaggggaagtggagaaggacgaggatcactgctgacatgcggcgacgtggaggaaaaccctggcccaatggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgactaataaggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 77 - protein, 92 aa, synthetic construct
MLPAPKNLVVSRVTEDSARLSWAIDEQRDWFESFLIQYQESEKVGEAIVLTVPGSERSYDLTGLKPGTEYTVSIYGVYHVYRSNPLSAIFTT

SEQ ID NO: 78 - DNA, 411 bp, synthetic construct
  source 1..411: mol_type = other DNA
atgctgcctgcaccaaagaacctggtggtgtctcgggtgaccgaggactctgccagactgagctgggccatcgacgagcagagggattggttcgagagctttctgatccagtatcaggagtccgagaaagtgggcgaggccatcgtgctgacagtgcctggcagcgagcggtcctatgatctgaccggcctgaagccaggcacagagtacaccgtgtccatctacggcgtgtatcacgtgtacaggtccaatcctctgtctgccatcttcaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgac

SEQ ID NO: 79 - protein, 336 aa, synthetic construct
MALPVTALLLPLALLLHAARPMLPAPKNLVVSRVTEDSARLSWAIDEQRDWFESFLIQYQESEKVGEAIVLTVPGSERSYDLTGLKPGTEYTVSIYGVYHVYRSNPLSAIFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1008 bp, synthetic construct
  source 1..1008: mol_type = other DNA
atggcactgccagtcaccgccctgctgctgcctctggctctgctgctgcacgcagctagaccaatgctgcctgcaccaaagaacctggtggtgtctcgggtgaccgaggactctgccagactgagctgggccatcgacgagcagagggattggttcgagagctttctgatccagtatcaggagtccgagaaagtgggcgaggccatcgtgctgacagtgcctggcagcgagcggtcctatgatctgaccggcctgaagccaggcacagagtacaccgtgtccatctacggcgtgtatcacgtgtacaggtccaatcctctgtctgccatcttcaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 81 - protein, 960 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGEGRGSLLTCGDVEENPGPMALPVTALLLPLALLLHAARPMLPAPKNLVVSRVTEDSARLSWAIDEQRDWFESFLIQYQESEKVGEAIVLTVPGSERSYDLTGLKPGTEYTVSIYGVYHVYRSNPLSAIFTTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 82 - DNA, 6204 bp, synthetic construct
  source 1..6204: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatggacagttagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattctaatacgactcactatagggtgtgctgtctcatcattttggcaaagattggccaccaagcttgccaccatgggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttccggatctggagagggaaggggaagcctgctgacctgtggagacgtggaggaaaacccaggaccaatggcactgccagtcaccgccctgctgctgcctctggctctgctgctgcacgcagctagaccaatgctgcctgcaccaaagaacctggtggtgtctcgggtgaccgaggactctgccagactgagctgggccatcgacgagcagagggattggttcgagagctttctgatccagtatcaggagtccgagaaagtgggcgaggccatcgtgctgacagtgcctggcagcgagcggtcctatgatctgaccggcctgaagccaggcacagagtacaccgtgtccatctacggcgtgtatcacgtgtacaggtccaatcctctgtctgccatcttcaccacaaccacaacccctgcccccagacctcccacacccgcccctaccatcgcgagtcagcccctgagtctgagacctgaggcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaaggggaagtggagaaggacgaggatcactgctgacatgcggcgacgtggaggaaaaccctggcccaatggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgactaataaggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 83 - protein, 118 aa, synthetic construct
EVQLLESGGGLVQPGGSLRLSCAASGFTFSNYAMTWIRQAPGKGLEWVSGITGDGGSTFYADSVKGRFTISRDNSKNTLYLQMNSLRAEDTAVYYCVKDWNTTMITERGQGTLVTVSS

SEQ ID NO: 84 - DNA, 489 bp, synthetic construct
  source 1..489: mol_type = other DNA
gaagttcagctgcttgaatctggcggaggcctggttcaacctggcggatctctgagactgagctgtgccgccagcggcttcaccttcagcaattacgccatgacctggatcagacaggcccctggcaaaggcctggaatgggtgtccggaattacaggcgacggcggcagcaccttttacgccgattctgtgaagggcagattcaccatcagccgggacaacagcaagaacaccctgtacctgcagatgaacagcctgagagccgaggacaccgccgtgtactactgcgtgaaggactggaacaccaccatgatcaccgagagaggccagggcacactggtcaccgtgtcctctacaacaacaccggcgcctagacctccaacaccagctcctacaatcgcgagtcagcccctgtctctcagacccgaagcctgcaggccagctgcaggaggagctgtgcacaccaggggcctggacttcgcctgcgac

SEQ ID NO: 85 - protein, 362 aa, synthetic construct
MALPVTALLLPLALLLHAARPEVQLLESGGGLVQPGGSLRLSCAASGFTFSNYAMTWIRQAPGKGLEWVSGITGDGGSTFYADSVKGRFTISRDNSKNTLYLQMNSLRAEDTAVYYCVKDWNTTMITERGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - DNA, 1086 bp, synthetic construct
  source 1..1086: mol_type = other DNA
atggctctgcctgtgacagctctgcttctgcctctggcactgcttcttcatgcggcgcgccctgaagttcagctgcttgaatctggcggaggcctggttcaacctggcggatctctgagactgagctgtgccgccagcggcttcaccttcagcaattacgccatgacctggatcagacaggcccctggcaaaggcctggaatgggtgtccggaattacaggcgacggcggcagcaccttttacgccgattctgtgaagggcagattcaccatcagccgggacaacagcaagaacaccctgtacctgcagatgaacagcctgagagccgaggacaccgccgtgtactactgcgtgaaggactggaacaccaccatgatcaccgagagaggccagggcacactggtcaccgtgtcctctacaacaacaccggcgcctagacctccaacaccagctcctacaatcgcgagtcagcccctgtctctcagacccgaagcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 87 - protein, 570 aa, synthetic construct
MALPVTALLLPLALLLHAARPEVQLLESGGGLVQPGGSLRLSCAASGFTFSNYAMTWIRQAPGKGLEWVSGITGDGGSTFYADSVKGRFTISRDNSKNTLYLQMNSLRAEDTAVYYCVKDWNTTMITERGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 88 - DNA, 6248 bp, synthetic construct
  source 1..6248: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatggacagttagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattctaatacgactcactatagggtgaagcttgccaccatgggggtccaggtggaaacaatctctccgggggatgggcggacattccctaaaaggggccagacctgcgtggtgcattacaccggcatgctggaagatggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagctggaaggcggcggaggatctggctttggagatgtgggagccctggaaagcctgagaggcaatgccgatctggcctacatcctgagcatggaaccttgcggccactgcctgattatcaacaacgtgaacttctgtagagagagcggcctgcggaccagaaccggcagcaatatcgattgcgagaagctgcggcggagattcagcagcctgcacttcatggtggaagtgaagggcgacctgaccgccaagaaaatggtgctggctctgctggaactggcccagcaagatcatggcgccctggattgctgtgtggtcgtgatcctgtctcacggctgtcaggccagccaccttcaattccctggcgccgtgtatggcacagatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagtctcctggaagcaaccctgagcctgacgccacacctttccaagagggcctgagaaccttcgaccagctggacgctatcagctccctgcctacacctagcgacatcttcgtgtcctacagcacattccccggctttgtgtcttggcgggaccctaagtctggctcttggtacgtggaaaccctggacgacatctttgagcagtgggctcacagcgaggacctccagtctctgctgctgagagtggccaatgccgtgtccgtgaagggcatctacaagcagatgcctggctgcttcaacttcctgcggaagaagctgtttttcaagaccagcggcagcggcgaaggcagaggatcccttttgacatgcggcgatgtggaagagaaccccggacctatggctctgcctgtgacagctctgcttctgcctctggcactgcttcttcatgcggcgcgccctgaagttcagctgcttgaatctggcggaggcctggttcaacctggcggatctctgagactgagctgtgccgccagcggcttcaccttcagcaattacgccatgacctggatcagacaggcccctggcaaaggcctggaatgggtgtccggaattacaggcgacggcggcagcaccttttacgccgattctgtgaagggcagattcaccatcagccgggacaacagcaagaacaccctgtacctgcagatgaacagcctgagagccgaggacaccgccgtgtactactgcgtgaaggactggaacaccaccatgatcaccgagagaggccagggcacactggtcaccgtgtcctctacaacaacaccggcgcctagacctccaacaccagctcctacaatcgcgagtcagcccctgtctctcagacccgaagcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaaggggaagtggagaaggacgaggatcactgctgacatgcggcgacgtggaggaaaaccctggcccaatggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgactaataaggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 89 - protein, 241 aa, synthetic construct
EILLTQSPDFQSVTPKEKVTFTCRASQSIGTSIHWYQQKPNQSPKLLIKYASESISGVPSRFSGSGSGTDFTLTINSLESEDIATYYCQQSNNWPLTFGQGTKLEIKGGGGSGGGGSGGGGSQVQLVQSGAEVKKPGSSVKVSCKTSGYAFSNFWMNWVRQAPGQGLEWIGQIYPGDGDTNYNAKFKGRVTLTADKSTSTAYMELSSLRSEDTAVYFCARSYYRSAWFAYWGQGTLVTVSS

SEQ ID NO: 90 - DNA, 723 bp, synthetic construct
  source 1..723: mol_type = other DNA
gagattctgctgacacagagccctgacttccagagcgtgacccctaaagaaaaagtgaccttcacctgtcgcgccagccagagcatcggcacctccatccactggtatcagcagaagcccaatcagagccccaagctgctgattaagtacgccagcgagtccatcagcggcgtgccaagcagattttctggcagcggctctggcaccgacttcaccctgaccatcaacagcctggaatccgaggatatcgccacctactactgccagcagtccaacaactggcccctgacatttggccagggcaccaagctggaaatcaaaggcggaggcggtagcggtggcggaggtagcggaggtggtggatctcaggttcagctggttcagtctggcgccgaagtgaagaaacctggcagcagcgtgaaggtgtcctgcaagacaagcggctacgccttcagcaacttctggatgaactgggtccgacaggcccctggacaaggcctggaatggatcggccaaatctatcccggcgacggcgacaccaactacaacgccaagttcaagggcagagtgacactgaccgccgacaagagcacaagcaccgcctacatggaactgtccagcctgagaagcgaggatacagccgtgtacttctgcgcccggtcctactacagaagcgcttggtttgcctattggggccagggaaccctggtcaccgtgtctagt

SEQ ID NO: 91 - protein, 485 aa, synthetic construct
MALPVTALLLPLALLLHAARPEILLTQSPDFQSVTPKEKVTFTCRASQSIGTSIHWYQQKPNQSPKLLIKYASESISGVPSRFSGSGSGTDFTLTINSLESEDIATYYCQQSNNWPLTFGQGTKLEIKGGGGSGGGGSGGGGSQVQLVQSGAEVKKPGSSVKVSCKTSGYAFSNFWMNWVRQAPGQGLEWIGQIYPGDGDTNYNAKFKGRVTLTADKSTSTAYMELSSLRSEDTAVYFCARSYYRSAWFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - DNA, 1455 bp, synthetic construct
  source 1..1455: mol_type = other DNA
atggctctgcctgtgacagctctgcttctgcctctggcactgcttcttcatgcggcgcgccctgagattctgctgacacagagccctgacttccagagcgtgacccctaaagaaaaagtgaccttcacctgtagagccagccagagcatcggcacctccatccactggtatcagcagaagcccaatcagagccccaagctgctgattaagtacgccagcgagtccatcagcggcgtgccaagcagattttctggcagcggaagcggcaccgacttcaccctgaccatcaacagcctggaatccgaggacattgccacctactactgccagcagtccaacaactggcccctgacatttggccagggcaccaagctggaaatcaaaggcggaggcggtagcggtggcggaggtagcggaggtggtggatctcaggttcagctggttcagtctggcgccgaagtgaagaaacctggcagcagcgtgaaggtgtcctgcaagacaagcggctacgccttcagcaacttctggatgaactgggtccgacaggcccctggacaaggcctggaatggatcggccaaatctatcccggcgacggcgacaccaactacaacgccaagttcaagggcagagtgacactgaccgccgacaagagcacaagcaccgcctacatggaactgagcagcctgagaagcgaggataccgccgtgtacttctgcgcccggtcctactacagaagcgcttggtttgcctattggggccagggaaccctcgtgaccgttagctctaccacaacaccggcgcctagacctccaacaccagctcctacaatcgcgagtcagcccctgtctctcagacccgaagcctgcaggccagctgcaggaggagctgtgcacaccaggggcctggacttcgcctgcgacatctacatttgggcaccactggccgggacctgtggagtgctgctgctgagcctggtcatcacactgtactgcaagagaggcaggaagaaactgctgtatattttcaaacagcccttcatgcgccccgtgcagactacccaggaggaagacgggtgctcctgtcgattccctgaggaagaggaaggcgggtgtgagctgcgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 93 - protein, 693 aa, synthetic construct
MALPVTALLLPLALLLHAARPEILLTQSPDFQSVTPKEKVTFTCRASQSIGTSIHWYQQKPNQSPKLLIKYASESISGVPSRFSGSGSGTDFTLTINSLESEDIATYYCQQSNNWPLTFGQGTKLEIKGGGGSGGGGSGGGGSQVQLVQSGAEVKKPGSSVKVSCKTSGYAFSNFWMNWVRQAPGQGLEWIGQIYPGDGDTNYNAKFKGRVTLTADKSTSTAYMELSSLRSEDTAVYFCARSYYRSAWFAYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 94 - DNA, 6616 bp, synthetic construct
  source 1..6616: mol_type = other DNA
tgtacatagattaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttatcgaatacctgcagcccgggggatgcagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagttgactgtgcctttcgatcgaaccatggacagttagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattctaatacgactcactatagggtgcggccgccaccatgggcgtgcaggtcgagacaatttctcctggcgacggccggacattccctaagcgaggacagacatgcgtggtgcactacaccggcatgctggaagatggcaagaaggtggacagcagccgggacagaaacaagcccttcaagttcatgctgggcaagcaagaagtgatcagaggctgggaagagggcgtcgcccagatgtctgttggacagagagccaagctgacaatcagccccgattacgcctatggcgccacaggacaccctggcatcattcctccacatgccacactggtgttcgacgtggaactgctgaagctggaaggcggcggaggatctggctttggagatgtgggagccctggaaagcctgagaggcaatgccgatctggcctacatcctgagcatggaaccttgcggccactgcctgattatcaacaacgtgaacttctgcagagagagcggcctgagaaccagaaccggcagcaacatcgactgcgagaagctgcggagaagattcagcagcctgcacttcatggtggaagtgaagggcgacctgaccgccaagaaaatggtgctggctctgctggaactggcccagcaagatcatggcgccctggattgctgtgtggtcgtgatcctgtctcacggctgtcaggccagccatctgcaattccctggcgccgtgtatggcaccgatggctgtcctgtgtccgtggaaaagatcgtgaacatcttcaacggcaccagctgtcctagcctcggcggaaagcccaagctgttcttcatccaagcctgtggcggcgagcagaaggatcacggatttgaggtggccagcacaagccccgaggatgagtctcctggaagcaaccctgagcctgacgccacacctttccaagagggactgagaaccttcgaccagctggacgctatcagctccctgcctacacctagcgacatcttcgtgtcctacagcacattccccggctttgtgtcttggcgggaccctaagtctggctcttggtatgtggaaaccctggacgatatcttcgagcagtgggcccatagcgaggacctgcaatctctgctgctgagagtggccaatgccgtgtccgtgaagggcatctacaagcagatgcctggctgcttcaacttcctgcggaagaagctgtttttcaagaccagcggcagcggcgaaggcagaggatcccttttgacatgcggcgacgtcgaagagaaccccggacctatggctctgcctgtgacagctctgcttctgcctctggcactgcttcttcatgcggcgcgccctgagattctgctgacacagagccctgacttccagagcgtgacccctaaagaaaaagtgaccttcacctgtcgcgccagccagagcatcggcacctccatccactggtatcagcagaagcccaatcagagccccaagctgctgattaagtacgccagcgagtccatcagcggcgtgccaagcagattttctggcagcggctctggcaccgacttcaccctgaccatcaacagcctggaatccgaggatatcgccacctactactgccagcagtccaacaactggcccctgacatttggccagggcaccaagctggaaatcaaaggcggaggcggtagcggtggcggaggtagcggaggtggtggatctcaggttcagctggttcagtctggcgccgaagtgaagaaacctggcagcagcgtgaaggtgtcctgcaagacaagcggctacgccttcagcaacttctggatgaactgggtccgacaggcccctggacaaggcctggaatggatcggccaaatctatcccggcgacggcgacaccaactacaacgccaagttcaagggcagagtgacactgaccgccgacaagagcacaagcaccgcctacatggaactgtccagcctgagaagcgaggatacagccgtgtacttctgcgcccggtcctactacagaagcgcttggtttgcctattggggccagggaaccctggtcaccgtgtctagtacaacaacaccggcgcctcggcctccaacaccagctcctacaattgctagccagccactgtctctgaggcccgaggcttgtagacctgctgctggcggagctgtgcacacaagaggactggatttcgcctgcgacatctatatctgggcccctctggccggaacatgtggcgttctgctgctcagcctggtcatcaccctgtactgcaagcggggcagaaagaagctgctgtacatctttaagcagcccttcatgaggcccgtgcagaccacacaagaagaggacggctgctcctgccgcttccccgaggaagaagaaggcggttgcgaactgagagtgaagttcagcagatccgccgacgcacccgcctataagcagggacagaatcagctgtacaacgagctgaatctggggcgcagagaagagtacgacgtgctggacaagagaagaggcagggaccctgagatgggcggcaagcccagaagaaagaaccctcaagagggcctgtataatgagctgcagaaagacaagatggccgaggcctacagcgagatcggaatgaagggcgagcgcagaagaggcaagggtcacgatggactgtaccagggcctgagcaccgccaccaaggatacctatgatgccctgcacatgcaggccctgcctccaagaggaagtggcgaaggacggggatctcttctcacgtgcggggatgttgaagagaatcctggtccaatggtcggaagcctgaactgcatcgtggccgtgtctcagaacatgggcatcggcaagaacggcgacttcccttggcctcctctgagaaacgagagccgctacttccagcggatgaccaccacaagctccgtggaaggcaagcagaacctcgtgatcatgggcaagaaaacctggttcagcatccctgagaagaacagacccctgaagggcagaatcaacctggtgctgagcagagagctgaaagagcctcctcaaggcgcccacttcctgagcagatctctggacgatgccctgaagctgaccgagcagccagaactggccaacaaagtggacatggtctggatcgtcggcggcagctccgtgtacaaagaagccatgaatcaccccggccacctgaaactgttcgtgaccagaatcatgcaggacttcgagagcgacacattcttcccggagatcgacctggaaaagtacaaactgctgcctgagtaccccggcgtgctgagcgacgtgcaagaggaaaagggcatcaagtacaagttcgaggtgtacgagaagaacgactaataaggtaccgatcacatatgcctttaattaaacactagttctatagtgtcacctaaattccctttagtgagggttaatggccgtaggccgccagaattgggtccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggactctaggacctgcgcatgcgcttggcgtaatcatggtcatagctgtttcctgttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctcaccgcggtggagctccagcttttgttcgaattggggccccccctcgagggtatcgatgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctagccttaagggcgcagcccgcctaatgagcgggcttttttttggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatccacgttgtggtagaattggtaaagagagtcgtgtaaaatatcgagttcgcacatcttgttgtctgattattgatttttggcgaaaccatttgatcatatgacaagatgtgtatctaccttaacttaatgattttgataaaaatcattaggta

SEQ ID NO: 95 - protein, 21 aa, synthetic construct
MALPVTALLLPLALLLHAARP

SEQ ID NO: 96 - DNA, 63 bp, synthetic construct
  source 1..63: mol_type = other DNA
atggctctgcctgtgacagctctgcttctgcctctggcactgcttcttcatgcggcgcgccct

SEQ ID NO: 97 - protein, 45 aa, synthetic construct
TTTPAPRPPTPAPTIASQPLSLRPEACRPAAGGAVHTRGLDFACD

SEQ ID NO: 98 - DNA, 135 bp, synthetic construct
  source 1..135: mol_type = other DNA
accacaacaccggcgcctagacctccaacaccagctcctacaatcgcgagtcagcccctgtctctcagacccgaagcctgcaggccagctgcaggaggagctgtgcacaccaggggcctggacttcgcctgcgac

SEQ ID NO: 99 - protein, 24 aa, synthetic construct
IYIWAPLAGTCGVLLLSLVITLYC

SEQ ID NO: 100 - DNA, 72 bp, synthetic construct
  source 1..72: mol_type = other DNA
atctacatttgggcaccactggccgggacctgtggagtgctgctgctgagcctggtcatcacactgtactgc

SEQ ID NO: 101 - protein, 42 aa, synthetic construct
KRGRKKLLYIFKQPFMRPVQTTQEEDGCSCRFPEEEEGGCEL

SEQ ID NO: 102 - DNA, 126 bp, synthetic construct
  source 1..126: mol_type = other DNA
aagagaggcaggaagaaactgctgtatattttcaaacagcccttcatgcgccccgtgcagactacccaggaggaagacgggtgctcctgtcgattccctgaggaagaggaaggcgggtgtgagctg

SEQ ID NO: 103 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - DNA, 336 bp, synthetic construct
  source 1..336: mol_type = other DNA
cgcgtgaagtttagtcgatcagcagatgccccagcttacaaacagggacagaaccagctgtataacgagctgaatctgggccgccgagaggaatatgacgtgctggataagcggagaggacgcgaccccgaaatgggaggcaagcccaggcgcaaaaaccctcaggaaggcctgtataacgagctgcagaaggacaaaatggcagaagcctattctgagatcggcatgaagggggagcgacggagaggcaaagggcacgatgggctgtaccagggactgagcaccgccacaaaggacacctatgatgctctgcatatgcaggcactgcctccaagg

SEQ ID NO: 105 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGYKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 106 - protein, 594 aa, synthetic construct
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 107 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 108 - DNA, 1818 bp, synthetic construct
  source 1..1818: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggatggacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctga

SEQ ID NO: 109 - protein, 605 aa, synthetic construct
MAPKKKRKVGGGGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRFDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 110 - DNA, 1818 bp, synthetic construct
  source 1..1818: mol_type = other DNA
atggctcccaagaagaagcggaaagttggcggcggaggcagcagcctggatgatgagcatattctgagcgccctgctgcagagcgacgatgaactcgtgggcgaagatagcgacagcgaggtgtccgatcacgtgtccgaggatgacgtgcagtccgataccgaggaagccttcatcgacgaggtgcacgaagtgcagcctacaagcagcggcagcgagatcctggacgagcagaatgtgatcgagcagccaggatctagcctggccagcaacagaatcctgacactgccccagagaaccatccggggcaagaacaagcactgctggtccaccagcaagagcaccagacggtctagagtgtctgccctgaacatcgtgcgaagccagaggggccctaccagaatgtgccggaacatctacgaccctctgctgtgcttcaagctgttcttcaccgacgagatcatctccgagatcgtgaagtggaccaacgccgagatcagcctgaagcggagagaatccatgaccagcgccaccttcagagacaccaacgaggacgagatctacgccttcttcggcatcctggtcatgacagccgtgcggaaggacaaccacatgagcaccgacgacctgttcgaccgcagcctgtctatggtgtacgtgtccgtgatgagccgggacagattcgacttcctgatccggtgcctgcggttcgacgacaagtccatcagacccacactgcgcgagaacgacgtgttcacacctgtgcggaagatctgggacctgttcatccaccagtgcatccagaactacacccctggcgctcacctgaccatcgacgaacagctgctgggcttcagaggcagatgccccttcagagtgtacatccccaacaagccctctaagtacggcatcaagatcctgatgatgtgcgacagcggcaccaagtacatgatcaacggcatgccctacctcggcagaggcacccaaacaaatggcgtgccactgggcgagtactacgtgaaagaactgagcaagcctgtgcacggcagctgcagaaacatcacctgtgacaactggtttaccagcattcccctggccaagaacctgctgcaagaaccctacaagctgacaatcgtgggcaccgtgcggagcaacaagagggaaattcccgaggtgctgaagaactctcggagcagacctgtgggcaccagcatgttctgcttcgacggacctctgacactggtgtcctacaagcccaagcctgccaagatggtgtacctgctgagcagctgtgacgaggacgccagcatcaatgagagcaccggcaagccccagatggtcatgtactacaaccagaccaaaggcggcgtggacaccctggatcagatgtgcagcgtgatgacctgcagcagaaagaccaacagatggcccatggctctgctgtacggcatgatcaatatcgcctgcatcaacagcttcatcatctacagccacaacgtgtccagcaagggcgagaaggtgcagagccggaagaaattcatgcggaacctgtacatgagcctgaccagcagcttcatgagaaagcggctggaagcccctacactgaagagatacctgcgggacaacatcagcaacatcctgcctaaagaggtgcccggcaccagcgacgatagcacagaggaacccgtgatgaagaagaggacctactgcacctactgtcccagcaagatccggcggaaggccaacgccagctgcaaaaagtgcaagaaagtgatctgccgcgagcacaacatcgatatgtgccagagctgcttctga

SEQ ID NO: 111 - protein, 340 aa, synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 112 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 113 - DNA, 28 bp, synthetic construct
  source 1..28: mol_type = other DNA
aaaaaaaaaaaaaaaaaaaaaaaaaaaa

SEQ ID NO: 114 - DNA, 10731 bp, synthetic construct
  source 1..10731: mol_type = other DNA
cctgaggaactctggggccaactaggttatcagaaccagtgtcccacacagctgtgacattcatgtccctcacctcatcagctcataagtgtgagtgaaggtcagggagggagggagagggtcagaaattacataacttctaccacactatcatccttattctgtgttatagttggtgacagttcctctcctactgtgattaagtcactaaagcaactgcaccatggaagtgcagacacagagaagtccagtggagacagcgatcagtgccctgctgtctgtggtctgcacaggggtctctcttcagtggacaagggggtctcctgtgctgaaacaatgtcccaggtccacagagacaaactcaggactcagaatgaagatccttgtttcaaatacacacacacacacacacacacacacacacacctgcagccacagactcttcatctaggaattaacacaaggaatctgtgtctcagcacagggctgagaagacagatcctgaggggagaggcgaagtctacacttaacagatgagtcctgcactcaggcttggcagtgtgagccacccattgcaggtgaacagagcctggtctctgtggggtccgtgtggggcttgcagcccagagcctcgactttaaggaaaagcctctctccactgcatccctaagcgcttgtgtcaccattgtattcccggaagaggcttttcttctagaagactccagggtctgacttctgaagagaagaaggaagaggaagagtggaagagaggacacagagagtctggcctgcgggtctctcctggtgttttgagagtttctggatcagaactcggagacgacagcacagggttcaggcaaagtcttagttgccaggcggtgaggtcaggggtggggaagcccagggctggggattccccatctccacagtttcacttctgctcctaacctgggtcaggtccttctgtccggacactgttgacgcgcagtcagctcttacccccattgggtggcgcgatcaccaagaaccaatcagtgtcgccgcggacgctggctataaagtccacgcagcccgcggaactcagaagtctcgaatcgccgaccggtgcgatggcaccctgcatgctgctcctgctgttggcggcagccctggccccgactcagacccgcgcgggcccacattcgttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaagcttatcgataccgtcgacctcgagggggggcccggtacccaattcgccctatagtgagtcgtattaagatctaattaaccctcactaaagggaacaaaagctggagctccaccgcggtggcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatcctagcagaagcacaggctgcagggtgacggtccatcccgctctcctgggcacaagacatgggcagcgtgccatcatcctgctcctccacctccggcgggaagccatggctaagcttcttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagaactagtggatcccccgggctgcaggaattcgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaggtatttccacaccgccgtgtcccggcccggcctcgggaagccccggttcatctctgtcggctacgtggacgacacgcagttcgtgcgcttcgacagcgacgcggagaatccgaggtatgagccgcgggtgcggtggatggagcaggtggagcccgagtattgggagcggaacacgcagatcgccaagggcaatgagcagattttccgagtgaacctgaggaccgcgctgcgctactacaaccagagcgcgggcggctctcacacgttccaacggatgtacggctgtgaggtggggtcggactggcgcctcctccgcgggtacgagcagtacgcatacgacggctgcgattacatcgccctgaacgaagacctgaaaacgtggacggcggccgacatggcggcgctgatcaccaaacacaagtgggagcaggctggtgatgcagagagagaccgggcctacctggagggcacgtgcgtggagtggctccgcagatacctgcagctcgggaacgcgacgctgccgcgcacagattccccaaaggcccatgtgacccgtcacagcagacctgaagataaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacacggtaatcattgctgttctggttgtccttggagctgcaatagtcactggagctgtggtggcttttgtgatgaagatgagaaggagaaacacaggtggaaaaggagggtagatccccgatccaataaaagatctttattttcattagatctgtgtgttggttttttgtgtggggtaccgaacatacgctctccatcaaaacaaaacgaaacaaaacaaactagcaaaataggctgtccccagtgcaagtgcaggtgccagaacatttctctagagatccaataaaagatccgaattggccgataagctagggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgaagcttcacctcttgaaggcgatataggccaccaggcccaccaccacggcggccaggatgctacagtacacggggatcaggttgtctgtggtgccccttgtcaccactggctgggagctgcccatcactgtggtcaccacgccggccacggtggaggcgatcaggtcctgctctggtggtgcctctggctcctgtgtagaaggtgcggtggaatcgctgccctcaggtggtgtggatctggtgatccaccggcctgggatctcctcgcactctgcatctgcccaccgggtacactccctgagctgcctctctgtgtcctcgcacacggtacagggcaggcaggggtccacgtgattggcctcatcggaatatgtgccgtcaggacactcctcgcacacggtgttctgcttgtcctggcagctaaacaccaggccagagcctgcctcgcacacccggcaggcctcacacctgcctgttgtctcatcctgatagtagccgtaggcacacctgcacacggcatcgtctgcctccacgcaaggggcagacatggactgcaggcccacgcactctgtacaaggcttgcagggctcggtggcggacaccacatcgctgaatgtcacagagtccagacaaggctcgcacacggtctgattggctccgcatggctgtgccactccctcgcccaggttacaggccttacagcactcgccggagtgtgtgtacaggccggtagggcaggcctccttggctccgcccaggctcacgcccagcagcagtaataacagcagtctgggtccgtccatagccctgccggttgccccggcccccatggtggctgatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgccaacatggcggtcatattggacatgagccaatataaatgtacatattatgatatagatacaacgtatgcaatggccaatagccaatattgatttatgctatataaccaatgaataatatggctaatggccaatattgctagcttatcggcccatgacattaacctataaaaataggcgtatcacgaggccctttcgtcttcaagaattggtcgatcgaccaattctcatgtttgacagcttatcatcggccaattcc

SEQ ID NO: 115 - DNA, 8920 bp, synthetic construct
  source 1..8920: mol_type = other DNA
tccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctcgtccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaaagcttggcaatccggtactgttggtaaagccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcaagcttatcgataccgtcgacctcgagggggggcccggtacccaattcgccctatagtgagtcgtattaagatctaattaaccctcactaaagggaacaaaagctggagctccaccgcggtggcggccgctctagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgctgcctgggcccccctgagatggtgcctcagcacggcagggctcactgattttttgggggtactgccaacaccttggaattcgcggccaccctcaaaggcatcaccgcgggccaggtgaatatcaaatcctcctcgtttttggaaactgacaatcttagcgcagaagtcatgcccgcttttgagagggagtactcaccccaacagctggcccttgcagatgagaccatggtgcatggcacatgggcgtcgagcggatgtatgctatacgaagttatccgggttgtggcgatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcgagaccatgcatcacacattgatgcgagctgaagcacaagcggctgggtgccggtccataccgctttcttgtgcgcaggacataggcaatgtaccggcggcttgctcttcaacctcaggcggaaagccgtgagaattcgccagaatgcgttcgcacagccgccagccggtcactccgttgatggttactcggaacagcagggagccgtcggggttgatcaggcgctcgtcgataattttgttgccgttccacagggtccctgttacagtgatctttttgccgtcgaacacggcgatgccttcatacggccgtccgaaatagtcgatcatgttcggcgtaaccccgtcgattaccagtgtgccatagtgcaggatcaccttaaagtgatgatcatccacagggtacaccaccttaaaaattttttcgatctggcccatttggtcgccgctcagaccttcatacgggatgatgacatggatgtcgatcttcagcccattttcaccgctcaggacaatcctttggatcggagttacggacaccccgagattctgaaacaaactggacacacctccctgttcaaggacttggtccaggttgtagccggctgtctgtcgccagtccccaacgaaatcttcgagtgtgaagaccatggtggccacacaggcctcacgacacctgaaatggaagaaaaaaactttgaaccactgtctgaggcttgagaatgaaccaagatccaaactcaaaaagggcaaattccaaggagaattacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacggcggcgcgaggccaggcaccagagcaggccggccagcttgagactacccccgtccgattctcggtggccgcgctcgcaggccccgcctcgccgaacatgtgcgctgggacgcacgggccccgtcgccgcccgcggccccaaaaaccgaaataccagtgtgcagatcttggcccgcatttacaagactatcttgccagaaaaaaagcgtcgcagcaggtcatcaaaaattttaaatggctagagacttatcgaaagcagcgagacaggcgcgaaggtgccaccagattcgcacgcggcggccccagcgcccaggccaggcctcaactcaagcacgaggcgaaggggctccttaagcgcaaggcctcgaactctcccacccacttccaacccgaagctcgggatcaagaatcacgtactgcagccaggtggaagtaattcaaggcacgcaagggccataacccgtaaagaggccaggcccgcgggaaccacacacggcacttacctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccactgacgggcaccggagccaattcccactcctttcaagacctagaaggtccattagctgcaaagattcctctctgtttaaaactttatccatctttgcaaagctggccttggaggccttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctctagaactagtggatcccccgggctgcaggaattcgataaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctggcctaactggccggtacctgagtct

SEQ ID NO: 116 - DNA, 5296 bp, synthetic construct
  source 1..5296: mol_type = other DNA
tcctatataataaaagagaaacatgcaaattgaccatccctccgctacgctcaagccacgcccaccagccaatcagaagtgactatgcaaattaacccaacaaagatggcagttaaatttgcatacgcaggtgtcaagcgccccaggaggcaacggcggccgcgggctcccaggaccttcgctggccccgggaggcgaggccggccgcgcctagccacacccgcgggctcccgggaccttcgccagcagagagcagagcgggagagcgggcggagagcgggaggtttggaggacttggcagagcaggaggccgctggacatagagcagagcgagagagagggtggcttggagggcgtggctccctctgtcaccccagcttcctcatcacagctgtggaaactgacagcagggaggaggaagtcccacccccacagaatcagccagaatcagccgttggtcagacagctctcagcggcctgacagccaggactctcattcacctgcatctcagaccgtgacagtagagaggtgggactatgtctaaagaacaactgttgatacaacgtagctctgcagccgaaagatgccggcgttatcgacagaaaatgtctgcagagcaacgtgcgtctgatcttgaaagaaggcggcgcctgcaacagaatgtatctgaagagcagctactggaaaaacgtcgctctgaagccgaaaaacagcggcgtcatcgacagaaaatgtctaaagaccaacgtgcctttgaagttgaaagaaggcggtggcgacgacagaatatgtctagagaacagtcatcaacaagtactaccaataccggtaggaactgccttctcagcaaaaatggagtacatgaggatgcaattctcgaacatagttgtggtggaatgactgttcgatgtgaattttgcctatcactaaatttctctgatgaaaaaccatccgatgggaaatttactcgatgttgtagcaaagggaaagtctgtccaaatgatatacattttccagattacccggcatatttaaaaagattaatgacaaacgaagattctgacagtaaaaatttcatggaaaatattcgttccataaatagttcttttgcttttgcttccatgggtgcaaatattgcatcgccatcaggatatgggccatactgttttagaatacacggacaagtttatcaccgtactggaactttacatccttcggatggtgtttctcggaagtttgctcaactctatattttggatacagccgaagctacaagtaaaagattagcaatgccagaaaaccagggctgctcagaaagactcatgatcaacatcaacaacctcatgcatgaaataaatgaattaacaaaatcgtacaagatgctacatgaggtagaaaaggaagcccaatctgaagcagcagcaaaaggtattgctcccacagaagtaacaatggcgattaaatacgatcgtaacagtgacccaggtagatataattctccccgtgtaaccgaggttgctgtcatattcagaaacgaagatggagaacctccttttgaaagggacttgctcattcattgtaaaccagatcccaataatccaaatgccactaaaatgaaacaaatcagtatcctgtttcctacattagatgcaatgacatatcctattctttttccacatggtgaaaaaggctggggaacagatattgcattaagactcagagacaacagtgtaatcgacaataatactagacaaaatgtaaggacacgagtcacacaaatgcagtattatggatttcatctctctgtgcgggacacgttcaatcctattttaaatgcaggaaaattaactcaacagtttattgtggattcatattcaaaaatggaggccaatcggataaatttcatcaaagcaaaccaatctaagttgagagttgaaaaatatagtggtttgatggattatctcaaatctagatctgaaaatgacaatgtgccgattggtaaaatgataatacttccatcatcttttgagggtagtcccagaaatatgcagcagcgatatcaggatgctatggcaattgtaacgaagtatggcaagcccgatttattcataaccatgacatgcaaccccaaatgggcagatattacaaacaatttacaacgctggcaaaaagttgaaaacagacctgacttggtagccagagtttttaatattaagctgaatgctcttttaaatgatatatgtaaattccatttatttggcaaagtaatagctaaaattcatgtcattgaatttcagaaacgcggactgcctcacgctcacatattattgatattagatagtgagtccaaattacgttcagaagatgacattgaccgtatagttaaggcagaaattccagatgaagaccagtgtcctcgactttttcaaattgtaaaatcaaatatggtacatggaccatgtggaatacaaaatccaaatagtccatgtatggaaaatggaaaatgttcaaagggatatccaaaagaatttcaaaatgcgaccattggaaatattgatggatatcccaaatacaaacgaagatctggtagcaccatgtctattggaaataaagttgtcgataacacttggattgtcccttataacccgtatttgtgccttaaatataactgtcatataaatgttgaagtctgtgcatcaattaaaagtgtcaaatatttatttaaatacatctataaagggcacgattgtgcaaatattcaaatttctgaaaaaaatattatcaatcatgacgaagtacaggacttcattgactccaggtatgtgagcgctcctgaggctgtttggagactttttgcaatgcgaatgcatgaccaatctcatgcaatcacaagattagctattcatttgccaaatgatcagaatttgtattttcataccgatgattttgctgaagttttagatagggctaaaaggcataactcgactttgatggcttggttcttattgaatagagaagattctgatgcacgtaattattattattgggagattccacagcattatgtgtttaataattctttgtggacaaaacgccgaaagggtgggaataaagtattaggtagactgttcactgtgagctttagagaaccagaacgatattaccttagacttttgcttctgcatgtaaaaggtgcgataagttttgaggatctgcgaactgtaggaggtgtaacttatgatacatttcatgaagctgctaaacaccgaggattattacttgatgacactatctggaaagatacgattgacgatgcaatcatccttaatatgcccaaacaactacggcaactttttgcatatatatgtgtgtttggatgtccttctgctgcagacaaattatgggatgagaataaatctcattttattgaagatttctgttggaaattacaccgaagagaaggtgcctgtgtgaactgtgaaatgcatgcccttaacgaaattcaggaggtattcacattgcatggaatgaaatgttcacatttcaaacttccggactatcctttattaatgaatgcaaatacatgtgatcaattgtacgagcaacaacaggcagaggttttgataaattctctgaatgatgaacagttggcagcctttcagactataacttcagccatcgaagatcaaactgtacaccccaaatgctttttcttggatggtccaggtggtagtggaaaaacatatctgtataaagttttaacacattatattagaggtcgtggtggtactgttttacccacagcatctacaggaattgctgcaaatttacttcttggtggaagaacctttcattcccaatataaattaccaattccattaaatgaaacttcaatttctagactcgatataaagagtgaagttgctaaaaccattaaaaaggcccaacttctcattattgatgaatgcaccatggcatccagtcatgctataaacgccatagatagattactaagagaaattatgaatttgaatgttgcatttggtgggaaagttctccttctcggaggggattttcgacaatgtctcagtattgtaccacatgctatgcgatcggccatagtacaaacgagtttaaagtactgtaatgtttggggatgtttcagaaagttgtctcttaaaacaaatatgagatcagaggattctgcttatagtgaatggttagtaaaacttggagatggcaaacttgatagcagttttcatttaggaatggatattattgaaatcccccatgaaatgatttgtaacggatctattattgaagctacctttggaaatagtatatctatagataatattaaaaatatatctaaacgtgcaattctttgtccaaaaaatgagcatgttcaaaaattaaatgaagaaattttggatatacttgatggagattttcacacatatttgagtgatgattccattgattcaacagatgatgctgaaaaggaaaattttcccatcgaatttcttaatagtattactccttcgggaatgccgtgtcataaattaaaattgaaagtgggtgcaatcatcatgctattgagaaatcttaatagtaaatggggtctttgtaatggtactagatttattatcaaaagattacgacctaacattatcgaagctgaagtattaacaggatctgcagagggagaggttgttctgattccaagaattgatttgtccccatctgacactggcctcccatttaaattaattcgaagacagtttcccgtgatgccagcatttgcgatgactattaataaatcacaaggacaaactctagacagagtaggaatattcctacctgaacccgttttcgcacatggtcagttatatgttgctttctctcgagttcgaagagcatgtgacgttaaagttaaagttgtaaatacttcatcacaagggaaattagtcaagcactctgaaagtgtttttactcttaatgtggtatacagggagatattagaataagtttaatcactttatcagtcattgtttgcatcaatgttgtttttatatcatgtttttgttgtttttatatcatgtctttgttgttgttatatcatgttgttattgtttatttattaataaatttatgtattattttcatatacattttactcatttcctttcatctctcacacttctattatagagaaagggcaaatagcaatattaaaatatttcctctaattaattccctttcaatgtgcacgaatttcgtgcaccgggccactag

SEQ ID NO: 117 - protein, 1496 aa, synthetic construct
MSKEQLLIQRSSAAERCRRYRQKMSAEQRASDLERRRRLQQNVSEEQLLEKRRSEAEKQRRHRQKMSKDQRAFEVERRRWRRQNMSREQSSTSTTNTGRNCLLSKNGVHEDAILEHSCGGMTVRCEFCLSLNFSDEKPSDGKFTRCCSKGKVCPNDIHFPDYPAYLKRLMTNEDSDSKNFMENIRSINSSFAFASMGANIASPSGYGPYCFRIHGQVYHRTGTLHPSDGVSRKFAQLYILDTAEATSKRLAMPENQGCSERLMININNLMHEINELTKSYKMLHEVEKEAQSEAAAKGIAPTEVTMAIKYDRNSDPGRYNSPRVTEVAVIFRNEDGEPPFERDLLIHCKPDPNNPNATKMKQISILFPTLDAMTYPILFPHGEKGWGTDIALRLRDNSVIDNNTRQNVRTRVTQMQYYGFHLSVRDTFNPILNAGKLTQQFIVDSYSKMEANRINFIKANQSKLRVEKYSGLMDYLKSRSENDNVPIGKMIILPSSFEGSPRNMQQRYQDAMAIVTKYGKPDLFITMTCNPKWADITNNLQRWQKVENRPDLVARVFNIKLNALLNDICKFHLFGKVIAKIHVIEFQKRGLPHAHILLILDSESKLRSEDDIDRIVKAEIPDEDQCPRLFQIVKSNMVHGPCGIQNPNSPCMENGKCSKGYPKEFQNATIGNIDGYPKYKRRSGSTMSIGNKVVDNTWIVPYNPYLCLKYNCHINVEVCASIKSVKYLFKYIYKGHDCANIQISEKNIINHDEVQDFIDSRYVSAPEAVWRLFAMRMHDQSHAITRLAIHLPNDQNLYFHTDDFAEVLDRAKRHNSTLMAWFLLNREDSDARNYYYWEIPQHYVFNNSLWTKRRKGGNKVLGRLFTVSFREPERYYLRLLLLHVKGAISFEDLRTVGGVTYDTFHEAAKHRGLLLDDTIWKDTIDDAIILNMPKQLRQLFAYICVFGCPSAADKLWDENKSHFIEDFCWKLHRREGACVNCEMHALNEIQEVFTLHGMKCSHFKLPDYPLLMNANTCDQLYEQQQAEVLINSLNDEQLAAFQTITSAIEDQTVHPKCFFLDGPGGSGKTYLYKVLTHYIRGRGGTVLPTASTGIAANLLLGGRTFHSQYKLPIPLNETSISRLDIKSEVAKTIKKAQLLIIDECTMASSHAINAIDRLLREIMNLNVAFGGKVLLLGGDFRQCLSIVPHAMRSAIVQTSLKYCNVWGCFRKLSLKTNMRSEDSAYSEWLVKLGDGKLDSSFHLGMDIIEIPHEMICNGSIIEATFGNSISIDNIKNISKRAILCPKNEHVQKLNEEILDILDGDFHTYLSDDSIDSTDDAEKENFPIEFLNSITPSGMPCHKLKLKVGAIIMLLRNLNSKWGLCNGTRFIIKRLRPNIIEAEVLTGSAEGEVVLIPRIDLSPSDTGLPFKLIRRQFPVMPAFAMTINKSQGQTLDRVGIFLPEPVFAHGQLYVAFSRVRRACDVKVKVVNTSSQGKLVKHSESVFTLNVVYREILE

SEQ ID NO: 118 - DNA, 4682 bp, synthetic construct
  source 1..4682: mol_type = other DNA
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttaaatgttatttattctgcctatgaaaatcgttttcacattatatgaaattggtcagacatgttcattggtcctttggaagtgacgtcatgtcacatctattaccacaatgcacagcaccttgacctggaaattagggaaattataacagtcaatcagtggaagaaaatggaggaagtatgtgattcatcagcagctgcgagcagcacagtccaaaatcagccacaggatcaagagcacccgtggccgtatcttcgcgaattcttttctttaagtggtgtaaataaagattcattcaagatgaaatgtgtcctctgtctcccgcttaataaagaaatatcggccttcaaaagttcgccatcaaacctaaggaagcatattgaggtaagtacattaagtattttgttttactgatagtttttttttttttttttttttttttttttgggtgtgcatgttttgacgttgatggcgcgccttttatatgtgtagtaggcctattttcactaatgcatgcgattgacaatataaggctcacgtaataaaatgctaaaatgcatttgtaattggtaacgttaggtccacgggaaatttggcgcctattgcagctttgaataatcattatcattccgtgctctcattgtgtttgaattcatgcaaaacacaagaaaaccaagcgagaaatttttttccaaacatgttgtattgtcaaaacggtaacactttacaatgaggttgattagttcatgtattaactaacattaaataaccatgagcaatacatttgttactgtatctgttaatctttgttaacgttagttaatagaaatacagatgttcattgtttgttcatgttagttcacagtgcattaactaatgttaacaagatataaagtattagtaaatgttgaaattaacatgtatacgtgcagttcattattagttcatgttaactaatgtagttaactaacgaaccttattgtaaaagtgttaccatcaaaactaatgtaatgaaatcaattcaccctgtcatgtcagccttacagtcctgtgtttttgtcaatataatcagaaataaaattaatgtttgattgtcactaaatgctactgtatttctaaaatcaacaagtatttaacattataaagtgtgcaattggctgcaaatgtcagttttattaaagggttagttcacccaaaaatgaaaataatgtcattaatgactcgccctcatgtcgttccaagcccgtaagacctccgttcatcttcagaacacagtttaagatattttagatttagtccgagagctttctgtgcctccattgagaatgtatgtacggtatactgtccatgtccagaaaggtaataaaaacatcaaagtagtccatgtgacatcagtgggttagttagaattttttgaagcatcgaatacattttggtccaaaaataacaaaacctacgactttattcggcattgtattctcttccgggtctgttgtcaatccgcgttcacgacttcgcagtgacgctacaatgctgaataaagtcgtaggttttgttatttttggaccaaaatgtattttcgatgcttcaaataattctacctaacccactgatgtcacatggactactttgatgtttttattacctttctggacatggacagtataccgtacatacattttcagtggagggacagaaagctctcggactaaatctaaaatatcttaaactgtgttccgaagatgaacggaggtgttacgggcttggaacgacatgagggtgagtcattaatgacatcttttcatttttgggtgaactaaccctttaatgctgtaatcagagagtgtatgtgtaattgttacatttattgcatacaatataaatatttatttgttgtttttacagagaatgcacccaaattacctcaaaaactactctaaattgacagcacagaagagaaagatcgggacctccacccatgcttccagcagtaagcaactgaaagttgactcagttttcccagtcaaacatgtgtctccagtcactgtgaacaaagctatattaaggtacatcattcaaggacttcatcctttcagcactgttgatctgccatcatttaaagagctgattagtacactgcagcctggcatttctgtcattacaaggcctactttacgctccaagatagctgaagctgctctgatcatgaaacagaaagtgactgctgccatgagtgaagttgaatggattgcaaccacaacggattgttggactgcacgtagaaagtcattcattggtgtaactgctcactggatcaaccctggaagtcttgaaagacattccgctgcacttgcctgcaaaagattaatgggctctcatacttttgaggtactggccagtgccatgaatgatatccactcagagtatgaaatacgtgacaaggttgtttgcacaaccacagacagtggttccaactttatgaaggctttcagagtttttggtgtggaaaacaatgatatcgagactgaggcaagaaggtgtgaaagtgatgacactgattctgaaggctgtggtgagggaagtgatggtgtggaattccaagatgcctcacgagtcctggaccaagacgatggcttcgaattccagctaccaaaacatcaaaagtgtgcctgtcacttacttaacctagtctcaagcgttgatgcccaaaaagctctctcaaatgaacactacaagaaactctacagatctgtctttggcaaatgccaagctttatggaataaaagcagccgatcggctctagcagctgaagctgttgaatcagaaagccggcttcagcttttaaggccaaaccaaacgcggtggaattcaacttttatggctgttgacagaattcttcaaatttgcaaagaagcaggagaaggcgcacttcggaatatatgcacctctcttgaggttccaatgtaagtgtttttcccctctatcgatgtaaacaaatgtgggttgtttttgtttaatactctttgattatgctgatttctcctgtaggtttaatccagcagaaatgctgttcttgacagagtgggccaacacaatgcgtccagttgcaaaagtactcgacatcttgcaagcggaaacgaatacacagctggggtggctgctgcctagtgtccatcagttaagcttgaaacttcagcgactccaccattctctcaggtactgtgacccacttgtggatgccctacaacaaggaatccaaacacgattcaagcatatgtttgaagatcctgagatcatagcagctgccatccttctccctaaatttcggacctcttggacaaatgatgaaaccatcataaaacgaggtaaatgaatgcaagcaacatacacttgacgaattctaatctgggcaacctttgagccataccaaaattattcttttatttatttatttttgcactttttaggaatgttatatcccatctttggctgtgatctcaatatgaatattgatgtaaagtattcttgcagcaggttgtagttatccctcagtgtttcttgaaaccaaactcatatgtatcatatgtggtttggaaatgcagttagattttatgctaaaataagggatttgcatgattttagatgtagatgactgcacgtaaatgtagttaatgacaaaatccataaaatttgttcccagtcagaagcccctcaaccaaacttttctttgtgtctgctcactgtgcttgtaggcatggactacatcagagtgcatctggagcctttggaccacaagaaggaattggccaacagttcatctgatgatgaagattttttcgcttctttgaaaccgacaacacatgaagccagcaaagagttggatggatatctggcctgtgtttcagacaccagggagtctctgctcacgtttcctgctatttgcagcctctctatcaagactaatacacctcttcccgcatcggctgcctgtgagaggcttttcagcactgcaggattgcttttcagccccaaaagagctaggcttgacactaacaattttgagaatcagcttctactgaagttaaatctgaggttttacaactttgagtagcgtgtactggcattagattgtctgtcttatagtttgataattaaatacaaacagttctaaagcaggataaaaccttgtatgcatttcatttaatgttttttgagattaaaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 119 - protein, 649 aa, synthetic construct
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 120 - protein, 636 aa, synthetic construct
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLENAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 121 - DNA, 1911 bp, synthetic construct
  source 1..1911: mol_type = other DNA
atgatgttgaattggctgaaaagtggaaagcttgaaagtcaatcacaggaacagagttcctgctaccttgagaactctaactgcctgccaccaacgctcgattctacagatattatcggtgaagagaacaaagctggtaccacctctcgcaagaagcggaaatatgacgaggactatctgaacttcggttttacatggactggcgacaaggatgagcccaacggactttgtgtgatttgcgagcaggtagtcaacaattcctcacttaacccggccaaactgaaacgccatttggacacaaagcatccgacgcttaaaggcaagagcgaatacttcaaaagaaaatgtaacgagctcaatcaaaagaagcatacttttgagcgatacgtaagggacgataacaagaacctcctgaaagcttcttatctcgtcagtttgagaatagctaaacagggcgaggcatataccatagcggagaagttgatcaagccttgcaccaaggatctgacaacttgcgtatttggagaaaaattcgcgagcaaagttgatctcgtccccctgtccgacacgactatttcaaggcgaatcgaagacatgagttacttctgtgaagccgtgctggtgaacaggttgaaaaatgctaaatgtgggtttacgctgcagatggacgagtcaacagatgttgccggtcttgcaatcctgcttgtgtttgttaggtacatacatgaaagctcttttgaggaggatatgttgttctgcaaagcacttcccactcagacgacaggggaggagattttcaatcttctcaatgcctatttcgaaaagcactccatcccatggaatctgtgttaccacatttgcacagacggtgccaaggcaatggtaggagttattaaaggagtcatagcgagaataaaaaaactcgtccctgatataaaagctagccactgttgcctgcatcgccacgctttggctgtaaagcgaataccgaatgcattgcacgaggtgctcaatgacgctgttaaaatgatcaacttcatcaagtctcggccgttgaatgcgcgcgtcttcgctttgctgtgtgacgatttggggagcctgcataaaaatcttcttcttcataccgaagtgaggtggctgtctagaggaaaggtgctgacccgattttgggaactgagagatgaaattagaattttcttcaacgaaagggaatttgccgggaaattgaacgacaccagttggttgcaaaatttggcatatatagctgacatattcagttatctgaatgaagttaatctttccctgcaagggccgaatagcacaatcttcaaggtaaatagccgcattaacagtattaaatcaaagttgaagttgtgggaagagtgtataacgaaaaataacactgagtgttttgcgaacctcaacgattttttggaaacttcaaacactgcgttggatccaaacctgaagtctaatattttggaacatctcaacggtcttaagaacacctttctggagtattttccacctacgtgtaataatatctcctgggtggagaatcctttcaatgaatgcggtaacgtcgatacactcccaataaaagagagggaacaattgattgacatacggactgatacgacattgaaatcttcattcgtgcctgatggtataggaccattctggatcaaactgatggacgaatttccagaaattagcaaacgagctgtcaaagagctcatgccatttgtaaccacttacctctgtgagaaatcattttccgtctatgtagccacaaaaacaaaatatcgaaatagacttgatgctgaagacgatatgcgactccaacttactactatccatccagacattgacaacctttgtaacaacaagcaggctcagaaatcccactga

SEQ ID NO: 122 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 123 - protein, 1053 aa, synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 124 - protein, 1368 aa, synthetic construct
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 125 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 126 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 127 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 128 - DNA, 4776 bp, synthetic construct
  source 1..4776: mol_type = other DNA
atggcaccaaagaagaaaagaaaagtggagggcatcaagtcaaacatcagcctgctgaaagacgaactgcggggacagattagtcacatcagtcacgagtacctgtcactgattgatctggccttcgacagcaagcagaatagactgtttgagatgaaagtgctggaactgctggtcaacgagtatggcttcaagggcagacatctgggcgggtctaggaaacctgacggcatcgtgtacagtaccacactggaagacaacttcggaatcattgtcgataccaaggcttattccgagggctactctctgccaattagtcaggcagatgagatggaaaggtacgtgcgcgaaaactcaaatagggacgaggaagtcaaccccaataagtggtgggagaatttcagcgaggaagtgaagaaatactacttcgtctttatctcaggcagcttcaaagggaagtttgaggaacagctgcggagactgtccatgactaccggggtgaacggatctgctgtcaacgtggtcaatctgctgctgggcgcagaaaagatcaggtccggggagatgacaattgaggaactggaacgcgccatgttcaacaattctgagtttatcctgaagtatggaggcgggggaagcgataagaaatactccatcggactggccattggcaccaattccgtgggctgggctgtcatcacagacgagtacaaggtgccaagcaagaagttcaaggtcctggggaacaccgatcgccacagtatcaagaaaaatctgattggagccctgctgttcgactcaggcgagactgctgaagcaacccgactgaagcggactgctaggcgccgatatacccggagaaaaaatcggatctgctacctgcaggaaattttcagcaacgagatggccaaggtggacgatagtttctttcaccgcctggaggaatcattcctggtggaggaagataagaaacacgagcggcatcccatctttggcaacattgtggacgaagtcgcttatcacgagaagtaccctactatctatcatctgaggaagaaactggtggactccaccgataaggcagacctgcgcctgatctatctggccctggctcacatgatcaagttccgggggcattttctgatcgagggagatctgaaccctgacaattctgatgtggacaagctgttcatccagctggtccagacatacaatcagctgtttgaggaaaacccaattaatgcctcaggcgtggacgcaaaggccatcctgagcgccagactgtccaaatctaggcgcctggaaaacctgatcgctcagctgccaggagagaagaaaaacggcctgtttgggaatctgattgcactgtccctgggcctgacacccaacttcaagtctaattttgatctggccgaggacgctaagctgcagctgtccaaagacacttatgacgatgacctggataacctgctggctcagatcggcgatcagtacgcagacctgttcctggccgctaagaatctgagtgacgccatcctgctgtcagatattctgcgcgtgaacacagagattactaaggccccactgagtgcttcaatgatcaaaagatatgacgagcaccatcaggatctgaccctgctgaaggctctggtgaggcagcagctgcccgagaaatacaaggaaatcttctttgatcagagcaagaatggatacgccggctatattgacggcggggcttcccaggaggagttctacaagttcatcaagcccattctggaaaagatggacggcaccgaggaactgctggtgaagctgaatcgggaggacctgctgagaaaacagaggacatttgataacggaagcatccctcaccagattcatctgggcgaactgcacgccatcctgcgacggcaggaggacttctacccatttctgaaggataaccgcgagaaaatcgaaaagatcctgaccttcagaatcccctactatgtggggcctctggcacggggaaatagtagatttgcctggatgacaagaaagtcagaggaaactatcaccccctggaacttcgaggaagtggtcgataaaggcgctagcgcacagtccttcattgaaaggatgacaaattttgacaagaacctgccaaatgagaaggtgctgcccaaacacagcctgctgtacgaatatttcacagtgtataacgagctgactaaagtgaagtacgtcaccgaagggatgcgcaagcccgcattcctgtccggagagcagaagaaagccatcgtggacctgctgtttaagacaaatcggaaagtgactgtcaaacagctgaaggaagactatttcaagaaaattgagtgtttcgattcagtggaaatcagcggcgtcgaggacaggtttaacgcctccctggggacctaccacgatctgctgaagatcatcaaggataaggacttcctggacaacgaggaaaatgaggacatcctggaggacattgtgctgacactgactctgtttgaggatcgcgaaatgatcgaggaacgactgaagacttatgcccatctgttcgatgacaaagtgatgaagcagctgaaaagaaggcgctacaccggatggggacgcctgagccgaaaactgatcaatgggattagagacaagcagagcggaaaaactatcctggactttctgaagtccgatggcttcgccaacaggaacttcatgcagctgattcacgatgactctctgaccttcaaggaggacatccagaaagcacaggtgtctggccagggggacagtctgcacgagcatatcgcaaacctggccggcagccccgccatcaagaaagggattctgcagaccgtgaaggtggtggacgaactggtcaaggtcatgggacgacacaaacctgagaacatcgtgattgagatggcccgcgaaaatcagacaactcagaagggccagaaaaacagtcgagaacggatgaagagaatcgaggaaggcatcaaggagctggggtcacagatcctgaaggagcatcctgtggaaaacactcagctgcagaatgagaaactgtatctgtactatctgcagaatggacgggatatgtacgtggaccaggagctggatattaacagactgagtgattatgacgtggatgccatcgtccctcagagcttcctgaaggatgactccattgacaacaaggtgctgaccaggtccgacaagaaccgcggcaaatcagataatgtgccaagcgaggaagtggtcaagaaaatgaagaactactggaggcagctgctgaatgccaagctgatcacacagcggaaatttgataacctgactaaggcagaaagaggaggcctgtctgagctggacaaggccggcttcatcaagcggcagctggtggagacaagacagatcactaagcacgtcgctcagattctggatagcagaatgaacacaaagtacgatgaaaacgacaagctgatcagggaggtgaaagtcattactctgaaatccaagctggtgtctgactttagaaaggatttccagttttataaagtcagggagatcaacaactaccaccatgctcatgacgcatacctgaacgcagtggtcgggaccgccctgattaagaaataccccaagctggagtccgagttcgtgtacggagactataaagtgtacgatgtccggaagatgatcgccaaatctgagcaggaaattggcaaggccaccgctaagtatttcttttacagtaacatcatgaatttctttaagaccgaaatcacactggcaaatggggagatcagaaaaaggcctctgattgagaccaacggggagacaggagaaatcgtgtgggacaagggaagggattttgctaccgtgcgcaaagtcctgtccatgccccaagtgaatattgtcaagaaaactgaagtgcagaccgggggattctctaaggagagtattctgcctaagcgaaactctgataaactgatcgcccggaagaaagactgggaccccaagaagtatggcgggttcgactctccaacagtggcttacagtgtcctggtggtcgcaaaggtggaaaaggggaagtccaagaaactgaagtctgtcaaagagctgctgggaatcactattatggaacgcagctccttcgagaagaatcctatcgattttctggaagccaagggctataaagaggtgaagaaagacctgatcattaagctgccaaaatactcactgtttgagctggaaaacggacgaaagcgaatgctggcaagcgccggagaactgcagaagggcaatgagctggccctgccctccaaatacgtgaacttcctgtatctggctagccactacgagaaactgaaggggtcccctgaggataacgaacagaagcagctgtttgtggagcagcacaaacattatctggacgagatcattgaacagatttcagagttcagcaagagagtgatcctggctgacgcaaatctggataaagtcctgagcgcatacaacaagcaccgagacaaaccaatccgggagcaggccgaaaatatcattcatctgttcaccctgacaaacctgggcgcccctgcagccttcaagtattttgacaccacaatcgatcggaagagatacacttctaccaaagaggtgctggatgctaccctgatccaccagagtattaccggcctgtatgagacacgcatcgacctgtcacagctgggaggcgatgggagccccaagaaaaagcggaaggtgtctagttaa

SEQ ID NO: 129 - protein, 1588 aa, synthetic construct
MPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKV

SEQ ID NO: 130 - DNA, 4767 bp, synthetic construct
  source 1..4767: mol_type = other DNA
atgcctaagaagaagcggaaggtggaaggcatcaaaagcaacatctccctcctgaaagacgaactccgggggcagattagccacattagtcacgaatacctctccctcatcgacctggctttcgatagcaagcagaacaggctctttgagatgaaagtgctggaactgctcgtcaatgagtacgggttcaagggtcgacacctcggcggatctaggaaaccagacggcatcgtgtatagtaccacactggaagacaactttgggatcattgtggataccaaggcatactctgagggttatagtctgcccatttcacaggccgacgagatggaacggtacgtgcgcgagaactcaaatagagatgaggaagtcaaccctaacaagtggtgggagaacttctctgaggaagtgaagaaatactacttcgtctttatcagcgggtccttcaagggtaaatttgaggaacagctcaggagactgagcatgactaccggcgtgaatggcagcgccgtcaacgtggtcaatctgctcctgggcgctgaaaagattcggagcggagagatgaccatcgaagagctggagagggcaatgtttaataatagcgagtttatcctgaaatacggtggcggtggatccgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatgccattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgacggatcccccaagaagaagaggaaagtctga

SEQ ID NO: 131 - protein, 107 aa, synthetic construct
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 132 - DNA, 321 bp, synthetic construct
  source 1..321: mol_type = other DNA
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggag

SEQ ID NO: 133 - protein, 5 aa, synthetic construct
GGGGS

SEQ ID NO: 134 - DNA, 15 bp, synthetic construct
  source 1..15: mol_type = other DNA
ggaggaggaggatcc

SEQ ID NO: 135 - protein, 282 aa, synthetic construct
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 136 - DNA, 843 bp, synthetic construct
  source 1..843: mol_type = other DNA
tttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 137 - protein, 394 aa, synthetic construct
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 138 - DNA, 1182 bp, synthetic construct
  source 1..1182: mol_type = other DNA
ggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 139 - DNA, 1185 bp, synthetic construct
  source 1..1185: mol_type = other DNA
atgggggtccaggtcgagactatttcaccaggggatgggcgaacatttccaaaaaggggccagacttgcgtcgtgcattacaccgggatgctggaggacgggaagaaagtggacagctccagggatcgcaacaagcccttcaagttcatgctgggaaagcaggaagtgatccgaggatgggaggaaggcgtggcacagatgtcagtcggccagcgggccaaactgaccattagccctgactacgcttatggagcaacaggccacccagggatcattccccctcatgccaccctggtcttcgatgtggaactgctgaagctggagggaggaggaggatccggatttggggacgtgggggccctggagtctctgcgaggaaatgccgatctggcttacatcctgagcatggaaccctgcggccactgtctgatcattaacaatgtgaacttctgcagagaaagcggactgcgaacacggactggctccaatattgactgtgagaagctgcggagaaggttctctagtctgcactttatggtcgaagtgaaaggggatctgaccgccaagaaaatggtgctggccctgctggagctggctcagcaggaccatggagctctggattgctgcgtggtcgtgatcctgtcccacgggtgccaggcttctcatctgcagttccccggagcagtgtacggaacagacggctgtcctgtcagcgtggagaagatcgtcaacatcttcaacggcacttcttgccctagtctggggggaaagccaaaactgttctttatccaggcctgtggcggggaacagaaagatcacggcttcgaggtggccagcaccagccctgaggacgaatcaccagggagcaaccctgaaccagatgcaactccattccaggagggactgaggacctttgaccagctggatgctatctcaagcctgcccactcctagtgacattttcgtgtcttacagtaccttcccaggctttgtctcatggcgcgatcccaagtcagggagctggtacgtggagacactggacgacatctttgaacagtgggcccattcagaggacctgcagagcctgctgctgcgagtggcaaacgctgtctctgtgaagggcatctacaaacagatgcccgggtgcttcaattttctgagaaagaaactgttctttaagacttcc

SEQ ID NO: 140 - DNA, 1185 bp, synthetic construct; identical to SEQ ID NO: 139
  source 1..1185: mol_type = other DNA

SEQ ID NO: 141 - protein, 463 aa, synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 184 aa, synthetic construct
KEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGL

SEQ ID NO: 143 - protein, 463 aa, synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYHTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - DNA, 375 bp, synthetic construct
  source 1..375: mol_type = other DNA
ttatcccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatgatttactgtatcggttgtctttcgttcctttagttgagtttttttttattattttcagtttttgatcaaa

SEQ ID NO: 145 - DNA, 368 bp, synthetic construct
  source 1..368: mol_type = other DNA
tcatatttttagtttaaaaaaataattatatgttttataatgaaaagaatctcattatctttcagtattaggttgatttatattccaaagaataatatttttgttaaattgttgatttttgtaaacctctaaatgtttgttgctaaaattactgtgtttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccgggttat

SEQ ID NO: 146 - DNA, 214 bp, synthetic construct
  source 1..214: mol_type = other DNA
ttatcccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcat

SEQ ID NO: 147 - DNA, 195 bp, synthetic construct
  source 1..195: mol_type = other DNA
taaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccgggttat

SEQ ID NO: 148 - DNA, 375 bp, synthetic construct
  source 1..375: mol_type = other DNA
ttaacccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatgatttactgtatcggttgtctttcgttcctttagttgagtttttttttattattttcagtttttgatcaaa

SEQ ID NO: 149 - DNA, 797 bp, synthetic construct
  source 1..797: mol_type = other DNA
tcatatttttagtttaaaaaaataattatatgttttataatgaaaagaatctcattatctttcagtattaggttgatttatattccaaagaataatatttttgttaaattgttgatttttgtaaacctctaaatgtttgttgctaaaattactgtgtttaagaaaaagattaataaataataataatttcataattaaaaacttctttcattgaatgccattaaataattcattattttacaaaataagatcaacataattgagtaaataataataagaacaatattatagtacaacaaaatatgggtatgtcataccctttttttttttttttttttttttttttcgggtagagggccgaacctcctacgaggtccccgcgcaaaaggggcgcgcggggtatgtgagactcaacgatctgcatggtgttgtgagcagaccgcgggcccaaggattttagagcccacccactaaacgactcctctgcactcttacacccgacgtccgatcccctccgaggtcagaacccggatgaggtaggggggctaccgcggtcaacactacaaccagacggcgcggctcaccccaaggacgcccagccgacggagccttcgaggcgaatcgaaggctctgaaacgtcggccgtctcggtacggcagcccgtcgggccgcccagacggtgccgctggtgtcccggaataccccgctggaccagaaccagcctgccgggtcgggacgcgatacaccgtcgaccggtcgctctaatcactccacggcagcgcgctagagtgctggta

SEQ ID NO: 150 - DNA, 221 bp, synthetic construct
  source 1..221: mol_type = other DNA
cccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtat

SEQ ID NO: 151 - DNA, 208 bp, synthetic construct
  source 1..208: mol_type = other DNA
tttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccggg

SEQ ID NO: 152 - DNA, 371 bp, synthetic construct
  source 1..371: mol_type = other DNA
cccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatgatttactgtatcggttgtctttcgttcctttagttgagtttttttttattattttcagtttttgatcaaa

SEQ ID NO: 153 - DNA, 364 bp, synthetic construct
  source 1..364: mol_type = other DNA
tcatatttttagtttaaaaaaataattatatgttttataatgaaaagaatctcattatctttcagtattaggttgatttatattccaaagaataatatttttgttaaattgttgatttttgtaaacctctaaatgtttgttgctaaaattactgtgtttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttcacctcatgctcgccggg

SEQ ID NO: 154 - DNA, 301 bp, synthetic construct
  source 1..301: mol_type = other DNA
cccggcgagcatgaggcagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatg

SEQ ID NO: 155 - DNA, 285 bp, synthetic construct
  source 1..285: mol_type = other DNA
cagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtattttttttatgtaatttttccgattattaatttcaactgttttattggtatttttatgttatccattgttctttttttatg

SEQ ID NO: 156 - DNA, 205 bp, synthetic construct
  source 1..205: mol_type = other DNA
cagggtatctcataccctggtaaaattttaaagttgtgtattttataaaattttcgtctgacaacactagcgcgctcagtagctggaggcaggagcgtgcgggaggggatagtggcgtgatcgcagtgtggcacgggacaccggcgagatattcgtgtgcaaacctgtttcgggtatgttataccctgcctcattgttgacgtat

SEQ ID NO: 157 - skipped

SEQ ID NO: 158 - DNA, 192 bp, synthetic construct
  source 1..192: mol_type = other DNA
tttaagaaaaagattaataaataataataatttcataattaaaaacttctttcattgaatgccattaaataaaccattattttacaaaataagatcaacataattgagtaaataataataagaacaatattatagtacaacaaaatatgggtatgtcataccctgccacattcttgatgtaactttttttca

SEQ ID NO: 159 - skipped

SEQ ID NO: 160 - DNA, 35 bp, synthetic construct
  source 1..35: mol_type = other DNA
ccctagaaagatagtctgcgtaaaattgacttctg

SEQ ID NO: 161 - DNA, 35 bp, synthetic construct
  source 1..35: mol_type = other DNA
ccctagaaaaacagtctgcgtaaaattgacgcatg

SEQ ID NO: 162 - DNA, 35 bp, synthetic construct
  source 1..35: mol_type = other DNA
ccctagaaagatagcatgcgtaaaattgacgcatg

SEQ ID NO: 163 - DNA, 35 bp, synthetic construct
  source 1..35: mol_type = other DNA
ccctagtacgatagtctgcgtaaaattgacgcatg

SEQ ID NO: 164 - skipped

SEQ ID NO: 165 - DNA, 35 bp, synthetic construct
  source 1..35: mol_type = other DNA
ccctagaaagatagtctgcgtaaaattgacctctg

SEQ ID NO: 166 - DNA, 35 bp, synthetic construct
  source 1..35: mol_type = other DNA
ccctagtccgatagtctgcgtaaaattgacgcatg

SEQ ID NO: 167 - DNA, 35 bp, synthetic construct
  source 1..35: mol_type = other DNA
ccctagaaagatagtctgcgtaaaattgatctatg

SEQ ID NO: 168 - DNA, 35 bp, synthetic construct
  source 1..35: mol_type = other DNA
ccctagaaagatagtctgcgtaaaattgacttatg

SEQ ID NO: 169 - DNA, 35 bp, synthetic construct
  source 1..35: mol_type = other DNA
ccctagaaagatagtctgcgtaaaattggctcatg

SEQ ID NO: 170 - DNA, 35 bp, synthetic construct
  source 1..35: mol_type = other DNA
ccctagaaagatagtctgcgtaaaattgacgcatc

SEQ ID NO: 171 - DNA, 35 bp, synthetic construct
  source 1..35: mol_type = other DNA
ccctagcatgatagtctgcgtaaaattgacgcatg

SEQ ID NO: 172 - DNA, 35 bp, synthetic construct
  source 1..35: mol_type = other DNA
ccctagaaagatagtctgcgtaaaattgacgcgcc

SEQ ID NO: 173 - DNA, 35 bp, synthetic construct
  source 1..35: mol_type = other DNA
ccctagaaagaccatctgcgtaaaattgacgcatg

SEQ ID NO: 174 - DNA, 35 bp, synthetic construct
  source 1..35: mol_type = other DNA
ccctagaaagatcaactgcgtaaaattgacgcatg

SEQ ID NO: 175 - DNA, 35 bp, synthetic construct
  source 1..35: mol_type = other DNA
ccctagaaagatcttctgcgtaaaattgacgcatg

SEQ ID NO: 176 - DNA, 35 bp, synthetic construct
  source 1..35: mol_type = other DNA
ccctagtcagatagtctgcgtaaaattgacgcatg

SEQ ID NO: 177 - DNA, 35 bp, synthetic construct
  source 1..35: mol_type = other DNA
ccctagaaagatagtctgcgtaaaattggtgcatg

SEQ ID NO: 178 - DNA, 35 bp, synthetic construct
  source 1..35: mol_type = other DNA
ccctagaaagatagtctgcgtaaaattgtcacatg

SEQ ID NO: 179 - DNA, 35 bp, synthetic construct
  source 1..35: mol_type = other DNA
ccctagaaagatagtctgcgtaaaattgacgccct

SEQ ID NO: 180 - DNA, 35 bp, synthetic construct
  source 1..35: mol_type = other DNA
ccctagaaagggggtctgcgtaaaattgacgcatg

SEQ ID NO: 181 - DNA, 35 bp, synthetic construct
  source 1..35: mol_type = other DNA
ccctcagaagatagtctgcgtaaaattgacgcatg

SEQ ID NO: 182 - DNA, 35 bp, synthetic construct
  source 1..35: mol_type = other DNA
ccctagaaagatttactgcgtaaaattgacgcatg

SEQ ID NO: 183 - DNA, 35 bp, synthetic construct
  source 1..35: mol_type = other DNA
ccctagaaagatacaatgcgtaaaattgacgcatg

SEQ ID NO: 184 - DNA, 35 bp, synthetic construct
  source 1..35: mol_type = other DNA
ccctagagcaatagtctgcgtaaaattgacgcatg

SEQ ID NO: 185 - DNA, 35 bp, synthetic construct
  source 1..35: mol_type = other DNA
ccctagaaagatagtctgcgtaaaattgtaccatg

SEQ ID NO: 186 - DNA, 35 bp, synthetic construct; identical to SEQ ID NO: 170
  source 1..35: mol_type = other DNA

SEQ ID NO: 187 - DNA, 35 bp, synthetic construct
  source 1..35: mol_type = other DNA
ccctagaaagatagtatacgtaaaattgacgcatg

SEQ ID NO: 188 - DNA, 35 bp, synthetic construct
  source 1..35: mol_type = other DNA
ccctagaaagatagtgtacgtaaaattgacgcatg

SEQ ID NO: 189 - DNA, 35 bp, synthetic construct
  source 1..35: mol_type = other DNA
ccctagaaacccagtctgcgtaaaattgacgcatg

SEQ ID NO: 190 - DNA, 35 bp, synthetic construct
  source 1..35: mol_type = other DNA
ccctagaaatacagtctgcgtaaaattgacgcatg

SEQ ID NO: 191 - DNA, 35 bp, synthetic construct
  source 1..35: mol_type = other DNA
ccctagaaagatagtctgcgtaaaattgacgtcgg

SEQ ID NO: 192 - DNA, 35 bp, synthetic construct
  source 1..35: mol_type = other DNA
ccctagaaagatagtctgcgtaaaattgacgcacc

SEQ ID NO: 193 - DNA, 35 bp, synthetic construct
  source 1..35: mol_type = other DNA
ccctagaaagatagtctgcgtaaatctgacgcatg

SEQ ID NO: 194 - DNA, 35 bp, synthetic construct
  source 1..35: mol_type = other DNA
ccctagctagatagtctgcgtaaaattgacgcatg

SEQ ID NO: 195 - DNA, 35 bp, synthetic construct
  source 1..35: mol_type = other DNA
ccctagaacgttagtctgcgtaaaattgacgcatg

SEQ ID NO: 196 - DNA, 35 bp, synthetic construct
  source 1..35: mol_type = other DNA
ccctagaaagatagtctgcgtaatcttgacgcatg

SEQ ID NO: 197 - DNA, 35 bp, synthetic construct
  source 1..35: mol_type = other DNA
ccctagaaagatagtctgcgtaaaattgacgggcg

SEQ ID NO: 198 - DNA, 35 bp, synthetic construct
  source 1..35: mol_type = other DNA
ccctagaaagatagtctgcgtaaaattgacggggg

SEQ ID NO: 199 - DNA, 35 bp, synthetic construct
  source 1..35: mol_type = other DNA
cccttaaaagatagtctgcgtaaaattgacgcatg

SEQ ID NO: 200 - DNA, 35 bp, synthetic construct
  source 1..35: mol_type = other DNA
ccctagaaagatacagtgcgtaaaattgacgcatg

SEQ ID NO: 201 - DNA, 35 bp, synthetic construct
  source 1..35: mol_type = other DNA
ccctcgcaagatagtctgcgtaaaattgacgcatg

SEQ ID NO: 202 - DNA, 35 bp, synthetic construct
  source 1..35: mol_type = other DNA
ccctagaaagatagtctgcgtaaaatctgcgcatg

SEQ ID NO: 203 - DNA, 35 bp, synthetic construct
  source 1..35: mol_type = other DNA
ccctagaaagatagtctgcgtaattgtgacgcatg

SEQ ID NO: 204 - DNA, 35 bp, synthetic construct
  source 1..35: mol_type = other DNA
ccctagaaagaagatctgcgtaaaattgacgcatg

SEQ ID NO: 205 - DNA, 35 bp, synthetic construct; identical to SEQ ID NO: 4
  source 1..35: mol_type = other DNA

SEQ ID NO: 206 - DNA, 35 bp, synthetic construct
  source 1..35: mol_type = other DNA
ccctagaaagatagtctgcgtaaagcggacgcatg

SEQ ID NO: 207 - DNA, 35 bp, synthetic construct
  source 1..35: mol_type = other DNA
ccctagaaagatagtctgcgtaaaattgacgcaga

SEQ ID NO: 208 - DNA, 35 bp, synthetic construct
  source 1..35: mol_type = other DNA
ccctagaaagtaggtctgcgtaaaattgacgcatg

SEQ ID NO: 209 - DNA, 35 bp, synthetic construct
  source 1..35: mol_type = other DNA
cccctgaaagatagtctgcgtaaaattgacgcatg

SEQ ID NO: 210 - DNA, 35 bp, synthetic construct
  source 1..35: mol_type = other DNA
ccctagaggtatagtctgcgtaaaattgacgcatg

SEQ ID NO: 211 - DNA, 35 bp, synthetic construct
  source 1..35: mol_type = other DNA
ccctagaaagatagtctgcgtaaaactcacgcatg

SEQ ID NO: 212 - DNA, 35 bp, synthetic construct
  source 1..35: mol_type = other DNA
ccctaagcagatagtctgcgtaaaattgacgcatg

SEQ ID NO: 213 - DNA, 35 bp, synthetic construct
  source 1..35: mol_type = other DNA
ccctccaaagatagtctgcgtaaaattgacgcatg

SEQ ID NO: 214 - DNA, 35 bp, synthetic construct
  source 1..35: mol_type = other DNA
ccctagaaagatagtctgcgtaaattcgacgcatg

SEQ ID NO: 215 - DNA, 35 bp, synthetic construct
  source 1..35: mol_type = other DNA
ccccagaaagatagtctgcgtaaaattgacgcatg

SEQ ID NO: 216 - DNA, 35 bp, synthetic construct
  source 1..35: mol_type = other DNA
ccctagaaagatagtctgcgtaaaagccacgcatg

SEQ ID NO: 217 - DNA, 35 bp, synthetic construct
  source 1..35: mol_type = other DNA
ccctgcgaagatagtctgcgtaaaattgacgcatg

SEQ ID NO: 218 - DNA, 35 bp, synthetic construct
  source 1..35: mol_type = other DNA
ccctagaaagatagtctgcgtaaaattgacgcatt

SEQ ID NO: 219 - DNA, 35 bp, synthetic construct
  source 1..35: mol_type = other DNA
ccctagatccatagtctgcgtaaaattgacgcatg

SEQ ID NO: 220 - DNA, 35 bp, synthetic construct
  source 1..35: mol_type = other DNA
ccctagaaagatagtctgcgtaacgctgacgcatg

SEQ ID NO: 221 - DNA, 35 bp, synthetic construct
  source 1..35: mol_type = other DNA
ccctagaaagatagtctgcgtaaaatgggcgcatg

SEQ ID NO: 222 - DNA, 35 bp, synthetic construct
  source 1..35: mol_type = other DNA
ccctagaaagatagtctgcgtagaattgacgcatg

SEQ ID NO: 223 - DNA, 35 bp, synthetic construct
  source 1..35: mol_type = other DNA
ccctagaaagtcggtctgcgtaaaattgacgcatg

SEQ ID NO: 224 - DNA, 35 bp, synthetic construct
  source 1..35: mol_type = other DNA
cccctcaaagatagtctgcgtaaaattgacgcatg

SEQ ID NO: 225 - DNA, 35 bp, synthetic construct
  source 1..35: mol_type = other DNA
ccctagaactctagtctgcgtaaaattgacgcatg

SEQ ID NO: 226 - DNA, 35 bp, synthetic construct
  source 1..35: mol_type = other DNA
cccaagaaagatagtctgcgtaaaattgacgcatg

SEQ ID NO: 227 - DNA, 35 bp, synthetic construct
  source 1..35: mol_type = other DNA
ccctagaaagatagtacgcgtaaaattgacgcatg

SEQ ID NO: 228 - DNA, 35 bp, synthetic construct
  source 1..35: mol_type = other DNA
ccctagaaagatagtctgcgtgaaattgacgcatg

SEQ ID NO: 229 - DNA, 35 bp, synthetic construct
  source 1..35: mol_type = other DNA
ccctagaaagatagtctgcgtacggttgacgcatg

SEQ ID NO: 230 - DNA, 35 bp, synthetic construct
  source 1..35: mol_type = other DNA
ccctagaaagatagtctgcgtatccttgacgcatg

SEQ ID NO: 231 - DNA, 35 bp, synthetic construct
  source 1..35: mol_type = other DNA
ccctagaaagatggactgcgtaaaattgacgcatg

SEQ ID NO: 232 - DNA, 35 bp, synthetic construct
  source 1..35: mol_type = other DNA
ccctagaaagatagtctgcgtcagattgacgcatg

SEQ ID NO: 233 - DNA, 35 bp, synthetic construct
  source 1..35: mol_type = other DNA
ccctagaaagatagtctgcgtaaaattgacgcagt

SEQ ID NO: 234 - DNA, 35 bp, synthetic construct
  source 1..35: mol_type = other DNA
ccctagaaagatagtctgcgtctaattgacgcatg

SEQ ID NO: 235 - DNA, 35 bp, synthetic construct
  source 1..35: mol_type = other DNA
ccctagaaagatagtctgcgtaaaatctccgcatg

SEQ ID NO: 236 - DNA, 35 bp, synthetic construct
  source 1..35: mol_type = other DNA
cccaggaaagatagtctgcgtaaaattgacgcatg

SEQ ID NO: 237 - DNA, 35 bp, synthetic construct
  source 1..35: mol_type = other DNA
ccctagaaagatagtctgcgtaaaatatacgcatg

SEQ ID NO: 238 - DNA, 35 bp, synthetic construct
  source 1..35: mol_type = other DNA
ccctagaaagatagtctgcgtcgtattgacgcatg

SEQ ID NO: 239 - DNA, 35 bp, synthetic construct
  source 1..35: mol_type = other DNA
ccctagaaagatagtctgcgtaaaattgattaatg

SEQ ID NO: 240 - DNA, 35 bp, synthetic construct
  source 1..35: mol_type = other DNA
ccctagaaagatagtctgcgtaaactggacgcatg

SEQ ID NO: 241 - DNA, 35 bp, synthetic construct
  source 1..35: mol_type = other DNA
ccctagaaagatagtctgcgctaaattgacgcatg

SEQ ID NO: 242 - DNA, 35 bp, synthetic construct
  source 1..35: mol_type = other DNA
cccgagaaagatagtctgcgtaaaattgacgcatg

SEQ ID NO: 243 - DNA, 35 bp, synthetic construct
  source 1..35: mol_type = other DNA
ccctagaaggttagtctgcgtaaaattgacgcatg

SEQ ID NO: 244 - DNA, 35 bp, synthetic construct; identical to SEQ ID NO: 207
  source 1..35: mol_type = other DNA

SEQ ID NO: 245 - DNA, 35 bp, synthetic construct
  source 1..35: mol_type = other DNA
ccctagaaagatagtctgcggaaaattgacgcatg

SEQ ID NO: 246 - DNA, 35 bp, synthetic construct
  source 1..35: mol_type = other DNA
ccttagaaagatagtctgcgtaaaattgacgcatg

SEQ ID NO: 247 - DNA, 35 bp, synthetic construct
  source 1..35: mol_type = other DNA
ccgtggaaagatagtctgcgtaaaattgacgcatg

SEQ ID NO: 248 - DNA, 35 bp, synthetic construct
  source 1..35: mol_type = other DNA
ccctagaaagatagtctgcgtaaaattctagcatg

SEQ ID NO: 249 - DNA, 35 bp, synthetic construct
  source 1..35: mol_type = other DNA
ccctagaaagatagtccacgtaaaattgacgcatg

SEQ ID NO: 250 - DNA, 35 bp, synthetic construct
  source 1..35: mol_type = other DNA
ccctagaaagatagtctgcgccaaattgacgcatg

SEQ ID NO: 251 - DNA, 35 bp, synthetic construct
  source 1..35: mol_type = other DNA
ccctagaaagatagtccgcgtaaaattgacgcatg

SEQ ID NO: 252 - DNA, 35 bp, synthetic construct
  source 1..35: mol_type = other DNA
cggtagaaagatagtctgcgtaaaattgacgcatg

SEQ ID NO: 253 - DNA, 35 bp, synthetic construct
  source 1..35: mol_type = other DNA
ccctagaaagatagaccgcgtaaaattgacgcatg

SEQ ID NO: 254 - DNA, 35 bp, synthetic construct
  source 1..35: mol_type = other DNA
ccctagaaagatagtcacagtaaaattgacgcatg

SEQ ID NO: 255 - DNA, 35 bp, synthetic construct
  source 1..35: mol_type = other DNA
ctgtagaaagatagtctgcgtaaaattgacgcatg

SEQ ID NO: 256 - DNA, 35 bp, synthetic construct
  source 1..35: mol_type = other DNA
ccctagaaagatagtctgcgtccgattgacgcatg

SEQ ID NO: 257 - DNA, 35 bp, synthetic construct
  source 1..35: mol_type = other DNA
ccctagaaagatagtctgcgtaaaattttagcatg

SEQ ID NO: 258 - DNA, 35 bp, synthetic construct
  source 1..35: mol_type = other DNA
ccctagaaagatagtccgtgtaaaattgacgcatg

SEQ ID NO: 259 - DNA, 35 bp, synthetic construct
  source 1..35: mol_type = other DNA
ccctagaaagatagtctgcggtgaattgacgcatg

SEQ ID NO: 260 - DNA, 35 bp, synthetic construct
  source 1..35: mol_type = other DNA
ccctagaaagatagtcagtgtaaaattgacgcatg

SEQ ID NO: 261 - DNA, 35 bp, synthetic construct
  source 1..35: mol_type = other DNA
ccctagaaagatagtctgggcaaaattgacgcatg

SEQ ID NO: 262 - DNA, 35 bp, synthetic construct
  source 1..35: mol_type = other DNA
ccctagaaagatagtctggagaaaattgacgcatg

SEQ ID NO: 263 - DNA, 35 bp, synthetic construct
  source 1..35: mol_type = other DNA
ccctagaaagatagtctagttaaaattgacgcatg

SEQ ID NO: 264 - DNA, 35 bp, synthetic construct
  source 1..35: mol_type = other DNA
ccctagaaagatagtcttgataaaattgacgcatg

SEQ ID NO: 265 - DNA, 35 bp, synthetic construct
  source 1..35: mol_type = other DNA
ccctagaaagatagtctagataaaattgacgcatg

SEQ ID NO: 266 - skipped

SEQ ID NO: 267 - DNA, 63 bp, synthetic construct
  source 1..63: mol_type = other DNA
ccctagaaagataatcatattgtgacgtatgctaaagataatcatgcgtaaaattgacgcatg

SEQ ID NO: 268 - DNA, 63 bp, synthetic construct
  source 1..63: mol_type = other DNA
ccctagaaagataatcatattgtgacgtacgttaaagataatcatgcgttttattgacgcatg

SEQ ID NO: 269 - DNA, 63 bp, synthetic construct
  source 1..63: mol_type = other DNA
ccctagaaagataatcatattgtgacgtacgttaaagataatcatgcgtaattttgacgcatg

SEQ ID NO: 270 - DNA, 63 bp, synthetic construct
  source 1..63: mol_type = other DNA
ccctagaaagataatcatattgtgacgtacgttaaagataatcatgcgtaagcctgacgcatg

SEQ ID NO: 271 - DNA, 63 bp, synthetic construct
  source 1..63: mol_type = other DNA
ccctagaaagataatcatattgtgacgtacgttaaagataatcatgcgtaaaattgactggtg

SEQ ID NO: 272 - DNA, 63 bp, synthetic construct
  source 1..63: mol_type = other DNA
ccctagaaagataatcatattgtgacgtacgttaaagataatcatgcgtaaaatatacgcatg

SEQ ID NO: 273 - DNA, 63 bp, synthetic construct
  source 1..63: mol_type = other DNA
ccctagaaagataatcatattgtgacgtacgttaaagataatcatgcgccgaattgacgcatg

SEQ ID NO: 274 - DNA, 63 bp, synthetic construct
  source 1..63: mol_type = other DNA
ccctagaaagataatcatattgtgacgtacgttaaagataatcatgcgtaaaattgctacatg

SEQ ID NO: 275 - DNA, 63 bp, synthetic construct
  source 1..63: mol_type = other DNA
ccctagaaagataatcatattgtgacgtacgttaaagataatcatgcgtaaaatgaacgcatg

SEQ ID NO: 276 - DNA, 63 bp, synthetic construct
  source 1..63: mol_type = other DNA
ccctagaaagataatcatattgtgacgtacgttaaagataatcatgcgtaaaattggtccatg

SEQ ID NO: 277 - DNA, 63 bp, synthetic construct
  source 1..63: mol_type = other DNA
ccctagaaagataatcatattgtgatcgacgttaaagataatcatgcgtaaaattgacgcatg

SEQ ID NO: 278 - skipped

SEQ ID NO: 279 - DNA, 63 bp, synthetic construct
  source 1..63: mol_type = other DNA
ccctagaaagataatcatattgtgacgtacgttaaagataaacttgcgtaaaattgacgcatg

SEQ ID NO: 280 - DNA, 63 bp, synthetic construct
  source 1..63: mol_type = other DNA
ccctagaaagataatcatattgtgacgtacgttaaagataatcagtagtaaaattgacgcatg

SEQ ID NO: 281 - DNA, 63 bp, synthetic construct
  source 1..63: mol_type = other DNA
ccctagaaagataatcatattgtgacgtacgttaaagataatcatgcgtaatgatgacgcatg

SEQ ID NO: 282 - DNA, 63 bp, synthetic construct
  source 1..63: mol_type = other DNA
ccctagaaagataatcatattgtgacgtacgttaaagataatcatgcgtaaaattgactactg

SEQ ID NO: 283 - DNA, 63 bp, synthetic construct
  source 1..63: mol_type = other DNA
ccctagaaagataatcatattgtgacgtacgttaaagataatcatgcgtaaaggcgacgcatg

SEQ ID NO: 284 - DNA, 63 bp, synthetic construct
  source 1..63: mol_type = other DNA
ccctagaaagataatcatattgtgtgctacgttaaagataatcatgcgtaaaattgacgcatg

SEQ ID NO: 285 - DNA, 63 bp, synthetic construct
  source 1..63: mol_type = other DNA
ccctagaaagataatcatattgtgacgtacgttaaagataatcatgcgagtaattgacgcatg

SEQ ID NO: 286 - DNA, 63 bp, synthetic construct
  source 1..63: mol_type = other DNA
ccctccaaagataatcatattgtgacgtacgttaaagataatcatgcgtaaaattgacgcatg

SEQ ID NO: 287 - DNA, 63 bp, synthetic construct
  source 1..63: mol_type = other DNA
ccctagaaagataatcatattgtgacgtacgttaaagataatcagtcgtaaaattgacgcatg

SEQ ID NO: 288 - DNA, 63 bp, synthetic construct
  source 1..63: mol_type = other DNA
ccctagaaagataatcatattgtgacgtacgttaaagataatcatgcgtaaaattgacgcggt

SEQ ID NO: 289 - DNA, 63 bp, synthetic construct
  source 1..63: mol_type = other DNA
ccctagaaagataatcatattgtgacgtacgcagaagataatcatgcgtaaaattgacgcatg

SEQ ID NO: 290 - DNA, 63 bp, synthetic construct
  source 1..63: mol_type = other DNA
ccctagaaagataatcatattgtgggctacgttaaagataatcatgcgtaaaattgacgcatg

SEQ ID NO: 291 - DNA, 63 bp, synthetic construct
  source 1..63: mol_type = other DNA
ccctagaaagataatcatatttgcacgtacgttaaagataatcatgcgtaaaattgacgcatg

SEQ ID NO: 292 - DNA, 63 bp, synthetic construct
  source 1..63: mol_type = other DNA
ccctagaaagataatcatattgtgacgtacgttaaagataatcatgccctaaattgacgcatg

SEQ ID NO: 293 - DNA, 63 bp, synthetic construct
  source 1..63: mol_type = other DNA
ccctagaaagataatcatattgtgacgtacgctcaagataatcatgcgtaaaattgacgcatg

SEQ ID NO: 294 - DNA, 63 bp, synthetic construct
  source 1..63: mol_type = other DNA
ccctagaaagataatcatattgtgacgtacgttaaagatagccatgcgtaaaattgacgcatg

SEQ ID NO: 295 - DNA, 63 bp, synthetic construct
  source 1..63: mol_type = other DNA
ccctagaaagataatcatattgtgacgtacgttaaagataatcatgcgtaaaatctccgcatg

SEQ ID NO: 296 - DNA, 63 bp, synthetic construct
  source 1..63: mol_type = other DNA
ccctagaaagataatcatattgtgacgtacgttaaagctgatcatgcgtaaaattgacgcatg

SEQ ID NO: 297 - DNA, 63 bp, synthetic construct
  source 1..63: mol_type = other DNA
ccctagaaagataatcatattgtgacgtacgttaaagataccgatgcgtaaaattgacgcatg

SEQ ID NO: 298 - DNA, 63 bp, synthetic construct
  source 1..63: mol_type = other DNA
ccctagaaagataatcatattgtgacgtacgttaaagataatcatgcgtaaaattggtgcatg

SEQ ID NO: 299 - DNA, 63 bp, synthetic construct
  source 1..63: mol_type = other DNA
ccctagaaagataatcatatggcgacgtacgttaaagataatcatgcgtaaaattgacgcatg

SEQ ID NO: 300 - DNA, 63 bp, synthetic construct
  source 1..63: mol_type = other DNA
ccctagaaagataatcatattgtgacgtacgttaaagatggacatgcgtaaaattgacgcatg

SEQ ID NO: 301 - DNA, 63 bp, synthetic construct
  source 1..63: mol_type = other DNA
ccctagaaagataatcatattgtgacgcttgttaaagataatcatgcgtaaaattgacgcatg

SEQ ID NO: 302 - DNA, 63 bp, synthetic construct
  source 1..63: mol_type = other DNA
ccctagaaagataatcatattgtgacgtacgttgccgataatcatgcgtaaaattgacgcatg

SEQ ID NO: 303 - DNA, 63 bp, synthetic construct
  source 1..63: mol_type = other DNA
ccctagaaagataatcatattgtgacgtacgttaaagataatcatgcgtgctattgacgcatg

SEQ ID NO: 304 - DNA, 63 bp, synthetic construct
  source 1..63: mol_type = other DNA
ccctagaaagataatcatattgtgacgtacgttaaagccaatcatgcgtaaaattgacgcatg

SEQ ID NO: 305 - DNA, 63 bp, synthetic construct
  source 1..63: mol_type = other DNA
ccctagaaagataatcatattgtgacgttaattaaagataatcatgcgtaaaattgacgcatg

SEQ ID NO: 306 - DNA, 63 bp, synthetic construct
  source 1..63: mol_type = other DNA
ccctagaaagataatcatattgtgacgtacgttaaagataatcatgccccaaattgacgcatg

SEQ ID NO: 307 - DNA, 63 bp, synthetic construct
  source 1..63: mol_type = other DNA
ccctagaaagataatcatattgtgacgtacgttaaagataatctaacgtaaaattgacgcatg

SEQ ID NO: 308 - DNA, 63 bp, synthetic construct
  source 1..63: mol_type = other DNA
ccctagaaagataatcatattttaacgtacgttaaagataatcatgcgtaaaattgacgcatg

SEQ ID NO: 309 - DNA, 63 bp, synthetic construct
  source 1..63: mol_type = other DNA
ccctagaaagataatcatattgtgacgtacgttaaagataatcatagctaaaattgacgcatg

SEQ ID NO: 310 - DNA, 63 bp, synthetic construct
  source 1..63: mol_type = other DNA
ccctagaaagataatcatattgtgacgtacgttaaagataatcatgcgtaaaattgcggcatg

SEQ ID NO: 311 - DNA, 63 bp, synthetic construct
  source 1..63: mol_type = other DNA
ccctagaaagataatcatattgtgacgtacgttaaaggttatcatgcgtaaaattgacgcatg

SEQ ID NO: 312 - DNA, 63 bp, synthetic construct
  source 1..63: mol_type = other DNA
ccctagaaagataatcatattgtgaccaccgttaaagataatcatgcgtaaaattgacgcatg

SEQ ID NO: 313 - DNA, 63 bp, synthetic construct
  source 1..63: mol_type = other DNA
ccctagaaagataatcatattgtgacgtacgttaaagataatcatgcgtaaaatgcccgcatg

SEQ ID NO: 314 - DNA, 63 bp, synthetic construct
  source 1..63: mol_type = other DNA
ccctagaaagataatcatattgtgacgtacgttaatcataatcatgcgtaaaattgacgcatg

SEQ ID NO: 315 - DNA, 63 bp, synthetic construct
  source 1..63: mol_type = other DNA
ccctagaaagataatcatattgtgacgtacgttaaagataatcatgaggaaaattgacgcatg

SEQ ID NO: 316 - DNA, 63 bp, synthetic construct
  source 1..63: mol_type = other DNA
ccctagaaagataatcatattgtgacgtacgttaaagataatcatgcgtattgttgacgcatg

SEQ ID NO: 317 - DNA, 63 bp, synthetic construct
  source 1..63: mol_type = other DNA
ccctagaaagataatcatattgtgacgtacgtgtgagataatcatgcgtaaaattgacgcatg

SEQ ID NO: 318 - DNA, 63 bp, synthetic construct
  source 1..63: mol_type = other DNA
ccctagaaagataatcatattgtgacgtacgttaaagataatcatgcgtaaaattgacgccgg

SEQ ID NO: 319 - DNA, 63 bp, synthetic construct
  source 1..63: mol_type = other DNA
ccctagaaagataatcatattgtgaggcacgttaaagataatcatgcgtaaaattgacgcatg

SEQ ID NO: 320 - DNA, 63 bp, synthetic construct
  source 1..63: mol_type = other DNA
ccctagaaagataatcatattgtgacgtacgttaaagataatcatgcgtaaaattgacgagcg

SEQ ID NO: 321 - DNA, 63 bp, synthetic construct
  source 1..63: mol_type = other DNA
ccctagaaagataatcatattgtgaagcacgttaaagataatcatgcgtaaaattgacgcatg

SEQ ID NO: 322 - DNA, 63 bp, synthetic construct
  source 1..63: mol_type = other DNA
ccctagaaagataatcatattgtgacgtacgttaaagataatcatgcgtaattgtgacgcatg

SEQ ID NO: 323 - DNA, 63 bp, synthetic construct
  source 1..63: mol_type = other DNA
ccctagaaagataatcatattgtgacgtcctttaaagataatcatgcgtaaaattgacgcatg

SEQ ID NO: 324 - DNA, 63 bp, synthetic construct
  source 1..63: mol_type = other DNA
ccctagaaagataatcatattgtgacgtacgttagccataatcatgcgtaaaattgacgcatg

SEQ ID NO: 325 - DNA, 63 bp, synthetic construct
  source 1..63: mol_type = other DNA
ccctagaaagataatcatattgtgacgtactccaaagataatcatgcgtaaaattgacgcatg

SEQ ID NO: 326 - DNA, 63 bp, synthetic construct
  source 1..63: mol_type = other DNA
ccctagaaagataatcatattgtgacaaccgttaaagataatcatgcgtaaaattgacgcatg

SEQ ID NO: 327 - DNA, 63 bp, synthetic construct
  source 1..63: mol_type = other DNA
ccctagaaagataatcatattgtgacgtacgttggagataatcatgcgtaaaattgacgcatg

SEQ ID NO: 328 - DNA, 63 bp, synthetic construct
  source 1..63: mol_type = other DNA
ccctagaaagataatcatactttgacgtacgttaaagataatcatgcgtaaaattgacgcatg

SEQ ID NO: 329 - DNA, 63 bp, synthetic construct
  source 1..63: mol_type = other DNA
ccctagaaagataatcatattgtgacgtacgttaaagataatcatgcgtaaaattgcttcatg

SEQ ID NO: 330 - skipped

SEQ ID NO: 331 - DNA, 63 bp, synthetic construct
  source 1..63: mol_type = other DNA
ccctagaaagataatcatattgtgacgtacgttaaagataatcatgcgtaaaattgatcaatg

SEQ ID NO: 332 - DNA, 63 bp, synthetic construct
  source 1..63: mol_type = other DNA
ccccggaaagataatcatattgtgacgtacgttaaagataatcatgcgtaaaattgacgcatg

SEQ ID NO: 333 - DNA, 63 bp, synthetic construct
  source 1..63: mol_type = other DNA
ccctagaaagataatcatattgtgacgtacgttaaagtgaatcatgcgtaaaattgacgcatg

SEQ ID NO: 334 - DNA, 63 bp, synthetic construct
  source 1..63: mol_type = other DNA
ccctagaaagataatcatattggtccgtacgttaaagataatcatgcgtaaaattgacgcatg

SEQ ID NO: 335 - DNA, 63 bp, synthetic construct
  source 1..63: mol_type = other DNA
ccctagaaagataatcatattgtgcattacgttaaagataatcatgcgtaaaattgacgcatg

SEQ ID NO: 336 - DNA, 63 bp, synthetic construct
  source 1..63: mol_type = other DNA
ccctagaaagataatcatattgtgacgtacgttaaagataatcatgcgtaaaatgggcgcatg

SEQ ID NO: 337 - DNA, 63 bp, synthetic construct
  source 1..63: mol_type = other DNA
ccctagaaagataatcatattgtgacgtaaactaaagataatcatgcgtaaaattgacgcatg

SEQ ID NO: 338 - DNA, 63 bp, synthetic construct
  source 1..63: mol_type = other DNA
ccctagaaagataatcatattgtgacgtacgttaaagataatcatgcgtccaattgacgcatg

SEQ ID NO: 339 - DNA, 63 bp, synthetic construct
  source 1..63: mol_type = other DNA
ccctagaaagataatcatattgtgacgtacgttaaagataatcatgcgtaaaattaatgcatg

SEQ ID NO: 340 - DNA, 63 bp, synthetic construct
  source 1..63: mol_type = other DNA
ccctagaaagataatcatattgtgacgtacgttaaagataattacgcgtaaaattgacgcatg

SEQ ID NO: 341 - DNA, 63 bp, synthetic construct
  source 1..63: mol_type = other DNA
ccctagaaagataatcatattgtgacgtacgtttgcgataatcatgcgtaaaattgacgcatg

SEQ ID NO: 342 - DNA, 63 bp, synthetic construct
  source 1..63: mol_type = other DNA
ccctagaaagataatcatattgtgacgtacatcaaagataatcatgcgtaaaattgacgcatg

SEQ ID NO: 343 - DNA, 63 bp, synthetic construct
  source 1..63: mol_type = other DNA
ccctagaaagataatcatattgtgacgtacgttaaagataatcagaggtaaaattgacgcatg

SEQ ID NO: 344 - DNA, 63 bp, synthetic construct
  source 1..63: mol_type = other DNA
ccctagaaagataatcatattgtgacgtacgttaaagataatcatgcgtaaaattgagaaatg

SEQ ID NO: 345 - DNA, 63 bp, synthetic construct
  source 1..63: mol_type = other DNA
ccctcagaagataatcatattgtgacgtacgttaaagataatcatgcgtaaaattgacgcatg

SEQ ID NO: 346 - DNA, 63 bp, synthetic construct
  source 1..63: mol_type = other DNA
ccctagaaagataatcatattgtgacgtacgtatgagataatcatgcgtaaaattgacgcatg

SEQ ID NO: 347 - DNA, 63 bp, synthetic construct
  source 1..63: mol_type = other DNA
ccctagaaagataatcatattgtgacgcgtgttaaagataatcatgcgtaaaattgacgcatg

SEQ ID NO: 348 - DNA, 63 bp, synthetic construct
  source 1..63: mol_type = other DNA
ccctagaaagataatcatattgtggtgtacgttaaagataatcatgcgtaaaattgacgcatg

SEQ ID NO: 349 - DNA, 63 bp, synthetic construct
  source 1..63: mol_type = other DNA
ccctagaaagataatcatagcctgacgtacgttaaagataatcatgcgtaaaattgacgcatg

SEQ ID NO: 350 - DNA, 63 bp, synthetic construct
  source 1..63: mol_type = other DNA
ccctagaaagataatcatattgtgacgtacgttaaagataactctgcgtaaaattgacgcatg

SEQ ID NO: 351 - DNA, 63 bp, synthetic construct
  source 1..63: mol_type = other DNA
ccctagaaagataatcatattgtgacgtacgttaaagataatcatgcgtagccttgacgcatg

SEQ ID NO: 352 - DNA, 63 bp, synthetic construct
  source 1..63: mol_type = other DNA
ccctagaaagataatcatattgtgacgtacgttaaagataatcatgcgtaaaaaccacgcatg

SEQ ID NO: 353 - DNA, 63 bp, synthetic construct
  source 1..63: mol_type = other DNA
ccctagaaagataatcatattgtgacgtacgttaaagataatcaacagtaaaattgacgcatg

SEQ ID NO: 354 - DNA, 63 bp, synthetic construct
  source 1..63: mol_type = other DNA
ccctagaaagataatcatattgtgacgtacgttaaagataatctcgcgtaaaattgacgcatg

SEQ ID NO: 355 - DNA, 63 bp, synthetic construct
  source 1..63: mol_type = other DNA
ccctagaaagataatcatattgtgacgtacgttaaagataaccctgcgtaaaattgacgcatg

SEQ ID NO: 356 - DNA, 63 bp, synthetic construct
  source 1..63: mol_type = other DNA
ccctagaaagataatcatattgtgacgtacgttaaagataatcatgcgtaaaattgacagctg

SEQ ID NO: 357 - DNA, 63 bp, synthetic construct
  source 1..63: mol_type = other DNA
ccctagaaagataatcatattgtgacgctcgttaaagataatcatgcgtaaaattgacgcatg

SEQ ID NO: 358 - DNA, 63 bp, synthetic construct
  source 1..63: mol_type = other DNA
ccctagaaagataatcatattgtgacgtacgttttcgataatcatgcgtaaaattgacgcatg

SEQ ID NO: 359 - DNA, 63 bp, synthetic construct
  source 1..63: mol_type = other DNA
ccctagaaagataatcatattgtgacgtacgttaaagagattcatgcgtaaaattgacgcatg

SEQ ID NO: 360 - DNA, 63 bp, synthetic construct
  source 1..63: mol_type = other DNA
ccctagaaagataatcatattgtgacgtacgttaaagataatcatgcgtgacattgacgcatg

SEQ ID NO: 361 - DNA, 63 bp, synthetic construct
  source 1..63: mol_type = other DNA
ccctagaaagataatcatattgtgacgtacgttaaagataatcatgcgtaaaattgacgccca

SEQ ID NO: 362 - DNA, 63 bp, synthetic construct
  source 1..63: mol_type = other DNA
ccctagaaagataatcatattgtgacgtacgttaagcgtaatcatgcgtaaaattgacgcatg

SEQ ID NO: 363 - DNA, 63 bp, synthetic construct
  source 1..63: mol_type = other DNA
ccctagaaagataatcatattgtaccgtacgttaaagataatcatgcgtaaaattgacgcatg

SEQ ID NO: 364 - DNA, 63 bp, synthetic construct
  source 1..63: mol_type = other DNA
ccctagaaagataatcatattgtgacgtacgttaaagataatcattgctaaaattgacgcatg

SEQ ID NO: 365 - DNA, 63 bp, synthetic construct
  source 1..63: mol_type = other DNA
ccctagaaagataatcatattgtgacgtacgttaaagataatcatgcgtaaaattgacgcccg

SEQ ID NO: 366 - DNA, 63 bp, synthetic construct
  source 1..63: mol_type = other DNA
ccctagaaagataatcatattgtgacgtacgttaaagataatcatgcttgaaattgacgcatg

SEQ ID NO: 367 - DNA, 63 bp, synthetic construct
  source 1..63: mol_type = other DNA
ccctagaaagataatcatattcacacgtacgttaaagataatcatgcgtaaaattgacgcatg

SEQ ID NO: 368 - DNA, 63 bp, synthetic construct
  source 1..63: mol_type = other DNA
ccctagaaagataatcatattgtgacgtaggataaagataatcatgcgtaaaattgacgcatg

SEQ ID NO: 369 - DNA, 63 bp, synthetic construct
  source 1..63: mol_type = other DNA
ccctagaaagataatctcattgtgacgtacgttaaagataatcatgcgtaaaattgacgcatg

SEQ ID NO: 370 - DNA, 63 bp, synthetic construct
  source 1..63: mol_type = other DNA
ccctggaaagataatcatattgtgacgtacgttaaagataatcatgcgtaaaattgacgcatg

SEQ ID NO: 371 - DNA, 63 bp, synthetic construct
  source 1..63: mol_type = other DNA
ccctagaaagataatcatattgtgacgtacgttaaagagggtcatgcgtaaaattgacgcatg

SEQ ID NO: 372 - DNA, 63 bp, synthetic construct
  source 1..63: mol_type = other DNA
ccctagaaagataatcatattgtgacgtacgttaaacggaatcatgcgtaaaattgacgcatg

SEQ ID NO: 373 - DNA, 63 bp, synthetic construct
  source 1..63: mol_type = other DNA
cccttacaagataatcatattgtgacgtacgttaaagataatcatgcgtaaaattgacgcatg

SEQ ID NO: 374 - DNA, 63 bp, synthetic construct
  source 1..63: mol_type = other DNA
ccctagaaagataatcatattgtgacgtactagaaagataatcatgcgtaaaattgacgcatg

SEQ ID NO: 375 - DNA, 63 bp, synthetic construct
  source 1..63: mol_type = other DNA
ccctagaaagataatctagttgtgacgtacgttaaagataatcatgcgtaaaattgacgcatg

SEQ ID NO: 376 - DNA, 63 bp, synthetic construct
  source 1..63: mol_type = other DNA
ccctagaaagataatcatatacggacgtacgttaaagataatcatgcgtaaaattgacgcatg

SEQ ID NO: 377 - DNA, 63 bp, synthetic construct
  source 1..63: mol_type = other DNA
ccctagaaagataatcatattgtgacgtacgttaaagataatcatgcgtaaatacgacgcatg

SEQ ID NO: 378 - DNA, 63 bp, synthetic construct
  source 1..63: mol_type = other DNA
ccctagaaagataatcatattgtgacgtacgttaaagataatctggcgtaaaattgacgcatg

SEQ ID NO: 379 - DNA, 63 bp, synthetic construct
  source 1..63: mol_type = other DNA
ccctgcaaagataatcatattgtgacgtacgttaaagataatcatgcgtaaaattgacgcatg

SEQ ID NO: 380 - DNA, 63 bp, synthetic construct
  source 1..63: mol_type = other DNA
ccctagaaagataatcatattgtgacgtacgttaaagataatcatgcgtaaaattgaagaatg

SEQ ID NO: 381 - DNA, 63 bp, synthetic construct
  source 1..63: mol_type = other DNA
ccctagaaagataatcatattgtgacgtacgttaaagatgctcatgcgtaaaattgacgcatg

SEQ ID NO: 382 - DNA, 63 bp, synthetic construct
  source 1..63: mol_type = other DNA
ccctagaaagataatcatattgtgacgtacgttaaagataatcatgattaaaattgacgcatg

SEQ ID NO: 383 - DNA, 63 bp, synthetic construct
  source 1..63: mol_type = other DNA
ccctagaaagataatcatattgtgacgtacgttaaaggggatcatgcgtaaaattgacgcatg

SEQ ID NO: 384 - DNA, 63 bp, synthetic construct
  source 1..63: mol_type = other DNA
ccctagaaagataatcatattgtgacgtacgttaaagataatcatggcaaaaattgacgcatg

SEQ ID NO: 385 - DNA, 63 bp, synthetic construct
  source 1..63: mol_type = other DNA
ccctagaaagataatcatattgcctcgtacgttaaagataatcatgcgtaaaattgacgcatg

SEQ ID NO: 386 - DNA, 63 bp, synthetic construct
  source 1..63: mol_type = other DNA
ccccagaaagataatcatattgtgacgtacgttaaagataatcatgcgtaaaattgacgcatg

SEQ ID NO: 387 - DNA, 63 bp, synthetic construct
  source 1..63: mol_type = other DNA
ccctagaaagataatcatattgtgacgtacgttaaagataatcatgcgtaaaattgacgcgtc

SEQ ID NO: 388 - DNA, 63 bp, synthetic construct
  source 1..63: mol_type = other DNA
ccctagaaagataatctatttgtgacgtacgttaaagataatcatgcgtaaaattgacgcatg

SEQ ID NO: 389 - DNA, 63 bp, synthetic construct
  source 1..63: mol_type = other DNA
ccctagaaagataatcatattgtgacgtacgcataagataatcatgcgtaaaattgacgcatg

SEQ ID NO: 390 - DNA, 63 bp, synthetic construct
  source 1..63: mol_type = other DNA
ccctcgaaagataatcatattgtgacgtacgttaaagataatcatgcgtaaaattgacgcatg

SEQ ID NO: 391 - DNA, 63 bp, synthetic construct
  source 1..63: mol_type = other DNA
ccctagtgagataatcatattgtgacgtacgttaaagataatcatgcgtaaaattgacgcatg

SEQ ID NO: 392 - DNA, 63 bp, synthetic construct
  source 1..63: mol_type = other DNA
ccctagaaagataatcatattgtgacgtacgttaaagataatccttcgtaaaattgacgcatg

SEQ ID NO: 393 - DNA, 63 bp, synthetic construct
  source 1..63: mol_type = other DNA
ccctagaaagataatcatagtttgacgtacgttaaagataatcatgcgtaaaattgacgcatg

SEQ ID NO: 394 - DNA, 63 bp, synthetic construct
  source 1..63: mol_type = other DNA
ccctagaaagataatcctattgtgacgtacgttaaagataatcatgcgtaaaattgacgcatg

SEQ ID NO: 395 - DNA, 63 bp, synthetic construct
  source 1..63: mol_type = other DNA
ccctagaaagataatcatattgtgacgtacgttaaagataatcatgatgaaaattgacgcatg

SEQ ID NO: 396 - DNA, 63 bp, synthetic construct
  source 1..63: mol_type = other DNA
ccctagaaagataatcatattgtgacgcaggttaaagataatcatgcgtaaaattgacgcatg

SEQ ID NO: 397 - DNA, 63 bp, synthetic construct
  source 1..63: mol_type = other DNA
ccctagaaagataatcttattgtgacgtacgttaaagataatcatgcgtaaaattgacgcatg

SEQ ID NO: 398 - DNA, 63 bp, synthetic construct
  source 1..63: mol_type = other DNA
ccctagaaagataatctttttgtgacgtacgttaaagataatcatgcgtaaaattgacgcatg

SEQ ID NO: 399 - DNA, 63 bp, synthetic construct
  source 1..63: mol_type = other DNA
ccctagaaagataatcatattgtgacgtttcttaaagataatcatgcgtaaaattgacgcatg

SEQ ID NO: 400 - DNA, 63 bp, synthetic construct
  source 1..63: mol_type = other DNA
ccctagaaagataatcatattgtcatgtacgttaaagataatcatgcgtaaaattgacgcatg

SEQ ID NO: 401 - DNA, 63 bp, synthetic construct
  source 1..63: mol_type = other DNA
cccaagaaagataatcatattgtgacgtacgttaaagataatcatgcgtaaaattgacgcatg

SEQ ID NO: 402 - DNA, 63 bp, synthetic construct
  source 1..63: mol_type = other DNA
ccctagaaagataatcatattgtgacgtacgttaaagatacttatgcgtaaaattgacgcatg

SEQ ID NO: 403 - DNA, 63 bp, synthetic construct
  source 1..63: mol_type = other DNA
ccctagaaagataatcattaggtgacgtacgttaaagataatcatgcgtaaaattgacgcatg

SEQ ID NO: 404 - DNA, 63 bp, synthetic construct
  source 1..63: mol_type = other DNA
ccctagaaagataatcaatgtgtgacgtacgttaaagataatcatgcgtaaaattgacgcatg

SEQ ID NO: 405 - DNA, 63 bp, synthetic construct
  source 1..63: mol_type = other DNA
ccctagaaagataatcatattgtgacgtacgttaaagataatcatgcgtaaattagacgcatg

SEQ ID NO: 406 - DNA, 63 bp, synthetic construct
  source 1..63: mol_type = other DNA
ccgtagaaagataatcatattgtgacgtacgttaaagataatcatgcgtaaaattgacgcatg

SEQ ID NO: 407 - DNA, 63 bp, synthetic construct
  source 1..63: mol_type = other DNA
ccctttaaagataatcatattgtgacgtacgttaaagataatcatgcgtaaaattgacgcatg

SEQ ID NO: 408 - DNA, 63 bp, synthetic construct
  source 1..63: mol_type = other DNA
ccctagatggataatcatattgtgacgtacgttaaagataatcatgcgtaaaattgacgcatg

SEQ ID NO: 409 - DNA, 63 bp, synthetic construct
  source 1..63: mol_type = other DNA
ccctagaaagataatcactctgtgacgtacgttaaagataatcatgcgtaaaattgacgcatg

SEQ ID NO: 410 - DNA, 63 bp, synthetic construct
  source 1..63: mol_type = other DNA
ccctagaaagataatcatattgtgacgtacgttaaagatatagatgcgtaaaattgacgcatg

SEQ ID NO: 411 - DNA, 63 bp, synthetic construct
  source 1..63: mol_type = other DNA
ccctagaaagataatcatattgtgacgtacgttaagtgtaatcatgcgtaaaattgacgcatg

SEQ ID NO: 412 - DNA, 63 bp, synthetic construct
  source 1..63: mol_type = other DNA
ccctagaaagataatcatgctgtgacgtacgttaaagataatcatgcgtaaaattgacgcatg

SEQ ID NO: 413 - DNA, 63 bp, synthetic construct
  source 1..63: mol_type = other DNA
ccctagaaagataatcatattgttgagtacgttaaagataatcatgcgtaaaattgacgcatg

SEQ ID NO: 414 - DNA, 63 bp, synthetic construct
  source 1..63: mol_type = other DNA
ccctaccaagataatcatattgtgacgtacgttaaagataatcatgcgtaaaattgacgcatg

SEQ ID NO: 415 - DNA, 63 bp, synthetic construct
  source 1..63: mol_type = other DNA
ccctagaaagataatcatagggtgacgtacgttaaagataatcatgcgtaaaattgacgcatg

SEQ ID NO: 416 - DNA, 63 bp, synthetic construct
  source 1..63: mol_type = other DNA
ccctagaaagataatcatcaggtgacgtacgttaaagataatcatgcgtaaaattgacgcatg

SEQ ID NO: 417 - DNA, 63 bp, synthetic construct
  source 1..63: mol_type = other DNA
ccctagtcggataatcatattgtgacgtacgttaaagataatcatgcgtaaaattgacgcatg

SEQ ID NO: 418 - DNA, 63 bp, synthetic construct
  source 1..63: mol_type = other DNA
ccttagaaagataatcatattgtgacgtacgttaaagataatcatgcgtaaaattgacgcatg

SEQ ID NO: 419 - DNA, 63 bp, synthetic construct
  source 1..63: mol_type = other DNA
ccctacatagataatcatattgtgacgtacgttaaagataatcatgcgtaaaattgacgcatg

SEQ ID NO: 420 - DNA, 63 bp, synthetic construct
  source 1..63: mol_type = other DNA
ccctagaaagataaacatattgtgacgtacgttaaagataatcatgcgtaaaattgacgcatg

SEQ ID NO: 421 - DNA, 63 bp, synthetic construct
  source 1..63: mol_type = other DNA
ccctagaaagataatcatatgtagacgtacgttaaagataatcatgcgtaaaattgacgcatg

SEQ ID NO: 422 - DNA, 63 bp, synthetic construct
  source 1..63: mol_type = other DNA
ccctagaaagataatctaattgtgacgtacgttaaagataatcatgcgtaaaattgacgcatg

SEQ ID NO: 423 - DNA, 63 bp, synthetic construct
  source 1..63: mol_type = other DNA
ccctagctagataatcatattgtgacgtacgttaaagataatcatgcgtaaaattgacgcatg

SEQ ID NO: 424 - DNA, 63 bp, synthetic construct
  source 1..63: mol_type = other DNA
ccctagaaagattatcatattgtgacgtacgttaaagataatcatgcgtaaaattgacgcatg

SEQ ID NO: 425 - DNA, 63 bp, synthetic construct
  source 1..63: mol_type = other DNA
ccctagaaagataattatattgtgacgtacgttaaagataatcatgcgtaaaattgacgcatg

SEQ ID NO: 426 - DNA, 63 bp, synthetic construct
  source 1..63: mol_type = other DNA
ccctagaaagttaatcatattgtgacgtacgttaaagataatcatgcgtaaaattgacgcatg

SEQ ID NO: 427 - DNA, 63 bp, synthetic construct
  source 1..63: mol_type = other DNA
ccctagcgggataatcatattgtgacgtacgttaaagataatcatgcgtaaaattgacgcatg

SEQ ID NO: 428 - DNA, 63 bp, synthetic construct
  source 1..63: mol_type = other DNA
ccctagaaaggtaatcatattgtgacgtacgttaaagataatcatgcgtaaaattgacgcatg

SEQ ID NO: 429 - DNA, 63 bp, synthetic construct
  source 1..63: mol_type = other DNA
caccagaaagataatcatattgtgacgtacgttaaagataatcatgcgtaaaattgacgcatg

SEQ ID NO: 430 - DNA, 63 bp, synthetic construct
  source 1..63: mol_type = other DNA
ccctagaaagatattcatattgtgacgtacgttaaagataatcatgcgtaaaattgacgcatg

SEQ ID NO: 431 - DNA, 63 bp, synthetic construct
  source 1..63: mol_type = other DNA
ccctagaaagataatgctattgtgacgtacgttaaagataatcatgcgtaaaattgacgcatg

SEQ ID NO: 432 - DNA, 63 bp, synthetic construct
  source 1..63: mol_type = other DNA
cgctagaaagataatcatattgtgacgtacgttaaagataatcatgcgtaaaattgacgcatg

SEQ ID NO: 433 - DNA, 63 bp, synthetic construct
  source 1..63: mol_type = other DNA
ccctagaaagataatgatattgtgacgtacgttaaagataatcatgcgtaaaattgacgcatg

SEQ ID NO: 434 - DNA, 63 bp, synthetic construct
  source 1..63: mol_type = other DNA
ccctagaaagctaatcatattgtgacgtacgttaaagataatcatgcgtaaaattgacgcatg

SEQ ID NO: 435 - DNA, 63 bp, synthetic construct
  source 1..63: mol_type = other DNA
ccctagactcataatcatattgtgacgtacgttaaagataatcatgcgtaaaattgacgcatg

SEQ ID NO: 436 - DNA, 63 bp, synthetic construct
  source 1..63: mol_type = other DNA
ccctagaaagataatcatattgtgacgtacgttaaccttaatcatgcgtaaaattgacgcatg

SEQ ID NO: 437 - DNA, 63 bp, synthetic construct
  source 1..63: mol_type = other DNA
ccctagaaagataatcatattgtgacgtacgttacctataatcatgcgtaaaattgacgcatg

SEQ ID NO: 438 - DNA, 63 bp, synthetic construct
  source 1..63: mol_type = other DNA
ccctagaaagataatccaattgtgacgtacgttaaagataatcatgcgtaaaattgacgcatg

SEQ ID NO: 439 - DNA, 63 bp, synthetic construct
  source 1..63: mol_type = other DNA
ccctagaaagagaatcatattgtgacgtacgttaaagataatcatgcgtaaaattgacgcatg

SEQ ID NO: 440 - DNA, 63 bp, synthetic construct
  source 1..63: mol_type = other DNA
ccctagaaagataatcatattgtgacgtacgttaaagatcgacatgcgtaaaattgacgcatg

SEQ ID NO: 441 - DNA, 63 bp, synthetic construct
  source 1..63: mol_type = other DNA
ccctagaaagatgatcatattgtgacgtacgttaaagataatcatgcgtaaaattgacgcatg

SEQ ID NO: 442 - DNA, 63 bp, synthetic construct
  source 1..63: mol_type = other DNA
ccctagaaagggaatcatattgtgacgtacgttaaagataatcatgcgtaaaattgacgcatg

SEQ ID NO: 443 - DNA, 63 bp, synthetic construct
  source 1..63: mol_type = other DNA
ccctagaaagtgaatcatattgtgacgtacgttaaagataatcatgcgtaaaattgacgcatg

SEQ ID NO: 444 - DNA, 63 bp, synthetic construct
  source 1..63: mol_type = other DNA
ccctagagatataatcatattgtgacgtacgttaaagataatcatgcgtaaaattgacgcatg

SEQ ID NO: 445 - DNA, 63 bp, synthetic construct
  source 1..63: mol_type = other DNA
ccctagaagtataatcatattgtgacgtacgttaaagataatcatgcgtaaaattgacgcatg

SEQ ID NO: 446 - DNA, 63 bp, synthetic construct
  source 1..63: mol_type = other DNA
ccctagaaagataaatatattgtgacgtacgttaaagataatcatgcgtaaaattgacgcatg

SEQ ID NO: 447 - DNA, 63 bp, synthetic construct
  source 1..63: mol_type = other DNA
ccctagaaagagtatcatattgtgacgtacgttaaagataatcatgcgtaaaattgacgcatg

SEQ ID NO: 448 - DNA, 63 bp, synthetic construct
  source 1..63: mol_type = other DNA
ccctagaaagatagtcatattgtgacgtacgttaaagataatcatgcgtaaaattgacgcatg

SEQ ID NO: 449 - DNA, 63 bp, synthetic construct
  source 1..63: mol_type = other DNA
ccctagaaagatgaacatattgtgacgtacgttaaagataatcatgcgtaaaattgacgcatg

SEQ ID NO: 450 - DNA, 63 bp, synthetic construct
  source 1..63: mol_type = other DNA
ccctagaaagagattcatattgtgacgtacgttaaagataatcatgcgtaaaattgacgcatg

SEQ ID NO: 451 - DNA, 63 bp, synthetic construct
  source 1..63: mol_type = other DNA
ccctagaaagataagcatattgtgacgtacgttaaagataatcatgcgtaaaattgacgcatg

SEQ ID NO: 452 - DNA, 10731 bp, synthetic construct; identical to SEQ ID NO: 114
  source 1..10731: mol_type = other DNA

SEQ ID NO: 453 - DNA, 8920 bp, synthetic construct; identical to SEQ ID NO: 115
  source 1..8920: mol_type = other DNA

SEQ ID NO: 454 - DNA, 35 bp, synthetic construct
  source 1..35: mol_type = other DNA
nnntagaaagatagtctgcgtaaaattgacgcatg

SEQ ID NO: 455 - DNA, 35 bp, synthetic construct
  source 1..35: mol_type = other DNA
cnnnagaaagatagtctgcgtaaaattgacgcatg

SEQ ID NO: 456 - DNA, 35 bp, synthetic construct
  source 1..35: mol_type = other DNA
ccnnngaaagatagtctgcgtaaaattgacgcatg

SEQ ID NO: 457 - DNA, 35 bp, synthetic construct
  source 1..35: mol_type = other DNA
cccnnnaaagatagtctgcgtaaaattgacgcatg

SEQ ID NO: 458 - DNA, 35 bp, synthetic construct
  source 1..35: mol_type = other DNA
ccctnnnaagatagtctgcgtaaaattgacgcatg

SEQ ID NO: 459 - DNA, 35 bp, synthetic construct
  source 1..35: mol_type = other DNA
ccctannnagatagtctgcgtaaaattgacgcatg

SEQ ID NO: 460 - DNA, 35 bp, synthetic construct
  source 1..35: mol_type = other DNA
ccctagnnngatagtctgcgtaaaattgacgcatg

SEQ ID NO: 461 - DNA, 35 bp, synthetic construct
  source 1..35: mol_type = other DNA
ccctagannnatagtctgcgtaaaattgacgcatg

SEQ ID NO: 462 - DNA, 35 bp, synthetic construct
  source 1..35: mol_type = other DNA
ccctagaannntagtctgcgtaaaattgacgcatg

SEQ ID NO: 463 - DNA, 35 bp, synthetic construct
  source 1..35: mol_type = other DNA
ccctagaaannnagtctgcgtaaaattgacgcatg

SEQ ID NO: 464 - DNA, 35 bp, synthetic construct
  source 1..35: mol_type = other DNA
ccctagaaagnnngtctgcgtaaaattgacgcatg

SEQ ID NO: 465 - DNA, 35 bp, synthetic construct
  source 1..35: mol_type = other DNA
ccctagaaagannntctgcgtaaaattgacgcatg

SEQ ID NO: 466 - DNA, 35 bp, synthetic construct
  source 1..35: mol_type = other DNA
ccctagaaagatnnnctgcgtaaaattgacgcatg

SEQ ID NO: 467 - DNA, 35 bp, synthetic construct
  source 1..35: mol_type = other DNA
ccctagaaagatannntgcgtaaaattgacgcatg

SEQ ID NO: 468 - DNA, 35 bp, synthetic construct
  source 1..35: mol_type = other DNA
ccctagaaagatagnnngcgtaaaattgacgcatg

SEQ ID NO: 469 - DNA, 35 bp, synthetic construct
  source 1..35: mol_type = other DNA
ccctagaaagatagtnnncgtaaaattgacgcatg

SEQ ID NO: 470 - DNA, 35 bp, synthetic construct
  source 1..35: mol_type = other DNA
ccctagaaagatagtcnnngtaaaattgacgcatg

SEQ ID NO: 471 - DNA, 35 bp, synthetic construct
  source 1..35: mol_type = other DNA
ccctagaaagatagtctnnntaaaattgacgcatg

SEQ ID NO: 472 - DNA, 35 bp, synthetic construct
  source 1..35: mol_type = other DNA
ccctagaaagatagtctgnnnaaaattgacgcatg

SEQ ID NO: 473 - DNA, 35 bp, synthetic construct
  source 1..35: mol_type = other DNA
ccctagaaagatagtctgcnnnaaattgacgcatg

SEQ ID NO: 474 - DNA, 35 bp, synthetic construct
  source 1..35: mol_type = other DNA
ccctagaaagatagtctgcgnnnaattgacgcatg

SEQ ID NO: 475 - DNA, 35 bp, synthetic construct
  source 1..35: mol_type = other DNA
ccctagaaagatagtctgcgtnnnattgacgcatg

SEQ ID NO: 476 - DNA, 35 bp, synthetic construct
  source 1..35: mol_type = other DNA
ccctagaaagatagtctgcgtannnttgacgcatg

SEQ ID NO: 477 - DNA, 35 bp, synthetic construct
  source 1..35: mol_type = other DNA
ccctagaaagatagtctgcgtaannntgacgcatg

SEQ ID NO: 478 - DNA, 35 bp, synthetic construct
  source 1..35: mol_type = other DNA
ccctagaaagatagtctgcgtaaannngacgcatg

SEQ ID NO: 479 - DNA, 35 bp, synthetic construct
  source 1..35: mol_type = other DNA
ccctagaaagatagtctgcgtaaaannnacgcatg

SEQ ID NO: 480 - DNA, 35 bp, synthetic construct
  source 1..35: mol_type = other DNA
ccctagaaagatagtctgcgtaaaatnnncgcatg

SEQ ID NO: 481 - DNA, 35 bp, synthetic construct
  source 1..35: mol_type = other DNA
ccctagaaagatagtctgcgtaaaattnnngcatg

SEQ ID NO: 482 - DNA, 35 bp, synthetic construct
  source 1..35: mol_type = other DNA
ccctagaaagatagtctgcgtaaaattgnnncatg

SEQ ID NO: 483 - DNA, 35 bp, synthetic construct
  source 1..35: mol_type = other DNA
ccctagaaagatagtctgcgtaaaattgannnatg

SEQ ID NO: 484 - DNA, 35 bp, synthetic construct
  source 1..35: mol_type = other DNA
ccctagaaagatagtctgcgtaaaattgacnnntg

SEQ ID NO: 485 - DNA, 35 bp, synthetic construct
  source 1..35: mol_type = other DNA
ccctagaaagatagtctgcgtaaaattgacgnnng

SEQ ID NO: 486 - DNA, 35 bp, synthetic construct
  source 1..35: mol_type = other DNA
ccctagaaagatagtctgcgtaaaattgacgcnnn

SEQ ID NO: 487 - DNA, 63 bp, synthetic construct
  source 1..63: mol_type = other DNA
nnntagaaagataatcatattgtgacgtacgttaaagataatcatgcgtaaaattgacgcatg

SEQ ID NO: 488 - DNA, 63 bp, synthetic construct
  source 1..63: mol_type = other DNA
cnnnagaaagataatcatattgtgacgtacgttaaagataatcatgcgtaaaattgacgcatg

SEQ ID NO: 489 - DNA, 63 bp, synthetic construct
  source 1..63: mol_type = other DNA
ccnnngaaagataatcatattgtgacgtacgttaaagataatcatgcgtaaaattgacgcatg

SEQ ID NO: 490 - DNA, 63 bp, synthetic construct
  source 1..63: mol_type = other DNA
cccnnnaaagataatcatattgtgacgtacgttaaagataatcatgcgtaaaattgacgcatg

SEQ ID NO: 491 - DNA, 63 bp, synthetic construct
  source 1..63: mol_type = other DNA
ccctnnnaagataatcatattgtgacgtacgttaaagataatcatgcgtaaaattgacgcatg

SEQ ID NO: 492 - DNA, 63 bp, synthetic construct
  source 1..63: mol_type = other DNA
ccctannnagataatcatattgtgacgtacgttaaagataatcatgcgtaaaattgacgcatg

SEQ ID NO: 493 - DNA, 63 bp, synthetic construct
  source 1..63: mol_type = other DNA
ccctagnnngataatcatattgtgacgtacgttaaagataatcatgcgtaaaattgacgcatg

SEQ ID NO: 494 - DNA, 63 bp, synthetic construct
  source 1..63: mol_type = other DNA
ccctagannnataatcatattgtgacgtacgttaaagataatcatgcgtaaaattgacgcatg

SEQ ID NO: 495 - DNA, 63 bp, synthetic construct
  source 1..63: mol_type = other DNA
ccctagaannntaatcatattgtgacgtacgttaaagataatcatgcgtaaaattgacgcatg

SEQ ID NO: 496 - DNA, 63 bp, synthetic construct
  source 1..63: mol_type = other DNA
ccctagaaannnaatcatattgtgacgtacgttaaagataatcatgcgtaaaattgacgcatg

SEQ ID NO: 497 - DNA, 63 bp, synthetic construct
  source 1..63: mol_type = other DNA
ccctagaaagnnnatcatattgtgacgtacgttaaagataatcatgcgtaaaattgacgcatg

SEQ ID NO: 498 - DNA, 63 bp, synthetic construct
  source 1..63: mol_type = other DNA
ccctagaaagannntcatattgtgacgtacgttaaagataatcatgcgtaaaattgacgcatg

SEQ ID NO: 499 - DNA, 63 bp, synthetic construct
  source 1..63: mol_type = other DNA
ccctagaaagatnnncatattgtgacgtacgttaaagataatcatgcgtaaaattgacgcatg

SEQ ID NO: 500 - DNA, 63 bp, synthetic construct
  source 1..63: mol_type = other DNA
ccctagaaagatannnatattgtgacgtacgttaaagataatcatgcgtaaaattgacgcatg

SEQ ID NO: 501 - DNA, 63 bp, synthetic construct
  source 1..63: mol_type = other DNA
ccctagaaagataannntattgtgacgtacgttaaagataatcatgcgtaaaattgacgcatg

SEQ ID NO: 502 - DNA, 63 bp, synthetic construct
  source 1..63: mol_type = other DNA
ccctagaaagataatnnnattgtgacgtacgttaaagataatcatgcgtaaaattgacgcatg

SEQ ID NO: 503 - DNA, 63 bp, synthetic construct
  source 1..63: mol_type = other DNA
ccctagaaagataatcnnnttgtgacgtacgttaaagataatcatgcgtaaaattgacgcatg

SEQ ID NO: 504 - DNA, 63 bp, synthetic construct
  source 1..63: mol_type = other DNA
ccctagaaagataatcannntgtgacgtacgttaaagataatcatgcgtaaaattgacgcatg

SEQ ID NO: 505 - DNA, 63 bp, synthetic construct
  source 1..63: mol_type = other DNA
ccctagaaagataatcatnnngtgacgtacgttaaagataatcatgcgtaaaattgacgcatg

SEQ ID NO: 506 - DNA, 63 bp, synthetic construct
  source 1..63: mol_type = other DNA
ccctagaaagataatcatannntgacgtacgttaaagataatcatgcgtaaaattgacgcatg

SEQ ID NO: 507 - DNA, 63 bp, synthetic construct
  source 1..63: mol_type = other DNA
ccctagaaagataatcatatnnngacgtacgttaaagataatcatgcgtaaaattgacgcatg

SEQ ID NO: 508 - DNA, 63 bp, synthetic construct
  source 1..63: mol_type = other DNA
ccctagaaagataatcatattnnnacgtacgttaaagataatcatgcgtaaaattgacgcatg

SEQ ID NO: 509 - DNA, 63 bp, synthetic construct
  source 1..63: mol_type = other DNA
ccctagaaagataatcatattgnnncgtacgttaaagataatcatgcgtaaaattgacgcatg

SEQ ID NO: 510 - DNA, 63 bp, synthetic construct
  source 1..63: mol_type = other DNA
ccctagaaagataatcatattgtnnngtacgttaaagataatcatgcgtaaaattgacgcatg

SEQ ID NO: 511 - DNA, 63 bp, synthetic construct
  source 1..63: mol_type = other DNA
ccctagaaagataatcatattgtgnnntacgttaaagataatcatgcgtaaaattgacgcatg

SEQ ID NO: 512 - DNA, 63 bp, synthetic construct
  source 1..63: mol_type = other DNA
ccctagaaagataatcatattgtgannnacgttaaagataatcatgcgtaaaattgacgcatg

SEQ ID NO: 513 - DNA, 63 bp, synthetic construct
  source 1..63: mol_type = other DNA
ccctagaaagataatcatattgtgacnnncgttaaagataatcatgcgtaaaattgacgcatg

SEQ ID NO: 514 - DNA, 63 bp, synthetic construct
  source 1..63: mol_type = other DNA
ccctagaaagataatcatattgtgacgnnngttaaagataatcatgcgtaaaattgacgcatg

SEQ ID NO: 515 - DNA, 63 bp, synthetic construct
  source 1..63: mol_type = other DNA
ccctagaaagataatcatattgtgacgtnnnttaaagataatcatgcgtaaaattgacgcatg

SEQ ID NO: 516 - DNA, 63 bp, synthetic construct
  source 1..63: mol_type = other DNA
ccctagaaagataatcatattgtgacgtannntaaagataatcatgcgtaaaattgacgcatg

SEQ ID NO: 517 - DNA, 63 bp, synthetic construct
  source 1..63: mol_type = other DNA
ccctagaaagataatcatattgtgacgtacnnnaaagataatcatgcgtaaaattgacgcatg

SEQ ID NO: 518 - DNA, 63 bp, synthetic construct
  source 1..63: mol_type = other DNA
ccctagaaagataatcatattgtgacgtacgnnnaagataatcatgcgtaaaattgacgcatg

SEQ ID NO: 519 - DNA, 63 bp, synthetic construct
  source 1..63: mol_type = other DNA
ccctagaaagataatcatattgtgacgtacgtnnnagataatcatgcgtaaaattgacgcatg

SEQ ID NO: 520 - DNA, 63 bp, synthetic construct
  source 1..63: mol_type = other DNA
ccctagaaagataatcatattgtgacgtacgttnnngataatcatgcgtaaaattgacgcatg

SEQ ID NO: 521 - DNA, 63 bp, synthetic construct
  source 1..63: mol_type = other DNA
ccctagaaagataatcatattgtgacgtacgttannnataatcatgcgtaaaattgacgcatg

SEQ ID NO: 522 - DNA, 63 bp, synthetic construct
  source 1..63: mol_type = other DNA
ccctagaaagataatcatattgtgacgtacgttaannntaatcatgcgtaaaattgacgcatg

SEQ ID NO: 523 - DNA, 63 bp, synthetic construct
  source 1..63: mol_type = other DNA
ccctagaaagataatcatattgtgacgtacgttaaannnaatcatgcgtaaaattgacgcatg

SEQ ID NO: 524 - DNA, 63 bp, synthetic construct
  source 1..63: mol_type = other DNA
ccctagaaagataatcatattgtgacgtacgttaaagnnnatcatgcgtaaaattgacgcatg

SEQ ID NO: 525 - DNA, 63 bp, synthetic construct
  source 1..63: mol_type = other DNA
ccctagaaagataatcatattgtgacgtacgttaaagannntcatgcgtaaaattgacgcatg

SEQ ID NO: 526 - DNA, 63 bp, synthetic construct
  source 1..63: mol_type = other DNA
ccctagaaagataatcatattgtgacgtacgttaaagatnnncatgcgtaaaattgacgcatg

SEQ ID NO: 527 - DNA, 63 bp, synthetic construct
  source 1..63: mol_type = other DNA
ccctagaaagataatcatattgtgacgtacgttaaagatannnatgcgtaaaattgacgcatg

SEQ ID NO: 528 - DNA, 63 bp, synthetic construct
  source 1..63: mol_type = other DNA
ccctagaaagataatcatattgtgacgtacgttaaagataannntgcgtaaaattgacgcatg

SEQ ID NO: 529 - DNA, 63 bp, synthetic construct
  source 1..63: mol_type = other DNA
ccctagaaagataatcatattgtgacgtacgttaaagataatnnngcgtaaaattgacgcatg

SEQ ID NO: 530 - DNA, 63 bp, synthetic construct
  source 1..63: mol_type = other DNA
ccctagaaagataatcatattgtgacgtacgttaaagataatcnnncgtaaaattgacgcatg

SEQ ID NO: 531 - DNA, 63 bp, synthetic construct
  source 1..63: mol_type = other DNA
ccctagaaagataatcatattgtgacgtacgttaaagataatcannngtaaaattgacgcatg

SEQ ID NO: 532 - DNA, 63 bp, synthetic construct
  source 1..63: mol_type = other DNA
ccctagaaagataatcatattgtgacgtacgttaaagataatcatnnntaaaattgacgcatg

SEQ ID NO: 533 - DNA, 63 bp, synthetic construct
  source 1..63: mol_type = other DNA
ccctagaaagataatcatattgtgacgtacgttaaagataatcatgnnnaaaattgacgcatg

SEQ ID NO: 534 - DNA, 63 bp, synthetic construct
  source 1..63: mol_type = other DNA
ccctagaaagataatcatattgtgacgtacgttaaagataatcatgcnnnaaattgacgcatg

SEQ ID NO: 535 - DNA, 63 bp, synthetic construct
  source 1..63: mol_type = other DNA
ccctagaaagataatcatattgtgacgtacgttaaagataatcatgcgnnnaattgacgcatg

SEQ ID NO: 536 - DNA, 63 bp, synthetic construct
  source 1..63: mol_type = other DNA
ccctagaaagataatcatattgtgacgtacgttaaagataatcatgcgtnnnattgacgcatg

SEQ ID NO: 537 - DNA, 63 bp, synthetic construct
  source 1..63: mol_type = other DNA
ccctagaaagataatcatattgtgacgtacgttaaagataatcatgcgtannnttgacgcatg

SEQ ID NO: 538 - DNA, 63 bp, synthetic construct
  source 1..63: mol_type = other DNA
ccctagaaagataatcatattgtgacgtacgttaaagataatcatgcgtaannntgacgcatg

SEQ ID NO: 539 - DNA, 63 bp, synthetic construct
  source 1..63: mol_type = other DNA
ccctagaaagataatcatattgtgacgtacgttaaagataatcatgcgtaaannngacgcatg

SEQ ID NO: 540 - DNA, 63 bp, synthetic construct
  source 1..63: mol_type = other DNA
ccctagaaagataatcatattgtgacgtacgttaaagataatcatgcgtaaaannnacgcatg

SEQ ID NO: 541 - DNA, 63 bp, synthetic construct
  source 1..63: mol_type = other DNA
ccctagaaagataatcatattgtgacgtacgttaaagataatcatgcgtaaaatnnncgcatg

SEQ ID NO: 542 - DNA, 63 bp, synthetic construct
  source 1..63: mol_type = other DNA
ccctagaaagataatcatattgtgacgtacgttaaagataatcatgcgtaaaattnnngcatg

SEQ ID NO: 543 - DNA, 63 bp, synthetic construct
  source 1..63: mol_type = other DNA
ccctagaaagataatcatattgtgacgtacgttaaagataatcatgcgtaaaattgnnncatg

SEQ ID NO: 544 - DNA, 63 bp, synthetic construct
  source 1..63: mol_type = other DNA
ccctagaaagataatcatattgtgacgtacgttaaagataatcatgcgtaaaattgannnatg

SEQ ID NO: 545 - DNA, 63 bp, synthetic construct
  source 1..63: mol_type = other DNA
ccctagaaagataatcatattgtgacgtacgttaaagataatcatgcgtaaaattgacnnntg

SEQ ID NO: 546 - DNA, 63 bp, synthetic construct
  source 1..63: mol_type = other DNA
ccctagaaagataatcatattgtgacgtacgttaaagataatcatgcgtaaaattgacgnnng

SEQ ID NO: 547 - DNA, 63 bp, synthetic construct
  source 1..63: mol_type = other DNA
ccctagaaagataatcatattgtgacgtacgttaaagataatcatgcgtaaaattgacgcnnn

SEQ ID NO: 548 - DNA, 35 bp, synthetic construct
  source 1..35: mol_type = other DNA
ccctagtccgatagtctgcgtaaaattgactcctg

SEQ ID NO: 549 - DNA, 35 bp, synthetic construct
  source 1..35: mol_type = other DNA
ccctggacagctggtctgcgtaaaattgacgcatg

SEQ ID NO: 550 - DNA, 35 bp, synthetic construct
  source 1..35: mol_type = other DNA
ccctggaccgatggtctgcgtaaaattgacgcatg

SEQ ID NO: 551 - DNA, 35 bp, synthetic construct
  source 1..35: mol_type = other DNA
ccctagtacgatggtctgcgtaaaattgacgcatc

SEQ ID NO: 552 - DNA, 35 bp, synthetic construct
  source 1..35: mol_type = other DNA
ccctcagaagatagtctgcgtaaaattgacttctg

SEQ ID NO: 553 - DNA, 63 bp, synthetic construct
  source 1..63: mol_type = other DNA
ccctagaaagataatcatattgtgacgtacgttaaagataatcatgcgccgaatgaacgcatg

SEQ ID NO: 554 - DNA, 63 bp, synthetic construct
  source 1..63: mol_type = other DNA
ccctagaaagataatcatattgtgacgtatgctaaagataatcatgcgtaagcctgacgcatg

SEQ ID NO: 555 - DNA, 63 bp, synthetic construct
  source 1..63: mol_type = other DNA
ccctagaaagataatcatattgtgacgtacgttaaagataatcatgcgtaagccatactggtg

SEQ ID NO: 556 - DNA, 63 bp, synthetic construct
  source 1..63: mol_type = other DNA
ccctagaaagataatcatattgtgacgtacgttaaagataatcatgccctaaattgacgcagc

SEQ ID NO: 557 - DNA, 35 bp, synthetic construct
  source 1..35: mol_type = other DNA
cccnnnnnnnnnnnnntgcgtaaaattgacgcatg

SEQ ID NO: 558 - DNA, 35 bp, synthetic construct
  source 1..35: mol_type = other DNA
cccccccccccccccctgcgtaaaattgacgcatg

SEQ ID NO: 559 - DNA, 46 bp, synthetic construct
  source 1..46: mol_type = other DNA
atcttaaaagtcacgtaaaagataatcatgcgtcattttgactcac

SEQ ID NO: 560 - DNA, 13 bp, synthetic construct
  source 1..13: mol_type = other DNA
tagaaagatagtc

SEQ ID NO: 561 - DNA, 35 bp, synthetic construct
  source 1..35: mol_type = other DNA
ncctagaaagatagtctgcgtaaaattgacgcatg

SEQ ID NO: 562 - DNA, 35 bp, synthetic construct
  source 1..35: mol_type = other DNA
cnctagaaagatagtctgcgtaaaattgacgcatg

SEQ ID NO: 563 - DNA, 35 bp, synthetic construct
  source 1..35: mol_type = other DNA
ccntagaaagatagtctgcgtaaaattgacgcatg

SEQ ID NO: 564 - DNA, 35 bp, synthetic construct
  source 1..35: mol_type = other DNA
cccnagaaagatagtctgcgtaaaattgacgcatg

SEQ ID NO: 565 - DNA, 35 bp, synthetic construct
  source 1..35: mol_type = other DNA
ccctngaaagatagtctgcgtaaaattgacgcatg

SEQ ID NO: 566 - DNA, 35 bp, synthetic construct
  source 1..35: mol_type = other DNA
ccctanaaagatagtctgcgtaaaattgacgcatg

SEQ ID NO: 567 - DNA, 35 bp, synthetic construct
  source 1..35: mol_type = other DNA
ccctagnaagatagtctgcgtaaaattgacgcatg

SEQ ID NO: 568 - DNA, 35 bp, synthetic construct
  source 1..35: mol_type = other DNA
ccctaganagatagtctgcgtaaaattgacgcatg

SEQ ID NO: 569 - DNA, 35 bp, synthetic construct
  source 1..35: mol_type = other DNA
ccctagaangatagtctgcgtaaaattgacgcatg

SEQ ID NO: 570 - DNA, 35 bp, synthetic construct
  source 1..35: mol_type = other DNA
ccctagaaanatagtctgcgtaaaattgacgcatg

SEQ ID NO: 571 - DNA, 35 bp, synthetic construct
  source 1..35: mol_type = other DNA
ccctagaaagntagtctgcgtaaaattgacgcatg

SEQ ID NO: 572 - DNA, 35 bp, synthetic construct
  source 1..35: mol_type = other DNA
ccctagaaaganagtctgcgtaaaattgacgcatg

SEQ ID NO: 573 - DNA, 35 bp, synthetic construct
  source 1..35: mol_type = other DNA
ccctagaaagatngtctgcgtaaaattgacgcatg

SEQ ID NO: 574 - DNA, 35 bp, synthetic construct
  source 1..35: mol_type = other DNA
ccctagaaagatantctgcgtaaaattgacgcatg

SEQ ID NO: 575 - DNA, 35 bp, synthetic construct
  source 1..35: mol_type = other DNA
ccctagaaagatagnctgcgtaaaattgacgcatg

SEQ ID NO: 576 - DNA, 35 bp, synthetic construct
  source 1..35: mol_type = other DNA
ccctagaaagatagtntgcgtaaaattgacgcatg

SEQ ID NO: 577 - DNA, 35 bp, synthetic construct
  source 1..35: mol_type = other DNA
ccctagaaagatagtcngcgtaaaattgacgcatg

SEQ ID NO: 578 - DNA, 35 bp, synthetic construct
  source 1..35: mol_type = other DNA
ccctagaaagatagtctncgtaaaattgacgcatg

SEQ ID NO: 579 - DNA, 35 bp, synthetic construct
  source 1..35: mol_type = other DNA
ccctagaaagatagtctgngtaaaattgacgcatg

SEQ ID NO: 580 - DNA, 35 bp, synthetic construct
  source 1..35: mol_type = other DNA
ccctagaaagatagtctgcntaaaattgacgcatg

SEQ ID NO: 581 - DNA, 35 bp, synthetic construct
  source 1..35: mol_type = other DNA
ccctagaaagatagtctgcgnaaaattgacgcatg

SEQ ID NO: 582 - DNA, 35 bp, synthetic construct
  source 1..35: mol_type = other DNA
ccctagaaagatagtctgcgtnaaattgacgcatg

SEQ ID NO: 583 - DNA, 35 bp, synthetic construct
  source 1..35: mol_type = other DNA
ccctagaaagatagtctgcgtanaattgacgcatg

SEQ ID NO: 584 - DNA, 35 bp, synthetic construct; identical to SEQ ID NO: 561
  source 1..35: mol_type = other DNA

SEQ ID NO: 585 - DNA, 35 bp, synthetic construct
  source 1..35: mol_type = other DNA
nnctagaaagatagtctgcgtaaaattgacgcatg

SEQ ID NO: 586 - DNA, 35 bp, synthetic construct
  source 1..35: mol_type = other DNA
cnntagaaagatagtctgcgtaaaattgacgcatg

SEQ ID NO: 587 - DNA, 35 bp, synthetic construct
  source 1..35: mol_type = other DNA
ccnnagaaagatagtctgcgtaaaattgacgcatg

SEQ ID NO: 588 - DNA, 35 bp, synthetic construct
  source 1..35: mol_type = other DNA
cccnngaaagatagtctgcgtaaaattgacgcatg

SEQ ID NO: 589 - DNA, 35 bp, synthetic construct
  source 1..35: mol_type = other DNA
ccctnnaaagatagtctgcgtaaaattgacgcatg

SEQ ID NO: 590 - DNA, 35 bp, synthetic construct
  source 1..35: mol_type = other DNA
ccctannaagatagtctgcgtaaaattgacgcatg

SEQ ID NO: 591 - DNA, 35 bp, synthetic construct
  source 1..35: mol_type = other DNA
ccctagnnagatagtctgcgtaaaattgacgcatg

SEQ ID NO: 592 - DNA, 35 bp, synthetic construct
  source 1..35: mol_type = other DNA
ccctaganngatagtctgcgtaaaattgacgcatg

SEQ ID NO: 593 - DNA, 35 bp, synthetic construct
  source 1..35: mol_type = other DNA
ccctagaannatagtctgcgtaaaattgacgcatg

SEQ ID NO: 594 - DNA, 35 bp, synthetic construct
  source 1..35: mol_type = other DNA
ccctagaaanntagtctgcgtaaaattgacgcatg

SEQ ID NO: 595 - DNA, 35 bp, synthetic construct
  source 1..35: mol_type = other DNA
ccctagaaagnnagtctgcgtaaaattgacgcatg

SEQ ID NO: 596 - DNA, 35 bp, synthetic construct
  source 1..35: mol_type = other DNA
ccctagaaaganngtctgcgtaaaattgacgcatg

SEQ ID NO: 597 - DNA, 35 bp, synthetic construct
  source 1..35: mol_type = other DNA
ccctagaaagatnntctgcgtaaaattgacgcatg

SEQ ID NO: 598 - DNA, 35 bp, synthetic construct
  source 1..35: mol_type = other DNA
ccctagaaagatannctgcgtaaaattgacgcatg

SEQ ID NO: 599 - DNA, 35 bp, synthetic construct
  source 1..35: mol_type = other DNA
ccctagaaagatagnntgcgtaaaattgacgcatg

SEQ ID NO: 600 - DNA, 35 bp, synthetic construct
  source 1..35: mol_type = other DNA
ccctagaaagatagtnngcgtaaaattgacgcatg

SEQ ID NO: 601 - DNA, 35 bp, synthetic construct
  source 1..35: mol_type = other DNA
ccctagaaagatagtcnncgtaaaattgacgcatg

SEQ ID NO: 602 - DNA, 35 bp, synthetic construct
  source 1..35: mol_type = other DNA
ccctagaaagatagtctnngtaaaattgacgcatg

SEQ ID NO: 603 - DNA, 35 bp, synthetic construct
  source 1..35: mol_type = other DNA
ccctagaaagatagtctgnntaaaattgacgcatg

SEQ ID NO: 604 - DNA, 35 bp, synthetic construct
  source 1..35: mol_type = other DNA
ccctagaaagatagtctgcnnaaaattgacgcatg

SEQ ID NO: 605 - DNA, 35 bp, synthetic construct
  source 1..35: mol_type = other DNA
ccctagaaagatagtctgcgnnaaattgacgcatg

SEQ ID NO: 606 - DNA, 35 bp, synthetic construct
  source 1..35: mol_type = other DNA
ccctagaaagatagtctgcgtnnaattgacgcatg

SEQ ID NO: 607 - DNA, 35 bp, synthetic construct
  source 1..35: mol_type = other DNA
ccctagaaagatagtctgcgtannattgacgcatg

SEQ ID NO: 608 - DNA, 35 bp, synthetic construct; identical to SEQ ID NO: 561
  source 1..35: mol_type = other DNA

SEQ ID NO: 609 - DNA, 35 bp, synthetic construct; identical to SEQ ID NO: 585
  source 1..35: mol_type = other DNA

SEQ ID NO: 610 - DNA, 34 bp, synthetic construct
  source 1..34: mol_type = other DNA
cctagaaagatagtctgcgtaaaattgacgcatg

SEQ ID NO: 611 - DNA, 34 bp, synthetic construct
  source 1..34: mol_type = other DNA
cccagaaagatagtctgcgtaaaattgacgcatg

SEQ ID NO: 612 - DNA, 34 bp, synthetic construct
  source 1..34: mol_type = other DNA
ccctgaaagatagtctgcgtaaaattgacgcatg

SEQ ID NO: 613 - DNA, 34 bp, synthetic construct
  source 1..34: mol_type = other DNA
ccctaaaagatagtctgcgtaaaattgacgcatg

SEQ ID NO: 614 - DNA, 34 bp, synthetic construct
  source 1..34: mol_type = other DNA
ccctagaagatagtctgcgtaaaattgacgcatg

SEQ ID NO: 615 - DNA, 34 bp, synthetic construct
  source 1..34: mol_type = other DNA
ccctagaaaatagtctgcgtaaaattgacgcatg

SEQ ID NO: 616 - DNA, 34 bp, synthetic construct
  source 1..34: mol_type = other DNA
ccctagaaagtagtctgcgtaaaattgacgcatg

SEQ ID NO: 617 - DNA, 34 bp, synthetic construct
  source 1..34: mol_type = other DNA
ccctagaaagaagtctgcgtaaaattgacgcatg

SEQ ID NO: 618 - DNA, 34 bp, synthetic construct
  source 1..34: mol_type = other DNA
ccctagaaagatgtctgcgtaaaattgacgcatg

SEQ ID NO: 619 - DNA, 34 bp, synthetic construct
  source 1..34: mol_type = other DNA
ccctagaaagatatctgcgtaaaattgacgcatg

SEQ ID NO: 620 - DNA, 34 bp, synthetic construct
  source 1..34: mol_type = other DNA
ccctagaaagatagctgcgtaaaattgacgcatg

SEQ ID NO: 621 - DNA, 34 bp, synthetic construct
  source 1..34: mol_type = other DNA
ccctagaaagatagttgcgtaaaattgacgcatg

SEQ ID NO: 622 - DNA, 34 bp, synthetic construct
  source 1..34: mol_type = other DNA
ccctagaaagatagtcgcgtaaaattgacgcatg

SEQ ID NO: 623 - DNA, 34 bp, synthetic construct
  source 1..34: mol_type = other DNA
ccctagaaagatagtctcgtaaaattgacgcatg

SEQ ID NO: 624 - DNA, 34 bp, synthetic construct
  source 1..34: mol_type = other DNA
ccctagaaagatagtctggtaaaattgacgcatg

SEQ ID NO: 625 - DNA, 34 bp, synthetic construct
  source 1..34: mol_type = other DNA
ccctagaaagatagtctgctaaaattgacgcatg

SEQ ID NO: 626 - DNA, 34 bp, synthetic construct
  source 1..34: mol_type = other DNA
ccctagaaagatagtctgcgaaaattgacgcatg

SEQ ID NO: 627 - DNA, 34 bp, synthetic construct
  source 1..34: mol_type = other DNA
ccctagaaagatagtctgcgtaaattgacgcatg

SEQ ID NO: 628 - DNA, 34 bp, synthetic construct
  source 1..34: mol_type = other DNA
ccctagaaagatagtctgcgtaaaatgacgcatg

SEQ ID NO: 629 - DNA, 34 bp, synthetic construct
  source 1..34: mol_type = other DNA
ccctagaaagatagtctgcgtaaaattacgcatg

SEQ ID NO: 630 - DNA, 34 bp, synthetic construct
  source 1..34: mol_type = other DNA
ccctagaaagatagtctgcgtaaaattgcgcatg

SEQ ID NO: 631 - DNA, 34 bp, synthetic construct
  source 1..34: mol_type = other DNA
ccctagaaagatagtctgcgtaaaattgagcatg

SEQ ID NO: 632 - DNA, 34 bp, synthetic construct
  source 1..34: mol_type = other DNA
ccctagaaagatagtctgcgtaaaattgaccatg

SEQ ID NO: 633 - DNA, 34 bp, synthetic construct; identical to SEQ ID NO: 610
  source 1..34: mol_type = other DNA

SEQ ID NO: 634 - DNA, 33 bp, synthetic construct
  source 1..33: mol_type = other DNA
ctagaaagatagtctgcgtaaaattgacgcatg

SEQ ID NO: 635 - DNA, 33 bp, synthetic construct
  source 1..33: mol_type = other DNA
ccagaaagatagtctgcgtaaaattgacgcatg

SEQ ID NO: 636 - DNA, 33 bp, synthetic construct
  source 1..33: mol_type = other DNA
cccgaaagatagtctgcgtaaaattgacgcatg

SEQ ID NO: 637 - DNA, 33 bp, synthetic construct
  source 1..33: mol_type = other DNA
ccctaaagatagtctgcgtaaaattgacgcatg

SEQ ID NO: 638 - DNA, 33 bp, synthetic construct
  source 1..33: mol_type = other DNA
ccctagagatagtctgcgtaaaattgacgcatg

SEQ ID NO: 639 - DNA, 33 bp, synthetic construct
  source 1..33: mol_type = other DNA
ccctagaaatagtctgcgtaaaattgacgcatg

SEQ ID NO: 640 - DNA, 33 bp, synthetic construct
  source 1..33: mol_type = other DNA
ccctagaaagagtctgcgtaaaattgacgcatg

SEQ ID NO: 641 - DNA, 33 bp, synthetic construct
  source 1..33: mol_type = other DNA
ccctagaaagattctgcgtaaaattgacgcatg

SEQ ID NO: 642 - DNA, 33 bp, synthetic construct
  source 1..33: mol_type = other DNA
ccctagaaagatactgcgtaaaattgacgcatg

SEQ ID NO: 643 - DNA, 33 bp, synthetic construct
  source 1..33: mol_type = other DNA
ccctagaaagatagtgcgtaaaattgacgcatg

SEQ ID NO: 644 - DNA, 33 bp, synthetic construct
  source 1..33: mol_type = other DNA
ccctagaaagatagtccgtaaaattgacgcatg

SEQ ID NO: 645 - DNA, 33 bp, synthetic construct
  source 1..33: mol_type = other DNA
ccctagaaagatagtctgtaaaattgacgcatg

SEQ ID NO: 646 - DNA, 33 bp, synthetic construct
  source 1..33: mol_type = other DNA
ccctagaaagatagtctgcaaaattgacgcatg

SEQ ID NO: 647 - DNA, 33 bp, synthetic construct
  source 1..33: mol_type = other DNA
ccctagaaagatagtctgcgaaattgacgcatg

SEQ ID NO: 648 - DNA, 33 bp, synthetic construct
  source 1..33: mol_type = other DNA
ccctagaaagatagtctgcgtaattgacgcatg

SEQ ID NO: 649 - DNA, 33 bp, synthetic construct
  source 1..33: mol_type = other DNA
ccctagaaagatagtctgcgtaaatgacgcatg

SEQ ID NO: 650 - DNA, 33 bp, synthetic construct
  source 1..33: mol_type = other DNA
ccctagaaagatagtctgcgtaaaagacgcatg

SEQ ID NO: 651 - DNA, 33 bp, synthetic construct
  source 1..33: mol_type = other DNA
ccctagaaagatagtctgcgtaaaatacgcatg

SEQ ID NO: 652 - DNA, 33 bp, synthetic construct
  source 1..33: mol_type = other DNA
ccctagaaagatagtctgcgtaaaattcgcatg

SEQ ID NO: 653 - DNA, 33 bp, synthetic construct
  source 1..33: mol_type = other DNA
ccctagaaagatagtctgcgtaaaattggcatg

SEQ ID NO: 654 - DNA, 33 bp, synthetic construct
  source 1..33: mol_type = other DNA
ccctagaaagatagtctgcgtaaaattgacatg

SEQ ID NO: 655 - DNA, 33 bp, synthetic construct
  source 1..33: mol_type = other DNA
ccctagaaagatagtctgcgtaaaattgacgtg

SEQ ID NO: 656 - DNA, 33 bp, synthetic construct
  source 1..33: mol_type = other DNA
ccctagaaagatagtctgcgtaaaattgacgcg

SEQ ID NO: 657 - DNA, 34 bp, synthetic construct; identical to SEQ ID NO: 610
  source 1..34: mol_type = other DNA

SEQ ID NO: 658 - DNA, 33 bp, synthetic construct; identical to SEQ ID NO: 634
  source 1..33: mol_type = other DNA

SEQ ID NO: 659 - DNA, 32 bp, synthetic construct
  source 1..32: mol_type = other DNA
tagaaagatagtctgcgtaaaattgacgcatg

SEQ ID NO: 660 - DNA, 32 bp, synthetic construct
  source 1..32: mol_type = other DNA
cagaaagatagtctgcgtaaaattgacgcatg

SEQ ID NO: 661 - DNA, 32 bp, synthetic construct
  source 1..32: mol_type = other DNA
ccgaaagatagtctgcgtaaaattgacgcatg

SEQ ID NO: 662 - DNA, 32 bp, synthetic construct
  source 1..32: mol_type = other DNA
cccaaagatagtctgcgtaaaattgacgcatg

SEQ ID NO: 663 - DNA, 32 bp, synthetic construct
  source 1..32: mol_type = other DNA
ccctaagatagtctgcgtaaaattgacgcatg

SEQ ID NO: 664 - DNA, 32 bp, synthetic construct
  source 1..32: mol_type = other DNA
ccctaggatagtctgcgtaaaattgacgcatg

SEQ ID NO: 665 - DNA, 32 bp, synthetic construct
  source 1..32: mol_type = other DNA
ccctagaatagtctgcgtaaaattgacgcatg

SEQ ID NO: 666 - DNA, 32 bp, synthetic construct
  source 1..32: mol_type = other DNA
ccctagaaaagtctgcgtaaaattgacgcatg

SEQ ID NO: 667 - DNA, 32 bp, synthetic construct
  source 1..32: mol_type = other DNA
ccctagaaaggtctgcgtaaaattgacgcatg

SEQ ID NO: 668 - DNA, 32 bp, synthetic construct
  source 1..32: mol_type = other DNA
ccctagaaagatctgcgtaaaattgacgcatg

SEQ ID NO: 669 - DNA, 32 bp, synthetic construct
  source 1..32: mol_type = other DNA
ccctagaaagatatgcgtaaaattgacgcatg

SEQ ID NO: 670 - DNA, 32 bp, synthetic construct
  source 1..32: mol_type = other DNA
ccctagaaagataggcgtaaaattgacgcatg

SEQ ID NO: 671 - DNA, 32 bp, synthetic construct
  source 1..32: mol_type = other DNA
ccctagaaagatagtcgtaaaattgacgcatg

SEQ ID NO: 672 - DNA, 32 bp, synthetic construct
  source 1..32: mol_type = other DNA
ccctagaaagatagtcttaaaattgacgcatg

SEQ ID NO: 673 - DNA, 32 bp, synthetic construct
  source 1..32: mol_type = other DNA
ccctagaaagatagtctgaaaattgacgcatg

SEQ ID NO: 674 - DNA, 32 bp, synthetic construct
  source 1..32: mol_type = other DNA
ccctagaaagatagtctgcaaattgacgcatg

SEQ ID NO: 675 - DNA, 32 bp, synthetic construct
  source 1..32: mol_type = other DNA
ccctagaaagatagtctgcgaattgacgcatg

SEQ ID NO: 676 - DNA, 32 bp, synthetic construct
  source 1..32: mol_type = other DNA
ccctagaaagatagtctgcgtattgacgcatg

SEQ ID NO: 677 - DNA, 32 bp, synthetic construct
  source 1..32: mol_type = other DNA
ccctagaaagatagtctgcgtaatgacgcatg

SEQ ID NO: 678 - DNA, 32 bp, synthetic construct
  source 1..32: mol_type = other DNA
ccctagaaagatagtctgcgtaaagacgcatg

SEQ ID NO: 679 - DNA, 32 bp, synthetic construct
  source 1..32: mol_type = other DNA
ccctagaaagatagtctgcgtaaaaacgcatg

SEQ ID NO: 680 - DNA, 32 bp, synthetic construct
  source 1..32: mol_type = other DNA
ccctagaaagatagtctgcgtaaaatcgcatg

SEQ ID NO: 681 - DNA, 32 bp, synthetic construct
  source 1..32: mol_type = other DNA
ccctagaaagatagtctgcgtaaaattgcatg

SEQ ID NO: 682 - DNA, 44 bp, synthetic construct
  source 1..44: mol_type = other DNA
ttaaccctnnnnnnnnnnnntgcgtaaaattgacgcatgcattc

SEQ ID NO: 683 - DNA, 44 bp, synthetic construct
  source 1..44: mol_type = other DNA
ttaaccctagaaagatagtcnnnnnnaaattgacgcatgcattc

SEQ ID NO: 684 - DNA, 44 bp, synthetic construct
  source 1..44: mol_type = other DNA
ttaaccctagaaagatagtctgcgtaannnnnnnnnnngcattc

SEQ ID NO: 685 - DNA, 45 bp, synthetic construct
  source 1..45: mol_type = other DNA
ttaaccctagaaagatagtctgcgtaaaattgacgcatgcattct

SEQ ID NO: 686 - DNA, 57 bp, synthetic construct
  source 1..57: mol_type = other DNA
ttaaatcttaaaagtcacgtaaaagataatcatgcgtcattttgactcacgcggtcg

SEQ ID NO: 687 - DNA, 73 bp, synthetic construct
  source 1..73: mol_type = other DNA
ttaaccctagaaagataatcatattgtgacgtacgttaaagataatcatgcgtaaaattgacgcatgtgtttt